SEQUENCE LISTING (291 sequences)

SEQ ID NO: 1 - DNA, 121 bp, synthetic construct
  misc_feature 1..121: note = Description of Artificial Sequence: Synthetic polynucleotide
  source 1..121: mol_type = other DNA
cagtctggggagtgtgcttctggaagtgaaagtgagggatgagaggtgtgtggtttgcagnttgggaaacggaaatcacatttgcatcagctctttgcaaagtgctgcctagccctctgtc

SEQ ID NO: 2 - DNA, 101 bp, synthetic construct
  misc_feature 1..101: note = Description of Artificial Sequence: Synthetic polynucleotide
  source 1..101: mol_type = other DNA
atgaaaggaaagtatttccagtctgcattgaccattgtttaatcagagtangaggccacagatcgaggtgactgtctgtgagggtagaacattaaccacta

SEQ ID NO: 3 - DNA, 101 bp, synthetic construct
  misc_feature 1..101: note = Description of Artificial Sequence: Synthetic polynucleotide
  source 1..101: mol_type = other DNA
tttatagtacattagatggccttaagtgatttagaaaaaaaaaagagatanaatgatcttaattgcaattgaaaatagagttgtcagaatagacctcattg

SEQ ID NO: 4 - DNA, 101 bp, synthetic construct
  misc_feature 1..101: note = Description of Artificial Sequence: Synthetic polynucleotide
  source 1..101: mol_type = other DNA
ggctattccattgaaatgtgtgttttgatgatcatggctaagtgggacttnagtgactcaaaccctgtgttcagatgaagcctgctcagatttctcctata

SEQ ID NO: 5 - DNA, 101 bp, synthetic construct
  misc_feature 1..101: note = Description of Artificial Sequence: Synthetic polynucleotide
  source 1..101: mol_type = other DNA
ccggccagattttgtttttaattgtatttctgtaatgtaagcatgctgtgntagctctctgattcttaattctctctttggaaaaatacaggtagccttac

SEQ ID NO: 6 - DNA, 101 bp, synthetic construct
  misc_feature 1..101: note = Description of Artificial Sequence: Synthetic polynucleotide
  source 1..101: mol_type = other DNA
gtccaatctcattttgtcttggcattcaaagtcctaacttatcccagtctngctatccattatttacttctctctaagccctctgtgttcccagccatgag

SEQ ID NO: 7 - DNA, 101 bp, synthetic construct
  misc_feature 1..101: note = Description of Artificial Sequence: Synthetic polynucleotide
  source 1..101: mol_type = other DNA
agagcaggtactaagtcataacccttccccatgactattgctcctaactgntattcaatagactcattaccatctactaaaataagcatctaacgtatttt

SEQ ID NO: 8 - DNA, 101 bp, synthetic construct
  misc_feature 1..101: note = Description of Artificial Sequence: Synthetic polynucleotide
  source 1..101: mol_type = other DNA
agatctgcctgaaggcctcagatggaaccaaaagtgagctctttcttcacngttaggaggttaatgacatccatttccatcaaatggtaattggtatattt

SEQ ID NO: 9 - DNA, 101 bp, synthetic construct
  misc_feature 1..101: note = Description of Artificial Sequence: Synthetic polynucleotide
  source 1..101: mol_type = other DNA
tcctctgtccagagctgaaatagttgccactcactgcagagagtccactgntcctccccaaggtcagagtgccatgtggcttacctggaactgcacacagg

SEQ ID NO: 10 - DNA, 101 bp, synthetic construct
  misc_feature 1..101: note = Description of Artificial Sequence: Synthetic polynucleotide
  source 1..101: mol_type = other DNA
taatgatcaactagaacacatggaagtcaatgaacaaaaggccacataatnatagatttgaaaaagacctcagagatcctttggcttaatctctcccccaa

SEQ ID NO: 11 - DNA, 101 bp, synthetic construct
  misc_feature 1..101: note = Description of Artificial Sequence: Synthetic polynucleotide
  source 1..101: mol_type = other DNA
acacatacgtagtccccctgctgcactgttggtgtgcactcacccatggcntcctgctaccctgttggtgtgccctgttggtgtgcactcgctggcagctc

SEQ ID NO: 12 - DNA, 101 bp, synthetic construct
  misc_feature 1..101: note = Description of Artificial Sequence: Synthetic polynucleotide
  source 1..101: mol_type = other DNA
aacatggctccgaggtagatgggctggaaccagttgctacctacttcgcanacagacttggtccccatgaggagctgggttggctcagggtagctgtctgt

SEQ ID NO: 13 - DNA, 101 bp, synthetic construct
  misc_feature 1..101: note = Description of Artificial Sequence: Synthetic polynucleotide
  source 1..101: mol_type = other DNA
gtgatgaaggtctgcggtcctagagctacagatgcatggtaagaaattagnacaaactggagatgggccatggactctgcctaggagtgttaggaaaatac

SEQ ID NO: 14 - DNA, 101 bp, synthetic construct
  misc_feature 1..101: note = Description of Artificial Sequence: Synthetic polynucleotide
  source 1..101: mol_type = other DNA
gaggtacagtcttttagaaggcagggatgaattcatgggatggaattcagngcccaagaggagagttggccttcattaggcctggggacacatcctccacc

SEQ ID NO: 15 - DNA, 101 bp, synthetic construct
  misc_feature 1..101: note = Description of Artificial Sequence: Synthetic polynucleotide
  source 1..101: mol_type = other DNA
agagagttgtgggtcctagagtatgtaagagagaatagagaagagaggagnagagagagagaggaagagaaagcaagaccaaccaataaaccaacataatc

SEQ ID NO: 16 - DNA, 102 bp, synthetic construct
  misc_feature 1..102: note = Description of Artificial Sequence: Synthetic polynucleotide
  source 1..102: mol_type = other DNA
gtcagtgggatgttaaagtctgccagtgttattctgtgggaaactaagtctntttgtagatctctaagaacttgctttataaatctgagtgcttcctgtatt

SEQ ID NO: 17 - DNA, 101 bp, synthetic construct
  misc_feature 1..101: note = Description of Artificial Sequence: Synthetic polynucleotide
  source 1..101: mol_type = other DNA
tttcctttggtaaacttgaatatcctccaactctgtagtatccccaagatnctttccaacccaaagtctataatccttctaggcattgtcgtcctcttagg

SEQ ID NO: 18 - DNA, 101 bp, synthetic construct
  misc_feature 1..101: note = Description of Artificial Sequence: Synthetic polynucleotide
  source 1..101: mol_type = other DNA
atcaatacctacctcccttacaaacatcaagagcaagaaagaaggcagacntggaaagcccaaatcttccgataactgaaaaacatccatatttgaataag

SEQ ID NO: 19 - DNA, 101 bp, synthetic construct
  misc_feature 1..101: note = Description of Artificial Sequence: Synthetic polynucleotide
  source 1..101: mol_type = other DNA
gaaagaggattaattttctcattgggaaactgtagactttgcttaaaaagngtctcatatcattttcaaaatagactaaagtgatcgaatatacctaacag

SEQ ID NO: 20 - DNA, 101 bp, synthetic construct
  misc_feature 1..101: note = Description of Artificial Sequence: Synthetic polynucleotide
  source 1..101: mol_type = other DNA
tgctccactctccaaaactgcgaaactgcgaaggtgtcttgaaccaccttncttacgttgaattgttggcttgtcacttaagtacctgagctaatttatac

SEQ ID NO: 21 - DNA, 101 bp, synthetic construct
  misc_feature 1..101: note = Description of Artificial Sequence: Synthetic polynucleotide
  source 1..101: mol_type = other DNA
atgtcagtgttctcaattctgtctgggcctgtggagttttaaaatatacantttttcgcattctagtcccaagatcgttgagtgtacttggaaggggacca

SEQ ID NO: 22 - DNA, 106 bp, synthetic construct
  misc_feature 1..106: note = Description of Artificial Sequence: Synthetic polynucleotide
  source 1..106: mol_type = other DNA
acgatgagcctggagcatttatccatcaactgccatccatctctgggtcaggcttnactctgcaaatgtcactattttcaaactgccaggctgcacttgcattagg

SEQ ID NO: 23 - DNA, 101 bp, synthetic construct
  misc_feature 1..101: note = Description of Artificial Sequence: Synthetic polynucleotide
  source 1..101: mol_type = other DNA
ttaaggcaccatctggtctcttctaaactcccttgagtggttgattgaagnaaaacatctaagaacaaataattttcttggaacagtacattctaagtcta

SEQ ID NO: 24 - DNA, 101 bp, synthetic construct
  misc_feature 1..101: note = Description of Artificial Sequence: Synthetic polynucleotide
  source 1..101: mol_type = other DNA
agagcggagattgagataaataaagtaaggggacttttagatgaccaagcngaggcaattaataggtagatgaacggttatttggggcttccaggcagagg

SEQ ID NO: 25 - DNA, 101 bp, synthetic construct
  misc_feature 1..101: note = Description of Artificial Sequence: Synthetic polynucleotide
  source 1..101: mol_type = other DNA
aataaaaggcacaacatcccaatctcatagcaagattataggacgtcaccnggcaatcagagagcctgatgtggagttggtgctcagttcttcattcaaca

SEQ ID NO: 26 - DNA, 100 bp, synthetic construct
  misc_feature 1..100: note = Description of Artificial Sequence: Synthetic polynucleotide
  source 1..100: mol_type = other DNA
ctggtgccataaaatattcagctataggactgagtgtccatgggttatanaataggaatgaaagaatggaaaagcctaaacaattacatttgacttgatt

SEQ ID NO: 27 - DNA, 101 bp, synthetic construct
  misc_feature 1..101: note = Description of Artificial Sequence: Synthetic polynucleotide
  source 1..101: mol_type = other DNA
aacatgtacctttgtggataaaagccttaagttccccatgaatgacttttnccccctcctttataaaattgacacccatgcttgtgatgaaaccacattta

SEQ ID NO: 28 - DNA, 101 bp, synthetic construct
  misc_feature 1..101: note = Description of Artificial Sequence: Synthetic polynucleotide
  source 1..101: mol_type = other DNA
gttgttcaaggccttagaatttgccagtttggcagacccgggcctggagcncaggacagcagactcctgatccattgcatcttcttcagttccatcttggg

SEQ ID NO: 29 - DNA, 101 bp, synthetic construct
  misc_feature 1..101: note = Description of Artificial Sequence: Synthetic polynucleotide
  source 1..101: mol_type = other DNA
actgctgagctagacacaaaaagaatatgacatgttccctgcctttatggnactcacagtgtagtgggggtgacagatgcagacactaaaaatttgactac

SEQ ID NO: 30 - DNA, 101 bp, synthetic construct
  misc_feature 1..101: note = Description of Artificial Sequence: Synthetic polynucleotide
  source 1..101: mol_type = other DNA
aaagagtgcatggagggcttgggatctgaacctttagaccaagtacagacnctggcacatattgggagcttcataaacatcagctcagtgtacaatagatg

SEQ ID NO: 31 - DNA, 101 bp, synthetic construct
  misc_feature 1..101: note = Description of Artificial Sequence: Synthetic polynucleotide
  source 1..101: mol_type = other DNA
tgccagattatccaactggcaaaatgcacagattctcaggcatcaggaagncagaggcagacaaagagagtcagagagggggtgaggatgcagtgacttca

SEQ ID NO: 32 - DNA, 101 bp, synthetic construct
  misc_feature 1..101: note = Description of Artificial Sequence: Synthetic polynucleotide
  source 1..101: mol_type = other DNA
ctgtgatgtacggcagaaaccagttttactagcgcctccatccagttgctncttctggttatgtcacagcctggactcttcaggctacttggaaaggcctt

SEQ ID NO: 33 - DNA, 121 bp, synthetic construct
  misc_feature 1..121: note = Description of Artificial Sequence: Synthetic polynucleotide
  source 1..121: mol_type = other DNA
atatattctataaaagagacacttcagtaactcaaaaagtctatgttcttcaagtgccccnccacaaagggttatagcccttggatgaagcatctttctagtcctcttctgaacttaccca

SEQ ID NO: 34 - DNA, 121 bp, synthetic construct
  misc_feature 1..121: note = Description of Artificial Sequence: Synthetic polynucleotide
  source 1..121: mol_type = other DNA
tgtgtcaagagcttattgtntggggaatgttggtgggcatttgacctctatcctcatttcntcttcatcacagtgctccgggaaaaatcgcaatcacccccattttagagatgaggatatg

SEQ ID NO: 35 - DNA, 121 bp, synthetic construct
  misc_feature 1..121: note = Description of Artificial Sequence: Synthetic polynucleotide
  source 1..121: mol_type = other DNA
ttacctctcatgagggaaataccctcatacagttggccatcacttaacaatagagacaacnatgatagatgggatggtagcaactttaggttttgttgtttcctatttttcagtggtgaat

SEQ ID NO: 36 - DNA, 121 bp, synthetic construct
  misc_feature 1..121: note = Description of Artificial Sequence: Synthetic polynucleotide
  source 1..121: mol_type = other DNA
ctggagaccaaggactatgttgcaccataactatcacctcccaggtatgcagaactgagcnattttcaaaggtcttcaccattcatagtctcatttgagcctgaaactactttgacagcta

SEQ ID NO: 37 - DNA, 121 bp, synthetic construct
  misc_feature 1..121: note = Description of Artificial Sequence: Synthetic polynucleotide
  source 1..121: mol_type = other DNA
cagtctggggagtgtgcttctggaagtgaaagtgagggatgagaggtgtgtggtttgcagrttgggaaacggaaatcacatttgcatcagctctttgcaaagtgctgcctagccctctgtc

SEQ ID NO: 38 - DNA, 121 bp, synthetic construct
  misc_feature 1..121: note = Description of Artificial Sequence: Synthetic polynucleotide
  source 1..121: mol_type = other DNA
aatcagggagtagtggttaatgttctaccctcacagacagtcacctcgatctgtggcctcrtactctgattaaacaatggtcaatgcagactggaaatactttcctttcatgggcagtcat

SEQ ID NO: 39 - DNA, 123 bp, synthetic construct
  misc_feature 1..123: note = Description of Artificial Sequence: Synthetic polynucleotide
  misc_feature 61..63: note = This region may be absent
  source 1..123: mol_type = other DNA
aataagttaatttatagtacattagatggccttaagtgatttagaaaaaaaaaagagatagaaaatgatcttaattgcaattgaaaatagagttgtcagaatagacctcattgagaggagaca

SEQ ID NO: 40 - DNA, 121 bp, synthetic construct
  misc_feature 1..121: note = Description of Artificial Sequence: Synthetic polynucleotide
  source 1..121: mol_type = other DNA
cttctacgcttataggagaaatctgagcaggcttcatctgaacacagggtttgagtcactraagtcccacttagccatgatcatcaaaacacacatttcaatggaatagcccactccccag

SEQ ID NO: 41 - DNA, 121 bp, synthetic construct
  misc_feature 1..121: note = Description of Artificial Sequence: Synthetic polynucleotide
  source 1..121: mol_type = other DNA
ccagcagagagtaaggctacctgtatttttccaaagagagaattaagaatcagagagctarcacagcatgcttacattacagaaatacaattaaaaacaaaatctggccgggcacagtggc

SEQ ID NO: 42 - DNA, 121 bp, synthetic construct
  misc_feature 1..121: note = Description of Artificial Sequence: Synthetic polynucleotide
  source 1..121: mol_type = other DNA
gtggttgcctctcatggctgggaacacagagggcttagagagaagtaaataatggatagcragactgggataagttaggactttgaatgccaagacaaaatgagattggactgggtcttaa

SEQ ID NO: 43 - DNA, 121 bp, synthetic construct
  misc_feature 1..121: note = Description of Artificial Sequence: Synthetic polynucleotide
  source 1..121: mol_type = other DNA
gaattctttgaaaatacgttagatgcttattttagtagatggtaatgagtctattgaatamcagttaggagcaatagtcatggggaagggttatgacttagtacctgctctcccagacctg

SEQ ID NO: 44 - DNA, 121 bp, synthetic construct
  misc_feature 1..121: note = Description of Artificial Sequence: Synthetic polynucleotide
  source 1..121: mol_type = other DNA
aatcacatgcaaatataccaattaccatttgatggaaatggatgtcattaacctcctaacmgtgaagaaagagctcacttttggttccatctgaggccttcaggcagatcttcatggccca

SEQ ID NO: 45 - DNA, 121 bp, synthetic construct
  misc_feature 1..121: note = Description of Artificial Sequence: Synthetic polynucleotide
  source 1..121: mol_type = other DNA
agaaactctatcctctgtccagagctgaaatagttgccactcactgcagagagtccactgmtcctccccaaggtcagagtgccatgtggcttacctggaactgcacacaggcctctccctg

SEQ ID NO: 46 - DNA, 121 bp, synthetic construct
  misc_feature 1..121: note = Description of Artificial Sequence: Synthetic polynucleotide
  source 1..121: mol_type = other DNA
ttcacagaggttgggggagagattaagccaaaggatctctgaggtctttttcaaatctatrattatgtggccttttgttcattgacttccatgtgttctagttgatcattacaaacctggc

SEQ ID NO: 47 - DNA, 121 bp, synthetic construct
  misc_feature 1..121: note = Description of Artificial Sequence: Synthetic polynucleotide
  source 1..121: mol_type = other DNA
gccaggatgcacacatacgtagtccccctgctgcactgttggtgtgcactcacccatggcrtcctgctaccctgttggtgtgccctgttggtgtgcactcgctggcagctccctgctgccc

SEQ ID NO: 48 - DNA, 121 bp, synthetic construct
  misc_feature 1..121: note = Description of Artificial Sequence: Synthetic polynucleotide
  source 1..121: mol_type = other DNA
caccaaggtaacagacagctaccctgagccaacccagctcctcatggggaccaagtctgtrtgcgaagtaggtagcaactggttccagcccatctacctcggagccatgttctccttgcaa

SEQ ID NO: 49 - DNA, 121 bp, synthetic construct
  misc_feature 1..121: note = Description of Artificial Sequence: Synthetic polynucleotide
  source 1..121: mol_type = other DNA
ggatacgattgtgatgaaggtctgcggtcctagagctacagatgcatggtaagaaattagracaaactggagatgggccatggactctgcctaggagtgttaggaaaatactttgactcca

SEQ ID NO: 50 - DNA, 121 bp, synthetic construct
  misc_feature 1..121: note = Description of Artificial Sequence: Synthetic polynucleotide
  source 1..121: mol_type = other DNA
aacctgttatggtggaggatgtgtccccaggcctaatgaaggccaactctcctcttgggcrctgaattccatcccatgaattcatccctgccttctaaaagactgtacctccttagttatg

SEQ ID NO: 51 - DNA, 121 bp, synthetic construct
  misc_feature 1..121: note = Description of Artificial Sequence: Synthetic polynucleotide
  source 1..121: mol_type = other DNA
agatatagtgagagagttgtgggtcctagagtatgtaagagagaatagagaagagaggagragagagagagaggaagagaaagcaagaccaaccaataaaccaacataatccaatttttta

SEQ ID NO: 52 - DNA, 121 bp, synthetic construct
  misc_feature 1..121: note = Description of Artificial Sequence: Synthetic polynucleotide
  source 1..121: mol_type = other DNA
atatgcactcaatacaggaagcactcagatttataaagcaagttcttagagatctacaaasagacttagtttcccacagaataacactggcagactttaacatcccactgacagtattaga

SEQ ID NO: 53 - DNA, 121 bp, synthetic construct
  misc_feature 1..121: note = Description of Artificial Sequence: Synthetic polynucleotide
  source 1..121: mol_type = other DNA
atagctgaggcctaagaggacgacaatgcctagaaggattatagactttgggttggaaagratcttggggatactacagagttggagnatattcaagtttaccaaaggaaacaatgagaaa

SEQ ID NO: 54 - DNA, 121 bp, synthetic construct
  misc_feature 1..121: note = Description of Artificial Sequence: Synthetic polynucleotide
  source 1..121: mol_type = other DNA
ttcaagtacaatcaatacctacctcccttacaaacatcaagagcaagaangaaggcagacmtggaaagcccaaatcttccgataactgaaaaacatccatatttgaataagcttatggtca

SEQ ID NO: 55 - DNA, 121 bp, synthetic construct
  misc_feature 1..121: note = Description of Artificial Sequence: Synthetic polynucleotide
  source 1..121: mol_type = other DNA
cagtttttagctgttaggtatattcgatcactttagtctattttgaaaatgatatgagacrctttttaagcaaagtctacagtttccnaatgagaaaattaatcctctttcttgtctttcc

SEQ ID NO: 56 - DNA, 121 bp, synthetic construct
  misc_feature 1..121: note = Description of Artificial Sequence: Synthetic polynucleotide
  source 1..121: mol_type = other DNA
atattcgtgggtataaattagctcaggtacttaagtgacaagccaacaattcaacgtaagraaggtggttcaagacancttcgcagtttcgcagttttggagagtggagcaactcctggag

SEQ ID NO: 57 - DNA, 121 bp, synthetic construct
  misc_feature 1..121: note = Description of Artificial Sequence: Synthetic polynucleotide
  source 1..121: mol_type = other DNA
acctaatagaatgtcagtgttctcaattctgtctgggcctntggagttttaaaatatacartttttcgcattctagtcccaagatcgttgagtgtacttggaaggggaccaaaaggcatca

SEQ ID NO: 58 - DNA, 121 bp, synthetic construct
  misc_feature 1..121: note = Description of Artificial Sequence: Synthetic polynucleotide
  source 1..121: mol_type = other DNA
gaagaacgatgagcctggagcatttatccatcaactgccatccatctctgggtcaggcttractctgcaaatgtcactattttcaaactgccaggctgcacttgcattagggcttagcaga

SEQ ID NO: 59 - DNA, 121 bp, synthetic construct
  misc_feature 1..121: note = Description of Artificial Sequence: Synthetic polynucleotide
  source 1..121: mol_type = other DNA
tcatatccttttaaggcaccatctggtctcttctaaactcccttgagtggttgattgaagraaaacatctaagaacaaataattttcttggaacagtacattctaagtctatattttagag

SEQ ID NO: 60 - DNA, 121 bp, synthetic construct
  misc_feature 1..121: note = Description of Artificial Sequence: Synthetic polynucleotide
  source 1..121: mol_type = other DNA
tagatgcttgagagcggagattgagataaataaagtaaggggacttttagatgaccaagnrgaggcaattaataggtagatgaacggttatttggggcttccaggcagaggcttgcatgga

SEQ ID NO: 61 - DNA, 121 bp, synthetic construct
  misc_feature 1..121: note = Description of Artificial Sequence: Synthetic polynucleotide
  source 1..121: mol_type = other DNA
actcagtgtctgttgaatgaagaactgagcaccaactccacatcaggctctctgattgccrggtgacgtcctataatcttgctatgagattgggatgttgtgccttttattccctagacaa

SEQ ID NO: 62 - DNA, 121 bp, synthetic construct
  misc_feature 1..121: note = Description of Artificial Sequence: Synthetic polynucleotide
  source 1..121: mol_type = other DNA
atgccaatcaaatcaagtcaaatgtaattgtttaggcttttccattctttcattcctattmtataacccatggacactcagtcctatagctgaatattttatggcaccagtgtgatgaact

SEQ ID NO: 63 - DNA, 121 bp, synthetic construct
  misc_feature 1..121: note = Description of Artificial Sequence: Synthetic polynucleotide
  source 1..121: mol_type = other DNA
cccaaaaggttaaatgtggtttcatcacaagcatgggtgtcaattttataaaggagggggraaaagtcattcatggggaacttaaggcttttatccacaaaggtacatgttgagtgaactg

SEQ ID NO: 64 - DNA, 121 bp, synthetic construct
  misc_feature 1..121: note = Description of Artificial Sequence: Synthetic polynucleotide
  source 1..121: mol_type = other DNA
aataagaatgcccaagatggaactgaagaagatgcaatggatcaggagtctgctgtcctgrgctccaggcccgggtctgccaaactggcaaattctaaggccttgaacaaccatttcaaca

SEQ ID NO: 65 - DNA, 121 bp, synthetic construct
  misc_feature 1..121: note = Description of Artificial Sequence: Synthetic polynucleotide
  source 1..121: mol_type = other DNA
actatgtgccactgctgagctagacacaaaaagaatatgacatgttccctgcctttatggractcacagtgtagtgggggtgacagatgcagacactaaaaatttgactacagtatggcta

SEQ ID NO: 66 - DNA, 121 bp, synthetic construct
  misc_feature 1..121: note = Description of Artificial Sequence: Synthetic polynucleotide
  source 1..121: mol_type = other DNA
caggcttgttcatctattgtacactgagctgatgtttatgaagctcccaatatgtgccagrgtctgtacttggtctaaaggtncagatcccaagccctccatgcactctttgaccttggac

SEQ ID NO: 67 - DNA, 121 bp, synthetic construct
  misc_feature 1..121: note = Description of Artificial Sequence: Synthetic polynucleotide
  source 1..121: mol_type = other DNA
ctggcacttttgccagattatccaactggcaaaatgcacagattctcaggcatcaggaagrcagaggcagacaaagagagtcagagagggggtgaggatgcagtgacttcagccagagttt

SEQ ID NO: 68 - DNA, 121 bp, synthetic construct
  misc_feature 1..121: note = Description of Artificial Sequence: Synthetic polynucleotide
  source 1..121: mol_type = other DNA
aaggaatggcctgtgatgtacggcagaaaccagttttactagcgcctccatccagttgctrcttctggttatgtcacagcctggactcttcaggctacttggaaaggcctttcatggcttg

SEQ ID NO: 69 - DNA, 121 bp, synthetic construct
  misc_feature 1..121: note = Description of Artificial Sequence: Synthetic polynucleotide
  source 1..121: mol_type = other DNA
atatattctataaaagagacacttcagtaactcaaaaagtctatgttcttcaagtgccccrccacaaagggttatagcccttggatgaagcatctttctagtcctcttctgaacttaccca

SEQ ID NO: 70 - DNA, 121 bp, synthetic construct
  misc_feature 1..121: note = Description of Artificial Sequence: Synthetic polynucleotide
  source 1..121: mol_type = other DNA
tgtgtcaagagcttattgtntggggaatgttggtgggcatttgacctctatcctcatttcrtcttcatcacagtgctccgggaaaaatcgcaatcacccccattttagagatgaggatatg

SEQ ID NO: 71 - DNA, 121 bp, synthetic construct
  misc_feature 1..121: note = Description of Artificial Sequence: Synthetic polynucleotide
  source 1..121: mol_type = other DNA
ttacctctcatgagggaaataccctcatacagttggccatcacttaacaatagagacaacratgatagatgggatggtagcaactttaggttttgttgtttcctatttttcagtggtgaat

SEQ ID NO: 72 - DNA, 121 bp, synthetic construct
  misc_feature 1..121: note = Description of Artificial Sequence: Synthetic polynucleotide
  source 1..121: mol_type = other DNA
ctggagaccaaggactatgttgcaccataactatcacctcccaggtatgcagaactgagcrattttcaaaggtcttcaccattcatagtctcatttgagcctgaaactactttgacagcta

SEQ ID NO: 73 - DNA, 121 bp, synthetic construct
  misc_feature 1..121: note = Description of Artificial Sequence: Synthetic polynucleotide
  source 1..121: mol_type = other DNA
gtgaatgcctataaaataaagtaacattcgaacaacagcccagagggccgcactggtaaarccgtagcttcctctgtttctactttcattcaataaaaaccgtttcgtattcaactcaggg

SEQ ID NO: 74 - DNA, 121 bp, synthetic construct
  misc_feature 1..121: note = Description of Artificial Sequence: Synthetic polynucleotide
  source 1..121: mol_type = other DNA
acctcggtgtgggcaggacaccacatttattttaacctatgaaactctcatggttggtcamccttgcaatagggctgactctgccctgatagcacacatctggcaggtggccctaaaacag

SEQ ID NO: 75 - DNA, 121 bp, synthetic construct
  misc_feature 1..121: note = Description of Artificial Sequence: Synthetic polynucleotide
  source 1..121: mol_type = other DNA
atctctggtgacttcttaaaagaaccggttacctagaagacatcaggaggaaagagctatraagaaacccacttcctgacttgagcttcactggctcactgtccaagtttgtgtctgagtg

SEQ ID NO: 76 - DNA, 121 bp, synthetic construct
  misc_feature 1..121: note = Description of Artificial Sequence: Synthetic polynucleotide
  source 1..121: mol_type = other DNA
taacattgggctagacctcctcctctaaaaagaaaaaaaaaagtctcnattccctcatttrtacaatgggcataacagaaacttcctcatgtgatatttggtgaaggatttaaaaagtcag

SEQ ID NO: 77 - DNA, 121 bp, synthetic construct
  misc_feature 1..121: note = Description of Artificial Sequence: Synthetic polynucleotide
  source 1..121: mol_type = other DNA
aaatgcctgctacgccccatgacactgccagcaattactgcaattctataagtaaaatgcrttgttccctggcctcaaggaacttagaattatactggaaaaataaaaggtttggagaata

SEQ ID NO: 78 - DNA, 121 bp, synthetic construct
  misc_feature 1..121: note = Description of Artificial Sequence: Synthetic polynucleotide
  source 1..121: mol_type = other DNA
tctgtttctgcccttctcattcccaagctcttttcctcttatccaatcaggtactgcccarggatggtctacattgagactgtgatggcttcagcaagcctggaagccagccccagctttg

SEQ ID NO: 79 - skipped

SEQ ID NO: 80 - DNA, 121 bp, synthetic construct
  misc_feature 1..121: note = Description of Artificial Sequence: Synthetic polynucleotide
  source 1..121: mol_type = other DNA
caattattaaatcatcatctatatttatttatagatgaggaaacagacatgaagagacttmactaggatggtttgtaaaatgttcagttcctacgtttggggagaaggagctgttgaaaag

SEQ ID NO: 81 - DNA, 121 bp, synthetic construct
  misc_feature 1..121: note = Description of Artificial Sequence: Synthetic polynucleotide
  source 1..121: mol_type = other DNA
aaggacaagcctgtcattcctgctgctgccctagctggctacacaggtaggcgccctcccrctgcttaggccaactccatctgcacgtttctgtgggtggggtcctggaaggcactctgca

SEQ ID NO: 82 - DNA, 121 bp, synthetic construct
  misc_feature 1..121: note = Description of Artificial Sequence: Synthetic polynucleotide
  source 1..121: mol_type = other DNA
gggtccagaagcactaggggagggggtaggaaggagtgcacgtaagatgtcctgggtgtasggcgtgagggacagaaggcgggcaaggtgtccaggatggcgcncctggcagttggtggca

SEQ ID NO: 83-108 - skipped (26 entries)

SEQ ID NO: 109 - protein, 8 aa, synthetic construct
  REGION 1..8: note = Description of Artificial Sequence: Synthetic peptide
GFTFSTYG

SEQ ID NO: 110 - protein, 8 aa, synthetic construct
  REGION 1..8: note = Description of Artificial Sequence: Synthetic peptide
ISGTGRTT

SEQ ID NO: 111 - protein, 14 aa, synthetic construct
  REGION 1..14: note = Description of Artificial Sequence: Synthetic peptide
TKERGDYYYGVFDY

SEQ ID NO: 112 - protein, 6 aa, synthetic construct
  REGION 1..6: note = Description of Artificial Sequence: Synthetic peptide
QTISSW

SEQ ID NO: 113 - skipped

SEQ ID NO: 114 - protein, 8 aa, synthetic construct
  REGION 1..8: note = Description of Artificial Sequence: Synthetic peptide
QQYHRSWT

SEQ ID NO: 115 - protein, 121 aa, synthetic construct
  REGION 1..121: note = Description of Artificial Sequence: Synthetic polypeptide
EVQLLESGGGLVQPGKSLRLSCAVSGFTFSTYGMNWVRQAPGKGLEWVSSISGTGRTTYHADSVQGRFTVSRDNSKNILYLQMNSLRADDTAVYFCTKERGDYYYGVFDYWGQGTLVTVSS

SEQ ID NO: 116 - protein, 106 aa, synthetic construct
  REGION 1..106: note = Description of Artificial Sequence: Synthetic polypeptide
DIQMTQSPSTLSASVGDRVTITCRASQTISSWLAWYQQTPEKAPKLLIYAASNLQSGVPSRFSGSGSGTEFTLTISSLQPDDFATYYCQQYHRSWTFGQGTKVEIT

SEQ ID NO: 117 - protein, 8 aa, synthetic construct
  REGION 1..8: note = Description of Artificial Sequence: Synthetic peptide
GFTFSSYW

SEQ ID NO: 118 - protein, 8 aa, synthetic construct
  REGION 1..8: note = Description of Artificial Sequence: Synthetic peptide
IKEDGSEK

SEQ ID NO: 119 - protein, 15 aa, synthetic construct
  REGION 1..15: note = Description of Artificial Sequence: Synthetic peptide
AREDYDSYYKYGMDV

SEQ ID NO: 120 - protein, 12 aa, synthetic construct
  REGION 1..12: note = Description of Artificial Sequence: Synthetic peptide
QSILYSSNNKNY

SEQ ID NO: 121 - skipped

SEQ ID NO: 122 - protein, 9 aa, synthetic construct
  REGION 1..9: note = Description of Artificial Sequence: Synthetic peptide
QQYYSTPFT

SEQ ID NO: 123 - protein, 122 aa, synthetic construct
  REGION 1..122: note = Description of Artificial Sequence: Synthetic polypeptide
EVQLVESGGGLVQPGGSLRLSCAVSGFTFSSYWMSWVRQAPGKGLEWVANIKEDGSEKNYVDSVKGRFTLSSDNAKNSLYLQMNSLRAEDTAVYYCAREDYDSYYKYGMDVWGQGTAVIVSS

SEQ ID NO: 124 - protein, 113 aa, synthetic construct
  REGION 1..113: note = Description of Artificial Sequence: Synthetic polypeptide
DIVMTQSPDSLAVSLGERATINCKSSQSILYSSNNKNYLAWYQQKPGQPPKLLIYWASTRESGVPDRFSGSGSGTDFTLTISSLQAEDVSVYYCQQYYSTPFTFGPGTKVDIK

SEQ ID NO: 125 - protein, 8 aa, synthetic construct
  REGION 1..8: note = Description of Artificial Sequence: Synthetic peptide
GGSFTGFY

SEQ ID NO: 126 - protein, 7 aa, synthetic construct
  REGION 1..7: note = Description of Artificial Sequence: Synthetic peptide
INHRGNT

SEQ ID NO: 127 - protein, 13 aa, synthetic construct
  REGION 1..13: note = Description of Artificial Sequence: Synthetic peptide
ASPFYDFWSGSDY

SEQ ID NO: 128 - protein, 11 aa, synthetic construct
  REGION 1..11: note = Description of Artificial Sequence: Synthetic peptide
QSLVHSDGNTY

SEQ ID NO: 129 - skipped

SEQ ID NO: 130 - protein, 9 aa, synthetic construct
  REGION 1..9: note = Description of Artificial Sequence: Synthetic peptide
MQATQFPLT

SEQ ID NO: 131 - protein, 119 aa, synthetic construct
  REGION 1..119: note = Description of Artificial Sequence: Synthetic polypeptide
QVQLQQWGAGLLKPSETLSLTCAVYGGSFTGFYWSWIRQPPGKGLEWIGEINHRGNTNYNPSLKSRVTMSVDTSKNQFSLNMISVTAADTAMYFCASPFYDFWSGSDYWGQGTLVTVSS

SEQ ID NO: 132 - protein, 112 aa, synthetic construct
  REGION 1..112: note = Description of Artificial Sequence: Synthetic polypeptide
DIMLTQTPLTSPVTLGQPASISCKSSQSLVHSDGNTYLSWLQQRPGQPPRLLFYKISNRFSGVPDRFSGSGAGTDFTLKISRVEAEDVGVYYCMQATQFPLTFGGGTKVEIK

SEQ ID NO: 133 - protein, 10 aa, synthetic construct
  REGION 1..10: note = Description of Artificial Sequence: Synthetic peptide
  VARIANT 3: note = X can be P, S, D, Q, or N
  VARIANT 5: note = X can be T or R
  VARIANT 6: note = X can be N, T, Y, or H
GYXFXXYGIS

SEQ ID NO: 134 - protein, 17 aa, synthetic construct
  REGION 1..17: note = Description of Artificial Sequence: Synthetic peptide
  VARIANT 4: note = X can be T, P, S, or A
  VARIANT 8: note = X can be N, G, V, K, or A
  VARIANT 9: note = X can be T or K
  VARIANT 10: note = X can be H or N
  VARIANT 13: note = X can be Q or R
  VARIANT 14: note = X can be K or M
  VARIANT 15: note = X can be L or H
WISXYNGXXXYAXXXQG

SEQ ID NO: 135 - protein, 15 aa, synthetic construct
  REGION 1..15: note = Description of Artificial Sequence: Synthetic peptide
  VARIANT 8: note = X can be S or A
  VARIANT 9: note = X can be Y or P
  VARIANT 15: note = X can be V, A, or G
ENYYGSGXXRGGMDX

SEQ ID NO: 136 - protein, 10 aa, synthetic construct
  REGION 1..10: note = Description of Artificial Sequence: Synthetic peptide
GYDFTYYGIS

SEQ ID NO: 137 - protein, 17 aa, synthetic construct
  REGION 1..17: note = Description of Artificial Sequence: Synthetic peptide
WISTYNGNTHYARMLQG

SEQ ID NO: 138 - protein, 15 aa, synthetic construct
  REGION 1..15: note = Description of Artificial Sequence: Synthetic peptide
ENYYGSGAYRGGMDV

SEQ ID NO: 139 - protein, 11 aa, synthetic construct
  REGION 1..11: note = Description of Artificial Sequence: Synthetic peptide
RASQSVSSYLA

SEQ ID NO: 140 - protein, 7 aa, synthetic construct
  REGION 1..7: note = Description of Artificial Sequence: Synthetic peptide
DASNRAT

SEQ ID NO: 141 - protein, 9 aa, synthetic construct
  REGION 1..9: note = Description of Artificial Sequence: Synthetic peptide
QQRSNWPWT

SEQ ID NO: 142 - protein, 124 aa, synthetic construct
  REGION 1..124: note = Description of Artificial Sequence: Synthetic polypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 143 - protein, 107 aa, synthetic construct
  REGION 1..107: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 144 - protein, 453 aa, synthetic construct
  REGION 1..453: note = Description of Artificial Sequence: Synthetic polypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 214 aa, synthetic construct
  REGION 1..214: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 7 aa, synthetic construct
  REGION 1..7: note = Description of Artificial Sequence: Synthetic peptide
SRSYYWG

SEQ ID NO: 147 - protein, 16 aa, synthetic construct
  REGION 1..16: note = Description of Artificial Sequence: Synthetic peptide
SIYYNGRTYYNPSLKS

SEQ ID NO: 148 - protein, 11 aa, synthetic construct
  REGION 1..11: note = Description of Artificial Sequence: Synthetic peptide
EDYGDYGAFDI

SEQ ID NO: 149 - protein, 11 aa, synthetic construct
  REGION 1..11: note = Description of Artificial Sequence: Synthetic peptide
RASQGISSALA

SEQ ID NO: 150 - protein, 7 aa, synthetic construct
  REGION 1..7: note = Description of Artificial Sequence: Synthetic peptide
DASSLES

SEQ ID NO: 151 - protein, 9 aa, synthetic construct
  REGION 1..9: note = Description of Artificial Sequence: Synthetic peptide
QQFNSYPLT

SEQ ID NO: 152 - protein, 121 aa, synthetic construct
  REGION 1..121: note = Description of Artificial Sequence: Synthetic polypeptide
QLQLQESGPGLVKPSETLSLTCTVSGGSISSRSYYWGWIRQPPGKGLEWIGSIYYNGRTYYNPSLKSRVTISVDTSKNQFSLKLSSVTAADTAVYYCAREDYGDYGAFDIWGQGTMVTVSS

SEQ ID NO: 153 - protein, 107 aa, synthetic construct [sequence omitted]

SEQ ID NO: 154 - protein, 7 aa, synthetic construct
  REGION 1..7: note = Description of Artificial Sequence: Synthetic peptide
TSNMGVV

SEQ ID NO: 155 - protein, 16 aa, synthetic construct
  REGION 1..16: note = Description of Artificial Sequence: Synthetic peptide
HILWDDREYSNPALKS

SEQ ID NO: 156 - protein, 14 aa, synthetic construct
  REGION 1..14: note = Description of Artificial Sequence: Synthetic peptide
MSRNYYGSSYVMDY

SEQ ID NO: 157 - protein, 10 aa, synthetic construct
  REGION 1..10: note = Description of Artificial Sequence: Synthetic peptide
SASSSVNYMH

SEQ ID NO: 158 - protein, 7 aa, synthetic construct
  REGION 1..7: note = Description of Artificial Sequence: Synthetic peptide
STSNLAS

SEQ ID NO: 159 - protein, 8 aa, synthetic construct
  REGION 1..8: note = Description of Artificial Sequence: Synthetic peptide
HQWNNYGT

SEQ ID NO: 160 - protein, 124 aa, synthetic construct
  REGION 1..124: note = Description of Artificial Sequence: Synthetic polypeptide
QVTLKESGPALVKPTQTLTLTCTFSGFSLSTSNMGVVWIRQPPGKALEWLAHILWDDREYSNPALKSRLTISKDTSKNQVVLTMTNMDPVDTATYYCARMSRNYYGSSYVMDYWGQGTLVTVSS

SEQ ID NO: 161 - protein, 106 aa, synthetic construct
  REGION 1..106: note = Description of Artificial Sequence: Synthetic polypeptide
DIQLTQSPSFLSASVGDRVTITCSASSSVNYMHWYQQKPGKAPKLLIYSTSNLASGVPSRFSGSGSGTEFTLTISSLQPEDFATYYCHQWNNYGTFGQGTKVEIKR

SEQ ID NO: 162 - protein, 5 aa, synthetic construct
  REGION 1..5: note = Description of Artificial Sequence: Synthetic peptide
LYGMN

SEQ ID NO: 163 - protein, 5 aa, synthetic construct
  REGION 1..5: note = Description of Artificial Sequence: Synthetic peptide
NYGMN

SEQ ID NO: 164 - protein, 17 aa, synthetic construct
  REGION 1..17: note = Description of Artificial Sequence: Synthetic peptide
WINTYTGEPTYADDFKG

SEQ ID NO: 165 - protein, 10 aa, synthetic construct
  REGION 1..10: note = Description of Artificial Sequence: Synthetic peptide
DTAMDYAMAY

SEQ ID NO: 166 - protein, 13 aa, synthetic construct
  REGION 1..13: note = Description of Artificial Sequence: Synthetic peptide
DYGKYGDYYAMDY

SEQ ID NO: 167 - protein, 16 aa, synthetic construct
  REGION 1..16: note = Description of Artificial Sequence: Synthetic peptide
KSSQNIVHSDGNTYLE

SEQ ID NO: 168 - protein, 16 aa, synthetic construct
  REGION 1..16: note = Description of Artificial Sequence: Synthetic peptide
RSSQSIVHSNGNTYLD

SEQ ID NO: 169 - protein, 7 aa, synthetic construct
  REGION 1..7: note = Description of Artificial Sequence: Synthetic peptide
KVSNRFS

SEQ ID NO: 170 - protein, 9 aa, synthetic construct
  REGION 1..9: note = Description of Artificial Sequence: Synthetic peptide
FQGSHVPLT

SEQ ID NO: 171 - protein, 119 aa, synthetic construct
  REGION 1..119: note = Description of Artificial Sequence: Synthetic polypeptide
QVQLVQSGSELKKPGASVKVSCKASGYTFTLYGMNWVRQAPGQGLEWMGWINTYTGEPTYADDFKGRFVFSLDTSVSTAYLQISSLKAEDTAVYYCARDTAMDYAMAYWGQGTLVTVSS

SEQ ID NO: 172 - protein, 119 aa, synthetic construct
  REGION 1..119: note = Description of Artificial Sequence: Synthetic polypeptide
QVQLVQSGSELKKPGASVKVSCKASGYTFTLYGMNWVKQAPGKGLKWMGWINTYTGEPTYADDFKGRFVFSLDTSVSTAYLQISSLKAEDTAVYFCARDTAMDYAMAYWGQGTLVTVSS

SEQ ID NO: 173 - protein, 122 aa, synthetic construct
  REGION 1..122: note = Description of Artificial Sequence: Synthetic polypeptide
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARDYGKYGDYYAMDYWGQGTLVTVSS

SEQ ID NO: 174 - protein, 122 aa, synthetic construct
  REGION 1..122: note = Description of Artificial Sequence: Synthetic polypeptide
QVQLVQSGSELKKPGASVKVSCKASGYTFTNYGMNWVRQAPGKGLKWMGWINTYTGEPTYADDFKGRFVFSLDTSVSTAYLQISSLKAEDTAVYFCARDYGKYGDYYAMDYWGQGTLVTVSS

SEQ ID NO: 175 - protein, 113 aa, synthetic construct
  REGION 1..113: note = Description of Artificial Sequence: Synthetic polypeptide
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGGGTKVEIKR

SEQ ID NO: 176 - protein, 113 aa, synthetic construct
  REGION 1..113: note = Description of Artificial Sequence: Synthetic polypeptide
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGQGTKVEIKR

SEQ ID NO: 177 - protein, 113 aa, synthetic construct
  REGION 1..113: note = Description of Artificial Sequence: Synthetic polypeptide
DVVMTQTPLSLPVTPGEPASISCKSSQNIVHSDGNTYLEWYLQKPGQSPQLLIYKVSNRFSGVPDRFSGSGSGTDFTLKISRVEAEDLGVYYCFQGSHVPLTFGGGTKVEIKR

SEQ ID NO: 178 - protein, 113 aa, synthetic construct
  REGION 1..113: note = Description of Artificial Sequence: Synthetic polypeptide
DVVMTQTPLSLPVSLGDQASISCKSSQNIVHSDGNTYLEWYLQKPGQSPKVLIYKVSNRFSGVPDRFSGSGSGTDFTLKISRVEAEDLGVYYCFQGSHVPLTFGGGTKVEIKR

SEQ ID NO: 179 - protein, 113 aa, synthetic construct
  REGION 1..113: note = Description of Artificial Sequence: Synthetic polypeptide
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGGGTKVEIKR

SEQ ID NO: 180 - protein, 113 aa, synthetic construct
  REGION 1..113: note = Description of Artificial Sequence: Synthetic polypeptide
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGQGTKVEIKR

SEQ ID NO: 181 - protein, 113 aa, synthetic construct
  REGION 1..113: note = Description of Artificial Sequence: Synthetic polypeptide
DVVMTQTPLSLPVTPGEPASISCRSSQSIVHSNGNTYLDWYLQKPGQSPQLLIYKVSNRFSGVPDRFSGSGSGTDFTLKISRVEAEDLGVYYCFQGSHVPLTFGGGTKVEIKR

SEQ ID NO: 182 - protein, 113 aa, synthetic construct
  REGION 1..113: note = Description of Artificial Sequence: Synthetic polypeptide
DVVMTQTPLSLPVSLGDQASISCRSSQSIVHSNGNTYLDWYLQKPGQSPKVLIYKVSNRFSGVPDRFSGSGSGTDFTLKINRVEAEDLGVYFCFQGSHVPLTFGGGTKLEIKR

SEQ ID NO: 183 - protein, 10 aa, synthetic construct
  REGION 1..10: note = Description of Artificial Sequence: Synthetic peptide
GYTFTSSWMH

SEQ ID NO: 184 - protein, 8 aa, synthetic construct
  REGION 1..8: note = Description of Artificial Sequence: Synthetic peptide
IHPNSGGT

SEQ ID NO: 185 - protein, 14 aa, synthetic construct
  REGION 1..14: note = Description of Artificial Sequence: Synthetic peptide
ARGDYYGYVSWFAY

SEQ ID NO: 186 - protein, 6 aa, synthetic construct
  REGION 1..6: note = Description of Artificial Sequence: Synthetic peptide
QNINVL

SEQ ID NO: 187 - skipped

SEQ ID NO: 188 - protein, 9 aa, synthetic construct
  REGION 1..9: note = Description of Artificial Sequence: Synthetic peptide
QQGQSYPYT

SEQ ID NO: 189 - protein, 119 aa, synthetic construct
  REGION 1..119: note = Description of Artificial Sequence: Synthetic polypeptide
QVQLQQPGSVLVRPGASVKVSCKASGYTFTSSWMHWAKQRPGQGLEWIGEIHPNSGGTNYNEKFKGKATVDTSSSTAYVDLSSLTSEDSAVYYCARGDYYGYVSWFAYWGQGTLVTVSS

SEQ ID NO: 190 - protein, 121 aa, synthetic construct
  REGION 1..121: note = Description of Artificial Sequence: Synthetic polypeptide
QVQLVQSGAEVKKPGASVKVSCKASGYTFTSSWMHWARQAPGQGLEWIGEIHPNSGGTNYAQKFQGRATLTVDTSSSTAYMELSRLRSDDTAVYYCARGDYYGYVSWFAYWGQGTLVTVSS

SEQ ID NO: 191 - protein, 121 aa, synthetic construct
  REGION 1..121: note = Description of Artificial Sequence: Synthetic polypeptide
QVQLVQSGAEVKKPGASVKVSCKASGYTFTSSWMHWARQAPGQGLEWIGEIHPNSGGTNYAQKFQGRATMTVDTSISTAYMELSRLRSDDTAVYYCARGDYYGYVSWFAYWGQGTLVTVSS

SEQ ID NO: 192 - protein, 121 aa, synthetic construct
  REGION 1..121: note = Description of Artificial Sequence: Synthetic polypeptide
QVQLVQSGAEVKKPGASVKVSCKASGYTFTSSWMHWARQAPGQGLEWIGEIHPNSGGTNYAQKFQGRVTMTVDTSISTAYMELSRLRSDDTAVYYCARGDYYGYVSWFAYWGQGTLVTVSS

SEQ ID NO: 193 - protein, 121 aa, synthetic construct
  REGION 1..121: note = Description of Artificial Sequence: Synthetic polypeptide
QVQLVQSGAEVKKPGASVKVSCKASGYTFTSSWMHWARQAPGQGLEWMGEIHPNSGGTNYAQKFQGRVTMTVDTSISTAYMELSRLRSDDTAVYYCARGDYYGYVSWFAYWGQGTLVTVSS

SEQ ID NO: 194 - protein, 107 aa, synthetic construct
  REGION 1..107: note = Description of Artificial Sequence: Synthetic polypeptide
DIQMNQSPSSLSASLGDTITITCHASQNINVLLSWYQQKPGNIPKLLIYKASNLHTGVPSRFSGSGSGTGFTFTISSLQPEDIATYYCQQGQSYPYTFGGGTKLEIK

SEQ ID NO: 195 - protein, 107 aa, synthetic construct
  REGION 1..107: note = Description of Artificial Sequence: Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 196 - protein, 107 aa, synthetic construct
  REGION 1..107: note = Description of Artificial Sequence: Synthetic polypeptide
DIQMTQSPSSLSASVGDRVTITCQASQNINVLLNWYQQKPGKAPKLLIYKASNLHTGVPSRFSGSGSGTDFTFTISSLQPEDIATYYCQQGQSYPYTFGQGTKLEIK

SEQ ID NO: 197 - protein, 107 aa, synthetic construct
  REGION 1..107: note = Description of Artificial Sequence: Synthetic polypeptide
DIQMNQSPSSLSASVGDRVTITCQASQNINVLLSWYQQKPGKAPKLLIYKASNLHTGVPSRFSGSGSGTDFTFTISSLQPEDIATYYCQQGQSYPYTFGQGTKLEIK

SEQ ID NO: 198 - protein, 10 aa, synthetic construct
  REGION 1..10: note = Description of Artificial Sequence: Synthetic peptide
GYTFTSYDIN

SEQ ID NO: 199 - protein, 10 aa, synthetic construct
  REGION 1..10: note = Description of Artificial Sequence: Synthetic peptide
  VARIANT 8: note = X can be N or Y
WLNPNSGXTG

SEQ ID NO: 200 - protein, 10 aa, synthetic construct
  REGION 1..10: note = Description of Artificial Sequence: Synthetic peptide
EVPETAAFEY

SEQ ID NO: 201 - protein, 14 aa, synthetic construct
  REGION 1..14: note = Description of Artificial Sequence: Synthetic peptide
  VARIANT 10: note = X can be G or A
  VARIANT 11: note = X can be L, S, or Q
  VARIANT 14: note = X can be H or L
TSSSSDIGAXXGVX

SEQ ID NO: 202 - protein, 7 aa, synthetic construct
  REGION 1..7: note = Description of Artificial Sequence: Synthetic peptide
GYYNRPS

SEQ ID NO: 203 - protein, 10 aa, synthetic construct
  REGION 1..10: note = Description of Artificial Sequence: Synthetic peptide
  VARIANT 3: note = X can be Y, W, or F
QSXDGTLSAL

SEQ ID NO: 204 - protein, 119 aa, synthetic construct
  REGION 1..119: note = Description of Artificial Sequence: Synthetic polypeptide
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 205 - protein, 111 aa, synthetic construct
  REGION 1..111: note = Description of Artificial Sequence: Synthetic polypeptide
  VARIANT 32..33: note = X can be any amino acid
  VARIANT 36: note = X can be any amino acid
  VARIANT 93: note = X can be any amino acid
QSVLTQPPSVSGAPGQRVTISCTSSSSDIGAXXGVXWYQQLPGTAPKLLIEGYYNRPSGVPDRFSGSKSGTSASLTITGLLPEDEGDYYCQSXDGTLSALFGGGTKLTVLG

SEQ ID NO: 206 - protein, 119 aa, synthetic construct; identical to SEQ ID NO: 204
  REGION 1..119: note = Description of Artificial Sequence: Synthetic polypeptide

SEQ ID NO: 207 - protein, 111 aa, synthetic construct
  REGION 1..111: note = Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 208 - protein, 119 aa, synthetic construct
  REGION 1..119: note = Description of Artificial Sequence: Synthetic polypeptide
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 209 - protein, 111 aa, synthetic construct
  REGION 1..111: note = Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 210 - protein, 119 aa, synthetic construct; identical to SEQ ID NO: 204
  REGION 1..119: note = Description of Artificial Sequence: Synthetic polypeptide

SEQ ID NO: 211 - protein, 111 aa, synthetic construct
  REGION 1..111: note = Description of Artificial Sequence: Synthetic polypeptide
QSVLTQPPSVSGAPGQRVTISCTSSSSDIGAALGVHWYQQLPGTAPKLLIEGYYNRPSGVPDRFSGSKSGTSASLTITGLLPEDEGDYYCQSWDGTLSALFGGGTKLTVLG

SEQ ID NO: 212 - protein, 119 aa, synthetic construct; identical to SEQ ID NO: 204
  REGION 1..119: note = Description of Artificial Sequence: Synthetic polypeptide

SEQ ID NO: 213 - protein, 111 aa, synthetic construct
  REGION 1..111: note = Description of Artificial Sequence: Synthetic polypeptide
QSVLTQPPSVSGAPGQRVTISCTSSSSDIGAGSGVHWYQQLPGTAPKLLIEGYYNRPSGVPDRFSGSKSGTSASLTITGLLPEDEGDYYCQSWDGTLSALFGGGTKLTVLG

SEQ ID NO: 214 - protein, 119 aa, synthetic construct; identical to SEQ ID NO: 204
  REGION 1..119: note = Description of Artificial Sequence: Synthetic polypeptide

SEQ ID NO: 215 - protein, 111 aa, synthetic construct
  REGION 1..111: note = Description of Artificial Sequence: Synthetic polypeptide
QSVLTQPPSVSGAPGQRVTISCTSSSSDIGAGQGVHWYQQLPGTAPKLLIEGYYNRPSGVPDRFSGSKSGTSASLTITGLLPEDEGDYYCQSWDGTLSALFGGGTKLTVLG

SEQ ID NO: 216 - protein, 119 aa, synthetic construct; identical to SEQ ID NO: 204
  REGION 1..119: note = Description of Artificial Sequence: Synthetic polypeptide

SEQ ID NO: 217 - protein, 111 aa, synthetic construct
  REGION 1..111: note = Description of Artificial Sequence: Synthetic polypeptide
QSVLTQPPSVSGAPGQRVTISCTSSSSDIGAGLGVLWYQQLPGTAPKLLIEGYYNRPSGVPDRFSGSKSGTSASLTITGLLPEDEGDYYCQSWDGTLSALFGGGTKLTVLG

SEQ ID NO: 218 - protein, 119 aa, synthetic construct; identical to SEQ ID NO: 208
  REGION 1..119: note = Description of Artificial Sequence: Synthetic polypeptide

SEQ ID NO: 219 - protein, 111 aa, synthetic construct; identical to SEQ ID NO: 207
  REGION 1..111: note = Description of Artificial Sequence: Synthetic polypeptide

SEQ ID NO: 220 - protein, 119 aa, synthetic construct; identical to SEQ ID NO: 208
  REGION 1..119: note = Description of Artificial Sequence: Synthetic polypeptide

SEQ ID NO: 221 - protein, 111 aa, synthetic construct; identical to SEQ ID NO: 213
  REGION 1..111: note = Description of Artificial Sequence: Synthetic polypeptide

SEQ ID NO: 222 - protein, 119 aa, synthetic construct; identical to SEQ ID NO: 208
  REGION 1..119: note = Description of Artificial Sequence: Synthetic polypeptide

SEQ ID NO: 223 - protein, 111 aa, synthetic construct; identical to SEQ ID NO: 215
  REGION 1..111: note = Description of Artificial Sequence: Synthetic polypeptide

SEQ ID NO: 224 - protein, 119 aa, synthetic construct; identical to SEQ ID NO: 208
  REGION 1..119: note = Description of Artificial Sequence: Synthetic polypeptide

SEQ ID NO: 225 - protein, 111 aa, synthetic construct; identical to SEQ ID NO: 217
  REGION 1..111: note = Description of Artificial Sequence: Synthetic polypeptide

SEQ ID NO: 226 - protein, 119 aa, synthetic construct; identical to SEQ ID NO: 208
  REGION 1..119: note = Description of Artificial Sequence: Synthetic polypeptide

SEQ ID NO: 227 - protein, 111 aa, synthetic construct
  REGION 1..111: note = Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSFDGTLSALFGGGTKLTVLG

SEQ ID NO: 228 - protein, 5 aa, synthetic construct
  REGION 1..5: note = Description of Artificial Sequence: Synthetic peptide
SYFWS

SEQ ID NO: 229 - protein, 16 aa, synthetic construct
  REGION 1..16: note = Description of Artificial Sequence: Synthetic peptide
YIYYSGNTKYNPSLKS

SEQ ID NO: 230 - protein, 10 aa, synthetic construct
  REGION 1..10: note = Description of Artificial Sequence: Synthetic peptide
ETGSYYGFDY

SEQ ID NO: 231 - protein, 11 aa, synthetic construct
  REGION 1..11: note = Description of Artificial Sequence: Synthetic peptide
RASQSINNYLN

SEQ ID NO: 232 - protein, 7 aa, synthetic construct
  REGION 1..7: note = Description of Artificial Sequence: Synthetic peptide
AASSLQS

SEQ ID NO: 233 - protein, 9 aa, synthetic construct
  REGION 1..9: note = Description of Artificial Sequence: Synthetic peptide
QQSYSTPRT

SEQ ID NO: 234 - protein, 118 aa, synthetic construct
  REGION 1..118: note = Description of Artificial Sequence: Synthetic polypeptide
QVQLQESGPGLVKPSETLSLTCTVSGGSISSYFWSWIRQPPGKGLEWIGYIYYSGNTKYNPSLKSRVTISIDTSKNQFSLKLSSVTAADTAVYYCARETGSYYGFDYWGQGTLVTVSS

SEQ ID NO: 235 - protein, 107 aa, synthetic construct
  REGION 1..107: note = Description of Artificial Sequence: Synthetic polypeptide
DIQMTQSPSSLSASVGDRVTITCRASQSINNYLNWYQQRPGKAPKLLIYAASSLQSGVPSRFSGSGSGTDFTLTISSLQPGDFATYYCQQSYSTPRTFGQGTKLEIK

SEQ ID NO: 236 - protein, 5 aa, synthetic construct
  REGION 1..5: note = Description of Artificial Sequence: Synthetic peptide
GYYWN

SEQ ID NO: 237 - protein, 16 aa, synthetic construct
  REGION 1..16: note = Description of Artificial Sequence: Synthetic peptide
EINHAGNTNYNPSLKS

SEQ ID NO: 238 - protein, 12 aa, synthetic construct
  REGION 1..12: note = Description of Artificial Sequence: Synthetic peptide
GYCRSTTCYFDY

SEQ ID NO: 239 - protein, 12 aa, synthetic construct
  REGION 1..12: note = Description of Artificial Sequence: Synthetic peptide
RASQSVRSSYLA

SEQ ID NO: 240 - protein, 7 aa, synthetic construct
  REGION 1..7: note = Description of Artificial Sequence: Synthetic peptide
GASSRAT

SEQ ID NO: 241 - protein, 8 aa, synthetic construct
  REGION 1..8: note = Description of Artificial Sequence: Synthetic peptide
QQYGSSPT

SEQ ID NO: 242 - protein, 120 aa, synthetic construct
  REGION 1..120: note = Description of Artificial Sequence: Synthetic polypeptide
QVQLQQWGAGLLKPSETLSLTCAVHGGSFSGYYWNWIRQPPGKGLEWIGEINHAGNTNYNPSLKSRVTISLDTSKNQFSLTLTSVTAADTAVYYCARGYCRSTTCYFDYWGQGTLVTVSS

SEQ ID NO: 243 - protein, 107 aa, synthetic construct
  REGION 1..107: note = Description of Artificial Sequence: Synthetic polypeptide
EIVLTQSPGTLSLSPGERATLSCRASQSVRSSYLAWYQQKPGQAPRLLIYGASSRATGIPDRFSGSGSGTDFTLTISRLEPEDFAVYYCQQYGSSPTFGQGTRLEIK

SEQ ID NO: 244 - protein, 116 aa, synthetic construct
  REGION 1..116: note = Description of Artificial Sequence: Synthetic polypeptide
EVQLQQSGAELVKPGASVKLSCTASGFDIQDTYMHWVKQRPEQGLEWIGRIDPASGHTKYDPKFQVKATITTDTSSNTAYLQLSSLTSEDTAVYYCSRSGGLPDVWGAGTTVTVSS

SEQ ID NO: 245 - protein, 106 aa, synthetic construct
  REGION 1..106: note = Description of Artificial Sequence: Synthetic polypeptide
QIVLSQSPAILSASPGEKVTMTCRASSSVSYMYWYQQKPGSSPKPWIYATSNLASGVPDRFSGSGSGTSYSLTISRVEAEDAATYYCQQWSGNPRTFGGGTKLEIK

SEQ ID NO: 246 - protein, 10 aa, synthetic construct
  REGION 1..10: note = Description of Artificial Sequence: Synthetic peptide
GFDIQDTYMH

SEQ ID NO: 247 - protein, 17 aa, synthetic construct
  REGION 1..17: note = Description of Artificial Sequence: Synthetic peptide
RIDPASGHTKYDPKFQV

SEQ ID NO: 248 - protein, 7 aa, synthetic construct
  REGION 1..7: note = Description of Artificial Sequence: Synthetic peptide
SGGLPDV

SEQ ID NO: 249 - protein, 10 aa, synthetic construct
  REGION 1..10: note = Description of Artificial Sequence: Synthetic peptide
RASSSVSYMY

SEQ ID NO: 250 - protein, 7 aa, synthetic construct
  REGION 1..7: note = Description of Artificial Sequence: Synthetic peptide
ATSNLAS

SEQ ID NO: 251 - protein, 9 aa, synthetic construct
  REGION 1..9: note = Description of Artificial Sequence: Synthetic peptide
QQWSGNPRT

SEQ ID NO: 252 - protein, 116 aa, synthetic construct
  REGION 1..116: note = Description of Artificial Sequence: Synthetic polypeptide
QVQLVQSGAEVKKPGASVKLSCKASGFDIQDTYMHWVRQAPGQGLEWMGRIDPASGHTKYDPKFQVRVTMTTDTSTSTVYMELSSLRSEDTAVYYCSRSGGLPDVWGQGTTVTVSS

SEQ ID NO: 253 - protein, 106 aa, synthetic construct
  REGION 1..106: note = Description of Artificial Sequence: Synthetic polypeptide
EIVLTQSPGTLSLSPGERVTMSCRASSSVSYMYWYQQKPGQAPRPWIYATSNLASGVPDRFSGSGSGTDYTLTISRLEPEDFAVYYCQQWSGNPRTFGGGTKLEIK

SEQ ID NO: 254 - protein, 116 aa, synthetic construct
  REGION 1..116: note = Description of Artificial Sequence: Synthetic polypeptide
QVQLVQSGAEVKKPGASVKLSCKASGFDIQDTYMHWVRQAPGQGLEWMGRIDPASGHTKYDPKFQVRVTMTRDTSTSTVYMELSSLRSEDTAVYYCSRSGGLPDVWGQGTTVTVSS

SEQ ID NO: 255 - protein, 106 aa, synthetic construct
  REGION 1..106: note = Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRTFGGGTKLEIK

SEQ ID NO: 256 - protein, 116 aa, synthetic construct
  REGION 1..116: note = Description of Artificial Sequence: Synthetic polypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDVWGQGTTVTVSS

SEQ ID NO: 257 - protein, 106 aa, synthetic construct
  REGION 1..106: note = Description of Artificial Sequence: Synthetic polypeptide
EIVLTQSPGTLSLSPGERATLSCRASSSVSYMYWYQQKPGQAPRLLIYATSNLASGVPDRFSGSGSGTDYTLTISRLEPEDFAVYYCQQWSGNPRTFGGGTKLEIK

SEQ ID NO: 258 - protein, 121 aa, synthetic construct
  REGION 1..121: note = Description of Artificial Sequence: Synthetic polypeptide
EVMLVESGGGLVKPGGSLKLSCAASGFTFTNYAMSWVRQTPEKRLEWVATITSGGSYIYYLDSVKGRFTISRDNAKSTLYLQMSSLRSEDTAIYNCARRKDGNYYYAMDYWGQGTSVTVSS

SEQ ID NO: 259 - protein, 121 aa, synthetic construct
  REGION 1..121: note = Description of Artificial Sequence: Synthetic polypeptide
EVMLVESGGGLVKPGGSLKLSCAASGFTFTNYAMSWVRQTPEKRLEWVATITSGGSYIYYLDSVKGRFTISRDNAKSTLYLQMSSLRSEDTAIYYCARRKDGNYYYAMDYWGQGTSVTVSS

SEQ ID NO: 260 - protein, 121 aa, synthetic construct
  REGION 1..121: note = Description of Artificial Sequence: Synthetic polypeptide
EVQLVESGGGLVKPGGSLRLSCAASGFTFTNYAMSWVRQAPGQRLEWVSTITSGGSYIYYLDSVKGRFTISRDNAKSTLYLQMNSLRAEDTAVYNCARRKDGNYYYAMDYWGQGTTVTVSS

SEQ ID NO: 261 - protein, 121 aa, synthetic construct
  REGION 1..121: note = Description of Artificial Sequence: Synthetic polypeptide
EVQLVESGGGLVKPGGSLRLSCAASGFTFTNYAMSWVRQAPGQRLEWVSTITSGGSYIYYLDSVKGRFTISRDNAKSTLYLQMNSLRAEDTAVYYCARRKDGNYYYAMDYWGQGTTVTVSS

SEQ ID NO: 262 - protein, 121 aa, synthetic construct
  REGION 1..121: note = Description of Artificial Sequence: Synthetic polypeptide
EVQLLESGGGLVQPGRSLRLSCAASGFTFTNYAMSWVRQAPGQRLEWLATITSGGSYIYYLDSVKGRFTISRDNSKSTLYLQMGSLRAEDMAVYNCARRKDGNYYYAMDYWGQGTTVTVSS

SEQ ID NO: 263 - protein, 121 aa, synthetic construct
  REGION 1..121: note = Description of Artificial Sequence: Synthetic polypeptide
EVQLLESGGGLVQPGRSLRLSCAASGFTFTNYAMSWVRQAPGQRLEWLATITSGGSYIYYLDSVKGRFTISRDNSKSTLYLQMGSLRAEDMAVYYCARRKDGNYYYAMDYWGQGTTVTVSS

SEQ ID NO: 264 - protein, 121 aa, synthetic construct
  REGION 1..121: note = Description of Artificial Sequence: Synthetic polypeptide
QVQLVESGGGLIQPGGSLRLSCAASGFTFTNYAMSWVRQARGQRLEWVSTITSGGSYIYYLDSVKGRFTISRDNSKSTLYMELSSLRSEDTAVYNCARRKDGNYYYAMDYWGQGTTVTVSS

SEQ ID NO: 265 - protein, 121 aa, synthetic construct
  REGION 1..121: note = Description of Artificial Sequence: Synthetic polypeptide
QVQLVESGGGLIQPGGSLRLSCAASGFTFTNYAMSWVRQARGQRLEWVSTITSGGSYIYYLDSVKGRFTISRDNSKSTLYMELSSLRSEDTAVYYCARRKDGNYYYAMDYWGQGTTVTVSS

SEQ ID NO: 266 - protein, 121 aa, synthetic construct
  REGION 1..121: note = Description of Artificial Sequence: Synthetic polypeptide
QVQLVQSGSELKKPGASVKVSCKASGFTFTNYAMSWVRQAPGKRLEWVSTITSGGSYIYYLDSVKGRFTISRENAKSTLYLQMNSLRTEDTALYNCARRKDGNYYYAMDYWGQGTTVTVSS

SEQ ID NO: 267 - protein, 121 aa, synthetic construct
  REGION 1..121: note = Description of Artificial Sequence: Synthetic polypeptide
QVQLVQSGSELKKPGASVKVSCKASGFTFTNYAMSWVRQAPGKRLEWVATITSGGSYIYYLDSVKGRFTISRENAKSTLYLQMNSLRTEDTALYYCARRKDGNYYYAMDYWGQGTTVTVSS

SEQ ID NO: 268 - protein, 121 aa, synthetic construct
  REGION 1..121: note = Description of Artificial Sequence: Synthetic polypeptide
EVQLLQSGAEVKKPGASVKVSCKASGFTFTNYAMSWVRQAPGQRLEWVATITSGGSYIYYLDSVKGRFTISRDNAKSTLHLQMNSLRAEDTAVYNCARRKDGNYYYAMDYWGQGTTVTVSS

SEQ ID NO: 269 - protein, 121 aa, synthetic construct
  REGION 1..121: note = Description of Artificial Sequence: Synthetic polypeptide
EVQLLQSGAEVKKPGASVKVSCKASGFTFTNYAMSWVRQAPGQRLEWVATITSGGSYIYYLDSVKGRFTISRDNAKSTLHLQMNSLRAEDTAIYYCARRKDGNYYYAMDYWGQGTTVTVSS

SEQ ID NO: 270 - protein, 121 aa, synthetic construct
  REGION 1..121: note = Description of Artificial Sequence: Synthetic polypeptide
EVMLLQSGAEVKKPGASVKVSCKASGFTFTNYAMSWVRQAPGQRLEWVATITSGGSYIYYLDSVKGRFTISRDNAKSTLHLQMNSLRAEDTAVYYCARRKDGNYYYAMDYWGQGTTVTVSS

SEQ ID NO: 271 - protein, 111 aa, synthetic construct
  REGION 1..111: note = Description of Artificial Sequence: Synthetic polypeptide
DIVLTQSPASLAVSLGQRATISCRASESVDSYGNSFIHWYQQKAGQPPKLLIYRASNLESGIPARFSGSGSRTDFTLTINPVEADDVATYYCQQSYEDPWTFGGGTKLEIK

SEQ ID NO: 272 - protein, 111 aa, synthetic construct
  REGION 1..111: note = Description of Artificial Sequence: Synthetic polypeptide
  VARIANT 108: note = X can be any amino acid
DIVLTQSPATLSLSPGERATLSCRASESVDSYGNSFIHWYQQKPGQPPKLLIYRASNLESGIPARFSGSGSRTDFTLTISSLEPEDFAVYYCQQSYEDPWTFGGGTKXEIK

SEQ ID NO: 273 - protein, 111 aa, synthetic construct
  REGION 1..111: note = Description of Artificial Sequence: Synthetic polypeptide
  VARIANT 108: note = X can be any amino acid
DIVLTQSPSSLSASVGDRVTITCRASESVDSYGNSFIHWYQQKPGQPPKLLIYRASNLESGIPARFSGSGSRTDFTLTISSLQPEDFATYYCQQSYEDPWTFGGGTKXEIK

SEQ ID NO: 274 - protein, 111 aa, synthetic construct
  REGION 1..111: note = Description of Artificial Sequence: Synthetic polypeptide
  VARIANT 108: note = X can be any amino acid
DIVLTQSPDFQSVTPKEKVTITCRASESVDSYGNSFIHWYQQKPGQPPKLLIYRASNLESGIPARFSGSGSRTDFTLTISSLEAEDAATYYCQQSYEDPWTFGGGTKXEIK

SEQ ID NO: 275 - protein, 111 aa, synthetic construct
  REGION 1..111: note = Description of Artificial Sequence: Synthetic polypeptide
  VARIANT 108: note = X can be any amino acid
DIVLTQTPLSLSVTPGQPASISCRASESVDSYGNSFIHWYQQKPGQPPKLLIYRASNLESGIPARFSGSGSRTDFTLKISRVEAEDVGVYYCQQSYEDPWTFGGGTKXEIK

SEQ ID NO: 276 - protein, 5 aa, synthetic construct
  REGION 1..5: note = Description of Artificial Sequence: Synthetic peptide
TYGMS

SEQ ID NO: 277 - protein, 17 aa, synthetic construct
  REGION 1..17: note = Description of Artificial Sequence: Synthetic peptide
WMNTYSGVTTYADDFKG

SEQ ID NO: 278 - protein, 13 aa, synthetic construct
  REGION 1..13: note = Description of Artificial Sequence: Synthetic peptide
EGYVFDDYYATDY

SEQ ID NO: 279 - protein, 16 aa, synthetic construct
  REGION 1..16: note = Description of Artificial Sequence: Synthetic peptide
RSSQNIVHSDGNTYLE

SEQ ID NO: 280 - protein, 7 aa, synthetic construct
  REGION 1..7: note = Description of Artificial Sequence: Synthetic peptide
KVSNRFS

SEQ ID NO: 281 - protein, 9 aa, synthetic construct
  REGION 1..9: note = Description of Artificial Sequence: Synthetic peptide
FQGSHVPLT

SEQ ID NO: 282 - protein, 122 aa, synthetic construct
  REGION 1..122: note = Description of Artificial Sequence: Synthetic polypeptide
QIQLVQSGPELKKPGETVKISCKASGYTFTTYGMSWVKQAPGKGLKWMGWMNTYSGVTTYADDFKGRFAFSLETSASTAYMQIDNLKNEDTATYFCAREGYVFDDYYATDYWGQGTSVTVSS

SEQ ID NO: 283 - protein, 112 aa, synthetic construct
  REGION 1..112: note = Description of Artificial Sequence: Synthetic polypeptide
DVLMTQTPLSLPVSLGDQASISCRSSQNIVHSDGNTYLEWYLQKPGQSPKLLIYKVSNRFSGVPDRFSGSGSGTDFTLKISRVEAEDLGIYYCFQGSHVPLTFGAGTKLELK

SEQ ID NO: 284 - protein, 5 aa, synthetic construct
  REGION 1..5: note = Description of Artificial Sequence: Synthetic peptide
KYDIN

SEQ ID NO: 285 - protein, 17 aa, synthetic construct
  REGION 1..17: note = Description of Artificial Sequence: Synthetic peptide
WIFPGDGRTDYNEKFKG

SEQ ID NO: 286 - protein, 7 aa, synthetic construct
  REGION 1..7: note = Description of Artificial Sequence: Synthetic peptide
YGPAMDY

SEQ ID NO: 287 - protein, 16 aa, synthetic construct
  REGION 1..16: note = Description of Artificial Sequence: Synthetic peptide
RSSQTIVHSNGDTYLD

SEQ ID NO: 288 - protein, 7 aa, synthetic construct
  REGION 1..7: note = Description of Artificial Sequence: Synthetic peptide
KVSNRFS

SEQ ID NO: 289 - protein, 9 aa, synthetic construct
  REGION 1..9: note = Description of Artificial Sequence: Synthetic peptide
FQGSHVPYT

SEQ ID NO: 290 - protein, 135 aa, synthetic construct
  REGION 1..135: note = Description of Artificial Sequence: Synthetic polypeptide
MGWSWVFLFLLSVTAGVHSQVHLQQSGPELVKPGASVKLSCKASGYTFTKYDINWVRQRPEQGLEWIGWIFPGDGRTDYNEKFKGKATLTTDKSSSTAYMEVSRLTSEDSAVYFCARYGPAMDYWGQGTSVTVAS

SEQ ID NO: 291 - protein, 131 aa, synthetic construct
  REGION 1..131: note = Description of Artificial Sequence: Synthetic polypeptide
MKLPVRLLVLMFWIPASSSDVLMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPYTFGGGTKLEIK

(19) United States
(12) Patent Application Publication
(10) Pub. No.: US 2023/0304095 A1
(43) Pub. Date: Sep. 28, 2023

(54) TL1A THERAPY COMPOSITIONS AND METHODS OF TREATMENT THEREWITH

(71) Applicant: CEDARS-SINAI MEDICAL CENTER, Los Angeles, CA (US)

(72) Inventors: Dermot P. MCGOVERN, Los Angeles, CA (US); Janine BILSBOROUGH, Adelaide (AU); Stephan R. TARGAN, Santa Monica, CA (US); Alka POTDAR, Cumming, GA (US)

(21) Appl. No.: 18/334,221

(22) Filed: Jun. 13, 2023

Related U.S. Application Data
(63) Continuation of application No. PCT/US2021/064406, filed on Dec. 20, 2021.
(60) Provisional application No. 63/128,749, filed on Dec. 21, 2020.

(51) Int. Cl. C12Q 1/6883; C07K 16/28; A61P 1/00

(52) U.S. Cl. CPC C12Q 1/6883; C07K 16/2875; A61P 1/00; A61K 2039/505

(57) ABSTRACT

Disclosed herein are methods, kits and compositions for treating an inflammatory disease or condition, or fibrosis in a subject that has been determined to have increased fold-change in Tumor necrosis factor (TNF)-like cytokine 1A (TL1A) expression based, at least partially, on a presence of a combination of genotypes detected in a sample obtained from the subject. In some embodiments, the combination of genotypes is significantly associated with the increased fold-change in TL1A, and in some cases, may also be predictive of severe forms of the inflammatory disease or condition. In some embodiments, the inflammatory disease or condition is an inflammatory bowel disease, such as Crohn's disease or ulcerative colitis.

### CROSS-REFERENCE

This application is continuation of International Application No. PCT/US2021/064406, filed Dec. 20, 2021, which claims the benefit of U.S. Provisional Application No. 63/128,749, filed Dec. 21, 2020, each of which is incorporated herein by reference in its entirety.

### INCORPORATION BY REFERENCE OF SEQUENCE LISTING

The present application is being filed along with a Sequence Listing in electronic format. The Sequence Listing is provided as a file entitled 56884-741_301_SL.xml, created May 9, 2023, which is 370,677 bytes in size. The information in the electronic format of the Sequence Listing is incorporated by reference in its entirety.

### BACKGROUND

Inflammatory bowel disease (IBD) has two common forms, Crohn's disease (CD) and ulcerative colitis (UC), which are chronic, relapsing inflammatory disorders of the gastrointestinal tract. These diseases are prevalent, with about 1.86 billion people diagnosed globally with UC, and about 1.3 million people diagnosed globally with CD. Each of these forms varies in severity and have various sub-clinical phenotypes that are present in some CD and UC patients. There are a limited number of therapies available for IBD patients, and a significant number of them either do not respond to induction of therapies currently available, or experience a loss of response during treatment. Selecting a therapy that is appropriate for any individual patient at any given stage of their disease is complicated by each individual's genetic predisposition.

### SUMMARY

The inflammatory bowel diseases (IBD), including Crohn's disease (CD) and ulcerative colitis (UC), are chronic inflammatory diseases of the gastrointestinal tract of unknown pathogenesis. Familial aggregation of IBD implicates genetic background in the development of IBD. Dysregulated mucosal immune response to microbes in genetically susceptible individuals is thought to be the pathogenic mechanism of IBD.

Genome Wide Association Studies (GWAS) have enabled scientists to identify genetic variants in certain gene loci that are associated with IBD and sub-clinical phenotypes of IBD. GWAS compare the allele frequency in a given population of a particular genetic variant between unrelated cases and controls, each case representing a patient with IBD and each control representing an individual without IBD. GWAS, the Immunochip, and their meta-analysis have enabled the discovery of over 200 single nucleotide polymorphisms (SNPs) associated with IBD (CD or UC).

The first GWAS on IBD identified TNFSF15 as an IBD locus containing several SNPs associated with IBD. TNFSF15 protein, also known as tumor necrosis factor (TNF)-like cytokine 1A (TL1A), is a proinflammatory molecule which stimulates proliferation and effector functions of CD8 (+) cytotoxic T cells as well as Th1, Th2, and Th17 cells in the presence of TCR stimulation. TL1A is believed to be involved in the pathogenesis of IBD by bridging the innate and adaptive immune response, modulating adaptive immunity by augmenting Th1, Th2, and Th17 effector cell function, and T-cell accumulation and immunopathology of inflamed tissue. Studies have demonstrated that patients with IBD who carry certain risk alleles (SNPs) at the TNFSF15 show an increase TL1A expression and are more likely to develop severe forms of IBD, as compared to individuals who do not carry the risk alleles. These findings suggest that inhibiting TL1A expression or activity may be a promising therapeutic strategy in a variety of T cell-dependent autoimmune diseases, including IBD. These findings also suggest that certain TNFSF15 genotypes in patients that confer a risk of increase TL1A expression or severe forms of disease may prove useful in the prognosis, diagnosis and treatment of these individuals.

The present application discloses polymorphisms at various gene loci, and genotypes, associated with inflammatory diseases or conditions or fibrotic or fibrostenotic disease. In some embodiments, the polymorphisms and genotypes are associated with increased TL1A fold-change expression. The polymorphisms and genotypes disclosed herein may be useful for identifying subjects in need of a treatment of an inflammatory disease or condition or fibrotic or fibrostenotic disease with an inhibitor of TL1A expression of activity. As such, the present application further discloses methods of treatment of a subject with an inhibitor of TL1A expression or activity, provided one of the polymorphisms or genotypes is detected in a sample obtained from a subject. Further disclosed, are methods to characterize an inflammatory disease or condition or fibrotic or fibrostenotic disease of a subject based on the polymorphisms or genotypes detected in a sample obtained from the subject. Methods of detection of the polymorphisms, compositions and kits used in the detection of the polymorphisms and genotypes are also provided.

Aspects disclosed herein provide methods of treating a subject with an inflammatory disease or condition, the method comprising: administering a therapeutically effective amount of an inhibitor of TL1A expression or activity to the subject that has been determined to have an increased fold-change in TL1A expression based on detecting, in a sample obtained from the subject, a combination of genotypes that is associated with the increased fold-change in TL1A expression with a P value of at most about 10⁻³, wherein the increased fold-change in TL1A expression is relative to a baseline expression of TL1A in a reference subject. In some embodiments, the reference subject is a subject that (i) does not have the inflammatory disease or condition, or (ii) has the inflammatory disease or condition, but does not have the combination of genotypes. In some embodiments, the increased fold-change in TL1A expression comprises an increase of greater than or equal to about 20 fold-change in TL1A expression relative to the baseline expression of TL1A in the reference subject. In some embodiments, the increased fold-change in TL1A expression comprises an increase of greater than or equal to about 40 fold-change in TL1A expression relative to the baseline expression of TL1A in the reference subject. In some embodiments, the increased fold-change in TL1A expression comprises an increase of greater than or equal to about 90 fold-change in TL1A expression relative to the baseline expression of TL1A in the reference subject. In some embodiments, the combination of genotypes comprises homozygous “G” at rs6478109, or a polymorphism in LD therewith as determined by an r² of at least 0.80. In some embodiments, the combination of genotypes comprises: (i) a homozygous genotype at a TNFSF15 gene locus; and (ii) a heterozygous or homozygous genotype at an ETS1 gene locus, a LY86 gene locus, or a SCUBE1 gene locus. In some embodiments, the homozygous genotype at the TNFSF15 gene locus is at a polymorphism comprising rs6478109, or a polymorphism in LD therewith as determined by an r² of at least 0.80. In some embodiments, the homozygous genotype at the TNFSF15 gene locus comprises a “G” at rs6478109, or the polymorphism in LD therewith as determined by an r² of at least 0.80. In some embodiments, the heterozygous or homozygous genotype at the ETS1 gene locus is at a polymorphism comprising rs10790957, or a polymorphism in LD therewith as determined by an r² of at least 0.80. In some embodiments, the genotype at the ETS1 gene locus comprises a “G” at rs10790957, or the polymorphism in LD therewith as determined by an r² of at least 0.80. In some embodiments, the heterozygous or homozygous genotype at the LY86 gene locus is at a polymorphism comprising rs6921610, or a polymorphism in LD therewith as determined by an r² of at least 0.80. In some embodiments, the genotype at the LY86 gene locus comprises a “G” at rs6921610, or the polymorphism in LD therewith as determined by an r² of at least 0.80. In some embodiments, the heterozygous or homozygous genotype at the SCUBE1 gene locus is at a polymorphism comprising rs6003160, or a polymorphism in LD therewith as determined by an r² of at least 0.80. In some embodiments, the genotype at the SCUBE1 gene locus comprises a “G” at rs6003160, or the polymorphism in LD therewith as determined by an r² of at least 0.80. In some embodiments, (i) the heterozygous or homozygous genotype at the ETS1 gene locus is at a polymorphism comprising rs10790957, or a polymorphism in LD therewith; (ii) the heterozygous or homozygous genotype at the LY86 gene locus is at a polymorphism comprising rs6921610, or a polymorphism in LD therewith; and (iii) the heterozygous or homozygous genotype at the SCUBE1 gene locus is at a polymorphism comprising rs6003160, or a polymorphism in LD therewith, wherein the LD is determined by an r² of at least 0.80. In some embodiments, (i) the genotype at the ETS1 gene locus comprises a “G” at rs10790957 or the polymorphism in LD therewith as determined by an r² of at least 0.80; (ii) the genotype at the LY86 gene locus comprises a “G” at rs6921610 or the polymorphism in LD therewith as determined by an r² of at least 0.80; and (iii) the genotype at the SCUBE1 gene locus comprises a “G” at rs6003160 or the polymorphism in LD therewith as determined by an r² of at least 0.80. In some embodiments, the combination of genotypes comprises: (i) a heterozygous genotype at a TNFSF15 gene locus; and (ii) a heterozygous or homozygous genotype at an ARHGAP15 gene locus. In some embodiments, the heterozygous genotype at the TNFSF15 gene locus is at a polymorphism comprising rs6478109, or a polymorphism in LD therewith as determined by an r² of at least 0.80. In some embodiments, the heterozygous genotype at the TNFSF15 gene locus comprises a “G” at rs6478109, or the polymorphism in LD therewith as determined by an r² of at least 0.80. In some embodiments, the heterozygous or homozygous genotype at the ARHGAP15 gene locus is at a polymorphism comprising rs6757588, or a polymorphism in LD therewith as determined by an r² of at least 0.80. In some embodiments, the heterozygous or homozygous genotype at the ARHGAP15 gene locus comprises a “G” at rs6757588, or the polymorphism in LD therewith as determined by an r² of at least 0.80. In some embodiments, (i) the heterozygous genotype at the TNFSF15 gene locus is at a polymorphism comprising rs6478109, or a polymorphism in LD therewith as determined by an r² of at least 0.80; and (ii) the heterozygous or homozygous genotype at the ARHGAP15 gene locus is at a polymorphism comprising rs6757588, or a polymorphism in LD therewith as determined by an r² of at least 0.80. In some embodiments, (i) the heterozygous genotype at the TNFSF15 gene locus comprises a “G” at rs6478109, or the polymorphism in LD therewith as determined by an r² of at least 0.80; and (ii) the heterozygous or homozygous genotype at the ARHGAP15 gene locus comprises a “G” at rs6757588, or the polymorphism in LD therewith as determined by an r² of at least 0.80. In some embodiments, the methods further comprise characterizing the inflammatory disease or condition as an inflammatory bowel disease. In some embodiments, the inflammatory bowel disease comprises Crohn's disease. In some embodiments, the inflammatory bowel disease comprises ulcerative colitis. In some embodiments, the TL1A expression comprises TL1A protein expression. In some embodiments, the increased fold-change in TL1A expression is determined by: (i) introducing immune complex to peripheral blood mononuclear cells (PBMCs) in vitro under conditions suitable to stimulate the PBMCs, wherein the PBMCs were obtained from subjects with the inflammatory disease or condition; (ii) measuring by ELISA, the TL1A expression at a plurality of sequential time points comprising a first time point, a second time point and a third time point; and (iii) calculating the increased fold-change in TL1A expression by dividing the TL1A expression at the second time point and the TL1A expression at the third time point by the TL1A expression at the first time point. In some embodiments, the first time point is 6 hours following the introducing in (a), the second time point is 24 hours following the introducing in (a), and the third time point is 72 hours following the introducing in (a).

In one aspect, are methods of treating a subject with an inflammatory disease or condition, or fibrostenotic or fibrotic disease comprising administering a therapeutically effective amount of an inhibitor of TL1A expression or activity to the subject, provided a presence of a polymorphism located at a gene locus comprising LY86, ETS1, ARHGAP15, or SCUBE1 is detected in a sample obtained from the subject. In some embodiments, the polymorphism at the gene locus comprising LY86, ETS1, ARHGAP15, or SCUBE1 comprises rs6921610, rs10790957, rs6757588, or rs6003160, respectively, or any polymorphism in linkage disequilibrium therewith. In some embodiments, the polymorphism at the gene locus comprising ETS1 comprises rs11606640, rs73029052, rs11600915, rs61909068, rs12294634, rs73029062, rs11600746, rs61909072, or rs56086356, or any polymorphism in linkage disequilibrium therewith. In some embodiments, the polymorphism at the gene locus comprising LY86 comprises rs3851519 or any polymorphism in linkage disequilibrium therewith. In some embodiments, the polymorphism at the gene locus comprising LY86 comprises a “G” allele at nucleobase 700 within rs6921610. In some embodiments, the polymorphism at the gene locus comprising LY86 comprises a “A” allele at nucleobase 248 within rs3851519. In some embodiments, the polymorphism at the gene locus comprising ETS1 comprises a “G” allele at nucleobase 501 within rs10790957. In some embodiments, the polymorphism at the gene locus comprising ETS1 comprises a “A” allele at nucleobase 301 within rs11606640. In some embodiments, the polymorphism at the gene locus comprising ETS1 comprises a “A” allele at nucleobase 251 within rs73029052. In some embodiments, the polymorphism at the gene locus comprising ETS1 comprises a “G” allele at nucleobase 301 within rs11600915. In some embodiments, the polymorphism at the gene locus comprising ETS1 comprises a “G” allele at nucleobase 251 within rs61909068. In some embodiments, the polymorphism at the gene locus comprising ETS1 comprises a “A” allele at nucleobase 323 within rs12294634. In some embodiments, the polymorphism at the gene locus comprising ETS1 comprises a “G” allele at nucleobase 251 within rs73029062. In some embodiments, the polymorphism at the gene locus comprising ETS1 comprises a “G” allele at nucleobase 301 within rs11600746. In some embodiments, the polymorphism at the gene locus comprising ETS1 comprises a “A” allele at nucleobase 251 within rs61909072. In some embodiments, the polymorphism at the gene locus comprising ETS1 comprises a “C” allele at nucleobase 501 within rs56086356. In some embodiments, the polymorphism at the gene locus comprising ARHGAP15 comprises a “G” allele at nucleobase 501 within rs6757588. In some embodiments, the polymorphism at the gene locus comprising SCUBE1 comprises a “G” allele at nucleobase 501 within rs6003160. In some embodiments, the polymorphism at the gene locus comprising LY86 comprises SEQ ID NO: 33. In some embodiments, the polymorphism at the gene locus comprising LY86 comprises SEQ ID NO: 80. In some embodiments, the polymorphism at the gene locus ETS1 comprises SEQ ID NO: 34. In some embodiments, the gene locus ETS1 comprises SEQ ID NO: 73. In some embodiments, the polymorphism at the gene locus comprising ETS1 comprises SEQ ID NO: 74. In some embodiments, the polymorphism at the gene locus comprising ETS1 comprises SEQ ID NO: 75. In some embodiments, the polymorphism at the gene locus comprising ETS1 comprises SEQ ID NO: 76. In some embodiments, the polymorphism at the gene locus comprising ETS1 comprises SEQ ID NO: 77. In some embodiments, the polymorphism at the gene locus comprising ETS1 comprises SEQ ID NO: 78. In some embodiments, the polymorphism at the gene locus comprising ETS1 comprises SEQ ID NO: 79. In some embodiments, the polymorphism at the gene locus comprising ETS1 comprises SEQ ID NO: 81. In some embodiments, the polymorphism at the gene locus comprising ETS1 comprises SEQ ID NO: 82. In some embodiments, the polymorphism at the gene locus comprising ARHGAP15 comprises SEQ ID NO: 35. In some embodiments, the polymorphism at the gene locus comprising SCUBE1 comprises SEQ ID NO: 36. In some embodiments, a polymorphism located at a TNFSF15 locus comprising rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, or any polymorphism in linkage disequilibrium therewith, is detected in the sample obtained from the subject. In some embodiments, the polymorphism comprising rs6478109 comprises a “G” allele at nucleobase 501 within rs6478109. In some embodiments, the polymorphism comprising rs7848647 comprises a “G” allele at nucleobase 501 within rs7848647. In some embodiments, the polymorphism comprising rs201292440 comprises an insertion of a nucleic acid, I, at nucleobase 501 within rs201292440. In some embodiments, the polymorphism comprising rs7869487 comprises an “A” allele at nucleobase 501 within rs7869487. In some embodiments, the polymorphism comprising rs4366152 comprises a “G” allele at nucleobase 501 within rs4366152. In some embodiments, the polymorphism comprising rs6478108 comprises an “A” allele at nucleobase 501 within rs6478108. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs10114470 comprises a “G” allele at nucleobase 501 within rs10114470. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs4574921 comprises an “A” allele at nucleobase 501 within rs4574921. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism at the TNFSF15 locus is represented with an “N” within any one of SEQ ID NOS: 1-32. In some embodiments, the polymorphism comprises a polymorphism of Table 3. In some embodiments, the polymorphism comprises a polymorphism of Tables 3, 4, or 5. In some embodiments, two copies of the polymorphism are detected in the sample obtained from the subject. In some embodiments, one copy of the polymorphism is detected in the sample obtained from the subject. In some embodiments, the polymorphism is associated with a disease phenotype comprising non-stricturing/non-penetrating, stricturing, stricturing and penetrating, or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the polymorphism is associated with an increase in expression of TL1A. In some embodiments, two copies of the polymorphism located at the TNFSF15 gene locus and the polymorphism located at a gene locus comprising LY86, ETS1, or SCUBE1 detected in the sample obtained from the subject is indicative of the subject having increase TL1A fold-change. In some embodiments, one copy of the polymorphism located at the TNFSF15 gene locus and the polymorphism located at the ARHGAP15 gene locus detected in the sample obtained from the subject is indicative of the subject having an increase TL1A fold-change. In some embodiments, the increase in TL1A fold-change comprises an increase of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, 60-fold, 70-fold, 80-fold, 90-fold, or 100-fold or more between the sample obtained from the subject and an expression of TL1A in an individual who does not express the polymorphism. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A. In some embodiments, the polymorphism is detected by using an assay comprising DNA sequencing, a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to about 10 contiguous nucleobases of any one of SEQ ID NOS: 1-36 under standard hybridization conditions. In some embodiments, the standard hybridization conditions comprise an annealing temperature between about 30° C. and about 65° C. In some embodiments, the nucleic acid sequence comprises any one of SEQ ID NOS: 37-72. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least 10 but not more than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588 or rs6003160, wherein one of the nucleobases is at position 501. In some embodiments, the nucleic acid amplification assay comprises amplification of DNA from the subject with a pair of primers capable of amplifying at least about 10 and less than 50 contiguous nucleobases within any one of SEQ ID NOS: 1-36. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, wherein the subject is non-responsive to a therapy comprising anti-TNF alpha therapy, anti-a4-b7 therapy (vedolizumab), anti-IL12p40 therapy (ustekinumab), Thalidomide, or Cytoxan.

In another aspect, are methods comprising: a) obtaining a sample from a subject with an inflammatory disease or condition, or fibrostenotic or fibrotic disease; b) assaying to detect in the sample a presence of a polymorphism located at a gene locus comprising LY86, ETS1, ARHGAP15, or SCUBE1; and c) administering a therapeutically effective amount of an inhibitor of TL1A expression or activity to the subject, provided the presence of the polymorphism is detected in the sample obtained from the subject. In some embodiments, the polymorphism at the gene locus comprising LY86, ETS1, ARHGAP15, or SCUBE1 comprises rs6921610, rs10790957, rs6757588, or rs6003160, respectively, or any polymorphism in linkage disequilibrium therewith. In some embodiments, the polymorphism at the gene locus comprising ETS1 comprises rs11606640, rs73029052, rs11600915, rs61909068, rs12294634, rs73029062, rs11600746, rs61909072, or rs56086356, or any polymorphism in linkage disequilibrium therewith. In some embodiments, the polymorphism at the gene locus comprising LY86 comprises rs3851519 or any polymorphism in linkage disequilibrium therewith. In some embodiments, the polymorphism comprises a polymorphism of Table 3. In some embodiments, the polymorphism comprises a polymorphism of Tables 3, 4, or 5. In some embodiments, the polymorphism at the gene locus comprising LY86 comprises a “G” allele at nucleobase 501 within rs6921610. In some embodiments, the polymorphism at the gene locus comprising LY86 comprises a “A” allele at nucleobase 248 within rs3851519. In some embodiments, the polymorphism at the gene locus comprising ETS1 comprises a “G” allele at nucleobase 501 within rs10790957. In some embodiments, the polymorphism at the gene locus comprising ETS1 comprises a “A” allele at nucleobase 301 within rs11606640. In some embodiments, the polymorphism at the gene locus comprising ETS1 comprises a “A” allele at nucleobase 251 within rs73029052. In some embodiments, the polymorphism at the gene locus comprising ETS1 comprises a “G” allele at nucleobase 301 within rs11600915. In some embodiments, the polymorphism at the gene locus comprising ETS1 comprises a “G” allele at nucleobase 251 within rs61909068. In some embodiments, the polymorphism at the gene locus comprising ETS1 comprises a “A” allele at nucleobase 323 within rs12294634. In some embodiments, the polymorphism at the gene locus comprising ETS1 comprises a “G” allele at nucleobase 251 within rs73029062. In some embodiments, the polymorphism at the gene locus comprising ETS1 comprises a “G” allele at nucleobase 301 within rs11600746. In some embodiments, the polymorphism at the gene locus comprising ETS1 comprises a “A” allele at nucleobase 251 within rs61909072. In some embodiments, the polymorphism at the gene locus comprising ETS1 comprises a “C” allele at nucleobase 501 within rs56086356. In some embodiments, the polymorphism at the gene locus comprising ARHGAP15 comprises a “G” allele at nucleobase 501 within rs6757588. In some embodiments, the polymorphism at the gene locus comprising SCUBE1 comprises a “G” allele at nucleobase 501 within rs6003160. In some embodiments, the polymorphism at the gene locus comprising LY86 comprises SEQ ID NO: 33. In some embodiments, the polymorphism at the gene locus comprising LY86 comprises SEQ ID NO: 80. In some embodiments, the polymorphism at the gene locus ETS1 comprises SEQ ID NO: 34. In some embodiments, the gene locus ETS1 comprises SEQ ID NO: 73. In some embodiments, the polymorphism at the gene locus comprising ETS1 comprises SEQ ID NO: 74. In some embodiments, the polymorphism at the gene locus comprising ETS1 comprises SEQ ID NO: 75. In some embodiments, the polymorphism at the gene locus comprising ETS1 comprises SEQ ID NO: 76. In some embodiments, the polymorphism at the gene locus comprising ETS1 comprises SEQ ID NO: 77. In some embodiments, the polymorphism at the gene locus comprising ETS1 comprises SEQ ID NO: 78. In some embodiments, the polymorphism at the gene locus comprising ETS1 comprises SEQ ID NO: 79. In some embodiments, the polymorphism at the gene locus comprising ETS1 comprises SEQ ID NO: 81. In some embodiments, the polymorphism at the gene locus comprising ETS1 comprises SEQ ID NO: 82. In some embodiments, the polymorphism at the gene locus comprising ARHGAP15 comprises SEQ ID NO: 35. In some embodiments, the polymorphism at the gene locus comprising SCUBE1 comprises SEQ ID NO: 36. In some embodiments, a polymorphism located at a TNFSF15 locus comprising rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, or any polymorphism in linkage disequilibrium therewith, is detected in the sample obtained from the subject. In some embodiments, the polymorphism comprising rs6478109 comprises a “G” allele at nucleobase 501 within rs6478109. In some embodiments, the polymorphism comprising rs7848647 comprises a “G” allele at nucleobase 501 within rs7848647. In some embodiments, the polymorphism comprising rs201292440 comprises an insertion of a nucleic acid, I, at nucleobase 501 within rs201292440. In some embodiments, the polymorphism comprising rs7869487 comprises an “A” allele at nucleobase 501 within rs7869487. In some embodiments, the polymorphism comprising rs4366152 comprises a “G” allele at nucleobase 501 within rs4366152. In some embodiments, the polymorphism comprising rs6478108 comprises an “A” allele at nucleobase 501 within rs6478108. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs10114470 comprises a “G” allele at nucleobase 501 within rs10114470. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs4574921 comprises an “A” allele at nucleobase 501 within rs4574921. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism at the TNFSF15 locus is represented with an “N” within any one of SEQ ID NOS: 1-32. In some embodiments, two copies of the polymorphism are detected in the sample obtained from the subject. In some embodiments, one copy of the polymorphism is detected in the sample obtained from the subject. In some embodiments, the polymorphism is associated with a disease phenotype comprising non-stricturing/non-penetrating, stricturing, stricturing and penetrating, or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the polymorphism is associated with an increase in expression of TL1A. In some embodiments, two copies of the polymorphism located at the TNFSF15 gene locus and the polymorphism located at a gene locus comprising LY86, ETS1, or SCUBE1 detected in the sample obtained from the subject is indicative of the subject having increase TL1A fold-change. In some embodiments, one copy of the polymorphism located at the TNFSF15 gene locus and the polymorphism located at the ARHGAP15 gene locus detected in the sample obtained from the subject is indicative of the subject having an increase TL1A fold-change. In some embodiments, the increase in TL1A fold-change comprises an increase of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, 60-fold, 70-fold, 80-fold, 90-fold, 100-fold, 60-fold, 70-fold, 80-fold, 90-fold, or 100-fold, or more between the sample obtained from the subject and an expression of TL1A in an individual who does not express the polymorphism. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A. In some embodiments, the polymorphism is detected by using an assay comprising DNA sequencing, a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to about 10 contiguous nucleobases of any one of SEQ ID NOS: 1-36 under standard hybridization conditions. In some embodiments, the standard hybridization conditions comprise an annealing temperature between about 30° C. and about 65° C. In some embodiments, the nucleic acid sequence comprises any one of SEQ ID NOS: 37-72. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least 10 but not more than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588 or rs6003160, wherein one of the nucleobases is at position 501. In some embodiments, the nucleic acid amplification assay comprises amplification of DNA from the subject with a pair of primers capable of amplifying at least about 10 and less than 50 contiguous nucleobases within any one of SEQ ID NOS: 1-36. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, wherein the subject is non-responsive to a therapy comprising anti-TNF alpha therapy, anti-a4-b7 therapy (vedolizumab), anti-IL12p40 therapy (ustekinumab), Thalidomide, or Cytoxan.

In another aspect, are methods of treating a subject with an inflammatory disease or condition, or fibrostenotic or fibrotic disease, the method comprising administering a therapeutically effective amount of an inhibitor of TL1A expression or activity to the subject, provided at least one copy of a polymorphism located at a TNFSF15 locus, and a polymorphism located at a gene locus comprising LY86, ETS1, or SCUBE1 or a polymorphism located at a gene locus comprising ARHGAP15, are detected in a sample obtained from the subject. In some embodiments, the polymorphism comprises a polymorphism of Table 3. In some embodiments, the polymorphism comprises a polymorphism of Tables 3, 4, or 5. In some embodiments, the polymorphism at the TNFSF15 locus comprises rs6478109, rs7848647, rs201292440, rs7869487, rs4366152, rs6478108, rs1407308, rs7866342, rs7030574, rs10114470, rs4979464, rs3810936, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, or any polymorphism in linkage disequilibrium therewith. In some embodiments, the polymorphism comprising rs6478109 comprises a “G” allele at nucleobase 501 within rs6478109. In some embodiments, the polymorphism comprising rs7848647 comprises a “G” allele at nucleobase 501 within rs7848647. In some embodiments, the polymorphism comprising rs201292440 comprises an insertion of a nucleic acid, I, at nucleobase 501 within rs201292440. In some embodiments, the polymorphism comprising rs7869487 comprises an “A” allele at nucleobase 501 within rs7869487. In some embodiments, the polymorphism comprising rs4366152 comprises a “G” allele at nucleobase 501 within rs4366152. In some embodiments, the polymorphism comprising rs6478108 comprises an “A” allele at nucleobase 501 within rs6478108. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs10114470 comprises a “G” allele at nucleobase 501 within rs10114470. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs4574921 comprises an “A” allele at nucleobase 501 within rs4574921. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism at the TNFSF15 locus is represented with an “N” within any one of SEQ ID NOS: 1-32. In some embodiments, two copies of the polymorphism are detected in the sample obtained from the subject. In some embodiments, one copy of the polymorphism is detected in the sample obtained from the subject. In some embodiments, the polymorphism at the gene locus comprising LY86, ETS1, ARHGAP15, or SCUBE1 comprises rs6921610, rs10790957, rs6757588, or rs6003160, respectively, or any polymorphism in linkage disequilibrium therewith. In some embodiments, the polymorphism at the gene locus comprising ETS1 comprises rs11606640, rs73029052, rs11600915, rs61909068, rs12294634, rs73029062, rs11600746, rs61909072, or rs56086356, or any polymorphism in linkage disequilibrium therewith. In some embodiments, the polymorphism at the gene locus comprising LY86 comprises rs3851519 or any polymorphism in linkage disequilibrium therewith. In some embodiments, the polymorphism at the gene locus comprising LY86 comprises a “G” allele at nucleobase 501 within rs6921610. In some embodiments, the polymorphism at the gene locus comprising LY86 comprises a “A” allele at nucleobase 248 within rs3851519. In some embodiments, the polymorphism at the gene locus comprising ETS1 comprises a “G” allele at nucleobase 501 within rs10790957. In some embodiments, the polymorphism at the gene locus comprising ETS1 comprises a “A” allele at nucleobase 301 within rs11606640. In some embodiments, the polymorphism at the gene locus comprising ETS1 comprises a “A” allele at nucleobase 251 within rs73029052. In some embodiments, the polymorphism at the gene locus comprising ETS1 comprises a “G” allele at nucleobase 301 within rs11600915. In some embodiments, the polymorphism at the gene locus comprising ETS1 comprises a “G” allele at nucleobase 251 within rs61909068. In some embodiments, the polymorphism at the gene locus comprising ETS1 comprises a “A” allele at nucleobase 323 within rs12294634. In some embodiments, the polymorphism at the gene locus comprising ETS1 comprises a “G” allele at nucleobase 251 within rs73029062. In some embodiments, the polymorphism at the gene locus comprising ETS1 comprises a “G” allele at nucleobase 301 within rs11600746. In some embodiments, the polymorphism at the gene locus comprising ETS1 comprises a “A” allele at nucleobase 251 within rs61909072. In some embodiments, the polymorphism at the gene locus comprising ETS1 comprises a “C” allele at nucleobase 501 within rs56086356. In some embodiments, the polymorphism at the gene locus comprising ARHGAP15 comprises a “G” allele at nucleobase 501 within rs6757588. In some embodiments, the polymorphism at the gene locus comprising SCUBE1 comprises a “G” allele at nucleobase 501 within rs6003160. In some embodiments, the polymorphism at the gene locus comprising LY86 comprises SEQ ID NO: 33. In some embodiments, the polymorphism at the gene locus comprising LY86 comprises SEQ ID NO: 80. In some embodiments, the polymorphism at the gene locus ETS1 comprises SEQ ID NO: 34. In some embodiments, the gene locus ETS1 comprises SEQ ID NO: 73. In some embodiments, the polymorphism at the gene locus comprising ETS1 comprises SEQ ID NO: 74. In some embodiments, the polymorphism at the gene locus comprising ETS1 comprises SEQ ID NO: 75. In some embodiments, the polymorphism at the gene locus comprising ETS1 comprises SEQ ID NO: 76. In some embodiments, the polymorphism at the gene locus comprising ETS1 comprises SEQ ID NO: 77. In some embodiments, the polymorphism at the gene locus comprising ETS1 comprises SEQ ID NO: 78. In some embodiments, the polymorphism at the gene locus comprising ETD comprises SEQ ID NO: 79. In some embodiments, the polymorphism at the gene locus comprising ETS1 comprises SEQ ID NO: 81. In some embodiments, the polymorphism at the gene locus comprising ETS1 comprises SEQ ID NO: 82. In some embodiments, the polymorphism at the gene locus comprising ARHGAP15 comprises SEQ ID NO: 35. In some embodiments, the polymorphism at the gene locus comprising SCUBE1 comprises SEQ ID NO: 36. In some embodiments, the polymorphism is associated with a disease phenotype comprising non-stricturing/non-penetrating, stricturing, stricturing and penetrating, or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the polymorphism is associated with an increase in expression of TL1A. In some embodiments, two copies of the polymorphism located at the TNFSF15 gene locus and the polymorphism located at a gene locus comprising LY86, ETS1, or SCUBE1 detected in the sample obtained from the subject is indicative of the subject having increase TL1A fold-change. In some embodiments, one copy of the polymorphism located at the TNFSF15 gene locus and the polymorphism located at the ARHGAP15 gene locus detected in the sample obtained from the subject is indicative of the subject having an increase TL1A fold-change. In some embodiments, the increase in TL1A fold-change comprises an increase of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, 60-fold, 70-fold, 80-fold, 90-fold, 100-fold, 60-fold, 70-fold, 80-fold, 90-fold, or 100-fold, or more between the sample obtained from the subject and an expression of TL1A in an individual who does not express the polymorphism. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A. In some embodiments, the polymorphism is detected by using an assay comprising DNA sequencing, a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to about 10 contiguous nucleobases of any one of SEQ ID NOS: 1-36 under standard hybridization conditions. In some embodiments, the standard hybridization conditions comprise an annealing temperature between about 30° C. and about 65° C. In some embodiments, the nucleic acid sequence comprises any one of SEQ ID NOS: 37-72. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least 10 but not more than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588 or rs6003160, wherein one of the nucleobases is at position 501. In some embodiments, the nucleic acid amplification assay comprises amplification of DNA from the subject with a pair of primers capable of amplifying at least about 10 and less than 50 contiguous nucleobases within any one of SEQ ID NOS: 1-36. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, wherein the subject is non-responsive to a therapy comprising anti-TNF alpha therapy, anti-a4-b7 therapy (vedolizumab), anti-IL12p40 therapy (ustekinumab), Thalidomide, or Cytoxan.

A method of treating a subject with an inflammatory disease or condition, or fibrostenotic or fibrotic disease comprising determining whether the subject has increased TL1A fold-change by performing or having performed an assay on a sample obtained from the subject to detect a presence of a polymorphism located at a gene locus comprising TNFSF15, LY86, ETS1, ARHGAP15, or SCUBE1; and if one copy of a polymorphism at the TNFSF15 gene locus, and at least one copy of a polymorphism at the ARHGAP15 gene locus are detected in the sample obtained from the subject, then administering a therapeutically effective amount of an inhibitor of TL1A expression or activity to the subject; and if two copies of a polymorphism at the TNFSF15 gene locus, and at least one copy of a polymorphism at the LY86, ETS1, or SCUBE1 gene loci are detected in the sample obtained from the subject, then administering a therapeutically effective amount of an inhibitor of TL1A expression or activity to the subject. In some embodiments, the polymorphism comprises a polymorphism of Table 3. In some embodiments, the polymorphism comprises a polymorphism of Tables 3, 4, or 5. In some embodiments, the polymorphism at the TNFSF15 locus comprises rs6478109, rs7848647, rs201292440, rs7869487, rs4366152, rs6478108, rs1407308, rs7866342, rs7030574, rs10114470, rs4979464, rs3810936, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, or any polymorphism in linkage disequilibrium therewith. In some embodiments, the polymorphism comprising rs6478109 comprises a “G” allele at nucleobase 501 within rs6478109. In some embodiments, the polymorphism comprising rs7848647 comprises a “G” allele at nucleobase 501 within rs7848647. In some embodiments, the polymorphism comprising rs201292440 comprises an insertion of a nucleic acid, I, at nucleobase 501 within rs201292440. In some embodiments, the polymorphism comprising rs7869487 comprises an “A” allele at nucleobase 501 within rs7869487. In some embodiments, the polymorphism comprising rs4366152 comprises a “G” allele at nucleobase 501 within rs4366152. In some embodiments, the polymorphism comprising rs6478108 comprises an “A” allele at nucleobase 501 within rs6478108. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs10114470 comprises a “G” allele at nucleobase 501 within rs10114470. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs4574921 comprises an “A” allele at nucleobase 501 within rs4574921. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism at the TNFSF15 locus is represented with an “N” within any one of SEQ ID NOS: 1-32. In some embodiments, two copies of the polymorphism at the LY86, ETS1, ARHGAP15, or SCUBE1 gene loci are detected in the sample obtained from the subject. In some embodiments, one copy of the polymorphism at the LY86, ETS1, ARHGAP15, or SCUBE1 gene loci is detected in the sample obtained from the subject. In some embodiments, the polymorphism at the gene locus comprising LY86, ETS1, ARHGAP15, or SCUBE1 comprises rs6921610, rs10790957, rs6757588, or rs6003160, respectively, or any polymorphism in linkage disequilibrium therewith. In some embodiments, the polymorphism at the gene locus comprising ETS1 comprises rs11606640, rs73029052, rs11600915, rs61909068, rs12294634, rs73029062, rs11600746, rs61909072, or rs56086356, or any polymorphism in linkage disequilibrium therewith. In some embodiments, the polymorphism at the gene locus comprising LY86 comprises rs3851519 or any polymorphism in linkage disequilibrium therewith. In some embodiments, the polymorphism at the gene locus comprising LY86 comprises a “G” allele at nucleobase 501 within rs6921610. In some embodiments, the polymorphism at the gene locus comprising LY86 comprises a “A” allele at nucleobase 248 within rs3851519. In some embodiments, the polymorphism at the gene locus comprising ETS1 comprises a “G” allele at nucleobase 501 within rs10790957. In some embodiments, the polymorphism at the gene locus comprising ETS1 comprises a “A” allele at nucleobase 301 within rs11606640. In some embodiments, the polymorphism at the gene locus comprising ETS1 comprises a “A” allele at nucleobase 251 within rs73029052. In some embodiments, the polymorphism at the gene locus comprising ETS1 comprises a “G” allele at nucleobase 301 within rs11600915. In some embodiments, the polymorphism at the gene locus comprising ETS1 comprises a “G” allele at nucleobase 251 within rs61909068. In some embodiments, the polymorphism at the gene locus comprising ETS1 comprises a “A” allele at nucleobase 323 within rs12294634. In some embodiments, the polymorphism at the gene locus comprising ETS1 comprises a “G” allele at nucleobase 251 within rs73029062. In some embodiments, the polymorphism at the gene locus comprising ETS1 comprises a “G” allele at nucleobase 301 within rs11600746. In some embodiments, the polymorphism at the gene locus comprising ETS1 comprises a “A” allele at nucleobase 251 within rs61909072. In some embodiments, the polymorphism at the gene locus comprising ETS1 comprises a “C” allele at nucleobase 501 within rs56086356. In some embodiments, the polymorphism at the gene locus comprising ARHGAP15 comprises a “G” allele at nucleobase 501 within rs6757588. In some embodiments, the polymorphism at the gene locus comprising SCUBE1 comprises a “G” allele at nucleobase 501 within rs6003160. In some embodiments, the polymorphism at the gene locus comprising LY86 comprises SEQ ID NO: 33. In some embodiments, the polymorphism at the gene locus comprising LY86 comprises SEQ ID NO: 80. In some embodiments, the polymorphism at the gene locus ETS1 comprises SEQ ID NO: 34. In some embodiments, the gene locus ETS1 comprises SEQ ID NO: 73. In some embodiments, the polymorphism at the gene locus comprising ETS1 comprises SEQ ID NO: 74. In some embodiments, the polymorphism at the gene locus comprising ETS1 comprises SEQ ID NO: 75. In some embodiments, the polymorphism at the gene locus comprising ETS1 comprises SEQ ID NO: 76. In some embodiments, the polymorphism at the gene locus comprising ETS1 comprises SEQ ID NO: 77. In some embodiments, the polymorphism at the gene locus comprising ETS1 comprises SEQ ID NO: 78. In some embodiments, the polymorphism at the gene locus comprising ETS1 comprises SEQ ID NO: 79. In some embodiments, the polymorphism at the gene locus comprising ETS1 comprises SEQ ID NO: 81. In some embodiments, the polymorphism at the gene locus comprising ETS1 comprises SEQ ID NO: 82. In some embodiments, the polymorphism at the gene locus comprising ARHGAP15 comprises SEQ ID NO: 35. In some embodiments, the polymorphism at the gene locus comprising SCUBE1 comprises SEQ ID NO: 36. In some embodiments, the polymorphism is associated with a disease phenotype comprising non-stricturing/non-penetrating, stricturing, stricturing and penetrating, or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the polymorphism is associated with an increase in TL1A fold-change. In some embodiments, two copies of the polymorphism located at the TNFSF15 gene locus and the polymorphism located at a gene locus comprising LY86, ETS1, or SCUBE1 detected in the sample obtained from the subject is indicative of the subject having increase TL1A fold-change. In some embodiments, one copy of the polymorphism located at the TNFSF15 gene locus and the polymorphism located at the ARHGAP15 gene locus detected in the sample obtained from the subject is indicative of the subject having an increase TL1A fold-change. In some embodiments, the increase in TL1A fold-change comprises an increase of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, 60-fold, 70-fold, 80-fold, 90-fold, 100-fold, 60-fold, 70-fold, 80-fold, 90-fold, or 100-fold, or more between the sample obtained from the subject and an expression of TL1A in an individual who does not express the polymorphism. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A. In some embodiments, the polymorphism is detected by using an assay comprising DNA sequencing a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to about 10 contiguous nucleobases of any one of SEQ ID NOS: 1-36 under standard hybridization conditions. In some embodiments, the standard hybridization conditions comprise an annealing temperature between about 30° C. and about 65° C. In some embodiments, the nucleic acid sequence comprises any one of SEQ ID NOS: 37-72. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least 10 but not more than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588 or rs6003160, wherein one of the nucleobases is at position 501. In some embodiments, the nucleic acid amplification assay comprises amplification of DNA from the subject with a pair of primers capable of amplifying at least about 10 and less than 50 contiguous nucleobases within any one of SEQ ID NOS: 1-36. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, wherein the subject is non-responsive to a therapy comprising anti-TNF alpha therapy, anti-a4-b7 therapy (vedolizumab), anti-IL12p40 therapy (ustekinumab), Thalidomide, or Cytoxan.

In one aspect, are methods of treating a subject with an inflammatory disease or condition, or fibrostenotic or fibrotic disease, the method comprising administering a therapeutically effective amount of an inhibitor of TL1A expression or activity to the subject, provided one copy of a polymorphism located at a TNFSF15 locus and a polymorphism located at a gene locus comprising ARHGAP15 is detected in a sample obtained from the subject. In some embodiments, the polymorphism at the TNFSF15 locus comprises rs6478109, rs7848647, rs201292440, rs7869487, rs4366152, rs6478108, rs1407308, rs7866342, rs7030574, rs10114470, rs4979464, rs3810936, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, or any polymorphism in linkage disequilibrium therewith. In some embodiments, the polymorphism comprising rs6478109 comprises a “G” allele at nucleobase 501 within rs6478109. In some embodiments, the polymorphism comprising rs7848647 comprises a “G” allele at nucleobase 501 within rs7848647. In some embodiments, the polymorphism comprising rs201292440 comprises an insertion of a nucleic acid, I, at nucleobase 501 within rs201292440. In some embodiments, the polymorphism comprising rs7869487 comprises an “A” allele at nucleobase 501 within rs7869487. In some embodiments, the polymorphism comprising rs4366152 comprises a “G” allele at nucleobase 501 within rs4366152. In some embodiments, the polymorphism comprising rs6478108 comprises an “A” allele at nucleobase 501 within rs6478108. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs10114470 comprises a “G” allele at nucleobase 501 within rs10114470. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs4574921 comprises an “A” allele at nucleobase 501 within rs4574921. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism at the TNFSF15 locus is represented with an “N” within any one of SEQ ID NOS: 1-32. In some embodiments, the polymorphism at the gene locus comprising ARHGAP15 comprises a “G” allele at nucleobase 501 within rs6757588. In some embodiments, the polymorphism at the gene locus comprising ARHGAP15 comprises SEQ ID NO: 36. In some embodiments, the polymorphism is associated with a disease phenotype comprising non-stricturing/non-penetrating, stricturing, stricturing and penetrating, or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the polymorphism is associated with an increase in expression of TL1A. In some embodiments, one copy of the polymorphism located at the TNFSF15 gene locus and the polymorphism located at the ARHGAP15 gene locus detected in the sample obtained from the subject is indicative of the subject having an increase TL1A fold-change. In some embodiments, the increase in TL1A fold-change comprises an increase of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, 60-fold, 70-fold, 80-fold, 90-fold, 100-fold, 60-fold, 70-fold, 80-fold, 90-fold, or 100-fold, or more between the sample obtained from the subject and an expression of TL1A in an individual who does not express the polymorphism. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A. In some embodiments, the polymorphism is detected by using an assay comprising DNA sequencing, a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to about 10 contiguous nucleobases of any one of SEQ ID NOS: 1-36 under standard hybridization conditions. In some embodiments, the standard hybridization conditions comprise an annealing temperature between about 30° C. and about 65° C. In some embodiments, the nucleic acid sequence comprises any one of SEQ ID NOS: 37-72. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least 10 but not more than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588 or rs6003160, wherein one of the nucleobases is at position 501. In some embodiments, the nucleic acid amplification assay comprises amplification of DNA from the subject with a pair of primers capable of amplifying at least about 10 and less than 50 contiguous nucleobases within any one of SEQ ID NOS: 1-36. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, wherein the subject is non-responsive to a therapy comprising anti-TNF alpha therapy, anti-a4-b7 therapy (vedolizumab), anti-IL12p40 therapy (ustekinumab), Thalidomide, or Cytoxan.

In another aspect, are methods of treating a subject with an inflammatory disease or condition, or fibrostenotic or fibrotic disease comprising administering a therapeutically effective amount of an inhibitor of TL1A expression or activity to the subject, provided two copies of a polymorphism located at a gene locus comprising TNFSF15 and a polymorphism located at a gene locus comprising LY86, ETS1, or SCUBE1 are detected in a sample obtained from the subject. In some embodiments, the polymorphism comprises a polymorphism of Table 3. In some embodiments, the polymorphism comprises a polymorphism of Tables 3, 4, or 5. In some embodiments, the polymorphism at the TNFSF15 locus comprises rs6478109, rs7848647, rs201292440, rs7869487, rs4366152, rs6478108, rs1407308, rs7866342, rs7030574, rs10114470, rs4979464, rs3810936, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, or any polymorphism in linkage disequilibrium therewith. In some embodiments, the polymorphism comprising rs6478109 comprises a “G” allele at nucleobase 501 within rs6478109. In some embodiments, the polymorphism comprising rs7848647 comprises a “G” allele at nucleobase 501 within rs7848647. In some embodiments, the polymorphism comprising rs201292440 comprises an insertion of a nucleic acid, I, at nucleobase 501 within rs201292440. In some embodiments, the polymorphism comprising rs7869487 comprises an “A” allele at nucleobase 501 within rs7869487. In some embodiments, the polymorphism comprising rs4366152 comprises a “G” allele at nucleobase 501 within rs4366152. In some embodiments, the polymorphism comprising rs6478108 comprises an “A” allele at nucleobase 501 within rs6478108. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs10114470 comprises a “G” allele at nucleobase 501 within rs10114470. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs4574921 comprises an “A” allele at nucleobase 501 within rs4574921. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism at the TNFSF15 locus is represented with an “N” within any one of SEQ ID NOS: 1-32. In some embodiments, two copies of the polymorphism are detected in the sample obtained from the subject. In some embodiments, one copy of the polymorphism is detected in the sample obtained from the subject. In some embodiments, the polymorphism at the gene locus comprising LY86, ETS1, or SCUBE1 comprises rs6921610, rs10790957, or rs6003160, respectively, or any polymorphism in linkage disequilibrium therewith. In some embodiments, the polymorphism at the gene locus comprising ETS1 comprises rs11606640, rs73029052, rs11600915, rs61909068, rs12294634, rs73029062, rs11600746, rs61909072, or rs56086356, or any polymorphism in linkage disequilibrium therewith. In some embodiments, the polymorphism at the gene locus comprising LY86 comprises rs3851519 or any polymorphism in linkage disequilibrium therewith. In some embodiments, the polymorphism at the gene locus comprising LY86 comprises a “G” allele at nucleobase 501 within rs6921610. In some embodiments, the polymorphism at the gene locus comprising LY86 comprises a “A” allele at nucleobase 248 within rs3851519. In some embodiments, the polymorphism at the gene locus comprising ETS1 comprises a “G” allele at nucleobase 501 within rs10790957. In some embodiments, the polymorphism at the gene locus comprising ETS1 comprises a “A” allele at nucleobase 301 within rs11606640. In some embodiments, the polymorphism at the gene locus comprising ETS1 comprises a “A” allele at nucleobase 251 within rs73029052. In some embodiments, the polymorphism at the gene locus comprising ETS1 comprises a “G” allele at nucleobase 301 within rs11600915. In some embodiments, the polymorphism at the gene locus comprising ETS1 comprises a “G” allele at nucleobase 251 within rs61909068. In some embodiments, the polymorphism at the gene locus comprising ETS1 comprises a “A” allele at nucleobase 323 within rs12294634. In some embodiments, the polymorphism at the gene locus comprising ETS1 comprises a “G” allele at nucleobase 251 within rs73029062. In some embodiments, the polymorphism at the gene locus comprising ETS1 comprises a “G” allele at nucleobase 301 within rs11600746. In some embodiments, the polymorphism at the gene locus comprising ETS1 comprises a “A” allele at nucleobase 251 within rs61909072. In some embodiments, the polymorphism at the gene locus comprising ETS1 comprises a “C” allele at nucleobase 501 within rs56086356. In some embodiments, the polymorphism at the gene locus comprising SCUBE1 comprises a “G” allele at nucleobase 501 within rs6003160. In some embodiments, the polymorphism at the gene locus comprising LY86 comprises SEQ ID NO: 33. In some embodiments, the polymorphism at the gene locus comprising LY86 comprises SEQ ID NO: 80. In some embodiments, the polymorphism at the gene locus ETS1 comprises SEQ ID NO: 34. In some embodiments, the gene locus ETS1 comprises SEQ ID NO: 73. In some embodiments, the polymorphism at the gene locus comprising ETS1 comprises SEQ ID NO: 74. In some embodiments, the polymorphism at the gene locus comprising ETS1 comprises SEQ ID NO: 75. In some embodiments, the polymorphism at the gene locus comprising ETS1 comprises SEQ ID NO: 76. In some embodiments, the polymorphism at the gene locus comprising ETS1 comprises SEQ ID NO: 77. In some embodiments, the polymorphism at the gene locus comprising ETS1 comprises SEQ ID NO: 78. In some embodiments, the polymorphism at the gene locus comprising ETS1 comprises SEQ ID NO: 79. In some embodiments, the polymorphism at the gene locus comprising ETS1 comprises SEQ ID NO: 81. In some embodiments, the polymorphism at the gene locus comprising ETS1 comprises SEQ ID NO: 82. In some embodiments, the polymorphism at the gene locus comprising SCUBE1 comprises SEQ ID NO: 36. In some embodiments, the polymorphism is associated with a disease phenotype comprising non-stricturing/non-penetrating, stricturing stricturing and penetrating, or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the polymorphism is associated with an increase in expression of TL1A. In some embodiments, two copies of the polymorphism located at the TNFSF15 gene locus and the polymorphism located at a gene locus comprising LY86, ETS1, or SCUBE1 detected in the sample obtained from the subject is indicative of the subject having increase TL1A fold-change. In some embodiments, the increase in TL1A fold-change comprises an increase of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, 60-fold, 70-fold, 80-fold, 90-fold, 100-fold, 60-fold, 70-fold, 80-fold, 90-fold, or 100-fold, or more between the sample obtained from the subject and an expression of TL1A in an individual who does not express the polymorphism. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A. In some embodiments, the polymorphism is detected by using an assay comprising DNA sequencing, a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to about 10 contiguous nucleobases of any one of SEQ ID NOS: 1-36 under standard hybridization conditions. In some embodiments, the standard hybridization conditions comprise an annealing temperature between about 30° C. and about 65° C. In some embodiments, the nucleic acid sequence comprises any one of SEQ ID NOS: 37-72. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least 10 but not more than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588 or rs6003160, wherein one of the nucleobases is at position 501. In some embodiments, the nucleic acid amplification assay comprises amplification of DNA from the subject with a pair of primers capable of amplifying at least about 10 and less than 50 contiguous nucleobases within any one of SEQ ID NOS: 1-36. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, wherein the subject is non-responsive to a therapy comprising anti-TNF alpha therapy, anti-a4-b7 therapy (vedolizumab), anti-IL12p40 therapy (ustekinumab), Thalidomide, or Cytoxan.

In one aspect, are methods comprising: a) providing a sample obtained from a subject with an inflammatory condition or disease or fibrosis; b) assaying to detect in the sample a presence of a polymorphism located at a gene locus comprising TNFSF15, LY86, ETS1, ARHGAP15, or SCUBE1; and d) administering a therapeutically effective amount of an inhibitor of TL1A expression or activity to the subject, provided the presence of at least one copy of the polymorphism at the gene locus comprising TNFSF15, and the presence of either (i) the polymorphism at the gene locus comprising LY86, ETS1, SCUBE1, or the polymorphism at the gene locus comprising ARHGAP15, are detected in the sample obtained from the subject. In some embodiments, the polymorphism comprises a polymorphism of Table 3. In some embodiments, the polymorphism comprises a polymorphism of Tables 3, 4, or 5. In some embodiments, the polymorphism at the TNFSF15 locus comprises rs6478109, rs7848647, rs201292440, rs7869487, rs4366152, rs6478108, rs1407308, rs7866342, rs7030574, rs10114470, rs4979464, rs3810936, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, or any polymorphism in linkage disequilibrium therewith. In some embodiments, the polymorphism comprising rs6478109 comprises a “G” allele at nucleobase 501 within rs6478109. In some embodiments, the polymorphism comprising rs7848647 comprises a “G” allele at nucleobase 501 within rs7848647. In some embodiments, the polymorphism comprising rs201292440 comprises an insertion of a nucleic acid, I, at nucleobase 501 within rs201292440. In some embodiments, the polymorphism comprising rs7869487 comprises an “A” allele at nucleobase 501 within rs7869487. In some embodiments, the polymorphism comprising rs4366152 comprises a “G” allele at nucleobase 501 within rs4366152. In some embodiments, the polymorphism comprising rs6478108 comprises an “A” allele at nucleobase 501 within rs6478108. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs10114470 comprises a “G” allele at nucleobase 501 within rs10114470. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs4574921 comprises an “A” allele at nucleobase 501 within rs4574921. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism at the TNFSF15 locus is represented with an “N” within any one of SEQ ID NOS: 1-32. In some embodiments, two copies of the polymorphism are detected in the sample obtained from the subject. In some embodiments, one copy of the polymorphism is detected in the sample obtained from the subject. In some embodiments, the polymorphism at the gene locus comprising LY86, ETS1, ARHGAP15, or SCUBE1 comprises rs6921610, rs10790957, rs6757588, or rs6003160, respectively, or any polymorphism in linkage disequilibrium therewith. In some embodiments, the polymorphism at the gene locus comprising ETS1 comprises rs11606640, rs73029052, rs11600915, rs61909068, rs12294634, rs73029062, rs11600746, rs61909072, or rs56086356, or any polymorphism in linkage disequilibrium therewith. In some embodiments, the polymorphism at the gene locus comprising LY86 comprises rs3851519 or any polymorphism in linkage disequilibrium therewith. In some embodiments, the polymorphism at the gene locus comprising LY86 comprises a “G” allele at nucleobase 501 within rs6921610. In some embodiments, the polymorphism at the gene locus comprising LY86 comprises a “A” allele at nucleobase 248 within rs3851519. In some embodiments, the polymorphism at the gene locus comprising ETS1 comprises a “G” allele at nucleobase 501 within rs10790957. In some embodiments, the polymorphism at the gene locus comprising ETS1 comprises a “A” allele at nucleobase 301 within rs11606640. In some embodiments, the polymorphism at the gene locus comprising ETS1 comprises a “A” allele at nucleobase 251 within rs73029052. In some embodiments, the polymorphism at the gene locus comprising ETS1 comprises a “G” allele at nucleobase 301 within rs11600915. In some embodiments, the polymorphism at the gene locus comprising ETS1 comprises a “G” allele at nucleobase 251 within rs61909068. In some embodiments, the polymorphism at the gene locus comprising ETS1 comprises a “A” allele at nucleobase 323 within rs12294634. In some embodiments, the polymorphism at the gene locus comprising ETS1 comprises a “G” allele at nucleobase 251 within rs73029062. In some embodiments, the polymorphism at the gene locus comprising ETS1 comprises a “G” allele at nucleobase 301 within rs11600746. In some embodiments, the polymorphism at the gene locus comprising ETS1 comprises a “A” allele at nucleobase 251 within rs61909072. In some embodiments, the polymorphism at the gene locus comprising ETS1 comprises a “C” allele at nucleobase 501 within rs56086356. In some embodiments, the polymorphism at the gene locus comprising ARHGAP15 comprises a “G” allele at nucleobase 501 within rs6757588. In some embodiments, the polymorphism at the gene locus comprising SCUBE1 comprises a “G” allele at nucleobase 501 within rs6003160. In some embodiments, the polymorphism at the gene locus comprising LY86 comprises SEQ ID NO: 33. In some embodiments, the polymorphism at the gene locus comprising LY86 comprises SEQ ID NO: 80. In some embodiments, the polymorphism at the gene locus ETS1 comprises SEQ ID NO: 34. In some embodiments, the gene locus ETS1 comprises SEQ ID NO: 73. In some embodiments, the polymorphism at the gene locus comprising ETS1 comprises SEQ ID NO: 74. In some embodiments, the polymorphism at the gene locus comprising ETS1 comprises SEQ ID NO: 75. In some embodiments, the polymorphism at the gene locus comprising ETS1 comprises SEQ ID NO: 76. In some embodiments, the polymorphism at the gene locus comprising ETS1 comprises SEQ ID NO: 77. In some embodiments, the polymorphism at the gene locus comprising MI comprises SEQ ID NO: 78. In some embodiments, the polymorphism at the gene locus comprising ETD comprises SEQ ID NO: 79. In some embodiments, the polymorphism at the gene locus comprising ETS1 comprises SEQ ID NO: 81. In some embodiments, the polymorphism at the gene locus comprising ETS1 comprises SEQ ID NO: 82. In some embodiments, the polymorphism at the gene locus comprising ARHGAP15 comprises SEQ ID NO: 35. In some embodiments, the polymorphism at the gene locus comprising SCUBE1 comprises SEQ ID NO: 36. In some embodiments, the polymorphism is associated with a disease phenotype comprising non-stricturing/non-penetrating, stricturing, stricturing and penetrating, or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the polymorphism is associated with an increase in expression of TL1A. In some embodiments, two copies of the polymorphism located at the TNFSF15 gene locus and the polymorphism located at a gene locus comprising LY86, ETS1, or SCUBE1 detected in the sample obtained from the subject is indicative of the subject having increase TL1A fold-change. In some embodiments, one copy of the polymorphism located at the TNFSF15 gene locus and the polymorphism located at the ARHGAP15 gene locus detected in the sample obtained from the subject is indicative of the subject having an increase TL1A fold-change. In some embodiments, the increase in TL1A fold-change comprises an increase of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, 60-fold, 70-fold, 80-fold, 90-fold, 100-fold, 60-fold, 70-fold, 80-fold, 90-fold, or 100-fold, or more between the sample obtained from the subject and an expression of TL1A in an individual who does not express the polymorphism. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A. In some embodiments, the polymorphism is detected by using an assay comprising DNA sequencing, a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to about 10 contiguous nucleobases of any one of SEQ ID NOS: 1-36 under standard hybridization conditions. In some embodiments, the standard hybridization conditions comprise an annealing temperature between about 30° C. and about 65° C. In some embodiments, the nucleic acid sequence comprises any one of SEQ ID NOS: 37-72. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least 10 but not more than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588 or rs6003160, wherein one of the nucleobases is at position 501. In some embodiments, the nucleic acid amplification assay comprises amplification of DNA from the subject with a pair of primers capable of amplifying at least about 10 and less than 50 contiguous nucleobases within any one of SEQ ID NOS: 1-36. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, wherein the subject is non-responsive to a therapy comprising anti-TNF alpha therapy, anti-a4-b7 therapy (vedolizumab), anti-IL12p40 therapy (ustekinumab), Thalidomide, or Cytoxan.

In another aspect, are methods comprising: a) providing a sample obtained from a subject with an inflammatory condition or disease or fibrostenotic or fibrotic disease; b) assaying to detect in the sample obtained from the subject a presence of a polymorphism located at a gene locus comprising TNFSF15, LY86, ETS1, ARHGAP15, or SCUBE1; and c) detecting the presence of the polymorphism by contacting the sample obtained from the subject with a nucleic acid capable of hybridizing to at least about 10 and less than 50 nucleotides of the polymorphism under standard hybridization conditions and detecting binding between the polymorphism and the nucleic acid sequence. In one embodiment, the polymorphism is detected by using an assay comprising DNA sequencing, a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In one embodiment, the standard hybridization conditions comprise an annealing temperature between about 30° C. and about 65° C. In some embodiments, the nucleic acid sequence comprises any one of SEQ ID NOS: 37-72. In one embodiment, the nucleic acid sequence is conjugated to a detectable molecule. In one embodiment, the detectable molecule comprises a fluorophore. In one embodiment, the nucleic acid sequence is conjugated to a quencher. In one embodiment, the sample obtained from the subject comprises gene material that is amplified using a nucleic acid amplification assay. In one embodiment, the nucleic acid amplification assay comprises amplification of DNA from the subject with a pair of primers capable of amplifying at least about 10 and less than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588 or rs6003160. In one embodiment, the nucleic acid amplification assay comprises amplification of DNA from the subject with a pair of primers capable of amplifying at least about 10 and less than 50 contiguous nucleobases within any one of SEQ ID NOS: 1-36. In one embodiment, the polymorphism at the gene locus comprising TNFSF15 comprises rs6478109, rs7848647, rs201292440, rs7869487, rs4366152, rs6478108, rs1407308, rs7866342, rs7030574, rs10114470, rs4979464, rs3810936, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, or any polymorphism in linkage disequilibrium therewith. In some embodiments, the polymorphism comprises a polymorphism of Table 3. In some embodiments, the polymorphism comprises a polymorphism of Tables 3, 4, or 5. In some embodiments, the polymorphism at the TNFSF15 locus comprises rs6478109, rs7848647, rs201292440, rs7869487, rs4366152, rs6478108, rs1407308, rs7866342, rs7030574, rs10114470, rs4979464, rs3810936, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, or any polymorphism in linkage disequilibrium therewith. In some embodiments, the polymorphism comprising rs6478109 comprises a “G” allele at nucleobase 501 within rs6478109. In some embodiments, the polymorphism comprising rs7848647 comprises a “G” allele at nucleobase 501 within rs7848647. In some embodiments, the polymorphism comprising rs201292440 comprises an insertion of a nucleic acid, I, at nucleobase 501 within rs201292440. In some embodiments, the polymorphism comprising rs7869487 comprises an “A” allele at nucleobase 501 within rs7869487. In some embodiments, the polymorphism comprising rs4366152 comprises a “G” allele at nucleobase 501 within rs4366152. In some embodiments, the polymorphism comprising rs6478108 comprises an “A” allele at nucleobase 501 within rs6478108. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs10114470 comprises a “G” allele at nucleobase 501 within rs10114470. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs4574921 comprises an “A” allele at nucleobase 501 within rs4574921. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism at the TNFSF15 locus is represented with an “N” within any one of SEQ ID NOS: 1-32. In some embodiments, two copies of the polymorphism are detected in the sample obtained from the subject. In some embodiments, one copy of the polymorphism is detected in the sample obtained from the subject. In some embodiments, the polymorphism at the gene locus comprising LY86, ETS1, ARHGAP15, or SCUBE1 comprises rs6921610, rs10790957, rs6757588, or rs6003160, respectively, or any polymorphism in linkage disequilibrium therewith. In some embodiments, the polymorphism at the gene locus comprising ETD comprises rs11606640, rs73029052, rs11600915, rs61909068, rs12294634, rs73029062, rs11600746, rs61909072, or rs56086356, or any polymorphism in linkage disequilibrium therewith. In some embodiments, the polymorphism at the gene locus comprising LY86 comprises rs3851519 or any polymorphism in linkage disequilibrium therewith. In some embodiments, the polymorphism at the gene locus comprising LY86 comprises a “G” allele at nucleobase 501 within rs6921610. In some embodiments, the polymorphism at the gene locus comprising LY86 comprises a “A” allele at nucleobase 248 within rs3851519. In some embodiments, the polymorphism at the gene locus comprising ETS1 comprises a “G” allele at nucleobase 501 within rs10790957. In some embodiments, the polymorphism at the gene locus comprising ETD comprises a “A” allele at nucleobase 301 within rs11606640. In some embodiments, the polymorphism at the gene locus comprising ETS1 comprises a “A” allele at nucleobase 251 within rs73029052. In some embodiments, the polymorphism at the gene locus comprising ETS1 comprises a “G” allele at nucleobase 301 within rs11600915. In some embodiments, the polymorphism at the gene locus comprising ETS1 comprises a “G” allele at nucleobase 251 within rs61909068. In some embodiments, the polymorphism at the gene locus comprising ETS1 comprises a “A” allele at nucleobase 323 within rs12294634. In some embodiments, the polymorphism at the gene locus comprising ETS1 comprises a “G” allele at nucleobase 251 within rs73029062. In some embodiments, the polymorphism at the gene locus comprising ETS1 comprises a “G” allele at nucleobase 301 within rs11600746. In some embodiments, the polymorphism at the gene locus comprising ETS1 comprises a “A” allele at nucleobase 251 within rs61909072. In some embodiments, the polymorphism at the gene locus comprising ETS1 comprises a “C” allele at nucleobase 501 within rs56086356. In some embodiments, the polymorphism at the gene locus comprising ARHGAP15 comprises a “G” allele at nucleobase 501 within rs6757588. In some embodiments, the polymorphism at the gene locus comprising SCUBE1 comprises a “G” allele at nucleobase 501 within rs6003160. In some embodiments, the polymorphism at the gene locus comprising LY86 comprises SEQ ID NO: 33. In some embodiments, the polymorphism at the gene locus comprising LY86 comprises SEQ ID NO: 80. In some embodiments, the polymorphism at the gene locus ETS1 comprises SEQ ID NO: 34. In some embodiments, the gene locus ETS1 comprises SEQ ID NO: 73. In some embodiments, the polymorphism at the gene locus comprising ETS1 comprises SEQ ID NO: 74. In some embodiments, the polymorphism at the gene locus comprising ETS1 comprises SEQ ID NO: 75. In some embodiments, the polymorphism at the gene locus comprising ETS1 comprises SEQ ID NO: 76. In some embodiments, the polymorphism at the gene locus comprising ETS1 comprises SEQ ID NO: 77. In some embodiments, the polymorphism at the gene locus comprising ETS1 comprises SEQ ID NO: 78. In some embodiments, the polymorphism at the gene locus comprising ETS1 comprises SEQ ID NO: 79. In some embodiments, the polymorphism at the gene locus comprising ETS1 comprises SEQ ID NO: 81. In some embodiments, the polymorphism at the gene locus comprising ETS1 comprises SEQ ID NO: 82. In some embodiments, the polymorphism at the gene locus comprising ARHGAP15 comprises SEQ ID NO: 35. In some embodiments, the polymorphism at the gene locus comprising SCUBE1 comprises SEQ ID NO: 36. In some embodiments, the polymorphism is associated with a disease phenotype comprising non-stricturing/non-penetrating, stricturing, stricturing and penetrating, or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the polymorphism is associated with an increase in expression of TL1A. In some embodiments, two copies of the polymorphism are detected in the sample obtained from the subject. In some embodiments, one copy of the polymorphism is detected in the sample obtained from the subject. In some embodiments, two copies of the polymorphism located at the TNFSF15 gene locus and the polymorphism located at a gene locus comprising LY86, ETS1, or SCUBE1 detected in the sample obtained from the subject is indicative of the subject having increase TL1A fold-change. In some embodiments, one copy of the polymorphism located at the TNFSF15 gene locus and the polymorphism located at the ARHGAP15 gene locus detected in the sample obtained from the subject is indicative of the subject having an increase TL1A fold-change. In some embodiments, the increase in TL1A fold-change comprises an increase of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, 60-fold, 70-fold, 80-fold, 90-fold, 100-fold, or more between the sample obtained from the subject and an expression of TL1A in an individual who does not express the polymorphism. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, a therapeutically effective amount of an inhibitor of TL1A expression or activity to the subject, provided the presence of a polymorphism is detected in the sample obtained from the subject. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, wherein the subject is non-responsive to a therapy comprising anti-TNF alpha therapy, anti-a4-b7 therapy (vedolizumab), anti-IL12p40 therapy (ustekinumab), Thalidomide, or Cytoxan.

In one aspect, are methods of characterizing an inflammatory condition or disease or fibrosis of a subject, the method comprising assaying a sample obtained from the subject to identify the presence of a genotype comprising a polymorphism at nucleobase 501 within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588 or rs6003160. In some embodiments, the polymorphism comprises any one of SEQ ID NOS: 1-36. In some embodiments, the genotype comprises two copies of the polymorphism. In some embodiments, the genotype comprises one copy of the polymorphism. In some embodiments, the polymorphism is associated with a disease phenotype comprising non-stricturing/non-penetrating, stricturing, stricturing and penetrating, or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, wherein the polymorphism is associated with an increase in TL1A fold-change. In some embodiments, the genotype comprises two copies of a first polymorphism comprising rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, and at least one copy of a second polymorphism comprising rs10790957, rs6921610, or rs6003160. In some embodiments, the genotype comprises one copy of a first polymorphism comprising rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, or rs56069985, and a at least one copy of a second polymorphism comprising rs6757588. In some embodiments, the increase in TL1A fold-change comprises an increase of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, 60-fold, 70-fold, 80-fold, 90-fold, or 100-fold, or more between the sample obtained from the subject and an expression of TL1A in an individual who does not express the polymorphism. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the polymorphism is detected by using an assay comprising DNA sequencing, a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to about 10 contiguous nucleobases of any one of SEQ ID NOS: 1-36 under standard hybridization conditions. In some embodiments, the standard hybridization conditions comprise an annealing temperature between about 30° C. and about 65° C. In some embodiments, the nucleic acid sequence comprises any one of SEQ ID NOS: 37-72. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least 10 but not more than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160 rs11606640, rs73029052, rs11600915, rs61909068, rs12294634, rs73029062, rs11600746, rs3851519, rs61909072, or rs56086356, wherein one of the nucleobases is at position 501. In some embodiments, the nucleic acid amplification assay comprises amplification of DNA from the subject with a pair of primers capable of amplifying at least about 10 and less than 50 contiguous nucleobases within any one of SEQ ID NOS: 1-36. In some embodiments, a therapeutically effective amount of an inhibitor of TL1A expression or activity to the subject, provided the presence of a polymorphism is detected in the sample obtained from the subject. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, wherein the subject is non-responsive to a therapy comprising anti-TNF alpha therapy, anti-a4-b7 therapy (vedolizumab), anti-IL12p40 therapy (ustekinumab), Thalidomide, or Cytoxan.

In another aspect are compositions comprising at least about 10 but less than 50 contiguous nucleobase residues of any one of SEQ ID NOS: 1-36, or reverse complement sequence thereof, wherein the contiguous nucleobase residues comprise the nucleobase at position 501 of any one of SEQ ID NOS: 1-36, and wherein the contiguous nucleobase residues are connected to a detectable molecule. In some embodiments, the detectable molecule is a fluorophore. In some embodiments the contiguous nucleobase residues are connected to a quencher. In another aspect, are kits comprising the compositions disclosed herein, and a primer pair capable of hybridizing to at least about 10 contiguous nucleobases of any one of SEQ ID NOS: 1-36 or reverse complement sequence thereof. Further provided are methods comprising contacting DNA from a subject with the compositions disclosed herein using the kits disclosed herein under conditions suitable to hybridize the composition to the DNA if the DNA comprises a sequence complementary to the composition, or reverse complement thereof. In another aspect, are methods comprising treating the subject of with an inhibitor of TL1A activity or expression, provided that the DNA from the subject comprises the sequence complementary to the composition. In some embodiments, the inhibitor of TL1A expression or activity comprises a TL1A antibody, or a TL1A-binding antibody fragment. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, wherein the subject is non-responsive to a therapy comprising anti-TNF alpha therapy, anti-a4-b7 therapy (vedolizumab), anti-IL12p40 therapy (ustekinumab), Thalidomide, or Cytoxan.

Aspects disclosed herein provide methods of treating a subject with an inflammatory disease or condition, or fibrostenotic or fibrotic disease comprising administering a therapeutically effective amount of an inhibitor of TL1A expression or activity to the subject, provided a presence of a polymorphism associated with increased TL1A fold-change and characterized by a p value of at most about 10⁻³ as determined by a TL1A fold-change enrichment analysis is detected in a sample obtained from the subject, wherein the polymorphism does not comprise a risk allele within a polymorphism comprising rs6478109, rs7848647, rs201292440, rs7869487, rs6478108, rs10114470, and rs4574921. In some embodiments, the p value comprises 10⁻⁴. In some embodiments, the p value comprises 10⁻⁵. In some embodiments, the p value comprises 10⁻⁶. In some embodiments, the TL1A fold-change enrichment analysis comprises the operations of: a) assaying, or having assayed, a plurality of samples obtained from a plurality of subjects to detect an increase in TL1A fold-change; b) obtaining, or having obtained, a plurality of genotypes of the plurality of subjects, wherein the plurality of genotypes comprise polymorphisms associated with the increase in TL1A fold-change using a linear regression model or logistic regression model, wherein the polymorphisms are characterized by having a p value of at most 10⁻³; c) selecting a criteria polymorphism from the polymorphisms associated with the increase in TL1A fold-change to serve as a predictor of the increase in TL1A fold-change in the plurality of subjects, the criteria polymorphism comprising rs6478109, wherein selection of the criterial polymorphism is based, at least, on the p value; and d) identifying the risk polymorphism, provided an enrichment of the increase in TL1A fold-change is observed in a subset of the plurality of samples in which the criteria polymorphism and the risk polymorphism are expressed, as compared to the increase in TL1A fold-change observed when the criteria polymorphism, alone, is expressed. In some embodiments, the polymorphism associated with increased TL1A fold-change comprises a “G” allele at nucleobase 501 within rs6912610. In some embodiments, the polymorphism associated with increased TL1A fold-change comprises a “G” allele at nucleobase 501 within rs10790957. In some embodiments, the polymorphism associated with increased TL1A fold-change comprises a “G” allele at nucleobase 501 within rs6757588. In some embodiments, the polymorphism associated with increased TL1A fold-change comprises a “G” allele at nucleobase 501 within rs6003160. In some embodiments, the polymorphism associated with increased TL1A fold-change comprises an “A” allele at nucleobase 301 within rs11606640. In some embodiments, the polymorphism associated with increased TL1A fold-change comprises “A” allele at nucleobase 251 within rs73029052. In some embodiments, the polymorphism associated with increased TL1A fold-change comprises a “G” allele at nucleobase 301 within rs11600915. In some embodiments, the polymorphism associated with increased TL1A fold-change comprises a “G” allele at nucleobase 251 within rs61909068. In some embodiments, the polymorphism associated with increased TL1A fold-change comprises an “A” allele at nucleobase 323 within rs12294634. In some embodiments, the polymorphism associated with increased TL1A fold-change comprises a “G” allele at nucleobase 251 within rs73029062. In some embodiments, the polymorphism associated with increased TL1A fold-change comprises a “G” allele at nucleobase 301 within rs11600746. In some embodiments, the polymorphism associated with increased TL1A fold-change comprises an “A” allele at nucleobase 251 within rs61909072. In some embodiments, the polymorphism associated with increased TL1A fold-change comprises a “C” allele at nucleobase 501 within rs56086356. In some embodiments, the polymorphism associated with increased TL1A fold-change comprises an “A” allele at nucleobase 248 within rs3851519. In some embodiments, the polymorphism associated with increased TL1A fold-change comprises SEQ ID NO: 33. In some embodiments, the polymorphism associated with increased TL1A fold-change comprises SEQ ID NO: 34. In some embodiments, the polymorphism associated with increased TL1A fold-change comprises SEQ ID NO: 35. In some embodiments, the polymorphism associated with increased TL1A fold-change comprises SEQ ID NO: 36. In some embodiments, the polymorphism associated with increased TL1A fold-change comprises SEQ ID NO: 73. In some embodiments, the polymorphism associated with increased TL1A fold-change comprises SEQ ID NO: 74. In some embodiments, the polymorphism associated with increased TL1A fold-change comprises SEQ ID NO: 75. In some embodiments, the polymorphism associated with increased TL1A fold-change comprises SEQ ID NO: 76. In some embodiments, the polymorphism associated with increased TL1A fold-change comprises SEQ ID NO: 77. In some embodiments, the polymorphism associated with increased TL1A fold-change comprises SEQ ID NO: 78. In some embodiments, the polymorphism associated with increased TL1A fold-change comprises SEQ ID NO: 79. In some embodiments, the polymorphism associated with increased TL1A fold-change comprises SEQ ID NO: 81. In some embodiments, the polymorphism associated with increased TL1A fold-change comprises SEQ ID NO: 82. In some embodiments, the polymorphism associated with increased TL1A fold-change comprises SEQ ID NO: 80. In some embodiments, the polymorphism associated with increased TL1A fold-change comprises rs4366152, rs1407308, rs7866342, rs7030574, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs6478106, rs7032238, rs55775610, rs7847158, or rs56069985, or any polymorphism in linkage disequilibrium therewith. In some embodiments, the polymorphism comprising rs4366152 comprises a “G” allele at nucleobase 501 within rs4366152. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments, the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism comprising rs6478109 comprises a “G” allele at nucleobase 501 within rs6478109. In some embodiments, the polymorphism comprising rs201292440 comprises an insertion of a nucleic acid, I, at nucleobase 501 within rs201292440. In some embodiments, the polymorphism comprising rs7848647 comprises a “G” allele at nucleobase 501 within rs7848647. In some embodiments, the polymorphism comprising rs7869487 comprises an “A” allele at nucleobase 501 within rs7869487. In some embodiments, the polymorphism comprising rs6478108 comprises an “A” allele at nucleobase 501 within rs6478108. In some embodiments, the polymorphism comprising rs10114470 comprises a “G” allele at nucleobase 501 within rs10114470. In some embodiments, the polymorphism comprising rs4574921 comprises an “A” allele at nucleobase 501 within rs4574921. In some embodiments, two copies of the polymorphism are detected in the sample obtained from the subject. In some embodiments, one copy of the polymorphism is detected in the sample obtained from the subject. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the polymorphism is detected by using an assay comprising DNA sequencing a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to at least about 10 but less than 50 contiguous nucleobases of any one of SEQ ID NOS: 5,7-9, 11-26, 28-36, and 73-82 or reverse complement sequence thereof, under standard hybridization conditions. In some embodiments, the standard hybridization conditions comprise an annealing temperature between about 30° C. and about 65° C. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least about 10 and less than 50 nucleobases within rs4366152, rs1407308, rs7866342, rs7030574, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs6478106, rs7032238, rs55775610, rs7847158, rs56069985, rs10790957, rs6921610, rs6757588 or rs6003160. In some embodiments, the nucleic acid amplification assay comprises amplification of DNA from the subject with a pair of primers capable of amplifying at least about 10 but less than 50 contiguous nucleobases within any one of SEQ ID NOS: 5,7-9, 11-26, 28-36, and 73-82. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, the subject is non-responsive to a therapy comprising anti-TNF alpha therapy, anti-a4-b7 therapy (vedolizumab), anti-IL12p40 therapy (ustekinumab), Thalidomide, or Cytoxan. In some embodiments, the polymorphism is associated with a disease phenotype comprising non-stricturing/non-penetrating, stricturing, stricturing and penetrating or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A.

Aspects disclosed herein provide methods of characterizing an inflammatory condition or disease or fibrosis of a subject, the method comprising assaying a sample obtained from the subject to identify the presence of a risk genotype comprising a risk polymorphism associated with increased TL1A fold-change and characterized by a p value of at most about 10⁻³ as determined by a TL1A fold-change enrichment analysis is detected in a sample obtained from the subject, wherein the polymorphism does not comprise a risk allele within a polymorphism comprising rs6478109, rs7848647, rs201292440, rs7869487, rs6478108, rs10114470, and rs4574921. In some embodiments, the p value comprises 10⁻⁵. In some embodiments, the p value comprises 10⁻⁶. In some embodiments, the TL1A fold-change enrichment analysis comprises: a) assaying, or having assayed, a plurality of samples obtained from a plurality of subjects to detect an increase in TL1A fold-change; b) obtaining, or having obtained, a plurality of genotypes of the plurality of subjects, wherein the plurality of genotypes comprise polymorphisms associated with the increase in TL1A fold-change using a linear regression model or logistic regression model, wherein the polymorphisms are characterized by having a p value of at most 10⁻³; c) selecting a criteria polymorphism from the polymorphisms associated with the increase in TL1A fold-change to serve as a predictor of the increase in TL1A fold-change in the plurality of subjects, the criteria polymorphism comprising rs6478109, wherein selection of the criterial polymorphism is based, at least, on the p value; and d) identifying the risk polymorphism, provided an enrichment of the increase in TL1A fold-change is observed in a subset of the plurality of samples in which the criteria polymorphism and the risk polymorphism are expressed, as compared to the increase in TL1A fold-change observed when the criteria polymorphism, alone, is expressed. In some embodiments, the polymorphism associated with increased TL1A fold-change comprises a “G” allele at nucleobase 501 within rs6912610. In some embodiments, the polymorphism associated with increased TL1A fold-change comprises a “G” allele at nucleobase 501 within rs10790957. In some embodiments, the polymorphism associated with increased TL1A fold-change comprises a “G” allele at nucleobase 501 within rs6757588. In some embodiments, the polymorphism associated with increased TL1A fold-change comprises a “G” allele at nucleobase 501 within rs6003160. In some embodiments, the polymorphism associated with increased TL1A fold-change comprises an “A” allele at nucleobase 301 within rs11606640. In some embodiments, the polymorphism associated with increased TL1A fold-change comprises “A” allele at nucleobase 251 within rs73029052. In some embodiments, the polymorphism associated with increased TL1A fold-change comprises a “G” allele at nucleobase 301 within rs11600915. In some embodiments, the polymorphism associated with increased TL1A fold-change comprises a “G” allele at nucleobase 251 within rs61909068. In some embodiments, the polymorphism associated with increased TL1A fold-change comprises an “A” allele at nucleobase 323 within rs12294634. In some embodiments, the polymorphism associated with increased TL1A fold-change comprises a “G” allele at nucleobase 251 within rs73029062. In some embodiments, the polymorphism associated with increased TL1A fold-change comprises a “G” allele at nucleobase 301 within rs11600746. In some embodiments, the polymorphism associated with increased TL1A fold-change comprises an “A” allele at nucleobase 251 within rs61909072. In some embodiments, the polymorphism associated with increased TL1A fold-change comprises a “C” allele at nucleobase 501 within rs56086356. In some embodiments, the polymorphism associated with increased TL1A fold-change comprises an “A” allele at nucleobase 248 within rs3851519. In some embodiments, the polymorphism associated with increased TL1A fold-change comprises SEQ ID NO: 33. In some embodiments, the polymorphism associated with increased TL1A fold-change comprises SEQ ID NO: 34. In some embodiments, the polymorphism associated with increased TL1A fold-change comprises SEQ ID NO: 35. In some embodiments, the polymorphism associated with increased TL1A fold-change comprises SEQ ID NO: 36. In some embodiments, the polymorphism associated with increased TL1A fold-change comprises SEQ ID NO: 73. In some embodiments, the polymorphism associated with increased TL1A fold-change comprises SEQ ID NO: 74. In some embodiments, the polymorphism associated with increased TL1A fold-change comprises SEQ ID NO: 75. In some embodiments, the polymorphism associated with increased TL1A fold-change comprises SEQ ID NO: 76. In some embodiments, the polymorphism associated with increased TL1A fold-change comprises SEQ ID NO: 77. In some embodiments, the polymorphism associated with increased TL1A fold-change comprises SEQ ID NO: 78. In some embodiments, the polymorphism associated with increased TL1A fold-change comprises SEQ ID NO: 79. In some embodiments, the polymorphism associated with increased TL1A fold-change comprises SEQ ID NO: 81. In some embodiments, the polymorphism associated with increased TL1A fold-change comprises SEQ ID NO: 82. In some embodiments, the polymorphism associated with increased TL1A fold-change comprises SEQ ID NO: 80. In some embodiments, the polymorphism associated with increased TL1A fold-change comprises rs4366152, rs1407308, rs7866342, rs7030574, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs6478106, rs7032238, rs55775610, rs7847158, or rs56069985, or any polymorphism in linkage disequilibrium therewith. In some embodiments, the polymorphism comprising rs4366152 comprises a “G” allele at nucleobase 501 within rs4366152. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments, the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism comprising rs6478109 comprises a “G” allele at nucleobase 501 within rs6478109. In some embodiments, the polymorphism comprising rs201292440 comprises an insertion of a nucleic acid, I, at nucleobase 501 within rs201292440. In some embodiments, the polymorphism comprising rs7848647 comprises a “G” allele at nucleobase 501 within rs7848647. In some embodiments, the polymorphism comprising rs7869487 comprises an “A” allele at nucleobase 501 within rs7869487. In some embodiments, the polymorphism comprising rs6478108 comprises an “A” allele at nucleobase 501 within rs6478108. In some embodiments, the polymorphism comprising rs10114470 comprises a “G” allele at nucleobase 501 within rs10114470. In some embodiments, the polymorphism comprising rs4574921 comprises an “A” allele at nucleobase 501 within rs4574921. In some embodiments, two copies of the polymorphism are detected in the sample obtained from the subject. In some embodiments, one copy of the polymorphism is detected in the sample obtained from the subject. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the polymorphism is detected by using an assay comprising DNA sequencing a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to at least about 10 but less than 50 contiguous nucleobases of any one of SEQ ID NOS: 5,7-9, 11-26, 28-36, and 73-82 or reverse complement sequence thereof, under standard hybridization conditions. In some embodiments, the standard hybridization conditions comprise an annealing temperature between about 30° C. and about 65° C. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least about 10 and less than 50 nucleobases within rs4366152, rs1407308, rs7866342, rs7030574, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs6478106, rs7032238, rs55775610, rs7847158, rs56069985, rs10790957, rs6921610, rs6757588 or rs6003160. In some embodiments, the nucleic acid amplification assay comprises amplification of DNA from the subject with a pair of primers capable of amplifying at least about 10 but less than 50 contiguous nucleobases within any one of SEQ ID NOS: 5,7-9, 11-26, 28-36, and 73-82. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, the subject is non-responsive to a therapy comprising anti-TNF alpha therapy, anti-a4-b7 therapy (vedolizumab), anti-IL12p40 therapy (ustekinumab), Thalidomide, or Cytoxan. In some embodiments, the polymorphism is associated with a disease phenotype comprising non-stricturing/non-penetrating, stricturing, stricturing and penetrating or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the methods further comprise administering to the subject an inhibitor of TL1A expression or activity. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A.

Aspects disclosed herein provide methods of treating a subject with an inflammatory disease or condition, or fibrostenotic or fibrotic disease comprising administering a therapeutically effective amount of an inhibitor of TL1A expression or activity to the subject, provided a presence of a polymorphism associated with increased TL1A fold-change that is in linkage disequilibrium with rs6478109 as defined by (i) a D′ value of at least about 0.80, or (ii) a D′ value of 0 and an R² value of at least about 0.90, wherein the polymorphism does not comprise a risk allele within a polymorphism comprising rs6478109, rs7848647, rs201292440, rs7869487, rs6478108, rs10114470, and rs4574921. In some embodiments, the linkage disequilibrium with rs6478109 is defined by a D′ value of at least about 0.80. In some embodiments, the linkage disequilibrium with rs6478109 is defined a D′ value of 0 and an R² value of at least about 0.90. In some embodiments, the polymorphism associated with increased TL1A fold-change comprises a “G” allele at nucleobase 501 within rs6912610. In some embodiments, the polymorphism associated with increased TL1A fold-change comprises a “G” allele at nucleobase 501 within rs10790957. In some embodiments, the polymorphism associated with increased TL1A fold-change comprises a “G” allele at nucleobase 501 within rs6757588. In some embodiments, the polymorphism associated with increased TL1A fold-change comprises a “G” allele at nucleobase 501 within rs6003160. In some embodiments, the polymorphism associated with increased TL1A fold-change comprises an “A” allele at nucleobase 301 within rs11606640. In some embodiments, the polymorphism associated with increased TL1A fold-change comprises “A” allele at nucleobase 251 within rs73029052. In some embodiments, the polymorphism associated with increased TL1A fold-change comprises a “G” allele at nucleobase 301 within rs11600915. In some embodiments, the polymorphism associated with increased TL1A fold-change comprises a “G” allele at nucleobase 251 within rs61909068. In some embodiments, the polymorphism associated with increased TL1A fold-change comprises an “A” allele at nucleobase 323 within rs12294634. In some embodiments, the polymorphism associated with increased TL1A fold-change comprises a “G” allele at nucleobase 251 within rs73029062. In some embodiments, the polymorphism associated with increased TL1A fold-change comprises a “G” allele at nucleobase 301 within rs11600746. In some embodiments, the polymorphism associated with increased TL1A fold-change comprises an “A” allele at nucleobase 251 within rs61909072. In some embodiments, the polymorphism associated with increased TL1A fold-change comprises a “C” allele at nucleobase 501 within rs56086356. In some embodiments, the polymorphism associated with increased TL1A fold-change comprises an “A” allele at nucleobase 248 within rs3851519. In some embodiments, the polymorphism associated with increased TL1A fold-change comprises SEQ ID NO: 33. In some embodiments, the polymorphism associated with increased TL1A fold-change comprises SEQ ID NO: 34. In some embodiments, the polymorphism associated with increased TL1A fold-change comprises SEQ ID NO: 35. In some embodiments, the polymorphism associated with increased TL1A fold-change comprises SEQ ID NO: 36. In some embodiments, the polymorphism associated with increased TL1A fold-change comprises SEQ ID NO: 73. In some embodiments, the polymorphism associated with increased TL1A fold-change comprises SEQ ID NO: 74. In some embodiments, the polymorphism associated with increased TL1A fold-change comprises SEQ ID NO: 75. In some embodiments, the polymorphism associated with increased TL1A fold-change comprises SEQ ID NO: 76. In some embodiments, the polymorphism associated with increased TL1A fold-change comprises SEQ ID NO: 77. In some embodiments, the polymorphism associated with increased TL1A fold-change comprises SEQ ID NO: 78. In some embodiments, the polymorphism associated with increased TL1A fold-change comprises SEQ ID NO: 79. In some embodiments, the polymorphism associated with increased TL1A fold-change comprises SEQ ID NO: 81. In some embodiments, the polymorphism associated with increased TL1A fold-change comprises SEQ ID NO: 82. In some embodiments, the polymorphism associated with increased TL1A fold-change comprises SEQ ID NO: 80. In some embodiments, the polymorphism associated with increased TL1A fold-change comprises rs4366152, rs1407308, rs7866342, rs7030574, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs6478106, rs7032238, rs55775610, rs7847158, or rs56069985, or any polymorphism in linkage disequilibrium therewith. In some embodiments, the polymorphism comprising rs4366152 comprises a “G” allele at nucleobase 501 within rs4366152. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments, the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism comprising rs6478109 comprises a “G” allele at nucleobase 501 within rs6478109. In some embodiments, the polymorphism comprising rs201292440 comprises an insertion of a nucleic acid, I, at nucleobase 501 within rs201292440. In some embodiments, the polymorphism comprising rs7848647 comprises a “G” allele at nucleobase 501 within rs7848647. In some embodiments, the polymorphism comprising rs7869487 comprises an “A” allele at nucleobase 501 within rs7869487. In some embodiments, the polymorphism comprising rs6478108 comprises an “A” allele at nucleobase 501 within rs6478108. In some embodiments, the polymorphism comprising rs10114470 comprises a “G” allele at nucleobase 501 within rs10114470. In some embodiments, the polymorphism comprising rs4574921 comprises an “A” allele at nucleobase 501 within rs4574921. In some embodiments, two copies of the polymorphism are detected in the sample obtained from the subject. In some embodiments, one copy of the polymorphism is detected in the sample obtained from the subject. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the polymorphism is detected by using an assay comprising DNA sequencing, a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to at least about 10 but less than 50 contiguous nucleobases of any one of SEQ ID NOS: 5,7-9, 11-26, 28-36, and 73-82 or reverse complement sequence thereof, under standard hybridization conditions. In some embodiments, the standard hybridization conditions comprise an annealing temperature between about 30° C. and about 65° C. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least about 10 and less than 50 nucleobases within rs4366152, rs1407308, rs7866342, rs7030574, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs6478106, rs7032238, rs55775610, rs7847158, rs56069985, rs10790957, rs6921610, rs6757588 or rs6003160. In some embodiments, the nucleic acid amplification assay comprises amplification of DNA from the subject with a pair of primers capable of amplifying at least about 10 but less than 50 contiguous nucleobases within any one of SEQ ID NOS: 5,7-9, 11-26, 28-36, and 73-82. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, the subject is non-responsive to a therapy comprising anti-TNF alpha therapy, anti-a4-b7 therapy (vedolizumab), anti-IL12p40 therapy (ustekinumab), Thalidomide, or Cytoxan. In some embodiments, the polymorphism is associated with a disease phenotype comprising non-stricturing/non-penetrating, stricturing stricturing and penetrating, or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A.

Aspects disclosed herein provide methods of characterizing an inflammatory condition or disease or fibrosis of a subject, the method comprising assaying a sample obtained from the subject to identify the presence of a genotype comprising a polymorphism associated with increased TL1A fold-change that is in linkage disequilibrium with rs6478109 as defined by (i) a D′ value of at least about 0.80, or (ii) a D′ value of 0 and an R² value of at least about 0.90, wherein the polymorphism does not comprise a risk allele within a polymorphism comprising rs6478109, rs7848647, rs201292440, rs7869487, rs6478108, rs10114470, and rs4574921. In some embodiments, the linkage disequilibrium with rs6478109 is defined by a D′ value of at least about 0.80. In some embodiments, the linkage disequilibrium with rs6478109 is defined a D′ value of 0 and an R² value of at least about 0.90. In some embodiments, the polymorphism associated with increased TL fold-change comprises a “G” allele at nucleobase 501 within rs6912610. In some embodiments, the polymorphism associated with increased TL1A fold-change comprises a “G” allele at nucleobase 501 within rs10790957. In some embodiments, the polymorphism associated with increased TL1A fold-change comprises a “G” allele at nucleobase 501 within rs6757588. In some embodiments, the polymorphism associated with increased TL1A fold-change comprises a “G” allele at nucleobase 501 within rs6003160. In some embodiments, the polymorphism associated with increased TL1A fold-change comprises an “A” allele at nucleobase 301 within rs11606640. In some embodiments, the polymorphism associated with increased TL1A fold-change comprises “A” allele at nucleobase 251 within rs73029052. In some embodiments, the polymorphism associated with increased TL1A fold-change comprises a “G” allele at nucleobase 301 within rs11600915. In some embodiments, the polymorphism associated with increased TL1A fold-change comprises a “G” allele at nucleobase 251 within rs61909068. In some embodiments, the polymorphism associated with increased TL1A fold-change comprises an “A” allele at nucleobase 323 within rs12294634. In some embodiments, the polymorphism associated with increased TL1A fold-change comprises a “G” allele at nucleobase 251 within rs73029062. In some embodiments, the polymorphism associated with increased TL1A fold-change comprises a “G” allele at nucleobase 301 within rs11600746. In some embodiments, the polymorphism associated with increased TL1A fold-change comprises an “A” allele at nucleobase 251 within rs61909072. In some embodiments, the polymorphism associated with increased TL1A fold-change comprises a “C” allele at nucleobase 501 within rs56086356. In some embodiments, the polymorphism associated with increased TL1A fold-change comprises an “A” allele at nucleobase 248 within rs3851519. In some embodiments, the polymorphism associated with increased TL1A fold-change comprises SEQ ID NO: 33. In some embodiments, the polymorphism associated with increased TL1A fold-change comprises SEQ ID NO: 34. In some embodiments, the polymorphism associated with increased TL fold-change comprises SEQ ID NO: 35. In some embodiments, the polymorphism associated with increased TL1A fold-change comprises SEQ ID NO: 36. In some embodiments, the polymorphism associated with increased TL1A fold-change comprises SEQ ID NO: 73. In some embodiments, the polymorphism associated with increased TL1A fold-change comprises SEQ ID NO: 74. In some embodiments, the polymorphism associated with increased TL1A fold-change comprises SEQ ID NO: 75. In some embodiments, the polymorphism associated with increased TL1A fold-change comprises SEQ ID NO: 76. In some embodiments, the polymorphism associated with increased TL1A fold-change comprises SEQ ID NO: 77. In some embodiments, the polymorphism associated with increased TL1A fold-change comprises SEQ ID NO: 78. In some embodiments, the polymorphism associated with increased TL1A fold-change comprises SEQ ID NO: 79. In some embodiments, the polymorphism associated with increased TL1A fold-change comprises SEQ ID NO: 81. In some embodiments, the polymorphism associated with increased TL1A fold-change comprises SEQ ID NO: 82. In some embodiments, the polymorphism associated with increased TL1A fold-change comprises SEQ ID NO: 80. In some embodiments, the polymorphism associated with increased TL1A fold-change comprises rs4366152, rs1407308, rs7866342, rs7030574, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs6478106, rs7032238, rs55775610, rs7847158, or rs56069985, or any polymorphism in linkage disequilibrium therewith. In some embodiments, the polymorphism comprising rs4366152 comprises a “G” allele at nucleobase 501 within rs4366152. In some embodiments, the polymorphism comprising rs1407308 comprises a “G” allele at nucleobase 501 within rs1407308. In some embodiments, the polymorphism comprising rs7866342 comprises an “A” allele at nucleobase 501 within rs7866342. In some embodiments, the polymorphism comprising rs7030574 comprises an “A” allele at nucleobase 501 within rs7030574. In some embodiments, the polymorphism comprising rs4979464 comprises a “G” allele at nucleobase 201 within rs4979464. In some embodiments, the polymorphism comprising rs3810936 comprises a “G” allele at nucleobase 501 within rs3810936. In some embodiments, the polymorphism comprising rs7028891 comprises a “G” allele at nucleobase 501 within rs7028891. In some embodiments, the polymorphism comprising rs7863183 comprises a “G” allele at nucleobase 1741 within rs78631831741 within rs7863183. In some embodiments, the polymorphism comprising rs4979469 comprises an “A” allele at nucleobase 201 within rs4979469201 within rs4979469. In some embodiments, the polymorphism comprising rs1853187 comprises a “G” allele at nucleobase 642 within rs1853187642 within rs1853187. In some embodiments, the polymorphism comprising rs7040029 comprises a “G” allele at nucleobase 201 within rs7040029. In some embodiments, the polymorphism comprising rs722126 comprises an “A” allele at nucleobase 501 within rs722126. In some embodiments, the polymorphism comprising rs4246905 comprises a “G” allele at nucleobase 501 within rs4246905. In some embodiments, the polymorphism comprising rs4979467 comprises an “A” allele at nucleobase 501 within rs4979467. In some embodiments, the polymorphism comprising rs4979466 comprises a “G” allele at nucleobase 501 within rs4979466. In some embodiments, the polymorphism comprising rs7043505 comprises an “A” allele at nucleobase 946 within rs7043505. In some embodiments, the polymorphism comprising rs911605 comprises an “A” allele at nucleobase 501 within rs911605. In some embodiments, the polymorphism comprising rs11793394 comprises an “A” allele at nucleobase 501 within rs11793394. In some embodiments, the polymorphism comprising rs17219926 comprises a “G” allele at nucleobase 501 within rs17219926. In some embodiments, the polymorphism comprising rs7874896 comprises an “A” allele at nucleobase 5370 within rs7874896. In some embodiments, the polymorphism comprising rs6478106 comprises an “A” allele at nucleobase 501 within rs6478106. In some embodiments, the polymorphism comprising rs7032238 comprises a “G” allele at nucleobase 501 within rs7032238. In some embodiments, the polymorphism comprising rs55775610 comprises an “A” allele at nucleobase 501 within rs55775610. In some embodiments, the polymorphism comprising rs7847158 comprises a “G” allele at nucleobase 501 within rs7847158. In some embodiments, the polymorphism comprising rs56069985 comprises a “G” allele at nucleobase 401 within rs56069985. In some embodiments, the polymorphism comprising rs6478109 comprises a “G” allele at nucleobase 501 within rs6478109. In some embodiments, the polymorphism comprising rs201292440 comprises an insertion of a nucleic acid, I, at nucleobase 501 within rs201292440. In some embodiments, the polymorphism comprising rs7848647 comprises a “G” allele at nucleobase 501 within rs7848647. In some embodiments, the polymorphism comprising rs7869487 comprises an “A” allele at nucleobase 501 within rs7869487. In some embodiments, the polymorphism comprising rs6478108 comprises an “A” allele at nucleobase 501 within rs6478108. In some embodiments, the polymorphism comprising rs10114470 comprises a “G” allele at nucleobase 501 within rs10114470. In some embodiments, the polymorphism comprising rs4574921 comprises an “A” allele at nucleobase 501 within rs4574921. In some embodiments, two copies of the polymorphism are detected in the sample obtained from the subject. In some embodiments, one copy of the polymorphism is detected in the sample obtained from the subject. In some embodiments, the inflammatory condition or disease comprises inflammatory bowel disease (IBD), Crohn's disease (CD), perianal Crohn's disease (pCD), ulcerative colitis (UC), rheumatoid arthritis, multiple sclerosis, psoriasis, chronic colitis, pancreatitis, leukopenia, chronic asthma, or a combination thereof. In some embodiments, the fibrostenotic or fibrotic disease comprises colonic fibrosis, pulmonary fibrosis, primary sclerosing cholangitis, progressive systemic sclerosis, or fibrostenosis of a small or large intestine. In some embodiments, the polymorphism is detected by using an assay comprising DNA sequencing a genotyping array, enzymatic amplification, allelic discrimination, restriction fragment length polymorphism analysis, allele-specific oligonucleotide hybridization, heteroduplex mobility assay, single strand conformational polymorphism, or denaturing gradient gel electrophoresis, or any combination thereof. In some embodiments, the polymorphism is detected by contacting the sample obtained from the subject with a nucleic acid sequence capable of hybridizing to at least about 10 but less than 50 contiguous nucleobases of any one of SEQ ID NOS: 5,7-9, 11-26, 28-36, and 73-82 or reverse complement sequence thereof, under standard hybridization conditions. In some embodiments, the standard hybridization conditions comprise an annealing temperature between about 30° C. and about 65° C. In some embodiments, the nucleic acid sequence is conjugated to a detectable molecule. In some embodiments, the detectable molecule comprises a fluorophore. In some embodiments, the nucleic acid sequence is conjugated to a quencher. In some embodiments, the sample obtained from the subject comprises gene material that is amplified using a nucleic acid amplification assay. In some embodiments, the nucleic acid amplification assay comprises amplification of DNA from the subject with a pair of primers capable of amplifying at least about 10 and less than 50 nucleobases within rs4366152, rs1407308, rs7866342, rs7030574, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs6478106, rs7032238, rs55775610, rs7847158, rs56069985, rs10790957, rs6921610, rs6757588 or rs6003160. In some embodiments, the nucleic acid amplification assay comprises amplification of DNA from the subject with a pair of primers capable of amplifying at least about 10 but less than 50 contiguous nucleobases within any one of SEQ ID NOS: 5,7-9, 11-26, 28-36, and 73-82. In some embodiments, the sample obtained from the subject comprises whole blood, blood plasma, blood serum, cheek swab, urine, saliva, or tissue. In some embodiments, the subject is a mammal. In some embodiments, the subject is a human. In some embodiments, the subject is susceptible to, or is inflicted with, thiopurine toxicity, or a disease caused by thiopurine toxicity. In some embodiments, the subject is non-responsive to a therapy comprising anti-TNF alpha therapy, anti-a4-b7 therapy (vedolizumab), anti-IL12p40 therapy (ustekinumab), Thalidomide, or Cytoxan. In some embodiments, the polymorphism is associated with a disease phenotype comprising non-stricturing/non-penetrating, stricturing, stricturing and penetrating or isolated internal penetrating. In some embodiments, the polymorphism is associated with perianal Crohn's disease (pCD). In some embodiments, the polymorphism is associated with an increase or a decrease in TL1A expression in a disease location comprising ileal, colonic, or ileocolonic, or a combination thereof. In some embodiments, the polymorphism is associated with a time to first surgery, or a time to second surgery, or a combination thereof. In some embodiments, the methods further comprise administering to the subject an inhibitor of TL1A expression or activity. In some embodiments, the inhibitor of TL1A expression or activity comprises a TL1A antibody, or a TL1A-binding antibody fragment. In some embodiments, the inhibitor of TL1A expression or activity comprises one or more of the sequences of Table 1. In some embodiments, the inhibitor of TL1A expression or activity comprises a blocking anti-TL1A antibody. In some embodiments, the inhibitor of TL1A expression or activity comprises a small molecule that binds to TL1A or DR3. In some embodiments, the inhibitor of TL1A expression or activity is effective to inhibit TL1A-DR3 binding. In some embodiments, the inhibitor of TL1A expression or activity comprises an allosteric modulator of TL1A.

### Certain Terminologies

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It can also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by about 10%, 5%, or 1%.

As used herein “consisting essentially of” when used to define compositions and methods, shall mean excluding other elements of any essential significance to the combination for the stated purpose. Thus, a composition consisting essentially of the elements as defined herein may not exclude other materials or steps that do not materially affect the basic and novel characteristic(s) of the claimed disclosure, such as compositions for treating skin disorders like acne, eczema, psoriasis, and rosacea.

The terms “homologous,” “homology,” or “percent homology” are used herein to generally mean an amino acid sequence or a nucleic acid sequence having the same, or similar sequence to a reference sequence. Percent homology of sequences can be determined using the most recent version of BLAST, as of the filing date of this application.

The terms “increased,” or “increase” are used herein to generally mean an increase by a statically significant amount; in some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, 60-fold, 70-fold, 80-fold, 90-fold, 100-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is in some embodiments down to a level accepted as within the range of normal for an individual without a given disease.

The term, “polymorphism,” as disclosed herein, refers to a variation in a polynucleotide sequence within a gene. The polymorphism may comprise a single nucleotide polymorphism (SNP) at an allele. The polymorphism may be a substitution, insertion, or deletion, of a nucleobase. In some embodiments, the polymorphism is represented by an “rs” number, which refers to the accession of refSNP cluster of one more submitted polymorphisms in the FASTA bioinformatics database, and which is characterized by a FASTA sequence that comprises the total number of nucleobases from 5′ to 3′, including the variation, that was submitted. In some embodiments, a polymorphism may be further defined by the position of the polymorphism (nucleobase) within this sequence, which is always the 5′ length of the sequence plus 1.

“Fold-change,” as used herein, refers to a change in a quantity or level of expression of a gene, or gene expression product thereof, from an initial to a final value. Fold-change may be measured over a period of time, or at a single point in time, or a combination thereof. Fold-change may be an increase or a decrease as compared to the initial value. In some embodiments, the gene comprises deoxynucleicribonucleic acid (DNA). In some embodiments, the gene expression product comprises ribonucleic acid (RNA), or protein, or both. In some embodiments, the RNA comprises messenger RNA (mRNA).

“Linkage disequilibrium,” or “LD,” as used herein refers to the non-random association of alleles at different loci in a population. LD may be defined by a D′ value corresponding to the difference between an observed and expected allele frequencies in the population (D=Pab−PaPb), which is scaled by the theoretical maximum value of D. LD may be defined by an r² value corresponding to the difference between an observed and expected allele frequencies in the population (D=Pab−PaPb), which is scaled by the individual frequencies of the different loci.

“Treatment” and “treating” as used herein refer to both therapeutic treatment and prophylactic or preventative measures, wherein the object is to prevent or slow down (lessen) the targeted pathologic condition, prevent the pathologic condition, pursue or obtain good overall survival, or lower the chances of the individual developing the condition even if the treatment is ultimately unsuccessful. In some aspects provided herein, subjects in need of treatment include those already with a disease or condition, as well as those susceptible to develop the disease or condition or those in whom the disease or condition is to be prevented. The disease or condition may comprise an inflammatory disease or condition, fibrostenotic or fibrotic disease, thiopurine toxicity or disease related to thiopurine toxicity, non-response to anti-TNF therapy, steroids or immunomodulators.

Non-limiting examples of “sample” include any material from which nucleic acids or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large or small intestine. In various embodiments, the large intestine sample comprises the cecum, colon (the ascending colon, the transverse colon, the descending colon, and the sigmoid colon), rectum or the anal canal. In some embodiments, the small intestine sample comprises the duodenum, jejunum, or the ileum. Alternatively, a sample can be obtained through primary patient derived cell lines, or archived patient samples in the form of preserved samples, or fresh frozen samples.

Provided throughout this application are kits, compositions and methods for the treatment of IBD. It may be understood that kits and compositions disclosed herein may be used according to, or for, methods described herein. Conversely, methods disclosed herein may appropriately employ compositions disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 shows association of TL1A fold-change levels with the TNFSF15 causal single nucleotide polymorphism (SNP). The major allele is risk SNP associated with increased TL1A fold-change levels while the minor allele is non-risk. The risk population contains homozygous or heterozygous risk. The horizontal line indicates the mean+/−standard deviation of TL1A fold-change level associated with TNFSF15 non-risk population.

### DETAILED DESCRIPTION

In one aspect, provided herein, are methods of obtaining a sample from a subject and assaying the sample to detect a presence of a polymorphism associated with expression of tumor necrosis factor ligand superfamily member 15 (TL1A) and nucleic acids encoding TL1A (e.g., TNFSF15). In one aspect, provided herein, are methods of treating an inflammatory disease or condition, or a fibrotic or fibrostenotic disease or condition, by administering to the subject a therapeutically effective amount of an inhibitor of TL1A expression or activity, provided the presence of the polymorphism is detected in the sample obtained from the subject. In one aspect, provided herein, are compositions and kits for the detection of the polymorphism associated with TL1A and nucleic acids encoding TL1A.

### Methods of Treating an Inflammatory Disease or Condition, or Fibrostenotic or Fibrotic Disease

In one aspect, provided herein are methods of treating an inflammatory disease or condition, or fibrostenotic or fibrotic disease in a subject, provided a polymorphism at a gene locus is detected in a sample obtained from the subject. In some embodiments, the subject is a mammal. In some embodiments, the subject is a human. In some embodiments, the inflammatory condition or disease comprises a condition that involves chronic inflammation of the body caused by pathogens, viruses, foreign bodies or overactive immune responses. Non-limiting examples of inflammatory conditions include, but are not limited to, inflammatory bowel disease (IBD), Crohn's disease (CD), perianal Crohn's disease (pCD), ulcerative colitis (UC), rheumatoid arthritis, multiple sclerosis, scleroderma, psoriasis, chronic colitis, pancreatitis, leukopenia, chronic asthma, or a combination thereof. In some embodiments, the fibrosis comprises colonic fibrosis, pulmonary fibrosis, primary sclerosing cholangitis, progressive systemic sclerosis, or fibrostenosis of a small or large intestine. In some embodiments, the subject is susceptible to, or is inflicted with, thiopurine toxicity, or a disease caused by thiopurine toxicity (such as pancreatitis or leukopenia). In further embodiments provided, the subject is non-responsive to a therapy comprising anti-tumor necrosis factor (TNF) alpha therapy, anti-a4-b7 therapy (e.g., vedolizumab), anti-IL12p40 therapy (e.g., ustekinumab), Thalidomide, or Cytoxan.

### Inhibitor of TL1A Expression or Activity

In one aspect, provided herein are methods of treating an inflammatory disease or condition, or fibrostenotic or fibrotic disease in a subject by administering a therapeutically effective amount of an inhibitor of TL1A expression or activity to the subject, provided a polymorphism at a gene locus is detected in a sample obtained from the subject. In some embodiments, the inhibitor of TL1A expression or activity is effective to inhibit TL1A-DR3 binding. In some embodiments, the inhibitor of TL1A expression or activity comprises an allosteric modulator of TL1A. An allosteric modulator of TL1A may indirectly influence the effects TL1A on DR3, or TR6/DcR3 on TL1A or DR3. The inhibitor of TL1A expression or activity may be a direct inhibitor or indirect inhibitor. Non-limiting examples of an inhibitor of TL1A expression include RNA to protein TL1A translation inhibitors, antisense oligonucleotides targeting the TNFSF15 mRNA (such as miRNAs, or siRNA), epigenetic editing (such as targeting the DNA-binding domain of TNFSF15, or post-translational modifications of histone tails or DNA molecules). Non-limiting examples of an inhibitor of TL1A activity include antagonists to the TL1A receptors, (DR3 and TR6/DcR3), antagonists to TL1A antigen, and antagonists to gene expression products involved in TL1A mediated disease. Antagonists as disclosed herein, may include, but are not limited to, an anti-TL1A antibody, an anti-TL1A-binding antibody fragment, or a small molecule. The small molecule may be a small molecule that binds to TL1A or DR3. The anti-TL1A antibody may be monoclonal or polyclonal. The anti-TL1A antibody may be humanized or chimeric. The anti-TL1A antibody may be a fusion protein. The anti-TL1A antibody may be a blocking anti-TL1A antibody. A blocking antibody blocks binding between two proteins, e.g., a ligand and its receptor. Therefore, a TL1A blocking antibody includes an antibody that prevents binding of TL1A to DR3 or TR6/DcR3 receptors. In a non-limiting example, the TL1A blocking antibody binds to DR3. In another example, the TL1A blocking antibody binds to DcR3. In some cases, the TL1A antibody is an anti-TL1A antibody that specifically binds to TL1A. The anti-TL1A antibody may comprise one or more of the antibody sequences of Table 1, Table 2, or Table 8. The anti-DR3 antibody may comprise an amino acid sequence that is at least 85% identical to any one of SEQ ID NOS: 258-270 and an amino acid sequence that is at least 85% identical to any one of SEQ ID NOS: 271-275. The anti-DR3 antibody may comprise an amino acid sequence comprising the HCDR1, HCDR2, HCDR3 domains of any one of SEQ ID NOS: 258-270 and the LCDR1, LCDR2, and LCDR3 domains of any one of SEQ ID NOS: 271-275.

In some embodiments, an anti-TL1A antibody comprises a heavy chain comprising three complementarity-determining regions: HCDR1, HCDR2, and HCDR3; and a light chain comprising three complementarity-determining regions: LCDR1, LCDR2, and LCDR3. In some embodiments, the anti-TL1A antibody comprises a HCDR1 comprising SEQ ID NO: 109, a HCDR2 comprising SEQ ID NO: 110, a HCDR3 comprising SEQ ID NO: 111, a LCDR1 comprising SEQ ID NO: 112, a LCDR2 comprising SEQ ID NO: 113, and a LCDR3 comprising SEQ ID NO: 114. In some cases, the anti-TL1A antibody comprises a heavy chain (HC) variable domain comprising SEQ ID NO: 115 and a light chain (LC) variable domain comprising SEQ ID NO: 116.

In some embodiments, the anti-TL1A antibody comprises a HCDR1 comprising SEQ ID NO: 117, a HCDR2 comprising SEQ ID NO: 118, a HCDR3 comprising SEQ ID NO: 119, a LCDR1 comprising SEQ ID NO: 120, a LCDR2 comprising SEQ ID NO: 121, and a LCDR3 comprising SEQ ID NO: 122. In some cases, the anti-TL1A antibody comprises a heavy chain (HC) variable domain comprising SEQ ID NO: 123 and a light chain (LC) variable domain comprising SEQ ID NO: 124.

In some embodiments, the anti-TL1A antibody comprises a HCDR1 comprising SEQ ID NO: 125, a HCDR2 comprising SEQ ID NO: 126, a HCDR3 comprising SEQ ID NO: 127, a LCDR1 comprising SEQ ID NO: 128, a LCDR2 comprising SEQ ID NO: 129, and a LCDR3 comprising SEQ ID NO: 130. In some cases, the anti-TL1A antibody comprises a heavy chain (HC) variable domain comprising SEQ ID NO: 131 and a light chain (LC) variable domain comprising SEQ ID NO: 132.

In some embodiments, the anti-TL1A antibody comprises a HCDR1 comprising SEQ ID NO: 133, a HCDR2 comprising SEQ ID NO: 134, a HCDR3 comprising SEQ ID NO: 135, a LCDR1 comprising SEQ ID NO: 139, a LCDR2 comprising SEQ ID NO: 140, and a LCDR3 comprising SEQ ID NO: 141. In some cases, the anti-TL1A antibody comprises a HCDR1 comprising SEQ ID NO: 136, a HCDR2 comprising SEQ ID NO: 137, a HCDR3 comprising SEQ ID NO: 138, a LCDR1 comprising SEQ ID NO: 139, a LCDR2 comprising SEQ ID NO: 140, and a LCDR3 comprising SEQ ID NO: 141. In some cases, the anti-TL1A antibody comprises a heavy chain (HC) variable domain comprising SEQ ID NO: 142 and a light chain (LC) variable domain comprising SEQ ID NO: 143. In some cases, the anti-TL1A antibody comprises a heavy chain comprising SEQ ID NO: 144. In some cases, the anti-TL1A antibody comprises a light chain comprising SEQ ID NO: 145.

In some embodiments, the anti-TL1A antibody comprises a HCDR1 comprising SEQ ID NO: 146, a HCDR2 comprising SEQ ID NO: 147, a HCDR3 comprising SEQ ID NO: 148, a LCDR1 comprising SEQ ID NO: 149, a LCDR2 comprising SEQ ID NO: 150, and a LCDR3 comprising SEQ ID NO: 151. In some cases, the anti-TL1A antibody comprises a heavy chain (HC) variable domain comprising SEQ ID NO: 152 and a light chain (LC) variable domain comprising SEQ ID NO: 153.

In some embodiments, the anti-TL1A antibody comprises a HCDR1 comprising SEQ ID NO: 154, a HCDR2 comprising SEQ ID NO: 155, a HCDR3 comprising SEQ ID NO: 156, a LCDR1 comprising SEQ ID NO: 157, a LCDR2 comprising SEQ ID NO: 158, and a LCDR3 comprising SEQ ID NO: 159. In some cases, the anti-TL1A antibody comprises a heavy chain (HC) variable domain comprising SEQ ID NO: 160 and a light chain (LC) variable domain comprising SEQ ID NO: 161.

In some embodiments, the anti-TL1A antibody comprises a HCDR1 comprising SEQ ID NO: 162, a HCDR2 comprising SEQ ID NO: 164, a HCDR3 comprising SEQ ID NO: 165, a LCDR1 comprising SEQ ID NO: 167, a LCDR2 comprising SEQ ID NO: 169, and a LCDR3 comprising SEQ ID NO: 170. In some cases, the anti-TL1A antibody comprises a heavy chain (HC) variable domain comprising SEQ ID NO: 171 and a light chain (LC) variable domain comprising SEQ ID NO: 175. In some cases, the anti-TL1A antibody comprises a heavy chain (HC) variable domain comprising SEQ ID NO: 171 and a light chain (LC) variable domain comprising SEQ ID NO: 176. In some cases, the anti-TL1A antibody comprises a heavy chain (HC) variable domain comprising SEQ ID NO: 171 and a light chain (LC) variable domain comprising SEQ ID NO: 177. In some cases, the anti-TL1A antibody comprises a heavy chain (HC) variable domain comprising SEQ ID NO: 171 and a light chain (LC) variable domain comprising SEQ ID NO: 178.

In some embodiments, the anti-TL1A antibody comprises a HCDR1 comprising SEQ ID NO: 162, a HCDR2 comprising SEQ ID NO: 164, a HCDR3 comprising SEQ ID NO: 165, a LCDR1 comprising SEQ ID NO: 168, a LCDR2 comprising SEQ ID NO: 169, and a LCDR3 comprising SEQ ID NO: 170. In some cases, the anti-TL1A antibody comprises a heavy chain (HC) variable domain comprising SEQ ID NO: 171 and a light chain (LC) variable domain comprising SEQ ID NO: 179. In some cases, the anti-TL1A antibody comprises a heavy chain (HC) variable domain comprising SEQ ID NO: 171 and a light chain (LC) variable domain comprising SEQ ID NO: 180. In some cases, the anti-TL1A antibody comprises a heavy chain (HC) variable domain comprising SEQ ID NO: 171 and a light chain (LC) variable domain comprising SEQ ID NO: 181. In some cases, the anti-TL1A antibody comprises a heavy chain (HC) variable domain comprising SEQ ID NO: 171 and a light chain (LC) variable domain comprising SEQ ID NO: 182.

In some embodiments, the anti-TL1A antibody comprises a HCDR1 comprising SEQ ID NO: 162, a HCDR2 comprising SEQ ID NO: 164, a HCDR3 comprising SEQ ID NO: 165, a LCDR1 comprising SEQ ID NO: 167, a LCDR2 comprising SEQ ID NO: 169, and a LCDR3 comprising SEQ ID NO: 170. In some cases, the anti-TL1A antibody comprises a heavy chain (HC) variable domain comprising SEQ ID NO: 172 and a light chain (LC) variable domain comprising SEQ ID NO: 175. In some cases, the anti-TL1A antibody comprises a heavy chain (HC) variable domain comprising SEQ ID NO: 172 and a light chain (LC) variable domain comprising SEQ ID NO: 176. In some cases, the anti-TL1A antibody comprises a heavy chain (HC) variable domain comprising SEQ ID NO: 172 and a light chain (LC) variable domain comprising SEQ ID NO: 177. In some cases, the anti-TL1A antibody comprises a heavy chain (HC) variable domain comprising SEQ ID NO: 172 and a light chain (LC) variable domain comprising SEQ ID NO: 178.

In some embodiments, the anti-TL1A antibody comprises a HCDR1 comprising SEQ ID NO: 162, a HCDR2 comprising SEQ ID NO: 164, a HCDR3 comprising SEQ ID NO: 165, a LCDR1 comprising SEQ ID NO: 168, a LCDR2 comprising SEQ ID NO: 169, and a LCDR3 comprising SEQ ID NO: 170. In some cases, the anti-TL1A antibody comprises a heavy chain (HC) variable domain comprising SEQ ID NO: 172 and a light chain (LC) variable domain comprising SEQ ID NO: 179. In some cases, the anti-TL1A antibody comprises a heavy chain (HC) variable domain comprising SEQ ID NO: 172 and a light chain (LC) variable domain comprising SEQ ID NO: 180. In some cases, the anti-TL1A antibody comprises a heavy chain (HC) variable domain comprising SEQ ID NO: 172 and a light chain (LC) variable domain comprising SEQ ID NO: 181. In some cases, the anti-TL1A antibody comprises a heavy chain (HC) variable domain comprising SEQ ID NO: 172 and a light chain (LC) variable domain comprising SEQ ID NO: 182.

In some embodiments, the anti-TL1A antibody comprises a HCDR1 comprising SEQ ID NO: 163, a HCDR2 comprising SEQ ID NO: 164, a HCDR3 comprising SEQ ID NO: 166, a LCDR1 comprising SEQ ID NO: 167, a LCDR2 comprising SEQ ID NO: 169, and a LCDR3 comprising SEQ ID NO: 170. In some cases, the anti-TL1A antibody comprises a heavy chain (HC) variable domain comprising SEQ ID NO: 173 and a light chain (LC) variable domain comprising SEQ ID NO: 175. In some cases, the anti-TL1A antibody comprises a heavy chain (HC) variable domain comprising SEQ ID NO: 173 and a light chain (LC) variable domain comprising SEQ ID NO: 176. In some cases, the anti-TL1A antibody comprises a heavy chain (HC) variable domain comprising SEQ ID NO: 173 and a light chain (LC) variable domain comprising SEQ ID NO: 177. In some cases, the anti-TL1A antibody comprises a heavy chain (HC) variable domain comprising SEQ ID NO: 173 and a light chain (LC) variable domain comprising SEQ ID NO: 178. In some cases, the anti-TL1A antibody comprises a heavy chain (HC) variable domain comprising SEQ ID NO: 173 and a light chain (LC) variable domain comprising SEQ ID NO: 179. In some cases, the anti-TL1A antibody comprises a heavy chain (HC) variable domain comprising SEQ ID NO: 173 and a light chain (LC) variable domain comprising SEQ ID NO: 180. In some cases, the anti-TL1A antibody comprises a heavy chain (HC) variable domain comprising SEQ ID NO: 173 and a light chain (LC) variable domain comprising SEQ ID NO: 181. In some cases, the anti-TL1A antibody comprises a heavy chain (HC) variable domain comprising SEQ ID NO: 173 and a light chain (LC) variable domain comprising SEQ ID NO: 182.

In some embodiments, the anti-TL1A antibody comprises a HCDR1 comprising SEQ ID NO: 163, a HCDR2 comprising SEQ ID NO: 164, a HCDR3 comprising SEQ ID NO: 166, a LCDR1 comprising SEQ ID NO: 168, a LCDR2 comprising SEQ ID NO: 169, and a LCDR3 comprising SEQ ID NO: 170. In some cases, the anti-TL1A antibody comprises a heavy chain (HC) variable domain comprising SEQ ID NO: 174 and a light chain (LC) variable domain comprising SEQ ID NO: 179. In some cases, the anti-TL1A antibody comprises a heavy chain (HC) variable domain comprising SEQ ID NO: 174 and a light chain (LC) variable domain comprising SEQ ID NO: 180. In some cases, the anti-TL1A antibody comprises a heavy chain (HC) variable domain comprising SEQ ID NO: 174 and a light chain (LC) variable domain comprising SEQ ID NO: 181. In some cases, the anti-TL1A antibody comprises a heavy chain (HC) variable domain comprising SEQ ID NO: 174 and a light chain (LC) variable domain comprising SEQ ID NO: 182. In some cases, the anti-TL1A antibody comprises a heavy chain (HC) variable domain comprising SEQ ID NO: 174 and a light chain (LC) variable domain comprising SEQ ID NO: 175. In some cases, the anti-TL1A antibody comprises a heavy chain (HC) variable domain comprising SEQ ID NO: 174 and a light chain (LC) variable domain comprising SEQ ID NO: 176. In some cases, the anti-TL1A antibody comprises a heavy chain (HC) variable domain comprising SEQ ID NO: 174 and a light chain (LC) variable domain comprising SEQ ID NO: 177. In some cases, the anti-TL1A antibody comprises a heavy chain (HC) variable domain comprising SEQ ID NO: 174 and a light chain (LC) variable domain comprising SEQ ID NO: 178.

In some embodiments, the anti-TL1A antibody comprises a HCDR1 comprising SEQ ID NO: 183, a HCDR2 comprising SEQ ID NO: 184, a HCDR3 comprising SEQ ID NO: 185, a LCDR1 comprising SEQ ID NO: 186, a LCDR2 comprising SEQ ID NO: 187, and a LCDR3 comprising SEQ ID NO: 188. In some cases, the anti-TL1A antibody comprises a heavy chain (HC) variable domain comprising SEQ ID NO: 189 and a light chain (LC) variable domain comprising SEQ ID NO: 194. In some cases, the anti-TL1A antibody comprises a heavy chain (HC) variable domain comprising SEQ ID NO: 189 and a light chain (LC) variable domain comprising SEQ ID NO: 195. In some cases, the anti-TL1A antibody comprises a heavy chain (HC) variable domain comprising SEQ ID NO: 189 and a light chain (LC) variable domain comprising SEQ ID NO: 196. In some cases, the anti-TL1A antibody comprises a heavy chain (HC) variable domain comprising SEQ ID NO: 189 and a light chain (LC) variable domain comprising SEQ ID NO: 197. In some cases, the anti-TL1A antibody comprises a heavy chain (HC) variable domain comprising SEQ ID NO: 190 and a light chain (LC) variable domain comprising SEQ ID NO: 194. In some cases, the anti-TL1A antibody comprises a heavy chain (HC) variable domain comprising SEQ ID NO: 190 and a light chain (LC) variable domain comprising SEQ ID NO: 195. In some cases, the anti-TL1A antibody comprises a heavy chain (HC) variable domain comprising SEQ ID NO: 190 and a light chain (LC) variable domain comprising SEQ ID NO: 196. In some cases, the anti-TL1A antibody comprises a heavy chain (HC) variable domain comprising SEQ ID NO: 190 and a light chain (LC) variable domain comprising SEQ ID NO: 197. In some cases, the anti-TL1A antibody comprises a heavy chain (HC) variable domain comprising SEQ ID NO: 191 and a light chain (LC) variable domain comprising SEQ ID NO: 194. In some cases, the anti-TL1A antibody comprises a heavy chain (HC) variable domain comprising SEQ ID NO: 191 and a light chain (LC) variable domain comprising SEQ ID NO: 195. In some cases, the anti-TL1A antibody comprises a heavy chain (HC) variable domain comprising SEQ ID NO: 191 and a light chain (LC) variable domain comprising SEQ ID NO: 196. In some cases, the anti-TL1A antibody comprises a heavy chain (HC) variable domain comprising SEQ ID NO: 191 and a light chain (LC) variable domain comprising SEQ ID NO: 197. In some cases, the anti-TL1A antibody comprises a heavy chain (HC) variable domain comprising SEQ ID NO: 192 and a light chain (LC) variable domain comprising SEQ ID NO: 194. In some cases, the anti-TL1A antibody comprises a heavy chain (HC) variable domain comprising SEQ ID NO: 192 and a light chain (LC) variable domain comprising SEQ ID NO: 195. In some cases, the anti-TL1A antibody comprises a heavy chain (HC) variable domain comprising SEQ ID NO: 192 and a light chain (LC) variable domain comprising SEQ ID NO: 196. In some cases, the anti-TL1A antibody comprises a heavy chain (HC) variable domain comprising SEQ ID NO: 192 and a light chain (LC) variable domain comprising SEQ ID NO: 197. In some cases, the anti-TL1A antibody comprises a heavy chain (HC) variable domain comprising SEQ ID NO: 193 and a light chain (LC) variable domain comprising SEQ ID NO: 194. In some cases, the anti-TL1A antibody comprises a heavy chain (HC) variable domain comprising SEQ ID NO: 193 and a light chain (LC) variable domain comprising SEQ ID NO: 195. In some cases, the anti-TL1A antibody comprises a heavy chain (HC) variable domain comprising SEQ ID NO: 193 and a light chain (LC) variable domain comprising SEQ ID NO: 196. In some cases, the anti-TL1A antibody comprises a heavy chain (HC) variable domain comprising SEQ ID NO: 193 and a light chain (LC) variable domain comprising SEQ ID NO: 197.

In some embodiments, the anti-TL1A antibody comprises a HCDR1 comprising SEQ ID NO: 198, a HCDR2 comprising SEQ ID NO: 199, a HCDR3 comprising SEQ ID NO: 200, a LCDR1 comprising SEQ ID NO: 201, a LCDR2 comprising SEQ ID NO: 202, and a LCDR3 comprising SEQ ID NO: 203. In some cases, the anti-TL1A antibody comprises a heavy chain (HC) variable domain comprising SEQ ID NO: 204 and a light chain (LC) variable domain comprising SEQ ID NO: 205. In some cases, the anti-TL1A antibody comprises a heavy chain (HC) variable domain comprising SEQ ID NO: 206 and a light chain (LC) variable domain comprising SEQ ID NO: 207. In some cases, the anti-TL1A antibody comprises a heavy chain (HC) variable domain comprising SEQ ID NO: 208 and a light chain (LC) variable domain comprising SEQ ID NO: 209. In some cases, the anti-TL1A antibody comprises a heavy chain (HC) variable domain comprising SEQ ID NO: 210 and a light chain (LC) variable domain comprising SEQ ID NO: 211. In some cases, the anti-TL1A antibody comprises a heavy chain (HC) variable domain comprising SEQ ID NO: 212 and a light chain (LC) variable domain comprising SEQ ID NO: 213. In some cases, the anti-TL1A antibody comprises a heavy chain (HC) variable domain comprising SEQ ID NO: 214 and a light chain (LC) variable domain comprising SEQ ID NO: 215. In some cases, the anti-TL1A antibody comprises a heavy chain (HC) variable domain comprising SEQ ID NO: 216 and a light chain (LC) variable domain comprising SEQ ID NO: 217. In some cases, the anti-TL1A antibody comprises a heavy chain (HC) variable domain comprising SEQ ID NO: 218 and a light chain (LC) variable domain comprising SEQ ID NO: 219. In some cases, the anti-TL1A antibody comprises a heavy chain (HC) variable domain comprising SEQ ID NO: 220 and a light chain (LC) variable domain comprising SEQ ID NO: 221. In some cases, the anti-TL1A antibody comprises a heavy chain (HC) variable domain comprising SEQ ID NO: 222 and a light chain (LC) variable domain comprising SEQ ID NO: 223. In some cases, the anti-TL1A antibody comprises a heavy chain (HC) variable domain comprising SEQ ID NO: 224 and a light chain (LC) variable domain comprising SEQ ID NO: 225. In some cases, the anti-TL1A antibody comprises a heavy chain (HC) variable domain comprising SEQ ID NO: 226 and a light chain (LC) variable domain comprising SEQ ID NO: 227.

In some embodiments, the anti-TL1A antibody comprises a HCDR1 comprising SEQ ID NO: 228, a HCDR2 comprising SEQ ID NO: 229, a HCDR3 comprising SEQ ID NO: 230, a LCDR1 comprising SEQ ID NO: 231, a LCDR2 comprising SEQ ID NO: 232, and a LCDR3 comprising SEQ ID NO: 233. In some cases, the anti-TL1A antibody comprises a heavy chain (HC) variable domain comprising SEQ ID NO: 234 and a light chain (LC) variable domain comprising SEQ ID NO: 235.

In some embodiments, the anti-TL1A antibody comprises a HCDR1 comprising SEQ ID NO: 236, a HCDR2 comprising SEQ ID NO: 237, a HCDR3 comprising SEQ ID NO: 238, a LCDR1 comprising SEQ ID NO: 239, a LCDR2 comprising SEQ ID NO: 240, and a LCDR3 comprising SEQ ID NO: 241. In some cases, the anti-TL1A antibody comprises a heavy chain (HC) variable domain comprising SEQ ID NO: 242 and a light chain (LC) variable domain comprising SEQ ID NO: 243.

In some embodiments, the anti-TL1A antibody comprises a HCDR1 comprising SEQ ID NO: 246, a HCDR2 comprising SEQ ID NO: 247, a HCDR3 comprising SEQ ID NO: 248, a LCDR1 comprising SEQ ID NO: 249, a LCDR2 comprising SEQ ID NO: 250, and a LCDR3 comprising SEQ ID NO: 251. In some cases, the anti-TL1A antibody comprises a heavy chain (HC) variable domain comprising SEQ ID NO: 244 and a light chain (LC) variable domain comprising SEQ ID NO: 245. In some cases, the anti-TL1A antibody comprises a heavy chain (HC) variable domain comprising SEQ ID NO: 252 and a light chain (LC) variable domain comprising SEQ ID NO: 253. In some cases, the anti-TL1A antibody comprises a heavy chain (HC) variable domain comprising SEQ ID NO: 254 and a light chain (LC) variable domain comprising SEQ ID NO: 255. In some cases, the anti-TL1A antibody comprises a heavy chain (HC) variable domain comprising SEQ ID NO: 256 and a light chain (LC) variable domain comprising SEQ ID NO: 257.

In some embodiments, the anti-TL1A antibody comprises a HCDR1 comprising SEQ ID NO: 276, a HCDR2 comprising SEQ ID NO: 277, a HCDR3 comprising SEQ ID NO: 278, a LCDR1 comprising SEQ ID NO: 279, a LCDR2 comprising SEQ ID NO: 280, and a LCDR3 comprising SEQ ID NO: 281. In some cases, the anti-TL1A antibody comprises a heavy chain (HC) variable domain comprising SEQ ID NO: 282 and a light chain (LC) variable domain comprising SEQ ID NO: 283.

In some embodiments, the anti-TL1A antibody comprises a HCDR1 comprising SEQ ID NO: 284, a HCDR2 comprising SEQ ID NO: 285, a HCDR3 comprising SEQ ID NO: 286, a LCDR1 comprising SEQ ID NO: 287, a LCDR2 comprising SEQ ID NO: 288, and a LCDR3 comprising SEQ ID NO: 299. In some cases, the anti-TL1A antibody comprises a heavy chain (HC) variable domain comprising SEQ ID NO: 290 and alight chain (LC) variable domain comprising SEQ ID NO: 291.

In some embodiments, the anti-TL1A antibody is A100. In some embodiments, the anti-TL1A antibody is A101. In some embodiments, the anti-TL1A antibody is A102. In some embodiments, the anti-TL1A antibody is A103. In some embodiments, the anti-TL1A antibody is A104. In some embodiments, the anti-TL1A antibody is A105. In some embodiments, the anti-TL1A antibody is A106. In some embodiments, the anti-TL1A antibody is A107. In some embodiments, the anti-TL1A antibody is A108. In some embodiments, the anti-TL1A antibody is A109. In some embodiments, the anti-TL1A antibody is A110. In some embodiments, the anti-TL1A antibody is A111. In some embodiments, the anti-TL1A antibody is A112. In some embodiments, the anti-TL1A antibody is A113. In some embodiments, the anti-TL1A antibody is A114. In some embodiments, the anti-TL1A antibody is A115. In some embodiments, the anti-TL1A antibody is A116. In some embodiments, the anti-TL1A antibody is A117. In some embodiments, the anti-TL1A antibody is A118. In some embodiments, the anti-TL1A antibody is A119. In some embodiments, the anti-TL1A antibody is A120. In some embodiments, the anti-TL1A antibody is A121. In some embodiments, the anti-TL1A antibody is A122. In some embodiments, the anti-TL1A antibody is A123. In some embodiments, the anti-TL1A antibody is A124. In some embodiments, the anti-TL1A antibody is A125. In some embodiments, the anti-TL1A antibody is A126. In some embodiments, the anti-TL1A antibody is A127. In some embodiments, the anti-TL1A antibody is A128. In some embodiments, the anti-TL1A antibody is A129. In some embodiments, the anti-TL1A antibody is A130. In some embodiments, the anti-TL1A antibody is A131. In some embodiments, the anti-TL1A antibody is A132. In some embodiments, the anti-TL1A antibody is A133. In some embodiments, the anti-TL1A antibody is A134. In some embodiments, the anti-TL1A antibody is A135. In some embodiments, the anti-TL1A antibody is A136. In some embodiments, the anti-TL1A antibody is A137. In some embodiments, the anti-TL1A antibody is A138. In some embodiments, the anti-TL1A antibody is A139. In some embodiments, the anti-TL1A antibody is A140. In some embodiments, the anti-TL1A antibody is A141. In some embodiments, the anti-TL1A antibody is A142. In some embodiments, the anti-TL1A antibody is A143. In some embodiments, the anti-TL1A antibody is A144. In some embodiments, the anti-TL1A antibody is A145. In some embodiments, the anti-TL1A antibody is A146. In some embodiments, the anti-TL1A antibody is A147. In some embodiments, the anti-TL1A antibody is A148. In some embodiments, the anti-TL1A antibody is A149. In some embodiments, the anti-TL1A antibody is A150. In some embodiments, the anti-TL1A antibody is A151. In some embodiments, the anti-TL1A antibody is A152. In some embodiments, the anti-TL1A antibody is A153. In some embodiments, the anti-TL1A antibody is A154. In some embodiments, the anti-TL1A antibody is A155. In some embodiments, the anti-TL1A antibody is A156. In some embodiments, the anti-TL1A antibody is A157. In some embodiments, the anti-TL1A antibody is A158. In some embodiments, the anti-TL1A antibody is A159. In some embodiments, the anti-TL1A antibody is A160. In some embodiments, the anti-TL1A antibody is A161. In some embodiments, the anti-TL1A antibody is A162. In some embodiments, the anti-TL1A antibody is A163. In some embodiments, the anti-TL1A antibody is A164. In some embodiments, the anti-TL1A antibody is A165. In some embodiments, the anti-TL1A antibody is A166. In some embodiments, the anti-TL1A antibody is A167. In some embodiments, the anti-TL1A antibody is A168. In some embodiments, the anti-TL1A antibody is A169. In some embodiments, the anti-TL1A antibody is A170. In some embodiments, the anti-TL1A antibody is A171. In some embodiments, the anti-TL1A antibody is A172. In some embodiments, the anti-TL1A antibody is A173. In some embodiments, the anti-TL1A antibody is A174. In some embodiments, the anti-TL1A antibody is A175. In some embodiments, the anti-TL1A antibody is A176. In some embodiments, the anti-TL1A antibody is A177.

In some embodiments, the anti-DR3 is A178. In some embodiments, the anti-DR3 is A179. In some embodiments, the anti-DR3 is A180. In some embodiments, the anti-DR3 is A181. In some embodiments, the anti-DR3 is A182. In some embodiments, the anti-DR3 is A183. In some embodiments, the anti-DR3 is A184. In some embodiments, the anti-DR3 is A185. In some embodiments, the anti-DR3 is A186. In some embodiments, the anti-DR3 is A187. In some embodiments, the anti-DR3 is A188. In some embodiments, the anti-DR3 is A189. In some embodiments, the anti-DR3 is A190. In some embodiments, the anti-DR3 is A191. In some embodiments, the anti-DR3 is A192. In some embodiments, the anti-DR3 is A193. In some embodiments, the anti-DR3 is A194. In some embodiments, the anti-DR3 is A195. In some embodiments, the anti-DR3 is A196. In some embodiments, the anti-DR3 is A197. In some embodiments, the anti-DR3 is A198. In some embodiments, the anti-DR3 is A199. In some embodiments, the anti-DR3 is A200. In some embodiments, the anti-DR3 is A201. In some embodiments, the anti-DR3 is A202. In some embodiments, the anti-DR3 is A203. In some embodiments, the anti-DR3 is A204. In some embodiments, the anti-DR3 is A205. In some embodiments, the anti-DR3 is A206. In some embodiments, the anti-DR3 is A207. In some embodiments, the anti-DR3 is A208. In some embodiments, the anti-DR3 is A209. In some embodiments, the anti-DR3 is A210. In some embodiments, the anti-DR3 is A211. In some embodiments, the anti-DR3 is A212. In some embodiments, the anti-DR3 is A213. In some embodiments, the anti-DR3 is A214. In some embodiments, the anti-DR3 is A215. In some embodiments, the anti-DR3 is A216. In some embodiments, the anti-DR3 is A217. In some embodiments, the anti-DR3 is A218. In some embodiments, the anti-DR3 is A219. In some embodiments, the anti-DR3 is A220. In some embodiments, the anti-DR3 is A221. In some embodiments, the anti-DR3 is A222. In some embodiments, the anti-DR3 is A223. In some embodiments, the anti-DR3 is A224. In some embodiments, the anti-DR3 is A225. In some embodiments, the anti-DR3 is A226. In some embodiments, the anti-DR3 is A227. In some embodiments, the anti-DR3 is A228. In some embodiments, the anti-DR3 is A229. In some embodiments, the anti-DR3 is A230. In some embodiments, the anti-DR3 is A231. In some embodiments, the anti-DR3 is A232. In some embodiments, the anti-DR3 is A233. In some embodiments, the anti-DR3 is A234. In some embodiments, the anti-DR3 is A235. In some embodiments, the anti-DR3 is A236. In some embodiments, the anti-DR3 is A237. In some embodiments, the anti-DR3 is A238. In some embodiments, the anti-DR3 is A239. In some embodiments, the anti-DR3 is A240. In some embodiments, the anti-DR3 is A241. In some embodiments, the anti-DR3 is A242.

In some cases, the anti-TL1A antibody binds to at least one or more of the same residues of human TL1A as an antibody described herein. For example, the anti-TL1A antibody binds to at least one or more of the same residues of human TL1A as an antibody selected from A100-A177. In some cases, the anti-TL1A antibody binds to the same epitope of human TL1A as an antibody selected from A100-A177. In some cases, the anti-TL1A antibody binds to the same region of human TL1A as an antibody selected from A100-A177.

Non-limiting methods for determining whether an anti-TL1A antibody binds to the same region of a reference antibody can be used. In an example, method comprises a competition assay. For instance, the method comprises determining whether a reference antibody can compete with binding between the reference antibody and the TL1A protein or portion thereof, or determining whether the reference antibody can compete with binding between the reference antibody and the TL1A protein or portion thereof. In an example, methods include use of surface plasmon resonance to evaluate whether an anti-TL1A antibody can compete with the binding between TL1A and another anti-TL1A antibody. In some cases, surface plasmon resonance is utilized in the competition assay.

In some embodiments, the anti-TL1A antibody comprises an antibody or antigen-binding fragment thereof provided in any one of the following patents: U.S. Pat. Nos. 10,322,174; 10,689,439; 10,968,279; 10,822,422; 10,138,296; 10,590,201; 8,263,743; 8,728,482; 9,416,185; 9,290,576; 9,683,998; 8,642,741; 9,068,003; and 9,896,511, each of which is hereby incorporated by reference in its entirety.

TABLE 1
Non-Limiting Examples of anti-TL1A and anti-DR3 Antibodies and Portions
Thereof

| | Antibody | |
|---|---|---|
| SEQ ID | Region | Sequence |
| 109 | HCDR1 | GFTFSTYG |
| 110 | HCDR2 | ISGTGRTT |
| 111 | HCDR3 | TKERGDYYYG VFDY |
| 112 | LCDR1 | QTISSW |
| 113 | LCDR2 | AAS |
| 114 | LCDR3 | QQYHRSWT |
| 115 | HC Variable | EVQLLESGGG LVQPGKSLRL SCAVSGFTFS TYGMNWVRQA PGKGLEWVSS |
| | | ISGTGRTTYH ADSVQGRFTV SRDNSKNILY LQMNSLRADD TAVYFCTKER |
| | | GDYYYGVFDY WGQGTLVTVS S |
| 116 | LC Variable | DIQMTQSPST LSASVGDRVT ITCRASQTIS SWLAWYQQTP |
| | | EKAPKLLIYA |
| | | ASNLQSGVPS RFSGSGSGTE FTLTISSLQP DDFATYYCQQ |
| | | YHRSWTFGQG |
| | | TKVEIT |
| 117 | HCDR1 | GFTFSSYW |
| 118 | HCDR2 | IKEDGSEK |
| 119 | HCDR3 | AREDYDSYYK YGMDV |
| 120 | LCDR1 | QSILYSSNNK NY |
| 121 | LCDR2 | WAS |
| 122 | LCDR3 | QQYYSTPFT |
| 123 | HC Variable | EVQLVESGGG LVQPGGSLRL SCAVSGFTFS SYWMSWVRQA |
| | | PGKGLEWVAN |
| | | IKEDGSEKNY VDSVKGRFTL SSDNAKNSLY LQMNSLRAED |
| | | TAVYYCARED |
| | | YDSYYKYGMD VWGQGTAVIV SS |
| 124 | LC Variable | DIVMTQSPDS LAVSLGERAT INCKSSQSIL YSSNNKNYLA |
| | | WYQQKPGQPP |
| | | KLLIYWASTR ESGVPDRFSG SGSGTDFTLT ISSLQAEDVS |
| | | VYYCQQYYST |
| | | PFTFGPGTKV DIK |
| 125 | HCDR1 | GGSFTGFY |
| 126 | HCDR2 | INHRGNT |
| 127 | HCDR3 | ASPFYDFWSG SDY |
| 128 | LCDR1 | QSLVHSDGNT Y |
| 129 | LCDR2 | KIS |
| 130 | LCDR3 | MQATQFPLT |
| 131 | HC Variable | QVQLQQWGAG LLKPSETLSL TCAVYGGSFT GFYWSWIRQP |
| | | PGKGLEWIGE |
| | | INHRGNTNYN PSLKSRVTMS VDTSKNQFSL NMISVTAADT |
| | | AMYFCASPFY |
| | | DFWSGSDYWG QGTLVTVSS |
| 132 | LC Variable | DIMLTQTPLT SPVTLGQPAS ISCKSSQSLV HSDGNTYLSW |
| | | LQQRPGQPPR |
| | | LLFYKISNRF SGVPDRFSGS GAGTDFTLKI SRVEAEDVGV |
| | | YYCMQATQFP |
| | | LTFGGGTKVE IK |
| 133 | HCDR1 | GY(X1)F(X2)(X3)YGIS; X1 = P, S, D, Q, N; X2 = T, R; X3 = N, T, Y, |
| | | H |
| 134 | HCDR2 | WIS(X1)YNG(X2)(X3)(X4)YA(X5)(X6)(X7)QG; X1 = T, P, S, A; X2 = N, |
| | | G, V, K, A; X3 = T, K; X4 = H, N; X5 = Q, R; X6 = K, M; X7 = L, |
| | | H |
| 135 | HCDR3 | ENYYGSG(X1)(X2)RGGMD(X3); X1 = S, A; X2 = Y, P; X3 = V, A, |
| | | G |
| 136 | HCDR1 | GYDFTYYGIS |
| 137 | HCDR2 | WISTYNGNTH YARMLQG |
| 138 | HCDR3 | ENYYGSGAYR GGMDV |
| 139 | LCDR1 | RASQSVSSYL A |
| 140 | LCDR2 | DASNRAT |
| 141 | LCDR3 | QQRSNWPWT |
| 142 | HC Variable | QVQLVQSGAE VKKPGASVKV SCKASGYDFT YYGISWVRQA |
| | | PGQGLEWMGW |
| | | ISTYNGNTHY ARMLQGRVTM TTDTSTRTAY MELRSLRSDD |
| | | TAVYYCAREN |
| | | YYGSGAYRGG MDVWGQGTTV TVSS |
| 143 | LC Variable | EIVLTQSPAT LSLSPGERAT LSCRASQSVS SYLAWYQQKP |
| | | GQAPRLLIYD |
| | | ASNRATGIPA RFSGSGSGTD FTLTISSLEP EDFAVYYCQQ |
| | | RSNWPWTFGQ |
| | | GTKVEIK |
| 144 | HC | QVQLVQSGAE VKKPGASVKV SCKASGYDFT YYGISWVRQA |
| | | PGQGLEWMGW |
| | | ISTYNGNTHY ARMLQGRVTM TTDTSTRTAY MELRSLRSDD |
| | | TAVYYCAREN |
| | | YYGSGAYRGG MDVWGQGTTV TVSSASTKGP SVFPLAPSSK |
| | | STSGGTAALG |
| | | CLVKDYFPEP VTVSWNSGAL TSGVHTFPAV LQSSGLYSLS |
| | | SVVTVPSSSL |
| | | GTQTYICNVN HKPSNTKVDK KVEPKSCDKT HTCPPCPAPE |
| | | AAGAPSVFLF |
| | | PPKPKDTLMI SRTPEVTCVV VDVSHEDPEV KFNWYVDGVE |
| | | VHNAKTKPRE |
| | | EQYNSTYRVV SVLTVLHQDW LNGKEYKCKV SNKALPAPIE |
| | | KTISKAKGQP |
| | | REPQVYTLPP SREEMTKNQV SLTCLVKGFY PSDIAVEWES |
| | | NGQPENNYKT |
| | | TPPVLDSDGS FFLYSKLTVD KSRWQQGNVF SCSVMHEALH |
| | | NHYTQKSLSL |
| | | SPG |
| 145 | LC | EIVLTQSPAT LSLSPGERAT LSCRASQSVS SYLAWYQQKP |
| | | GQAPRLLIYD |
| | | ASNRATGIPA RFSGSGSGTD FTLTISSLEP EDFAVYYCQQ |
| | | RSNWPWTFGQ |
| | | GTKVEIKRTV AAPSVFIFPP SDEQLKSGTA SVVCLLNNFY |
| | | PREAKVQWKV |
| | | DNALQSGNSQ ESVTEQDSKD STYSLSSTLT LSKADYEKHK |
| | | VYACEVTHQG |
| | | LSSPVTKSFN RGEC |
| 146 | HCDR1 | SRSYYWG |
| 147 | HCDR2 | SIYYNGRTYY NPSLKS |
| 148 | HCDR3 | EDYGDYGAFD I |
| 149 | LCDR1 | RASQGISSAL A |
| 150 | LCDR2 | DASSLES |
| 151 | LCDR3 | QQFNSYPLT |
| 152 | HC Variable | QLQLQESGPG LVKPSETLSL TCTVSGGSIS SRSYYWGWIR |
| | | QPPGKGLEWI SLKLSSVTAA |
| | | GSIYYNGRTY YNPSLKSRVT ISVDTSKNQF |
| | | DTAVYYCARE |
| | | DYGDYGAFDI WGQGTMVTVS S |
| 153 | LC Variable | AIQLTQSPSS LSASVGDRVT ITCRASQGIS SALAWYQQKP |
| | | GKAPKLLIYD EDFATYYCQQ |
| | | ASSLESGVPS RFSGSGSGTD FTLTISSLQP |
| | | FNSYPLTFGG |
| | | GTKVEIK |
| 154 | HCDR1 | TSNMGVV |
| 155 | HCDR2 | HILWDDREYSNPALKS |
| 156 | HCDR3 | MSRNYYGSSYVMDY |
| 157 | LCDR1 | SASSSVNYMH |
| 158 | LCDR2 | STSNLAS |
| 159 | LCDR3 | HQWNNYGT |
| 160 | HC Variable | QVTLKESGPALVKPTQTLTLTCTFSGFSLSTSNMGVVWIRQPPGK |
| | | ALEWLAHILWDD |
| | | REYSNPALKSRLTISKDTSKNQVVLTMTNMDPVDTATYYCARM |
| | | SRNYYGSSYVMD YWGQGTLVTVSS |
| 161 | LC Variable | DIQLTQSPSFLSASVGDRVTITCSASSSVNYMHWYQQKPGKAPK |
| | | LLIYSTSNLASGVP |
| | | SRFSGSGSGTEFTLTISSLQPEDFATYYCHQWNNYGTFGQGTKVE |
| | | IKR |
| 162 | HCDR1 | LYGMN |
| 163 | HCDR1 | NYGMN |
| 164 | HCDR2 | WINTYTGEPTYADDFKG |
| 165 | HCDR3 | DTAMDYAMAY |
| 166 | HCDR3 | DYGKYGDYYAMDY |
| 167 | LCDRI | KSSQNIVHSDGNTYLE |
| 168 | LCDR1 | RSSQSIVHSNGNTYLD |
| 169 | LCDR2 | KVSNRFS |
| 170 | LCDR3 | FQGSHVPLT |
| 171 | HC Variable | QVQLVQSGSELKKPGASVKVSCKASGYTFTLYGMNWVRQAPG |
| | | QGLEWMG |
| | | WINTYTGEPTYADDFKGRFVFSLDTSVSTAYLQISSLKAEDTAVY |
| | | YCAR DTAMDYAMAYWGQGTLVTVSS |
| 172 | HC Variable | QVQLVQSGSELKKPGASVKVSCKASGYTFTLYGMNWVKQAPG |
| | | KGLKWMG |
| | | WINTYTGEPTYADDFKGRFVFSLDTSVSTAYLQISSLKAEDTAVY |
| | | FCAR DTAMDYAMAYWGQGTLVTVSS |
| 173 | HC Variable | QVQLVQSGSELKKPGASVKVSCKASGYTFTNYGMNWVRQAPG |
| | | QGLEWMG |
| | | WINTYTGEPTYADDFKGRFVFSLDTSVSTAYLQISSLKAEDTAVY |
| | | YCAR DYGKYGDYYAMDYWGQGTLVTVSS |
| 174 | HC Variable | QVQLVQSGSELKKPGASVKVSCKASGYTFTNYGMNWVRQAPG |
| | | KGLKWMG |
| | | WINTYTGEPTYADDFKGRFVFSLDTSVSTAYLQISSLKAEDTAVY |
| | | FCAR DYGKYGDYYAMDYWGQGTLVTVSS |
| 175 | LC Variable | DVVMTQSPLSLPVTLGQPASISCKSSQNIVHSDGNTYLEWFQQRP |
| | | GQSP |
| | | RRLIYKVSNRFSGVPDRFSGSGSGTDFTLKISRVEAEDVGVYYCF |
| | | QGSH VPLTFGGGTKVEIKR |
| 176 | LC Variable | DVVMTQSPLSLPVTLGQPASISCKSSQNIVHSDGNTYLEWFQQRP |
| | | GQSP |
| | | RRLIYKVSNRFSGVPDRFSGSGSGTDFTLKISRVEAEDVGVYYCF |
| | | QGSH VPLTFGQGTKVEIKR |
| 177 | LC Variable | DVVMTQTPLSLPVTPGEPASISCKSSQNIVHSDGNTYLEWYLQKP |
| | | GQSP |
| | | QLLIYKVSNRFSGVPDRFSGSGSGTDFTLKISRVEAEDLGVYYCF |
| | | QGSH VPLTFGGGTKVEIKR |
| 178 | LC Variable | DVVMTQTPLSLPVSLGDQASISCKSSQNIVHSDGNTYLEWYLQK |
| | | PGQSP |
| | | KVLIYKVSNRFSGVPDRFSGSGSGTDFTLKISRVEAEDLGVYYCF |
| | | QGSH VPLTFGGGTKVEIKR |
| 179 | LC Variable | DVVMTQSPLSLPVTLGQPASISCRSSQSIVHSNGNTYLDWFQQRP |
| | | GQSP |
| | | RRLIYKVSNRFSGVPDRFSGSGSGTDFTLKISRVEAEDVGVYYCF |
| | | QGSH VPLTFGGGTKVEIKR |
| 180 | LC Variable | DVVMTQSPLSLPVTLGQPASISCRSSQSIVHSNGNTYLDWFQQRP |
| | | GQSP |
| | | RRLIYKVSNRFSGVPDRFSGSGSGTDFTLKISRVEAEDVGVYYCF |
| | | QGSH VPLTFGQGTKVEIKR |
| 181 | LC Variable | DVVMTQTPLSLPVTPGEPASISCRSSQSIVHSNGNTYLDWYLQKP |
| | | GQSP |
| | | QLLIYKVSNRFSGVPDRFSGSGSGTDFTLKISRVEAEDLGVYYCF |
| | | QGSH VPLTFGGGTKVEIKR |
| 182 | LC Variable | DVVMTQTPLSLPVSLGDQASISCRSSQSIVHSNGNTYLDWYLQK |
| | | PGQSP |
| | | KVLIYKVSNRFSGVPDRFSGSGSGTDFTLKINRVEAEDLGVYFCF |
| | | QGSH VPLTFGGGTKLEIKR |
| 183 | HCDR1 | GYTFTSSWMH |
| 184 | HCDR2 | IHPNSGGT |
| 185 | HCDR3 | ARGDYYGYVSWFAY |
| 186 | LCDR1 | QNINVL |
| 187 | LCDR2 | KAS |
| 188 | LCDR3 | QQGQSYPYT |
| 189 | HC Variable | QVQLQQPGSV LVRPGASVKV SCKASGYTFT SSWMHWAKQR |
| | | PGQGLEWIGE |
| | | IHPNSGGTNY NEKFKGKATV DTSSSTAYVD LSSLTSEDSA |
| | | VYYCARGDYY |
| | | GYVSWFAYWG QGTLVTVSS |
| 190 | HC Variable | QVQLVQSGAE VKKPGASVKV SCKASGYTFT SSWMHWARQA |
| | | PGQGLEWIGE |
| | | IHPNSGGTNY AQKFQGRATL TVDTSSSTAY MELSRLRSDD |
| | | TAVYYCARGD |
| | | YYGYVSWFAY WGQGTLVTVS S |
| 191 | HC Variable | QVQLVQSGAE VKKPGASVKV SCKASGYTFT SSWMHWARQA |
| | | PGQGLEWIGE |
| | | IHPNSGGTNY AQKFQGRATM TVDTSISTAY MELSRLRSDD |
| | | TAVYYCARGD |
| | | YYGYVSWFAY WGQGTLVTVS S |
| 192 | HC Variable | QVQLVQSGAE VKKPGASVKV SCKASGYTFT SSWMHWARQA |
| | | PGQGLEWIGE |
| | | IHPNSGGTNY AQKFQGRVTM TVDTSISTAY MELSRLRSDD |
| | | TAVYYCARGD |
| | | YYGYVSWFAY WGQGTLVTVS S |
| 193 | HC Variable | QVQLVQSGAE VKKPGASVKV SCKASGYTFT SSWMHWARQA |
| | | PGQGLEWMGE |
| | | IHPNSGGTNY AQKFQGRVTM TVDTSISTAY MELSRLRSDD |
| | | TAVYYCARGD |
| | | YYGYVSWFAY WGQGTLVTVS S |
| 194 | LC Variable | DIQMNQSPSS LSASLGDTIT ITCHASQNIN VLLSWYQQKP |
| | | GNIPKLLIYK |
| | | ASNLHTGVPS RFSGSGSGTG FTFTISSLQP EDIATYYCQQ |
| | | GQSYPYTFGG |
| | | GTKLEIK |
| 195 | LC Variable | DIQMTQSPSS LSASVGDRVT ITCQASQDIS NYLNWYQQKP |
| | | GKAPKLLIYD |
| | | ASNLETGVPS RFSGSGSGTD FTFTISSLQP EDIATYYCQQ |
| | | YDNLPYTFGQ |
| | | GTKLEIK |
| 196 | LC Variable | DIQMTQSPSS LSASVGDRVT ITCQASQNIN VLLNWYQQKP |
| | | GKAPKLLIYK |
| | | ASNLHTGVPS RFSGSGSGTD FTFTISSLQP EDIATYYCQQ |
| | | GQSYPYTFGQ |
| | | GTKLEIK |
| 197 | LC Variable | DIQMNQSPSS LSASVGDRVT ITCQASQNIN VLLSWYQQKP |
| | | GKAPKLLIYK |
| | | ASNLHTGVPS RFSGSGSGTD FTFTISSLQP EDIATYYCQQ |
| | | GQSYPYTFGQ |
| | | GTKLEIK |
| 198 | HCDR1 | GYTFTSYDIN |
| 199 | HCDR2 | WLNPNSGXTG; X = N, Y |
| 200 | HCDR3 | EVPETAAFEY |
| 201 | LCDR1 | TSSSSDIGA(X1)(X2)GV(X3); X1 = G, A; X2 = L, S, Q; X3 = H, L |
| 202 | LCDR2 | GYYNRPS |
| 203 | LCDR3 | QSXDGTLSAL; X = Y, W, F |
| 204 | HC Variable | QVQLVQSGAE VKKPGASVKV SCKASGYTFT SYDINWVRQA |
| | | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 205 | LC Variable | QSVLTQPPSV SGAPGQRVTI SCTSSSSDIG AXXGVXWYQQ |
| | | LPGTAPKLLI |
| | | EGYYNRPSGV PDRFSGSKSG TSASLTITGL LPEDEGDYYC |
| | | QSXDGTLSAL |
| | | FGGGTKLTVL G |
| 206 | HC Variable | QVQLVQSGAE VKKPGASVKV SCKASGYTFT SYDINWVRQA |
| | | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 207 | LC Variable | QSVLTQPPSV SGAPGQRVTI SCTSSSSDIG AGLGVHWYQQ |
| | | LPGTAPKLLI |
| | | EGYYNRPSGV PDRFSGSKSG TSASLTITGL LPEDEGDYYC |
| | | QSWDGTLSAL |
| | | FGGGTKLTVL G |
| 208 | HC Variable | QVQLVQSGAE VKKPGASVKV SCKASGYTFT SYDINWVRQA |
| | | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 209 | LC Variable | QSVLTQPPSV SGAPGQRVTI SCTSSSSDIG AGLGVHWYQQ |
| | | LPGTAPKLLI |
| | | EGYYNRPSGV PDRFSGSKSG TSASLTITGL LPEDEGDYYC |
| | | QSYDGTLSAL |
| | | FGGGTKLTVL G |
| 210 | HC Variable | QVQLVQSGAE VKKPGASVKV SCKASGYTFT SYDINWVRQA |
| | | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 211 | LC Variable | QSVLTQPPSV SGAPGQRVTI SCTSSSSDIG AALGVHWYQQ |
| | | LPGTAPKLLI |
| | | EGYYNRPSGV PDRFSGSKSG TSASLTITGL LPEDEGDYYC |
| | | QSWDGTLSAL |
| | | FGGGTKLTVL G |
| 212 | HC Variable | QVQLVQSGAE VKKPGASVKV SCKASGYTFT SYDINWVRQA |
| | | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 213 | LC Variable | QSVLTQPPSV SGAPGQRVTI SCTSSSSDIG AGSGVHWYQQ |
| | | LPGTAPKLLI |
| | | EGYYNRPSGV PDRFSGSKSG TSASLTITGL LPEDEGDYYC |
| | | QSWDGTLSAL |
| | | FGGGTKLTVL G |
| 214 | HC Variable | QVQLVQSGAE VKKPGASVKV SCKASGYTFT SYDINWVRQA |
| | | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 215 | LC Variable | QSVLTQPPSV SGAPGQRVTI SCTSSSSDIG AGQGVHWYQQ |
| | | LPGTAPKLLI |
| | | EGYYNRPSGV PDRFSGSKSG TSASLTITGL LPEDEGDYYC |
| | | QSWDGTLSAL |
| | | FGGGTKLTVL G |
| 216 | HC Variable | QVQLVQSGAE VKKPGASVKV SCKASGYTFT SYDINWVRQA |
| | | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 217 | LC Variable | QSVLTQPPSV SGAPGQRVTI SCTSSSSDIG AGLGVLWYQQ |
| | | LPGTAPKLLI |
| | | EGYYNRPSGV PDRFSGSKSG TSASLTITGL LPEDEGDYYC |
| | | QSWDGTLSAL |
| | | FGGGTKLTVL G |
| 218 | HC Variable | QVQLVQSGAE VKKPGASVKV SCKASGYTFT SYDINWVRQA |
| | | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 219 | LC Variable | QSVLTQPPSV SGAPGQRVTI SCTSSSSDIG AGLGVHWYQQ |
| | | LPGTAPKLLI |
| | | EGYYNRPSGV PDRFSGSKSG TSASLTITGL LPEDEGDYYC |
| | | QSWDGTLSAL |
| | | FGGGTKLTVL G |
| 220 | HC Variable | QVQLVQSGAE VKKPGASVKV SCKASGYTFT SYDINWVRQA |
| | | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 221 | LC Variable | QSVLTQPPSV SGAPGQRVTI SCTSSSSDIG AGSGVHWYQQ |
| | | LPGTAPKLLI |
| | | EGYYNRPSGV PDRFSGSKSG TSASLTITGL LPEDEGDYYC |
| | | QSWDGTLSAL |
| | | FGGGTKLTVL G |
| 222 | HC Variable | QVQLVQSGAE VKKPGASVKV SCKASGYTFT SYDINWVRQA |
| | | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 223 | LC Variable | QSVLTQPPSV SGAPGQRVTI SCTSSSSDIG AGQGVHWYQQ |
| | | LPGTAPKLLI |
| | | EGYYNRPSGV PDRFSGSKSG TSASLTITGL LPEDEGDYYC |
| | | QSWDGTLSAL |
| | | FGGGTKLTVL G |
| 224 | HC Variable | QVQLVQSGAE VKKPGASVKV SCKASGYTFT SYDINWVRQA |
| | | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 225 | LC Variable | QSVLTQPPSV SGAPGQRVTI SCTSSSSDIG AGLGVLWYQQ |
| | | LPGTAPKLLI |
| | | EGYYNRPSGV PDRFSGSKSG TSASLTITGL LPEDEGDYYC |
| | | QSWDGTLSAL |
| | | FGGGTKLTVL G |
| 226 | HC Variable | QVQLVQSGAE VKKPGASVKV SCKASGYTFT SYDINWVRQA |
| | | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 227 | LC Variable | QSVLTQPPSV SGAPGQRVTI SCTSSSSDIG AGLGVHWYQQ |
| | | LPGTAPKLLI |
| | | EGYYNRPSGV PDRFSGSKSG TSASLTITGL LPEDEGDYYC |
| | | QSFDGTLSAL |
| | | FGGGTKLTVL G |
| 228 | HCDR1 | SYFWS |
| 229 | HCDR2 | YIYYSGNTKYNPSLKS |
| 230 | HCDR3 | ETGSYYGFDY |
| 231 | LCDR1 | RASQSINNYLN |
| 232 | LCDR2 | AASSLQS |
| 233 | LCDR3 | QQSYSTPRT |
| 234 | HC Variable | QVQLQESGPGLVKPSETLSLTCTVSGGSISSYFWSWIRQPPGKGL |
| | | EWIGYIYYSGNTKYNPSLKSRVTISIDTSKNQFSLKLSSVTAADTA |
| | | VYYCARETGSYYGFDYWGQGTLVTVSS |
| 235 | LC Variable | DIQMTQSPSSLSASVGDRVTITCRASQSINNYLNWYQQRPGKAP |
| | | KLLIYAASSLQSGVPSRFSGSGSGTDFTLTISSLQPGDFATYYCQQ |
| | | SYSTPRTFGQGTKLEIK |
| 236 | HCDR1 | GYYWN |
| 237 | HCDR2 | EINHAGNTNYNPSLKS |
| 238 | HCDR3 | GYCRSTTCYFDY |
| 239 | LCDR1 | RASQSVRSSYLA |
| 240 | LCDR2 | GASSRAT |
| 241 | LCDR3 | QQYGSSPT |
| 242 | HC Variable | QVQLQQWGAGLLKPSETLSLTCAVHGGSFSGYYWNWIRQPPGK |
| | | GLEWIGEINHAGNTNYNPSLKSRVTISLDTSKNQFSLTLTSVTAA |
| | | DTAVYYCARGYCRSTTCYFDYWGQGTLVTVSS |
| 243 | LC Variable | EIVLTQSPGTLSLSPGERATLSCRASQSVRSSYLAWYQQKPGQAP |
| | | RLLIYGASSRATGIPDRFSGSGSGTDFTLTISRLEPEDFAVYYCQQ |
| | | YGSSPTFGQGTRLEIK |
| 244 | HC Variable | EVQLQQSGAELVKPGASVKLSCTASGFDIQDTYMHWVKQRPEQ |
| | | GLEWIGRIDPASGHTKYDPKFQVKATITTDTSSNTAYLQLSSLTS |
| | | EDTAVYYCSRSGGLPDVWGAGTTVTVSS |
| 245 | LC Variable | QIVLSQSPAILSASPGEKVTMTCRASSSVSYMYWYQQKPGSSPKP |
| | | WIYATSNLASGVPDRFSGSGSGTSYSLTISRVEAEDAATYYCQQ |
| | | WSGNPRTFGGGTKLEIK |
| 246 | HCDR1 | GFDIQDTYMH |
| 247 | HCDR2 | RIDPASGHTKYDPKFQV |
| 248 | HCDR3 | SGGLPDV |
| 249 | LCDR1 | RASSSVSYMY |
| 250 | LCDR2 | ATSNLAS |
| 251 | LCDR3 | QQWSGNPRT |
| 252 | HC Variable | QVQLVQSGAEVKKPGASVKLSCKASGFDIQDTYMHWVRQAPG |
| | | QGLEWMGRIDPASGHTKYDPKFQVRVTMTTDTSTSTVYMELSS |
| | | LRSEDTAVYYCSRSGGLPDVWGQGTTVTVSS |
| 253 | LC Variable | EIVLTQSPGTLSLSPGERVTMSCRASSSVSYMYWYQQKPGQAPR |
| | | PWIYATSNLASGVPDRFSGSGSGTDYTLTISRLEPEDFAVYYCQQ |
| | | WSGNPRTFGGGTKLEIK |
| 254 | (CDR-grafted | QVQLVQSGAEVKKPGASVKLSCKASGFDIQDTYMHWVRQAPG |
| | LC) HC variable | QGLEWMGRIDPASGHTKYDPKFQVRVTMTRDTSTSTVYMELSS |
| | region | LRSEDTAVYYCSRSGGLPDVWGQGTTVTVSS |
| 255 | (CDR-grafted | EIVLTQSPGTLSLSPGERATLSCRASSSVSYMYWYQQKPGQAPRL |
| | LC) HC variable | LIYATSNLASGIPDRFSGSGSGTDFTLTISRLEPEDFAVYYCQQWS |
| | region | GNPRTFGGGTKLEIK |
| 256 | (CDR-grafted | QVQLVQSGAEVKKPGASVKVSCKASGFDIQDTYMHWVRQAPG |
| | HC) HC variable | QGLEWMGRIDPASGHTKYDPKFQVRVTMTRDTSTSTVYMELSS |
| | region | LRSEDTAVYYCARSGGLPDVWGQGTTVTVSS |
| 257 | (CDR-grafted | EIVLTQSPGTLSLSPGERATLSCRASSSVSYMYWYQQKPGQAPRL |
| | HC) LC variable | LIYATSNLASGVPDRFSGSGSGTDYTLTISRLEPEDFAVYYCQQW |
| | region | SGNPRTFGGGTKLEIK |
| 258 | HC variable | EVMLVESGGGLVKPGGSLKLSCAASGFTFTNYAMSWVRQTPEK |
| | | RLEWVATITSGGSYIYYLDSVKGRFTISRDNAKSTLYLQMSSLRS |
| | | EDTAIYNCARRKDGNYYYAMDYWGQGTSVTVSS |
| 259 | HC variable | EVMLVESGGGLVKPGGSLKLSCAASGFTFTNYAMSWVRQTPEK |
| | | RLEWVATITSGGSYIYYLDSVKGRFTISRDNAKSTLYLQMSSLRS |
| | | EDTAIYYCARRKDGNYYYAMDYWGQGTSVTVSS |
| 260 | HC variable | EVQLVESGGGLVKPGGSLRLSCAASGFTFTNYAMSWVRQAPGQ |
| | | RLEWVSTITSGGSYIYYLDSVKGRFTISRDNAKSTLYLQMNSLRA |
| | | EDTAVYNCARRKDGNYYYAMDYWGQGTTVTVSS |
| 261 | HC variable | EVQLVESGGGLVKPGGSLRLSCAASGFTFTNYAMSWVRQAPGQ |
| | | RLEWVSTITSGGSYIYYLDSVKGRFTISRDNAKSTLYLQMNSLRA |
| | | EDTAVYYCARRKDGNYYYAMDYWGQGTTVTVSS |
| 262 | HC variable | EVQLLESGGGLVQPGRSLRLSCAASGFTFTNYAMSWVRQAPGQ |
| | | RLEWLATITSGGSYIYYLDSVKGRFTISRDNSKSTLYLQMGSLRA |
| | | EDMAVYNCARRKDGNYYYAMDYWGQGTTVTVSS |
| 263 | HC variable | EVQLLESGGGLVQPGRSLRLSCAASGFTFTNYAMSWVRQAPGQ |
| | | RLEWLATITSGGSYIYYLDSVKGRFTISRDNSKSTLYLQMGSLRA |
| | | EDMAVYYCARRKDGNYYYAMDYWGQGTTVTVSS |
| 264 | HC variable | QVQLVESGGGLIQPGGSLRLSCAASGFTFTNYAMSWVRQARGQ |
| | | RLEWVSTITSGGSYIYYLDSVKGRFTISRDNSKSTLYMELSSLRSE |
| | | DTAVYNCARRKDGNYYYAMDYWGQGTTVTVSS |
| 265 | HC variable | QVQLVESGGGLIQPGGSLRLSCAASGFTFTNYAMSWVRQARGQ |
| | | RLEWVSTITSGGSYIYYLDSVKGRFTISRDNSKSTLYMELSSLRSE |
| | | DTAVYYCARRKDGNYYYAMDYWGQGTTVTVSS |
| 266 | HC variable | QVQLVQSGSELKKPGASVKVSCKASGFTFTNYAMSWVRQAPGK |
| | | RLEWVSTITSGGSYIYYLDSVKGRFTISRENAKSTLYLQMNSLRT |
| | | EDTALYNCARRKDGNYYYAMDYWGQGTTVTVSS |
| 267 | HC variable | QVQLVQSGSELKKPGASVKVSCKASGFTFTNYAMSWVRQAPGK |
| | | RLEWVATITSGGSYIYYLDSVKGRFTISRENAKSTLYLQMNSLRT |
| | | EDTALYYCARRKDGNYYYAMDYWGQGTTVTVSS |
| 268 | HC variable | EVQLLQSGAEVKKPGASVKVSCKASGFTFTNYAMSWVRQAPGQ |
| | | RLEWVATITSGGSYIYYLDSVKGRFTISRDNAKSTLHLQMNSLR |
| | | AEDTAVYNCARRKDGNYYYAMDYWGQGTTVTVSS |
| 269 | HC variable | EVQLLQSGAEVKKPGASVKVSCKASGFTFTNYAMSWVRQAPGQ |
| | | RLEWVATITSGGSYIYYLDSVKGRFTISRDNAKSTLHLQMNSLR |
| | | AEDTAIYYCARRKDGNYYYAMDYWGQGTTVTVSS |
| 270 | HC variable | EVMLLQSGAEVKKPGASVKVSCKASGFTFTNYAMSWVRQAPG |
| | | QRLEWVATITSGGSYIYYLDSVKGRFTISRDNAKSTLHLQMNSL |
| | | RAEDTAVYYCARRKDGNYYYAMDYWGQGTTVTVSS |
| 271 | LC variable | DIVLTQSPASLAVSLGQRATISCRASESVDSYGNSFIHWYQQKAG |
| | | QPPKLLIYRASNLESGIPARFSGSGSRTDFTLTINPVEADDVATYY |
| | | CQQSYEDPWTFGGGTKLEIK |
| 272 | LC variable | DIVLTQSPATLSLSPGERATLSCRASESVDSYGNSFIHWYQQKPG |
| | | QPPKLLIYRASNLESGIPARFSGSGSRTDFTLTISSLEPEDFAVYYC |
| | | QQSYEDPWTFGGGTKXEIK |
| 273 | LC variable | DIVLTQSPSSLSASVGDRVTITCRASESVDSYGNSFIHWYQQKPG |
| | | QPPKLLIYRASNLESGIPARFSGSGSRTDFTLTISSLQPEDFATYYC |
| | | QQSYEDPWTFGGGTKXEIK |
| 274 | LC variable | DIVLTQSPDFQSVTPKEKVTITCRASESVDSYGNSFIHWYQQKPG |
| | | QPPKLLIYRASNLESGIPARFSGSGSRTDFTLTISSLEAEDAATYY |
| | | CQQSYEDPWTFGGGTKXEIK |
| 275 | LC variable | DIVLTQTPLSLSVTPGQPASISCRASESVDSYGNSFIHWYQQKPG |
| | | QPPKLLIYRASNLESGIPARFSGSGSRTDFTLKISRVEAEDVGVYY |
| | | CQQSYEDPWTFGGGTKXEIK |
| 276 | HCDR1 | TYGMS |
| 277 | HCDR2 | WMNTYSGVTTYADDFKG |
| 278 | HCDR3 | EGYVFDDYYATDY |
| 279 | LCDR1 | RSSQNIVHSDGNTYLE |
| 280 | LCDR2 | KVSNRFS |
| 281 | LCDR3 | FQGSHVPLT |
| 282 | HC Variable | QIQLVQSGPELKKPGETVKISCKASGYTFTTYGMSWVKQAPGKG |
| | | LKWMGWMNTYSGVTTYADDFKGRFAFSLETSASTAYMQIDNL |
| | | KNEDTATYFCAREGYVFDDYYATDYWGQGTSVTVSS |
| 283 | LC Variable | DVLMTQTPLSLPVSLGDQASISCRSSQNIVHSDGNTYLEWYLQK |
| | | PGQSPKLLIYKVSNRFSGVPDRFSGSGSGTDFTLKISRVEAEDLGI |
| | | YYCFQGSHVPLTFGAGTKLELK |
| 284 | HCDR1 | KYDIN |
| 285 | HCDR2 | WIFPGDGRTDYNEKFKG |
| 286 | HCDR3 | YGPAMDY |
| 287 | LCDR1 | RSSQTIVHSNGDTYLD |
| 288 | LCDR2 | KVSNRFS |
| 289 | LCDR3 | FQGSHVPYT |
| 290 | HC Variable | MGWSWVFLFLLSVTAGVHSQVHLQQSGPELVKPGASVKLSCKA |
| | | SGYTFTKYDINWVRQRPEQGLEWIGWIFPGDGRTDYNEKFKGK |
| | | ATLTTDKSSSTAYMEVSRLTSEDSAVYFCARYGPAMDYWGQGT |
| | | SVTVA S |
| 291 | LC Variable | MKLPVRLLVLMFWIPASSSDVLMTQTPLSLPVSLGDQASISCRSS |
| | | QTIVHSNGDTYLDWFLQKPGQSPKLLIYKVSNRFSGVPDRFSGSG |
| | | SGTDFTLKISRVEAEDLGVYYCFQGSHVPYTFGGGTKLEIK |

TABLE 2
Non-Limiting Examples of anti-TL1A and anti-DR3 Antibodies

| Antibody | HC Variable Domain | LC Variable Domain |
|---|---|---|
| Name | (SEQ ID NO) | (SEQ ID NO) |
| A100 | 115 | 116 |
| A101 | 123 | 124 |
| A102 | 131 | 132 |
| A103 | 142 | 143 |
| A104 | 152 | 153 |
| A105 | 160 | 161 |
| A106 | 171 | 175 |
| A107 | 171 | 176 |
| A108 | 171 | 177 |
| A109 | 171 | 178 |
| A110 | 171 | 179 |
| A111 | 171 | 180 |
| A112 | 171 | 181 |
| A113 | 171 | 182 |
| A114 | 172 | 175 |
| A115 | 172 | 176 |
| A116 | 172 | 177 |
| A117 | 172 | 178 |
| A118 | 172 | 179 |
| A119 | 172 | 180 |
| A120 | 172 | 181 |
| A121 | 172 | 182 |
| A122 | 173 | 175 |
| A123 | 173 | 176 |
| A124 | 173 | 177 |
| A125 | 173 | 178 |
| A126 | 173 | 179 |
| A127 | 173 | 180 |
| A128 | 173 | 181 |
| A129 | 173 | 182 |
| A130 | 174 | 175 |
| A131 | 174 | 176 |
| A132 | 174 | 177 |
| A133 | 174 | 178 |
| A134 | 174 | 179 |
| A135 | 174 | 180 |
| A136 | 174 | 181 |
| A137 | 174 | 182 |
| A138 | 189 | 194 |
| A139 | 189 | 195 |
| A140 | 189 | 196 |
| A141 | 189 | 197 |
| A142 | 190 | 194 |
| A143 | 190 | 195 |
| A144 | 190 | 196 |
| A145 | 190 | 197 |
| A146 | 191 | 194 |
| A147 | 191 | 195 |
| A148 | 191 | 196 |
| A149 | 191 | 197 |
| A150 | 192 | 194 |
| A151 | 192 | 195 |
| A152 | 192 | 196 |
| A153 | 192 | 197 |
| A154 | 193 | 194 |
| A155 | 193 | 195 |
| A156 | 193 | 196 |
| A157 | 193 | 197 |
| A158 | 204 | 205 |
| A159 | 206 | 207 |
| A160 | 208 | 209 |
| A161 | 210 | 211 |
| A162 | 212 | 213 |
| A163 | 214 | 215 |
| A164 | 216 | 217 |
| A165 | 218 | 219 |
| A166 | 220 | 221 |
| A167 | 222 | 223 |
| A168 | 224 | 225 |
| A169 | 226 | 227 |
| A170 | 234 | 235 |
| A171 | 242 | 243 |
| A172 | 244 | 245 |
| A173 | 252 | 253 |
| A174 | 254 | 255 |
| A175 | 256 | 257 |
| A176 | 282 | 283 |
| A177 | 290 | 291 |
| A178 | 258 | 271 |
| A179 | 258 | 272 |
| A180 | 258 | 273 |
| A181 | 258 | 274 |
| A182 | 258 | 275 |
| A183 | 259 | 271 |
| A184 | 259 | 272 |
| A185 | 259 | 273 |
| A186 | 259 | 274 |
| A187 | 259 | 275 |
| A188 | 260 | 271 |
| A189 | 260 | 272 |
| A190 | 260 | 273 |
| A191 | 260 | 274 |
| A192 | 260 | 275 |
| A193 | 261 | 271 |
| A194 | 261 | 272 |
| A195 | 261 | 273 |
| A196 | 261 | 274 |
| A197 | 261 | 275 |
| A198 | 262 | 271 |
| A199 | 262 | 272 |
| A200 | 262 | 273 |
| A201 | 262 | 274 |
| A202 | 262 | 275 |
| A203 | 263 | 271 |
| A204 | 263 | 272 |
| A205 | 263 | 273 |
| A206 | 263 | 274 |
| A207 | 263 | 275 |
| A208 | 264 | 271 |
| A209 | 264 | 272 |
| A210 | 264 | 273 |
| A211 | 264 | 274 |
| A212 | 264 | 275 |
| A213 | 265 | 271 |
| A214 | 265 | 272 |
| A215 | 265 | 273 |
| A216 | 265 | 274 |
| A217 | 265 | 275 |
| A218 | 266 | 271 |
| A219 | 266 | 272 |
| A220 | 266 | 273 |
| A221 | 266 | 274 |
| A222 | 266 | 275 |
| A223 | 267 | 271 |
| A224 | 267 | 272 |
| A225 | 267 | 273 |
| A226 | 267 | 274 |
| A227 | 267 | 275 |
| A228 | 268 | 271 |
| A229 | 268 | 272 |
| A230 | 268 | 273 |
| A231 | 268 | 274 |
| A232 | 268 | 275 |
| A233 | 269 | 271 |
| A234 | 269 | 272 |
| A235 | 269 | 273 |
| A236 | 269 | 274 |
| A237 | 269 | 275 |
| A238 | 270 | 271 |
| A239 | 270 | 272 |
| A240 | 270 | 273 |
| A241 | 270 | 274 |
| A242 | 270 | 275 |

### Polymorphisms

In an aspect, provided herein, a polymorphism detected in a sample obtained from the subject is located at a gene locus involved in the mammalian innate and adaptive immune responses. In some embodiments, the gene locus is involved in the pathogenesis of inflammatory bowel disease (IBD). In further embodiments, the gene locus is involved in autophagy, innate immunity, adaptive immunity, barrier function, or regulator pathways. In some embodiments, the gene locus is involved in tumor necrosis factor ligand superfamily member 15 (TL1A) mediated pathways, including enhanced cytokine production from T cells and innate lymphoid cells, down-regulation of T regulatory cell function, activation of fibroblasts to myofibroblasts, upregulation of antigen presenting cells following stimulation with microbial antigens, and T-helper 1 (Th1) or Th17 driven immune response. The gene locus may comprise TNFSF15, MAGI3, ZNRF3, SNED1, PTPN22, TTC7B, SEPT8, PKIA, RAD51B, LY86, UNC13B, ETS1, ARHGAP15, SMPD3, ANKRD55, or SCUBE1, or a combination thereof.

In one aspect, provided herein, polymorphisms detected in a sample obtained from the subject. Detection of the polymorphisms disclosed herein is useful for the diagnosis, treatment, and characterization of the inflammatory disease or condition or fibrotic or fibrostenotic diseases disclosed herein. The polymorphisms may comprise single nucleotide polymorphisms (SNPs). The polymorphisms may comprise an insertion, deletion, or a substitution, in a polynucleotide sequence. The polymorphism may fall within coding regions of genes, non-coding regions of genes, or in the intergenic regions between genes. A polymorphism within a coding region of a gene may, or may not, result in a different protein isoform produced due to redundancy in the genetic code. A polymorphism within a non-coding region or intergenic region of a gene may influence the expression or activity of the gene, or gene expression products expressed from the gene.

In one aspect, provided herein, a polymorphism located at the LY86 gene locus comprising rs6921610 (SEQ ID NO: 3), or rs3851519 (SEQ ID NO: 80) or any polymorphism in linkage disequilibrium therewith, is detected in a sample obtained from the subject. In some embodiments, linkage disequilibrium may be determined using a D′ value of at least 0.70, 0.75, or 0.80. In some embodiments, linkage disequilibrium may be determined using a D′ value of 0, and an revalue of at least 0.70, 0.75, 0.80, 0.85, 0.90, or 0.95. Lymphocyte Antigen 86 (LY86) is a gene encoding a protein involved in the innate immune system and activated Toll-Like Receptor 4 (TLR4) signaling. LY86, and nucleic acids encoding LY86, are characterized by NCBI Entrez Gene ID 9450. In some embodiments, the polymorphism at the gene locus comprising LY86 comprises a “G” allele at nucleobase 700 within rs6921610. In some embodiments, the polymorphism at the gene locus comprising LY86 comprises a “A” allele at nucleobase 248 within rs3851519. In further embodiments provided herein, the polymorphism at the gene locus comprising LY86 comprises SEQ ID NO: 33. In some embodiments, the polymorphism at the gene locus comprising LY86 comprises SEQ ID NO: 80. The polymorphism may be within an intron of the LY86 gene, and may affect LY86 expression or activity. The polymorphism may be in a protein-coding region of LY86, and may additionally affect LY86 protein function. A polymorphism in linkage disequilibrium with an LY86 polymorphism is inherited with the LY86 polymorphism. The polymorphism in linkage disequilibrium may not be located in the LY86 locus. One polymorphism, or any combination of polymorphisms, may be detected in a sample obtained from the subject. In some embodiments, two copies of the polymorphism are detected in the sample obtained from the subject. A subject carrying one copy of the polymorphism has a heterozygous risk genotype. In some embodiments, one copy of the polymorphism is detected in the sample obtained from the subject. A subject carrying two copies of the polymorphism has a homozygous risk genotype. In some embodiments the presence of the polymorphism located at the gene locus comprising LY86 is associated with an increase in expression of TL1A. In further embodiments provided, are methods of obtaining the sample from a subject with an inflammatory disease or condition, or fibrostenotic or fibrotic disease. The method of obtaining the sample may include acquisition of the sample from the subject directly, or indirectly. In some embodiments provided are methods of assaying to detect in the sample a presence of a polymorphism located at the gene locus.

In one aspect, provided herein, a polymorphism located ETS1 gene locus comprising rs10790957 (SEQ ID NO: 34), rs11606640 (SEQ ID NO: 73, rs73029052 (SEQ ID NO: 74), rs11600915 (SEQ ID NO: 75), rs61909068 (SEQ ID NO: 76), rs12294634 (SEQ ID NO: 77), rs73029062 (SEQ ID NO: 78), rs11600746 (SEQ ID NO:79), rs61909072 (SEQ ID NO: 81), or rs56086356 (SEQ ID NO: 82), or any polymorphism in linkage disequilibrium therewith, is detected in a sample obtained from the subject. In some embodiments, linkage disequilibrium may be determined using a D′ value of at least 0.70, 0.75, or 0.80. In some embodiments, linkage disequilibrium may be determined using a D′ value of 0, and an revalue of at least 0.70, 0.75, 0.80, 0.85, 0.90, or 0.95. In some embodiments, the polymorphism at the gene locus comprising ETS1 comprises a “G” allele at nucleobase 501 within rs10790957. In some embodiments, the polymorphism at the gene locus comprising LY86 comprises rs3851519 or any polymorphism in linkage disequilibrium therewith. In some embodiments, the polymorphism at the gene locus comprising ETS1 comprises a “A” allele at nucleobase 301 within rs11606640. In some embodiments, the polymorphism at the gene locus comprising ETS1 comprises a “A” allele at nucleobase 251 within rs73029052. In some embodiments, the polymorphism at the gene locus comprising ETS1 comprises a “G” allele at nucleobase 301 within rs11600915. In some embodiments, the polymorphism at the gene locus comprising ETS1 comprises a “G” allele at nucleobase 251 within rs61909068. In some embodiments, the polymorphism at the gene locus comprising ETS1 comprises a “A” allele at nucleobase 323 within rs12294634. In some embodiments, the polymorphism at the gene locus comprising ETS1 comprises a “G” allele at nucleobase 251 within rs73029062. In some embodiments, the polymorphism at the gene locus comprising ETS1 comprises a “G” allele at nucleobase 301 within rs11600746. In some embodiments, the polymorphism at the gene locus comprising ETS1 comprises a “A” allele at nucleobase 251 within rs61909072. In some embodiments, the polymorphism at the gene locus comprising ETS1 comprises a “C” allele at nucleobase 501 within rs56086356. In some embodiments, the polymorphism at the gene locus comprising LY86 comprises a “A” allele at nucleobase 248 within rs3851519. ETS Proto-Oncogene 1 (ETS1) is a gene encoding a transcription factor characterized by a conserved ETS DNA-binding domain that recognizes the core consensus DNA sequence GGAA/T in target genes. ETS1, and nucleic acids encoding ETS1, are characterized by NCBI Entrez Gene ID 2113. In further embodiments, the polymorphism at the gene locus ETS1 comprises SEQ ID NO: 34. In some embodiments, the gene locus ETS1 comprises SEQ ID NO: 73. In some embodiments, the polymorphism at the gene locus comprising ETS1 comprises SEQ ID NO: 74. In some embodiments, the polymorphism at the gene locus comprising ETS1 comprises SEQ ID NO: 75. In some embodiments, the polymorphism at the gene locus comprising ETS1 comprises SEQ ID NO: 76. In some embodiments, the polymorphism at the gene locus comprising ETS1 comprises SEQ ID NO: 77. In some embodiments, the polymorphism at the gene locus comprising ETD comprises SEQ ID NO: 78. In some embodiments, the polymorphism at the gene locus comprising ETS1 comprises SEQ ID NO: 79. In some embodiments, the polymorphism at the gene locus comprising ETS1 comprises SEQ ID NO: 81. In some embodiments, the polymorphism at the gene locus comprising ETS1 comprises SEQ ID NO: 82. The polymorphism may be within an intron of the ETS1 gene, and may affect ETS1 expression or activity. The polymorphism may be in a protein-coding region of ETS1, and may additionally affect ETS1 protein function. A polymorphism in linkage disequilibrium with an ETS1 polymorphism is inherited with the ETS1 polymorphism. The polymorphism in linkage disequilibrium may not be located in the ETS1 locus. One polymorphism, or any combination of polymorphisms, may be detected in a sample obtained from the subject. In some embodiments, two copies of the polymorphism are detected in the sample obtained from the subject. A subject carrying one copy of the polymorphism has a heterozygous risk genotype. In some embodiments, one copy of the polymorphism is detected in the sample obtained from the subject. A subject carrying two copies of the polymorphism has a homozygous risk genotype. In further embodiments provided, are methods of obtaining the sample from a subject with an inflammatory disease or condition, or fibrostenotic or fibrotic disease. The method of obtaining the sample may include acquisition of the sample from the subject directly, or indirectly. In some embodiments provided are methods of assaying to detect in the sample a presence of a polymorphism located at the gene locus.

In one aspect, provided herein, a polymorphism located at an ARHGAP15 locus comprising rs6757588 (SEQ ID NO: 35), or any polymorphism in linkage disequilibrium therewith, is detected in a sample obtained from the subject. In some embodiments, linkage disequilibrium may be determined using a D′ value of at least 0.70, 0.75, or 0.80. In some embodiments, linkage disequilibrium may be determined using a D′ value of 0, and an r² value of at least 0.70, 0.75, 0.80, 0.85, 0.90, or 0.95. Rho GTPase Activating Protein 15 (ARHGAP15) regulates diverse biological processes, and is involved in ectoderm differentiation and signaling by G-coupled protein receptors (GPCRs). ARHGAP15, and nucleic acids encoding ARHGAP15 are characterized by Entrez Gene ID 55843. The polymorphism at the gene locus comprising ARHGAP15 comprises a “G” allele at nucleobase 501 within rs6757588. In further embodiments, the polymorphism at the gene locus comprising ARHGAP15 comprises SEQ ID NO: 35. The polymorphism may be within an intron of the ARHGAP15 gene, and may affect ARHGAP15 expression or activity. The polymorphism may be in a protein-coding region of ARHGAP15, and may additionally affect ARHGAP15 protein function. A polymorphism in linkage disequilibrium with an ARHGAP15 polymorphism is inherited with the ARHGAP15 polymorphism. The polymorphism in linkage disequilibrium may not be located in the ARHGAP15 locus. One polymorphism, or any combination of polymorphisms, may be detected in a sample obtained from the subject. In some embodiments, two copies of the polymorphism are detected in the sample obtained from the subject. A subject carrying one copy of the polymorphism has a heterozygous risk genotype. In some embodiments, one copy of the polymorphism is detected in the sample obtained from the subject. A subject carrying two copies of the polymorphism has a homozygous risk genotype. In further embodiments provided, are methods of obtaining the sample from a subject with an inflammatory disease or condition, or fibrostenotic or fibrotic disease. The method of obtaining the sample may include acquisition of the sample from the subject directly, or indirectly. In some embodiments provided are methods of assaying to detect in the sample a presence of a polymorphism located at the gene locus.

In one aspect, provided herein, a polymorphism located at a SCUBE1 gene locus comprising rs6003160 (SEQ ID NO: 36), or any polymorphism in linkage disequilibrium therewith, is detected in a sample obtained from the subject. In some embodiments, linkage disequilibrium may be determined using a D′ value of at least 0.70, 0.75, or 0.80. In some embodiments, linkage disequilibrium may be determined using a D′ value of 0, and an r² value of at least 0.70, 0.75, 0.80, 0.85, 0.90, or 0.95. Signal Peptide, CUB Domain and Epidermal Growth Factor (EGF) Like Domain Containing 1 (SCUBE1) is a gene that encodes a cell surface glycoprotein that is a member of the SCUBE family. The polymorphism at the gene locus comprising SCUBE1 comprises a “G” allele at nucleobase 501 within rs6003160. In further embodiments, the polymorphism at the gene locus comprising SCUBE1 comprises SEQ ID NO: 36. The polymorphism may be in a protein-coding region of SCUBE1, and may additionally affect SCUBE1 protein function. A polymorphism in linkage disequilibrium with an SCUBE1 polymorphism is inherited with the SCUBE1 polymorphism. The polymorphism in linkage disequilibrium may not be located in the SCUBE1 locus. One polymorphism, or any combination of polymorphisms, may be detected in a sample obtained from the subject. In some embodiments, two copies of the polymorphism are detected in the sample obtained from the subject. A subject carrying one copy of the polymorphism has a heterozygous risk genotype. In some embodiments, one copy of the polymorphism is detected in the sample obtained from the subject. A subject carrying two copies of the polymorphism has a homozygous risk genotype. In further embodiments provided, are methods of obtaining the sample from a subject with an inflammatory disease or condition, or fibrostenotic or fibrotic disease. The method of obtaining the sample may include acquisition of the sample from the subject directly, or indirectly. In some embodiments provided are methods of assaying to detect in the sample a presence of a polymorphism located at the gene locus.

In one aspect, provided herein, a presence of a polymorphism located at a TNFSF15 gene locus is detected in a sample obtained from the subject. Tumor necrosis factor ligand superfamily, member 15 (TL1A) is a tumor necrosis factor (TNF) family cytokine that exerts pleiotropic effects on cell proliferation, activation, and differentiation of immune cells. TL1A, and nucleic acids encoding TL1A (TNFSF15), are characterized by NCBI Entrez Gene ID 9966. Polymorphisms of the TNFSF15 gene that encodes TL1A are associated with the pathogenesis of autoimmune diseases, such as Inflammatory Bowel Disease (IBD). In some embodiments, the polymorphism located at the gene locus comprising TNFSF15 comprises rs6478109 (SEQ ID NO: 1), rs7848647 (SEQ ID NO: 2), rs201292440 (SEQ ID NO: 3), rs7869487 (SEQ ID NO: 4), rs4366152 (SEQ ID NO: 5), rs6478108 (SEQ ID NO: 6), rs1407308 (SEQ ID NO: 7), rs7866342 (SEQ ID NO: 8), rs7030574 (SEQ ID NO: 9), rs10114470 (SEQ ID NO: 10), rs4979464 (SEQ ID NO: 11), rs3810936 (SEQ ID NO: 12), rs7028891 (SEQ ID NO: 13), rs7863183 (SEQ ID NO: 14), rs4979469 (SEQ ID NO: 15), rs1853187 (SEQ ID NO: 16), rs7040029 (SEQ ID NO: 17), rs722126 (SEQ ID NO: 18), rs4246905 (SEQ ID NO: 19), rs4979467 (SEQ ID NO: 20), rs4979466 (SEQ ID NO: 21), rs7043505 (SEQ ID NO: 22), rs911605 (SEQ ID NO: 23), rs11793394 (SEQ ID NO: 24), rs17219926 (SEQ ID NO: 25), rs7874896 (SEQ ID NO: 26), rs4574921 (SEQ ID NO: 27), rs6478106 (SEQ ID NO: 28), rs7032238 (SEQ ID NO: 29), rs55775610 (SEQ ID NO: 30), rs7847158 (SEQ ID NO: 31), or rs56069985 (SEQ ID NO: 32) or any polymorphism in linkage disequilibrium therewith. In some embodiments, linkage disequilibrium may be determined using a D′ value of at least 0.70, 0.75, or 0.80. In some embodiments, linkage disequilibrium may be determined using a D′ value of 0, and an r² value of at least 0.70, 0.75, 0.80, 0.85, 0.90, or 0.95. The polymorphism within rs201292440 has merged with rs59418409, which means rs201292440 and rs59418409 may be used interchangeably to refer to the same polymorphism. In some embodiments, the polymorphism at the TNFSF15 gene locus is represented with an “N” within any one of SEQ ID NOS: 1-32. One polymorphism, or any combination of polymorphisms, may be detected in a sample obtained from the subject. In some embodiments, two copies of the polymorphism are detected in the sample obtained from the subject. A subject carrying one copy of the polymorphism has a heterozygous risk genotype. A heterozygous risk genotype may be represented with a pair of nucleobases comprising nucleobases that differ from one another (for e.g., “GA”). In some embodiments, one copy of the polymorphism is detected in the sample obtained from the subject. A subject carrying two copies of the polymorphism has a homozygous risk genotype. A homozygous risk genotype may be represented with a pair of nucleobases comprising nucleobases that are identical to one another (for e.g., “GG”). In some cases, the risk genotype comprises an insertion sequence. An insertion sequence is represented either as a single insertion (for e.g., “G”) or as an insertion in a pair (for e.g., “AGA” or “GAA”). In further embodiments provided, are methods of obtaining the sample from a subject with an inflammatory disease or condition, or fibrostenotic or fibrotic disease. The method of obtaining the sample may include acquisition of the sample from the subject directly, or indirectly. In some embodiments provided are methods of assaying to detect in the sample a presence of a polymorphism located at the gene locus.

In one aspect, provided herein, a polymorphism located at a gene locus comprising TNFSF15, LY86, ETS1, ARHGAP15, or SCUBE, is detected in a sample obtained from the subject. In some embodiments, the polymorphism comprises a risk allele within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160, rs11606640, rs73029052, rs11600915, rs61909068, rs12294634, rs73029062, rs11600746, rs3851519, rs61909072, or rs56086356, of Table 5, or any polymorphism in linkage disequilibrium therewith. In some embodiments, linkage disequilibrium may be determined using a D′ value of at least 0.70, 0.75, or 0.80. In some embodiments, linkage disequilibrium may be determined using a D′ value of 0, and an r² value of at least 0.70, 0.75, 0.80, 0.85, 0.90, or 0.95. In some embodiments, the polymorphism comprises one or more sequences from SEQ ID. Nos.: 1-36, or 73-82. In some embodiments, two copies of the polymorphism are detected in the sample obtained from the subject. A subject carrying one copy of the polymorphism has a heterozygous risk genotype. In some embodiments, one copy of the polymorphism is detected in the sample obtained from the subject. A subject carrying two copies of the polymorphism has a homozygous risk genotype. One polymorphism, or any combination of polymorphisms, may be detected in a sample obtained from the subject. In further embodiments provided, are methods of obtaining the sample from a subject with an inflammatory disease or condition, or fibrostenotic or fibrotic disease. The method of obtaining the sample may include acquisition of the sample from the subject directly, or indirectly. In some embodiments provided are methods of assaying to detect in the sample a presence of a polymorphism located at the gene locus.

In one aspect, provided herein, a combination of polymorphisms located at gene loci comprising TNFSF15, LY86, ETS1, ARHGAP15, or SCUBE, is detected in a sample obtained from the subject. In some embodiments, the combination of polymorphisms comprises a risk allele within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160 rs11606640, rs73029052, rs11600915, rs61909068, rs12294634, rs73029062, rs11600746, rs3851519, rs61909072, or rs56086356, and any polymorphism in linkage disequilibrium therewith. In some embodiments, linkage disequilibrium may be determined using a D′ value of at least 0.70, 0.75, or 0.80. In some embodiments, linkage disequilibrium may be determined using a D′ value of 0, and an r² value of at least 0.70, 0.75, 0.80, 0.85, 0.90, or 0.95. The polymorphism within rs201292440 has merged with rs59418409, which means rs201292440 and rs59418409 may be used interchangeably to refer to the same polymorphism. In some embodiments, one copy of the polymorphism at the TNFSF15 gene locus and the polymorphism at the ARHGAP15 gene locus are detected in the sample obtained from the subject, the combinations comprising any one the combinations of Table 6. In some embodiments, two copies of the polymorphism at the TNFSF15 gene locus and the polymorphism at the LY86, ETS1, or SCUBE1 gene loci are detected in the sample obtained from the subject, the combinations comprising any one the combinations of Table 7.

In one aspect disclosed herein, the presence of the polymorphism rs6757588 at the ARHGAP15 locus and the TNFSF15 rs6478109 heterozygous (AG) risk genotype detected in a sample obtained from a subject is strongly associated with an enrichment of an increase in TL1A fold-change levels in the sample, as compared to the mean+/−standard deviation of TL1A fold-change level associated with TNFSF15 rs6478109 non-risk population, as shown in Example 4. In some embodiments, the enrichment of the increase in TL1A fold-change levels in the sample when the polymorphism rs6757588 at the ARHGAP15 locus and the TNFSF15 rs6478109 heterozygous risk genotype are detected in the sample obtained from a subject, is higher than the increase in TL1A fold-change observed when the TNFSF15 rs6478109 heterozygous risk genotype is detected in the sample alone.

In another aspect disclosed herein, the presence of the polymorphism rs6921610 at the LY86 locus and the TNFSF15 rs6478109 homozygous (GG) risk genotype detected in a sample obtained from a subject is strongly associated an enrichment of an increase in TL1A fold-change levels in the sample, as compared to the mean+/−standard deviation of TL1A fold-change level associated with TNFSF15 rs6478109 non-risk population as shown in Example 4. In yet another aspect disclosed herein, the presence of the polymorphism rs10790957 at the ETS1 locus and the TNFSF15 rs6478109 homozygous risk genotype detected in a sample obtained from a subject shows an enrichment of an increase in TL1A fold-change levels, as compared to as compared to the mean+/−standard deviation of TL1A fold-change level associated with TNFSF15 rs6478109 non-risk population. In yet another aspect disclosed herein, the presence of the polymorphism rs6003160 at the SCUBE1 locus and the TNFSF15 rs6478109 homozygous risk genotype detected in a sample obtained from a subject shows an enrichment of an increase in TL1A fold-change levels, as compared to as compared to the mean+/−standard deviation of TL1A fold-change level associated with TNFSF15 rs6478109 non-risk population. In some embodiments, a greater increase in TL1A fold-change is observed when the combination of the polymorphism rs6921610 at the LY86 locus and the polymorphism rs10790957 at the ETS1 locus, and the TNFSF15 rs6478109 homozygous risk genotype are detected in the sample, as compared to the enrichment in the increase in TL1A fold-change observed when one of the polymorphism rs6921610 at the LY86 locus and the polymorphism rs10790957 at the ETS1 locus is detected in the sample in combination with the TNFSF15 rs6478109 homozygous risk genotype. In some embodiments, the enrichment in the increase in TL1A fold-change is higher when the TNFSF15 rs6478109 homozygous risk genotype and at least one of the polymorphism rs6921610 at the LY86 locus and the polymorphism rs10790957 at the ETS1 locus is detected in a sample obtained from the subject, than when the TNFSF15 rs6478109 homozygous risk genotype, alone, is detected in the sample obtained from the subject. Any polymorphism at the TNFSF15 locus in linkage disequilibrium with the rs6478109 polymorphism may be used in combination with the rs6921610, 10790957, rs6003160, and rs6757588 polymorphisms to predict increased TL1A fold-change in a subject, however, non-limiting examples of combinations are provided in Tables 3 and 4. In some embodiments, linkage disequilibrium may be determined using a D′ value of at least 0.70, 0.75, or 0.80. In some embodiments, linkage disequilibrium may be determined using a D′ value of 0, and an revalue of at least 0.70, 0.75, 0.80, 0.85, 0.90, or 0.95.

TABLE 3
Non-Limiting Examples of Heterozygous
TNFSF15 Polymorphism Combinations

| | | | |
|---|---|---|---|
| rs4574921 | rs17219926 | rs7030574 | rs7040029 |
| rs6757588 | rs6757588 | rs6757588 | rs6757588 |
| rs7848647 | rs7874896 | rs10114470 | rs722126 |
| rs6757588 | rs6757588 | rs6757588 | rs6757588 |
| rs201292440 | rs6478109 | rs4979464 | rs4246905 |
| rs6757588 | rs6757588 | rs6757588 | rs6757588 |
| rs7869487 | rs6478106 | rs3810936 | rs4979467 |
| rs6757588 | rs6757588 | rs6757588 | rs6757588 |
| rs4366152 | rs7032238 | rs7028891 | rs4979466 |
| rs6757588 | rs6757588 | rs6757588 | rs6757588 |
| rs6478108 | rs55775610 | rs7863183 | rs7043505 |
| rs6757588 | rs6757588 | rs6757588 | rs6757588 |
| rs1407308 | rs7847158 | rs4979469 | rs911605 |
| rs6757588 | rs6757588 | rs6757588 | rs6757588 |
| rs7866342 | rs56069985 | rs1853187 | rs11793394 |
| rs6757588 | rs6757588 | rs6757588 | rs6757588 |

TABLE 4
Non-Limiting Examples of Homozygous TNFSF15 Polymorphism Combinations

| | | | | | | |
|---|---|---|---|---|---|---|
| rs10790957 | rs692610 | rs6003160 | rs692610 | rs692610 | rs10790957 | rs692610 |
| rs911605 | rs911605 | rs911605 | rs10790957 | rs6003160 | rs6003160 | rs10790957 |
| | | | rs911605 | rs911605 | rs911605 | rs6003160 |
| | | | | | | rs911605 |
| rs10790957 | rs692610 | rs6003160 | rs692610 | rs692610 | rs10790957 | rs692610 |
| rs11793394 | rs11793394 | rs11793394 | rs10790957 | rs6003160 | rs6003160 | rs10790957 |
| | | | rs11793394 | rs11793394 | rs11793394 | rs6003160 |
| | | | | | | rs11793394 |
| rs10790957 | rs692610 | rs6003160 | rs692610 | rs692610 | rs10790957 | rs692610 |
| rs17219926 | rs17219926 | rs17219926 | rs10790957 | rs6003160 | rs6003160 | rs10790957 |
| | | | rs17219926 | rs17219926 | rs17219926 | rs6003160 |
| | | | | | | rs17219926 |
| rs10790957 | rs692610 | rs6003160 | rs692610 | rs692610 | rs10790957 | rs692610 |
| rs7874896 | rs7874896 | rs7874896 | rs10790957 | rs6003160 | rs6003160 | rs10790957 |
| | | | rs7874896 | rs7874896 | rs7874896 | rs6003160 |
| | | | | | | rs7874896 |
| rs10790957 | rs692610 | rs6003160 | rs692610 | rs692610 | rs10790957 | rs692610 |
| rs4574921 | rs4574921 | rs4574921 | rs10790957 | rs6003160 | rs6003160 | rs10790957 |
| | | | rs4574921 | rs4574921 | rs4574921 | rs6003160 |
| | | | | | | rs4574921 |
| rs10790957 | rs692610 | rs6003160 | rs692610 | rs692610 | rs10790957 | rs692610 |
| rs6478106 | rs6478106 | rs6478106 | rs10790957 | rs6003160 | rs6003160 | rs10790957 |
| | | | rs6478106 | rs6478106 | rs6478106 | rs6003160 |
| | | | | | | rs6478106 |
| rs10790957 | rs692610 | rs6003160 | rs692610 | rs692610 | rs10790957 | rs692610 |
| rs7032238 | rs7032238 | rs7032238 | rs10790957 | rs6003160 | rs6003160 | rs10790957 |
| | | | rs7032238 | rs7032238 | rs7032238 | rs6003160 |
| | | | | | | rs7032238 |
| rs10790957 | rs692610 | rs6003160 | rs692610 | rs692610 | rs10790957 | rs692610 |
| rs7848647 | rs7848647 | rs7848647 | rs10790957 | rs6003160 | rs6003160 | rs10790957 |
| | | | rs7848647 | rs7848647 | rs7848647 | rs6003160 |
| | | | | | | rs7848647 |
| rs10790957 | rs692610 | rs6003160 | rs692610 | rs692610 | rs10790957 | rs692610 |
| rs201292440 | rs201292440 | rs201292440 | rs10790957 | rs6003160 | rs6003160 | rs10790957 |
| | | | rs201292440 | rs201292440 | rs201292440 | rs6003160 |
| | | | | | | rs201292440 |
| rs10790957 | rs692610 | rs6003160 | rs692610 | rs692610 | rs10790957 | rs692610 |
| rs6478109 | rs6478109 | rs6478109 | rs10790957 | rs6003160 | rs6003160 | rs10790957 |
| | | | rs6478109 | rs6478109 | rs6478109 | rs6003160 |
| | | | | | | rs6478109 |
| rs10790957 | rs692610 | rs6003160 | rs692610 | rs692610 | rs10790957 | rs692610 |
| rs7869487 | rs7869487 | rs7869487 | rs10790957 | rs6003160 | rs6003160 | rs10790957 |
| | | | rs7869487 | rs7869487 | rs7869487 | rs6003160 |
| | | | | | | rs7869487 |
| rs10790957 | rs692610 | rs6003160 | rs692610 | rs692610 | rs10790957 | rs692610 |
| rs4366152 | rs4366152 | rs4366152 | rs10790957 | rs6003160 | rs6003160 | rs10790957 |
| | | | rs4366152 | rs4366152 | rs4366152 | rs6003160 |
| | | | | | | rs4366152 |
| rs10790957 | rs692610 | rs6003160 | rs692610 | rs692610 | rs10790957 | rs692610 |
| rs6478108 | rs6478108 | rs6478108 | rs10790957 | rs6003160 | rs6003160 | rs10790957 |
| | | | rs6478108 | rs6478108 | rs6478108 | rs6003160 |
| | | | | | | rs6478108 |
| rs10790957 | rs692610 | rs6003160 | rs692610 | rs692610 | rs10790957 | rs692610 |
| rs1407308 | rs1407308 | rs1407308 | rs10790957 | rs6003160 | rs6003160 | rs10790957 |
| | | | rs1407308 | rs1407308 | rs1407308 | rs6003160 |
| | | | | | | rs1407308 |
| rs10790957 | rs692610 | rs6003160 | rs692610 | rs692610 | rs10790957 | rs692610 |
| rs7866342 | rs7866342 | rs7866342 | rs10790957 | rs6003160 | rs6003160 | rs10790957 |
| | | | rs7866342 | rs7866342 | rs7866342 | rs6003160 |
| | | | | | | rs7866342 |
| rs10790957 | rs692610 | rs6003160 | rs692610 | rs692610 | rs10790957 | rs692610 |
| rs7030574 | rs7030574 | rs7030574 | rs10790957 | rs6003160 | rs6003160 | rs10790957 |
| | | | rs7030574 | rs7030574 | rs7030574 | rs6003160 |
| | | | | | | rs7030574 |
| rs10790957 | rs692610 | rs6003160 | rs692610 | rs692610 | rs10790957 | rs692610 |
| rs10114470 | rs10114470 | rs10114470 | rs10790957 | rs6003160 | rs6003160 | rs10790957 |
| | | | rs10114470 | rs10114470 | rs10114470 | rs6003160 |
| | | | | | | rs10114470 |
| rs10790957 | rs692610 | rs6003160 | rs692610 | rs692610 | rs10790957 | rs692610 |
| rs55775610 | rs55775610 | rs55775610 | rs10790957 | rs6003160 | rs6003160 | rs10790957 |
| | | | rs55775610 | rs55775610 | rs55775610 | rs6003160 |
| | | | | | | rs55775610 |
| rs10790957 | rs692610 | rs6003160 | rs692610 | rs692610 | rs10790957 | rs692610 |
| rs7847158 | rs7847158 | rs7847158 | rs10790957 | rs6003160 | rs6003160 | rs10790957 |
| | | | rs7847158 | rs7847158 | rs7847158 | rs6003160 |
| | | | | | | rs7847158 |
| rs10790957 | rs692610 | rs6003160 | rs692610 | rs692610 | rs10790957 | rs692610 |
| rs56069985 | rs56069985 | rs56069985 | rs10790957 | rs6003160 | rs6003160 | rs10790957 |
| | | | rs56069985 | rs56069985 | rs56069985 | rs6003160 |
| | | | | | | rs56069985 |
| rs10790957 | rs692610 | rs6003160 | rs692610 | rs692610 | rs10790957 | rs692610 |
| rs6478109 | rs6478109 | rs6478109 | rs10790957 | rs6003160 | rs6003160 | rs10790957 |
| | | | rs6478109 | rs6478109 | rs6478109 | rs6003160 |
| | | | | | | rs6478109 |
| rs10790957 | rs692610 | rs6003160 | rs692610 | rs692610 | rs10790957 | rs692610 |
| rs7848647 | rs7848647 | rs7848647 | rs10790957 | rs6003160 | rs6003160 | rs10790957 |
| | | | rs7848647 | rs7848647 | rs7848647 | rs6003160 |
| | | | | | | rs7848647 |
| rs10790957 | rs692610 | rs6003160 | rs692610 | rs692610 | rs10790957 | rs692610 |
| rs201292440 | rs201292440 | rs201292440 | rs10790957 | rs6003160 | rs6003160 | rs10790957 |
| | | | rs201292440 | rs201292440 | rs201292440 | rs6003160 |
| | | | | | | rs201292440 |
| rs10790957 | rs692610 | rs6003160 | rs692610 | rs692610 | rs10790957 | rs692610 |
| rs7869487 | rs7869487 | rs7869487 | rs10790957 | rs6003160 | rs6003160 | rs10790957 |
| | | | rs7869487 | rs7869487 | rs7869487 | rs6003160 |
| | | | | | | rs7869487 |
| rs10790957 | rs692610 | rs6003160 | rs692610 | rs692610 | rs10790957 | rs692610 |
| rs4366152 | rs4366152 | rs4366152 | rs10790957 | rs6003160 | rs6003160 | rs10790957 |
| | | | rs4366152 | rs4366152 | rs4366152 | rs6003160 |
| | | | | | | rs4366152 |
| rs10790957 | rs692610 | rs6003160 | rs692610 | rs692610 | rs10790957 | rs692610 |
| rs6478108 | rs6478108 | rs6478108 | rs10790957 | rs6003160 | rs6003160 | rs10790957 |
| | | | rs6478108 | rs6478108 | rs6478108 | rs6003160 |
| | | | | | | rs6478108 |
| rs10790957 | rs692610 | rs6003160 | rs692610 | rs692610 | rs10790957 | rs692610 |
| rs1407308 | rs1407308 | rs1407308 | rs10790957 | rs6003160 | rs6003160 | rs10790957 |
| | | | rs1407308 | rs1407308 | rs1407308 | rs6003160 |
| | | | | | | rs1407308 |
| rs10790957 | rs692610 | rs6003160 | rs692610 | rs692610 | rs10790957 | rs692610 |
| rs7866342 | rs7866342 | rs7866342 | rs10790957 | rs6003160 | rs6003160 | rs10790957 |
| | | | rs7866342 | rs7866342 | rs7866342 | rs6003160 |
| | | | | | | rs7866342 |
| rs10790957 | rs692610 | rs6003160 | rs692610 | rs692610 | rs10790957 | rs692610 |
| rs7030574 | rs7030574 | rs7030574 | rs10790957 | rs6003160 | rs6003160 | rs10790957 |
| | | | rs7030574 | rs7030574 | rs7030574 | rs6003160 |
| | | | | | | rs7030574 |
| rs10790957 | rs692610 | rs6003160 | rs692610 | rs692610 | rs10790957 | rs692610 |
| rs10114470 | rs10114470 | rs10114470 | rs10790957 | rs6003160 | rs6003160 | rs10790957 |
| | | | rs10114470 | rs10114470 | rs10114470 | rs6003160 |
| | | | | | | rs10114470 |
| rs10790957 | rs692610 | rs6003160 | rs692610 | rs692610 | rs10790957 | rs692610 |
| rs4979464 | rs4979464 | rs4979464 | rs10790957 | rs6003160 | rs6003160 | rs10790957 |
| | | | rs4979464 | rs4979464 | rs4979464 | rs6003160 |
| | | | | | | rs4979464 |
| rs10790957 | rs692610 | rs6003160 | rs692610 | rs692610 | rs10790957 | rs692610 |
| rs3810936 | rs3810936 | rs3810936 | rs10790957 | rs6003160 | rs6003160 | rs10790957 |
| | | | rs3810936 | rs3810936 | rs3810936 | rs6003160 |
| | | | | | | rs3810936 |
| rs10790957 | rs692610 | rs6003160 | rs692610 | rs692610 | rs10790957 | rs692610 |
| rs7028891 | rs7028891 | rs7028891 | rs10790957 | rs6003160 | rs6003160 | rs10790957 |
| | | | rs7028891 | rs7028891 | rs7028891 | rs6003160 |
| | | | | | | rs7028891 |
| rs10790957 | rs692610 | rs6003160 | rs692610 | rs692610 | rs10790957 | rs692610 |
| rs7863183 | rs7863183 | rs7863183 | rs10790957 | rs6003160 | rs6003160 | rs10790957 |
| | | | rs7863183 | rs7863183 | rs7863183 | rs6003160 |
| | | | | | | rs7863183 |
| rs10790957 | rs692610 | rs6003160 | rs692610 | rs692610 | rs10790957 | rs692610 |
| rs4979469 | rs4979469 | rs4979469 | rs10790957 | rs6003160 | rs6003160 | rs10790957 |
| | | | rs4979469 | rs4979469 | rs4979469 | rs6003160 |
| | | | | | | rs4979469 |
| rs10790957 | rs692610 | rs6003160 | rs692610 | rs692610 | rs10790957 | rs692610 |
| rs1853187 | rs1853187 | rs1853187 | rs10790957 | rs6003160 | rs6003160 | rs10790957 |
| | | | rs1853187 | rs1853187 | rs1853187 | rs6003160 |
| | | | | | | rs1853187 |
| rs10790957 | rs692610 | rs6003160 | rs692610 | rs692610 | rs10790957 | rs692610 |
| rs7040029 | rs7040029 | rs7040029 | rs10790957 | rs6003160 | rs6003160 | rs10790957 |
| | | | rs7040029 | rs7040029 | rs7040029 | rs6003160 |
| | | | | | | rs7040029 |
| rs10790957 | rs692610 | rs6003160 | rs692610 | rs692610 | rs10790957 | rs692610 |
| rs722126 | rs722126 | rs722126 | rs10790957 | rs6003160 | rs6003160 | rs10790957 |
| | | | rs722126 | rs722126 | rs722126 | rs6003160 |
| | | | | | | rs722126 |
| rs10790957 | rs692610 | rs6003160 | rs692610 | rs692610 | rs10790957 | rs692610 |
| rs4246905 | rs4246905 | rs4246905 | rs10790957 | rs6003160 | rs6003160 | rs10790957 |
| | | | rs4246905 | rs4246905 | rs4246905 | rs6003160 |
| | | | | | | rs4246905 |
| rs10790957 | rs692610 | rs6003160 | rs692610 | rs692610 | rs10790957 | rs692610 |
| rs4979467 | rs4979467 | rs4979467 | rs10790957 | rs6003160 | rs6003160 | rs10790957 |
| | | | rs4979467 | rs4979467 | rs4979467 | rs6003160 |
| | | | | | | rs4979467 |
| rs10790957 | rs692610 | rs6003160 | rs692610 | rs692610 | rs10790957 | rs692610 |
| rs4979466 | rs4979466 | rs4979466 | rs10790957 | rs6003160 | rs6003160 | rs10790957 |
| | | | rs4979466 | rs4979466 | rs4979466 | rs6003160 |
| | | | | | | rs4979466 |
| rs10790957 | rs692610 | rs6003160 | rs692610 | rs692610 | rs10790957 | rs692610 |
| rs7043505 | rs7043505 | rs7043505 | rs10790957 | rs6003160 | rs6003160 | rs10790957 |
| | | | rs7043505 | rs7043505 | rs7043505 | rs6003160 |
| | | | | | | rs7043505 |

Aspects disclosed herein, provide methods of identifying polymorphisms useful for the treatment or characterization of the inflammatory diseases or conditions or fibrotic or fibrostenotic diseases disclosed herein using a TL1A fold-change enrichment analysis. In some embodiments, the TL1A fold-change enrichment analysis comprises: a) assaying, or having assayed, a plurality of samples obtained from a plurality of subjects to detect an increase in TL1A fold-change; b) obtaining, or having obtained, a plurality of genotypes of the plurality of subjects, wherein the plurality of genotypes comprise polymorphisms associated with the increase in TL1A fold-change using a linear regression model or logistic regression model, wherein the polymorphisms are characterized by having a p value of at most 10-3; c) selecting a criteria polymorphism from the polymorphisms associated with the increase in TL1A fold-change to serve as a predictor of the increase in TL1A fold-change in the plurality of subjects, the criteria polymorphism comprising rs6478109, wherein selection of the criterial polymorphism is based, at least, on the p value; and d) identifying the risk polymorphism, provided an enrichment of the increase in TL1A fold-change is observed in a subset of the plurality of samples in which the criteria polymorphism and the risk polymorphism are expressed, as compared to the increase in TL1A fold-change observed when the criteria polymorphism, alone, is expressed. Polymorphisms shown to enrich the increase in TL1A fold-change in a population of subjects using the TL1A fold-change enrichment analysis may be used in combination with the criteria polymorphism as patient selection markers to identify subjects suitable for treatment with the inhibitor of TL1A expression or activity disclosed herein. In addition, polymorphisms shown to enrich the increase in TL1A fold-change in a population of subjects using the TL1A fold-change enrichment analysis may be used to characterize a TL1A-associated inflammatory disease or condition or fibrotic or fibrostenotic disease disclosed herein.

In some embodiments, the polymorphism is associated with a subclinical phenotype of IBD. A subclinical phenotype of IBD may include specific diagnosable diseases or conditions, in addition to disease progression that is characteristic of severe or unusual forms of IBD. Non-limiting examples of IBD subclinical phenotypes include, but are not limited to, non-stricturing, stricturing, stricturing and penetrating, and isolated internal penetrating, disease, and perianal Crohn's disease (pCD). Stricturing is the progressive narrowing of the intestine. Internal penetrating disease creates abnormal passageways (fistulae) between the bowel and other structures. pCD is a form of Crohn's disease that causes inflammation around the anus. Further, patients with disease that is stricturing, penetrating and stricturing, or isolated internal penetrating, and patients with pCD are more likely to require surgery in a shorter time span than a patient who has IBD, but who does not exhibit these subclinical phenotypes. In some embodiments, the polymorphism is associated with a time to first surgery, or a time to second surgery, or a combination thereof. The time to first surgery may be from about 2 to 8 years. The time to first surgery may be from about 4 to 10 years. The time to first surgery may be from about 6 to 12 years. The time to first surgery may be from about 8 to 14 years. The time to first surgery may be from about 10 to 16 years. The time to second surgery may be about 20 to 120 months. The time to second surgery may be about 30 to 140 months. The time to second surgery may be about 50 to 160 months. The time to second surgery may be about 70 to 180 months. Subclinical phenotypes of IBD may manifest in specific disease locations. Non-limiting examples of disease location include the ileum, colon, region spanning the ileum and colon (ilealcolonic region), and small bowel. In some embodiments, the polymorphism is associated with stricturing disease in the ileum, colon, ilealcolonic region, or small bowel. In some embodiments, the polymorphism is associated with stricturing and penetrating disease in the ileum, colon, ilealcolonic region, or small bowel. In some embodiments, the polymorphism is associated with isolated penetrating disease in the ileum, colon, ilealcolonic region, or small bowel. Sub clinical phenotypes of IBD may also include non-response to current IBD therapies. In some embodiments, the polymorphism is associated with non-response to anti-TNF-alpha therapy, anti-a4-b7 therapy (vedolizumab), anti-IL12p40 therapy (ustekinumab), Thalidomide, or Cytoxan. In some embodiments, the polymorphism is associated with thiopurine toxicity, or a disease or condition caused by thiopurine toxicity (such as pancreatitis or leukopenia). A subject may exhibit one, or any combination of, the subclinical phenotypes of IBD disclosed herein, as well as others that may be readily apparent.

In some embodiments, the polymorphism, or combination of polymorphisms, of Tables 3, 4, and 5, is associated with an increase in TL1A expression. As disclosed herein, TL1A expression may comprise expression of the DNA or RNA molecule, TNFSF15, or protein molecule, TL1A. TL1A expression may be detected in a particular disease location. In some embodiments, the polymorphism is associated with an increase in TL1A expression in a region of the intestine comprising the ileum, colon, ileocolonic region, small bowel, or anus, or a combination thereof. In some embodiments, increased TL1A fold-change is observed. The increase in expression of TL1A may be an increase of 1.1-fold, 1.2-fold, 1.3-fold, 1.4-fold, 1.5 fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.0-fold, 3.0-fold, 3.1-fold, 3.2-fold, 3.3-fold, 3.4-fold, 3.5-fold, 4-fold, 5-fold, 10-fold, 20-fold, 30-fold, 40-fold, 50-fold, 60-fold, 70-fold, 80-fold, 90-fold, 100-fold, or more between the sample obtained from the subject and an expression of TL1A in an individual who does not express the polymorphism. In some embodiments, the expression of TL1A in an individual who does not express the polymorphism is a control or standard. In some embodiments, detection of one or any combination of the polymorphisms is associated with an increase in expression of TL1A.

TABLE 6
Polymorphism flanking sequence

| SEQ ID | |
|---|---|
| NO: | Polymorphism Flanking Sequence |
| 1 | CAGTCTGGGGAGTGTGCTTCTGGAAGTGAAAGTGAGGGATGAGAGGTGTGTGGT |
| | TTGCAG |
| | N |
| | TTGGGAAACGGAAATCACATTTGCATCAGCTCTTTGCAAAGTGCTGCCTAGCCC |
| | TCTGTC |
| 2 | ATGAAAGGAA AGTATTTCCA GTCTGCATTG |
| | ACCATTGTTT AATCAGAGTA |
| | N |
| | GAGGCCACAG ATCGAGGTGA CTGTCTGTGA GGGTAGAACA TTAACCACTA |
| 3 | TTTATAGTAC ATTAGATGGC CTTAAGTGAT |
| | TTAGAAAAAA AAAAGAGATA |
| | N |
| | AATGATCTTA ATTGCAATTG AAAATAGAGT TGTCAGAATA GACCTCATTG |
| 4 | GGCTATTCCA TTGAAATGTG TGTTTTGATG |
| | ATCATGGCTA AGTGGGACTT |
| | N |
| | AGTGACTCAA ACCCTGTGTT CAGATGAAGC CTGCTCAGAT TTCTCCTATA |
| 5 | CCGGCCAGAT TTTGTTTTTA ATTGTATTTC |
| | TGTAATGTAA GCATGCTGTG |
| | N |
| | TAGCTCTCTG ATTCTTAATT CTCTCTTTGG AAAAATACAG GTAGCCTTAC |
| 6 | GTCCAATCTC ATTTTGTCTT GGCATTCAAA |
| | GTCCTAACTT ATCCCAGTCT |
| | N |
| | GCTATCCATT ATTTACTTCT CTCTAAGCCC TCTGTGTTCC CAGCCATGAG |
| 7 | AGAGCAGGTA CTAAGTCATA ACCCTTCCCC |
| | ATGACTATTG CTCCTAACTG |
| | N |
| | TATTCAATAG ACTCATTACC ATCTACTAAA ATAAGCATCT AACGTATTTT |
| 8 | AGATCTGCCT GAAGGCCTCA GATGGAACCA |
| | AAAGTGAGCT CTTTCTTCAC |
| | N |
| | GTTAGGAGGT TAATGACATC CATTTCCATC AAATGGTAAT TGGTATATTT |
| 9 | TCCTCTGTCC AGAGCTGAAA TAGTTGCCAC |
| | TCACTGCAGA GAGTCCACTG |
| | N |
| | TCCTCCCCAA GGTCAGAGTG CCATGTGGCT TACCTGGAAC TGCACACAGG |
| 10 | TAATGATCAA CTAGAACACA TGGAAGTCAA |
| | TGAACAAAAG GCCACATAAT |
| | N |
| | ATAGATTTGA AAAAGACCTC AGAGATCCTT TGGCTTAATC TCTCCCCCAA |
| 11 | ACACATACGT AGTCCCCCTG CTGCACTGTT |
| | GGTGTGCACT CACCCATGGC |
| | N |
| | TCCTGCTACC CTGTTGGTGT GCCCTGTTGG TGTGCACTCG CTGGCAGCTC |
| 12 | AACATGGCTC CGAGGTAGAT GGGCTGGAAC |
| | CAGTTGCTAC CTACTTCGCA |
| | N |
| | ACAGACTTGG TCCCCATGAG GAGCTGGGTT GGCTCAGGGT AGCTGTCTGT |
| 13 | GTGATGAAGG TCTGCGGTCC TAGAGCTACA |
| | GATGCATGGT AAGAAATTAG |
| | N |
| | ACAAACTGGA GATGGGCCAT GGACTCTGCC TAGGAGTGTT AGGAAAATAC |
| 14 | GAGGTACAGT CTTTTAGAAG GCAGGGATGA ATTCATGGGA TGGAATTCAG |
| | N |
| | GCCCAAGAGG AGAGTTGGCC TTCATTAGGC CTGGGGACAC ATCCTCCACC |
| 15 | AGAGAGTTGT GGGTCCTAGA GTATGTAAGA |
| | GAGAATAGAG AAGAGAGGAG |
| | N |
| | AGAGAGAGAG AGGAAGAGAA AGCAAGACCA ACCAATAAAC CAACATAATC |
| 16 | GTCAGTGGGA |
| | TGTTAAAGTC TGCCAGTGTT ATTCTGTGGG AAACTAAGTC T |
| | N |
| | TTTGTAGATC TCTAAGAACT TGCTTTATAA ATCTGAGTGC TTCCTGTATT |
| 17 | TTTCCTTTGG TAAACTTGAA TATCCTCCAA |
| | CTCTGTAGTA TCCCCAAGAT |
| | N |
| | CTTTCCAACC CAAAGTCTAT AATCCTTCTA GGCATTGTCG TCCTCTTAGG |
| 18 | ATCAATACCT ACCTCCCTTA CAAACATCAA |
| | GAGCAAGAAA GAAGGCAGAC |
| | N |
| | TGGAAAGCCC AAATCTTCCG ATAACTGAAA AACATCCATA TTTGAATAAG |
| 19 | GAAAGAGGATTAATTTTCTC ATTGGGAAAC |
| | TGTAGACTTT GCTTAAAAAG |
| | N |
| | GTCTCATATC ATTTTCAAAA TAGACTAAAG TGATCGAATA TACCTAACAG |
| 20 | TGCTCCACTC TCCAAAACTG CGAAACTGCG |
| | AAGGTGTCTT GAACCACCTT |
| | N |
| | CTTACGTTGA ATTGTTGGCT TGTCACTTAA GTACCTGAGC TAATTTATAC |
| 21 | ATGTCAGTGT TCTCAATTCT GTCTGGGCCT |
| | GTGGAGTTTT AAAATATACA |
| | N |
| | TTTTTCGCAT TCTAGTCCCA AGATCGTTGA GTGTACTTGG AAGGGGACCA |
| 22 | ACGATGAGCC |
| | TGGAGCATTT ATCCATCAAC TGCCATCCAT CTCTGGGTCA GGCTT |
| | N |
| | ACTCTGCAAA TGTCACTATT TTCAAACTGC CAGGCTGCAC TTGCATTAGG |
| 23 | TTAAGGCACC ATCTGGTCTC TTCTAAACTC |
| | CCTTGAGTGG TTGATTGAAG |
| | N |
| | AAAACATCTA AGAACAAATA ATTTTCTTGG AACAGTACAT TCTAAGTCTA |
| 24 | AGAGCGGAGA TTGAGATAAA TAAAGTAAGG |
| | GGACTTTTAG ATGACCAAGC |
| | N |
| | GAGGCAATTA ATAGGTAGAT GAACGGTTAT TTGGGGCTTC CAGGCAGAGG |
| 25 | AATAAAAGGC ACAACATCCC AATCTCATAG |
| | CAAGATTATA GGACGTCACC |
| | N |
| | GGCAATCAGA GAGCCTGATG TGGAGTTGGT GCTCAGTTCT TCATTCAACA |
| 26 | CTGGTGCCAT AAAATATTCA |
| | GCTATAGGAC TGAGTGTCCA TGGGTTATA |
| | N |
| | AATAGGAATG AAAGAATGGA AAAGCCTAAA CAATTACATT TGACTTGATT |
| 27 | AACATGTACC TTTGTGGATA AAAGCCTTAA |
| | GTTCCCCATG AATGACTTTT |
| | N |
| | CCCCCTCCTT TATAAAATTG ACACCCATGC TTGTGATGAA ACCACATTTA |
| 28 | GTTGTTCAAG GCCTTAGAAT TTGCCAGTTT |
| | GGCAGACCCG GGCCTGGAGC |
| | N |
| | CAGGACAGCA GACTCCTGAT CCATTGCATC TTCTTCAGTT CCATCTTGGG |
| 29 | ACTGCTGAGC TAGACACAAA AAGAATATGA |
| | CATGTTCCCT GCCTTTATGG |
| | N |
| | ACTCACAGTG TAGTGGGGGT GACAGATGCA GACACTAAAA ATTTGACTAC |
| 30 | AAAGAGTGCA TGGAGGGCTT GGGATCTGAA |
| | CCTTTAGACC AAGTACAGAC |
| | N |
| | CTGGCACATA TTGGGAGCTT CATAAACATC AGCTCAGTGT ACAATAGATG |
| 31 | TGCCAGATTA TCCAACTGGC AAAATGCACA |
| | GATTCTCAGG CATCAGGAAG |
| | N |
| | CAGAGGCAGA CAAAGAGAGT CAGAGAGGGG GTGAGGATGC AGTGACTTCA |
| 32 | CTGTGATGTA |
| | CGGCAGAAAC CAGTTTTACT AGCGCCTCCA TCCAGTTGCT |
| | N |
| | CTTCTGGTTA TGTCACAGCC TGGACTCTTC AGGCTACTTG GAAAGGCCTT |
| 33 | ATATATTCTATAAAAGAGACACTTCAGTAACTCAAAAAGTCTATGTTCTTCAAGT |
| | GCCCC |
| | N |
| | CCACAAAGGGTTATAGCCCTTGGATGAAGCATCTTTCTAGTCCTCTTCTGAACTT |
| | ACCCA |
| 34 | TGTGTCAAGAGCTTATTGTNTGGGGAATGTTGGTGGGCATTTGACCTCTATCCTC |
| | ATTTC |
| | N |
| | TCTTCATCACAGTGCTCCGGGAAAAATCGCAATCACCCCCATTTTAGAGATGAG |
| | GATATG |
| 35 | TTACCTCTCATGAGGGAAATACCCTCATACAGTTGGCCATCACTTAACAATAGA |
| | GACAAC |
| | N |
| | ATGATAGATGGGATGGTAGCAACTTTAGGTTTTGTTGTTTCCTATTTTTCAGTGG |
| | TGAAT |
| 36 | CTGGAGACCAAGGACTATGTTGCACCATAACTATCACCTCCCAGGTATGCAGAA |
| | CTGAGC |
| | N |
| | ATTTTCAAAGGTCTTCACCATTCATAGTCTCATTTGAGCCTGAAACTACTTTGAC |
| | AGCTA |
| 37 | CAGTCTGGGGAGTGTGCTTCTGGAAGTGAAAGTGAGGGATGAGAGGTGTGTGGT |
| | TTGCAG[A/G]TTGGGAAACGGAAATCACATTTGCATCAGCTCTTTGCAAAGTGCT |
| | GCCTAGCCCTCTGTC |
| 38 | AATCAGGGAGTAGTGGTTAATGTTCTACCCTCACAGACAGTCACCTCGATCTGT |
| | GGCCTC[A/G]TACTCTGATTAAACAATGGTCAATGCAGACTGGAAATACTTTCCT |
| | TTCATGGGCAGTCAT |
| 39 | AATAAGTTAATTTATAGTACATTAGATGGCCTTAAGTGATTTAGAAAAAAAAAA |
| | GAGATA[-/GAA] |
| | AATGATCTTAATTGCAATTGAAAATAGAGTTGTCAGAATAGACCTCATTG |
| | AGAGGAGACA |
| 40 | CTTCTACGCTTATAGGAGAAATCTGAGCAGGCTTCATCTGAACACAGGGTTTGA |
| | GTCACT[A/G]AAGTCCCACTTAGCCATGATCATCAAAACACACATTTCAATGGAA |
| | TAGCCCACTCCCCAG |
| 41 | CCAGCAGAGAGTAAGGCTACCTGTATTTTTCCAAAGAGAGAATTAAGAATCAGA |
| | GAGCTA[A/G]CACAGCATGCTTACATTACAGAAATACAATTAAAAACAAAATCT |
| | GGCCGGGCACAGTGGC |
| 42 | GTGGTTGCCTCTCATGGCTGGGAACACAGAGGGCTTAGAGAGAAGTAAATAATG |
| | GATAGC[A/G]AGACTGGGATAAGTTAGGACTTTGAATGCCAAGACAAAATGAGA |
| | TTGGACTGGGTCTTAA |
| 43 | GAATTCTTTGAAAATACGTTAGATGCTTATTTTAGTAGATGGTAATGAGTCTATT |
| | GAATA[A/C]CAGTTAGGAGCAATAGTCATGGGGAAGGGTTATGACTTAGTACCT |
| | GCTCTCCCAGACCTG |
| 44 | AATCACATGCAAATATACCAATTACCATTTGATGGAAATGGATGTCATTAACCT |
| | CCTAAC[A/C]GTGAAGAAAGAGCTCACTTTTGGTTCCATCTGAGGCCTTCAGGCA |
| | GATCTTCATGGCCCA |
| 45 | AGAAACTCTATCCTCTGTCCAGAGCTGAAATAGTTGCCACTCACTGCAGAGAGT |
| | CCACTG[A/C]TCCTCCCCAAGGTCAGAGTGCCATGTGGCTTACCTGGAACTGCAC |
| | ACAGGCCTCTCCCTG |
| 46 | TTCACAGAGGTTGGGGGAGAGATTAAGCCAAAGGATCTCTGAGGTCTTTTTCAA |
| | ATCTAT[A/G]ATTATGTGGCCTTTTGTTCATTGACTTCCATGTGTTCTAGTTGATC |
| | ATTACAAACCTGGC |
| 47 | GCCAGGATGCACACATACGTAGTCCCCCTGCTGCACTGTTGGTGTGCACTCACC |
| | CATGGC[A/G]TCCTGCTACCCTGTTGGTGTGCCCTGTTGGTGTGCACTCGCTGGC |
| | AGCTCCCTGCTGCCC |
| 48 | CACCAAGGTAACAGACAGCTACCCTGAGCCAACCCAGCTCCTCATGGGGACCAA |
| | GTCTGT[A/G]TGCGAAGTAGGTAGCAACTGGTTCCAGCCCATCTACCTCGGAGCC |
| | ATGTTCTCCTTGCAA |
| 49 | GGATACGATTGTGATGAAGGTCTGCGGTCCTAGAGCTACAGATGCATGGTAAGA |
| | AATTAG[A/G]ACAAACTGGAGATGGGCCATGGACTCTGCCTAGGAGTGTTAGGA |
| | AAATACTTTGACTCCA |
| 50 | AACCTGTTATGGTGGAGGATGTGTCCCCAGGCCTAATGAAGGCCAACTCTCCTC |
| | TTGGGC[A/G]CTGAATTCCATCCCATGAATTCATCCCTGCCTTCTAAAAGACTGT |
| | ACCTCCTTAGTTATG |
| 51 | AGATATAGTGAGAGAGTTGTGGGTCCTAGAGTATGTAAGAGAGAATAGAGAAG |
| | AGAGGAG[A/G]AGAGAGAGAGAGGAAGAGAAAGCAAGACCAACCAATAAACC |
| | AACATAATCCAATTTTTTA |
| 52 | ATATGCACTCAATACAGGAAGCACTCAGATTTATAAAGCAAGTTCTTAGAGATC |
| | TACAAA[C/G]AGACTTAGTTTCCCACAGAATAACACTGGCAGACTTTAACATCCC |
| | ACTGACAGTATTAGA |
| 53 | ATAGCTGAGGCCTAAGAGGACGACAATGCCTAGAAGGATTATAGACTTTGGGTT |
| | GGAAAG[A/G]ATCTTGGGGATACTACAGAGTTGGAGNATATTCAAGTTTACCAA |
| | AGGAAACAATGAGAAA |
| 54 | TTCAAGTACAATCAATACCTACCTCCCTTACAAACATCAAGAGCAAGAANGAAG |
| | GCAGAC[A/C]TGGAAAGCCCAAATCTTCCGATAACTGAAAAACATCCATATTTG |
| | AATAAGCTTATGGTCA |
| 55 | CAGTTTTTAGCTGTTAGGTATATTCGATCACTTTAGTCTATTTTGAAAATGATAT |
| | GAGAC[A/G]CTTTTTAAGCAAAGTCTACAGTTTCCNAATGAGAAAATTAATCCTC |
| | TTTCTTGTCTTTCC |
| 56 | ATATTCGTGGGTATAAATTAGCTCAGGTACTTAAGTGACAAGCCAACAATTCAA |
| | CGTAAG[A/G]AAGGTGGTTCAAGACANCTTCGCAGTTTCGCAGTTTTGGAGAGT |
| | GGAGCAACTCCTGGAG |
| 57 | ACCTAATAGAATGTCAGTGTTCTCAATTCTGTCTGGGCCTNTGGAGTTTTAAAAT |
| | ATACA[A/G]TTTTTCGCATTCTAGTCCCAAGATCGTTGAGTGTACTTGGAAGGGG |
| | ACCAAAAGGCATCA |
| 58 | GAAGAACGATGAGCCTGGAGCATTTATCCATCAACTGCCATCCATCTCTGGGTC |
| | AGGCTT[A/G]ACTCTGCAAATGTCACTATTTTCAAACTGCCAGGCTGCACTTGCA |
| | TTAGGGCTTAGCAGA |
| 59 | TCATATCCTTTTAAGGCACCATCTGGTCTCTTCTAAACTCCCTTGAGTGGTTGATT |
| | GAAG[A/G]AAAACATCTAAGAACAAATAATTTTCTTGGAACAGTACATTCTAAG |
| | TCTATATTTTAGAG |
| 60 | TAGATGCTTGAGAGCGGAGATTGAGATAAATAAAGTAAGGGGACTTTTAGATG |
| | ACCAAGN[A/G]GAGGCAATTAATAGGTAGATGAACGGTTATTTGGGGCTTCCAG |
| | GCAGAGGCTTGCATGGA |
| 61 | ACTCAGTGTCTGTTGAATGAAGAACTGAGCACCAACTCCACATCAGGCTCTCTG |
| | ATTGCC[A/G]GGTGACGTCCTATAATCTTGCTATGAGATTGGGATGTTGTGCCTT |
| | TTATTCCCTAGACAA |
| 62 | ATGCCAATCAAATCAAGTCAAATGTAATTGTTTAGGCTTTTCCATTCTTTCATTC |
| | CTATT[A/C]TATAACCCATGGACACTCAGTCCTATAGCTGAATATTTTATGGCAC |
| | CAGTGTGATGAACT |
| 63 | CCCAAAAGGTTAAATGTGGTTTCATCACAAGCATGGGTGTCAATTTTATAAAGG |
| | AGGGGG[A/G]AAAAGTCATTCATGGGGAACTTAAGGCTTTTATCCACAAAGGTA |
| | CATGTTGAGTGAACTG |
| 64 | AATAAGAATGCCCAAGATGGAACTGAAGAAGATGCAATGGATCAGGAGTCTGC |
| | TGTCCTG[A/G]GCTCCAGGCCCGGGTCTGCCAAACTGGCAAATTCTAAGGCCTTG |
| | AACAACCATTTCAACA |
| 65 | ACTATGTGCCACTGCTGAGCTAGACACAAAAAGAATATGACATGTTCCCTGCCT |
| | TTATGG[A/G]ACTCACAGTGTAGTGGGGGTGACAGATGCAGACACTAAAAATTT |
| | GACTACAGTATGGCTA |
| 66 | CAGGCTTGTTCATCTATTGTACACTGAGCTGATGTTTATGAAGCTCCCAATATGT |
| | GCCAG[A/G]GTCTGTACTTGGTCTAAAGGTNCAGATCCCAAGCCCTCCATGCACT |
| | CTTTGACCTTGGAC |
| 57 | CTGGCACTTTTGCCAGATTATCCAACTGGCAAAATGCACAGATTCTCAGGCATC |
| | AGGAAG[A/G]CAGAGGCAGACAAAGAGAGTCAGAGAGGGGGTGAGGATGCAGT |
| | GACTTCAGCCAGAGTTT |
| 68 | AAGGAATGGCCTGTGATGTACGGCAGAAACCAGTTTTACTAGCGCCTCCATCCA |
| | GTTGCT[A/G]CTTCTGGTTATGTCACAGCCTGGACTCTTCAGGCTACTTGGAAAG |
| | GCCTTTCATGGCTTG |
| 69 | ATATATTCTATAAAAGAGACACTTCAGTAACTCAAAAAGTCTATGTTCTTCAAGT |
| | GCCCC[A/G]CCACAAAGGGTTATAGCCCTTGGATGAAGCATCTTTCTAGTCCTCT |
| | TCTGAACTTACCCA |
| 70 | TGTGTCAAGAGCTTATTGTNTGGGGAATGTTGGTGGGCATTTGACCTCTATCCTC |
| | ATTTC[A/G]TCTTCATCACAGTGCTCCGGGAAAAATCGCAATCACCCCCATTTTA |
| | GAGATGAGGATATG |
| 71 | TTACCTCTCATGAGGGAAATACCCTCATACAGTTGGCCATCACTTAACAATAGA |
| | GACAAC[A/G]ATGATAGATGGGATGGTAGCAACTTTAGGTTTTGTTGTTTCCTAT |
| | TTTTCAGTGGTGAAT |
| 72 | CTGGAGACCAAGGACTATGTTGCACCATAACTATCACCTCCCAGGTATGCAGAA |
| | CTGAGC[A/G]ATTTTCAAAGGTCTTCACCATTCATAGTCTCATTTGAGCCTGAAA |
| | CTACTTTGACAGCTA |
| 73 | GTGAATGCCTATAAAATAAAGTAACATTCGAACAACAGCCCAGAGGGCCGCAC |
| | TGGTAAA\|A/G]CCGTAGCTTCCTCTGTTTCTACTTTCATTCAATAAAAACCGTTTC |
| | GTATTCAACTCAGGG |
| 74 | ACCTCGGTGTGGGCAGGACACCACATTTATTTTAACCTATGAAACTCTCATGGTT |
| | GGTCA[A/C]CCTTGCAATAGGGCTGACTCTGCCCTGATAGCACACATCTGGCAGG |
| | TGGCCCTAAAACAG |
| 75 | ATCTCTGGTGACTTCTTAAAAGAACCGGTTACCTAGAAGACATCAGGAGGAAAG |
| | AGCTAT[A/G]AAGAAACCCACTTCCTGACTTGAGCTTCACTGGCTCACTGTCCAA |
| | GTTTGTGTCTGAGTG |
| 76 | TAACATTGGGCTAGACCTCCTCCTCTAAAAAGAAAAAAAAAAGTCTCNATTCCC |
| | TCATTT[A/G]TACAATGGGCATAACAGAAACTTCCTCATGTGATATTTGGTGAAG |
| | GATTTAAAAAGTCAG |
| 77 | AAATGCCTGCTACGCCCCATGACACTGCCAGCAATTACTGCAATTCTATAAGTA |
| | AAATGC[A/G]TTGTTCCCTGGCCTCAAGGAACTTAGAATTATACTGGAAAAATAA |
| | AAGGTTTGGAGAATA |
| 78 | TCTGTTTCTGCCCTTCTCATTCCCAAGCTCTTTTCCTCTTATCCAATCAGGTACTG |
| | CCCA[A/G]GGATGGTCTACATTGAGACTGTGATGGCTTCAGCAAGCCTGGAAGC |
| | CAGCCCCAGCTTTG |
| 80 | CAATTATTAAATCATCATCTATATTTATTTATAGATGAGGAAACAGACATGAAG |
| | AGACTT[A/C]ACTAGGATGGTTTGTAAAATGTTCAGTTCCTACGTTTGGGGAGAA |
| | GGAGCTGTTGAAAAG |
| 81 | AAGGACAAGCCTGTCATTCCTGCTGCTGCCCTAGCTGGCTACACAGGTAGGCGC |
| | CCTCCC[A/G]CTGCTTAGGCCAACTCCATCTGCACGTTTCTGTGGGTGGGGTCCT |
| | GGAAGGCACTCTGCA |
| 82 | GGGTCCAGAAGCACTAGGGGAGGGGGTAGGAAGGAGTGCACGTAAGATGTCCT |
| | GGGTGTA[C/G]GGCGTGAGGGACAGAAGGCGGGCAAGGTGTCCAGGATGGCGC |
| | NCCTGGCAGTTGGTGGCA |

*The International Union of Pure and Applied Chemistry (IUPAC) nucleotide code is used in the sequence listing to identify the nucleotide at the nucleotposition.

### Methods of Characterizing an Inflammatory Disease or Condition, or Fibrostenotic or Fibrotic Disease

In an aspect, provided herein, are methods of characterizing an inflammatory condition or disease or fibrostenotic or fibrotic disease of a subject, the method comprising assaying a sample obtained from the subject to identify the presence of a genotype comprising a polymorphism comprising a risk allele within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160, rs11606640, rs73029052, rs11600915, rs61909068, rs12294634, rs73029062, rs11600746, rs3851519, rs61909072, or rs56086356 of Table 5. The polymorphism within rs201292440 has merged with rs59418409, which means rs201292440 and rs59418409 may be used interchangeably to refer to the same polymorphism. In some embodiments, the polymorphism comprises any one of SEQ ID NOS: 1-36. In some embodiments, all of the polymorphisms of Table 5 are detected. In some embodiments, one copy of the polymorphism at the TNFSF15 gene locus is detected. In some embodiments, a combination of one copy of the polymorphism at the TNFSF15 gene locus and the polymorphism at the ARHGAP15 gene locus is detected, the combinations comprising any one the combinations of Table 3. In some embodiments, more than one combination from Table 3 are detected. In some embodiments, two copies of the polymorphism at the TNFSF15 gene locus are detected. In some embodiments, a combination of two copies of the polymorphism at the TNFSF15 gene locus and the polymorphism at the LY86, ETS1, or SCUBE1 gene loci are detected, the combinations comprising any one the combinations of Table 4. In some embodiments, the methods of detection disclosed herein are used to characterize the inflammatory condition or disease or fibrostenotic or fibrotic disease. In some embodiments, the methods of characterizing the inflammatory condition or disease or fibrostenotic or fibrotic disease are used to select a therapy for the subject, or treat the subject with a therapy. The therapy may include an inhibitor of TL1A activity or expression. The inhibitor of TL1A activity or expression may comprise one or more sequences provided in Table 1 or Table 8.

### Methods of Detection

In an aspect, provided herein, are methods of detecting the presence, absences or quantity of a polymorphism, which may be used for the purposes treating or characterizing the inflammatory disease or condition, or fibrosis of a subject, as described herein. Many nucleic acid-based detection techniques may be useful for the present methods.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid. In an example, the present disclosure provides probes that are hybridizable to a target nucleic acid sequence within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160, rs11606640, rs73029052, rs11600915, rs61909068, rs12294634, rs73029062, rs11600746, rs3851519, rs61909072, or rs56086356. In some embodiments, the nucleic acid probe comprises anyone of SEQ ID NOS: 37-72. The polymorphism within rs201292440 has merged with rs59418409, which means rs59418409 may be detected instead of rs201292440 to determine the presence of the same polymorphism.

In some instances, the methods involve hybridization or amplification assays that include, but are not limited to, Southern or Northern analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification. As discussed, reference to qPCR herein includes use of TaqMan™ methods. In an additional example, hybridization assay includes the use of nucleic acid probes conjugated or otherwise immobilized on a bead, multi-well plate, or other substrate, wherein the nucleic acid probes are configured to hybridize with a target nucleic acid sequence of a genotype provided herein. A non-limiting method is one employed in Anal Chem. 2013 Feb. 5; 85(3):1932-9.

In some embodiments, detecting the presence or absence of a genotype comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods may also be employed.

In some instances, a number of nucleotides that are sequenced are at least 5, 10, 15, 20,25,30,35,40,45,50,100, 150,200,300,400,500,2000,4000,6000, 8000, 10000,20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

In an aspect, provided herein, are methods comprising: a) providing a sample obtained from a subject with an inflammatory condition or disease or fibrostenotic or fibrotic disease; b) assaying to detect in the sample obtained from the subject a presence of a polymorphism located at a gene locus comprising TNFSF15, LY86, ETS1, ARHGAP15, or SCUBE1; and c) detecting the presence of the polymorphism by contacting the sample obtained from the subject with a nucleic acid capable of hybridizing at least about 10 and less than 50 contiguous nucleotides of the polymorphism, or reverse complement sequence thereof, under standard hybridization conditions and detecting binding between the polymorphism and the nucleic acid sequence. The standard hybridization conditions may comprise an annealing temperature between about 30° C. and about 65° C. In some embodiments, the nucleic acid comprises any one of SEQ ID NOS: 37-72.

In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises RNA. In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

In an aspect, provided herein, the detection of the polymorphism involves amplification of the subject's nucleic acid by the polymerase chain reaction (PCR). In some embodiments, the PCR assay involves use of a pair of primers capable of amplifying at least about 10 and less than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160 rs11606640, rs73029052, rs11600915, rs61909068, rs12294634, rs73029062, rs11600746, rs3851519, rs61909072, or rs56086356, the nucleobase comprising the risk allele. Additional primers include those having a sequence that is a reverse complement to those described herein, e.g., a reverse complement to any one of rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160 rs11606640, rs73029052, rs11600915, rs61909068, rs12294634, rs73029062, rs11600746, rs3851519, rs61909072, and rs56086356, the nucleobase comprising the risk allele. In some embodiments, the nucleic acid amplification assay comprises amplification of DNA from the subject with a pair of primers cap able of amplifying at least about 10 and less than 50 contiguous nucleobases within any one of SEQ ID NOS: 1-36. Additional primers include those having a sequence that is a reverse complement to those described herein, e.g., a reverse complement to any one of SEQ ID NOS: 1-36. In some embodiments, quantitative PCR may also be used. In some embodiments, a nucleic acid probe complementary to at least about 10 and less than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160, rs11606640, rs73029052, rs11600915, rs61909068, rs12294634, rs73029062, rs11600746, rs3851519, rs61909072, or rs56086356, including the nucleobase comprising the risk allele. Additional probes include those having a sequence that is a reverse complement to those described herein, e.g., a reverse complement to any one of rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160 rs11606640, rs73029052, rs11600915, rs61909068, rs12294634, rs73029062, rs11600746, rs3851519, rs61909072, or rs56086356, including the nucleobase comprising the risk allele. In some embodiments, the nucleic acid amplification assay comprises amplification of DNA from the subject with a nucleic acid probe complementary to at least about 10 and less than 50 contiguous nucleobases within any one of SEQ ID NOS: 1-36. In some embodiments, the nucleic acid probe comprises any one of SEQ ID NOS: 37-72. Additional probes include those having a sequence that is a reverse complement to those described herein, e.g., a reverse complement to any one of SEQ ID NOS: 1-36. In fluorogenic quantitative PCR, quantitation is based on amount of fluorescence signals (TaqMan and SYBR green). In some embodiments, the nucleic acid probe is conjugated to a detectable molecule. The detectable molecule may be a fluorophore. The nucleic acid probe may also be conjugated to a quencher.

### Compositions and Kits

An aspect, provided herein, are compositions comprising at least 10 but less than 50 contiguous nucleobase residues of any one of SEQ ID NOS: 1-36, wherein the contiguous nucleobase residues comprise the nucleobase at position 501 of any one of SEQ ID NOS: 1-36, and wherein the contiguous nucleobase residues are connected to a detectable molecule. The detectable molecule may be any molecule suitable for nucleic acid detection. In some embodiments, the detectable molecule is a fluorophore. In some embodiments, the composition is complementary to at least about 10 and less than 50 contiguous nucleobases within rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160, rs11606640, rs73029052, rs11600915, rs61909068, rs12294634, rs73029062, rs11600746, rs3851519, rs61909072, or rs56086356 wherein one of the nucleobases comprises the risk allele. Additional compositions include those having a sequence that is a reverse complement to those described herein, e.g., a reverse complement to any one of rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160, rs11606640, rs73029052, rs11600915, rs61909068, rs12294634, rs73029062, rs11600746, rs3851519, rs61909072, or rs56086356, wherein one of the nucleobases comprises the risk allele. In some embodiments the contiguous nucleobase residues are connected to a quencher.

An aspect provided herein are kits, comprising the composition disclosed herein, and a primer pair capable of amplifying at least about 10 contiguous nucleobases within SEQ ID NOS: 1-36. In some embodiments, the primer pair is capable of amplifying at least about 10 contiguous nucleobases within any one of rs6478109, rs7848647, rs201292440, rs7869487, rs4366152, rs6478108, rs1407308, rs7866342, rs7030574, rs10114470, rs4979464, rs3810936, rs7028891, rs7863183, rs4979469, rs1853187, rs7040029, rs722126, rs4246905, rs4979467, rs4979466, rs7043505, rs911605, rs11793394, rs17219926, rs7874896, rs4574921, rs6478106, rs7032238, rs55775610, rs7847158, rs56069985, rs10790957, rs6921610, rs6757588, rs6003160, rs11606640, rs73029052, rs11600915, rs61909068, rs12294634, rs73029062, rs11600746, rs3851519, rs61909072, or rs56086356, including nucleobase comprising the risk allele. In some embodiments, methods are provided for contacting DNA from a subject with the composition described herein, or using the kit described herein under conditions configured to hybridize the composition to the DNA if the DNA comprises a sequence complementary to the composition. In further embodiments, provided herein are methods of treating the subject with an inhibitor of TL1A activity or expression, provided that the DNA from the subject comprises the sequence complementary to the composition. The therapy may include an inhibitor of TL1A activity or expression. The inhibitor of TL1A activity or expression may comprise one or more sequences provided in Table 1 or Table 8.

### Biological Samples, Sample Preparation and Gene Expression Detection

As described further above, in various embodiments of the methods provided herein, the methods further comprise preparing the sample. In one embodiment, preparing sample comprises or consists of obtaining the sample from the subject. In another embodiments, preparing sample comprises or consists of releasing DNA from the sample. In a further embodiment, preparing sample comprises or consists of purifying the DNA. In yet another embodiments, preparing sample comprises or consists of amplifying the DN. In one embodiment, preparing sample comprises or consists of obtaining the sample from the subject and releasing DNA from the sample. In some embodiments, preparing sample comprises or consists of obtaining the sample from the subject and purifying the DNA. In certain embodiments, preparing sample comprises or consists of obtaining the sample from the subject and amplifying the DNA. In further embodiments, preparing sample comprises or consists of releasing DNA from the sample and purifying the DNA. In one embodiment, preparing sample comprises or consists of releasing DNA from the sample and amplifying the DNA. In other embodiments, preparing sample comprises or consists of purifying the DNA and amplifying the DNA. In yet other embodiments, preparing sample comprises or consists of obtaining the sample from the subject, releasing DNA from the sample, and purifying the DNA. In some embodiments, preparing sample comprises or consists of obtaining the sample from the subject, releasing DNA from the sample and amplifying the DNA. In certain embodiments, preparing sample comprises or consists of obtaining the sample from the subject, purifying the DNA and amplifying the DNA. In some embodiments, preparing sample comprises or consists of releasing DNA from the sample, purifying the DNA and amplifying the DNA. In other embodiments, preparing sample comprises or consists of obtaining the sample from the subject, releasing DNA from the sample, purifying the DNA, and amplifying the DNA.

Additionally, the disclosure provides various assays for determining or detecting the genotypes, combinations of genotypes, polymorphisms, or combinations of polymorphisms. As such, in various embodiments of the methods provided herein, comprise determining or detecting the genotypes, combinations of genotypes, polymorphisms, or combinations of polymorphisms comprises or consists of assaying for the genotypes, combinations of genotypes, polymorphisms, or combinations of polymorphisms via any assays as described elsewhere herein. Alternatively, in various embodiments of the methods provided herein, the method further comprises assaying for the genotypes, combinations of genotypes, polymorphisms, or combinations of polymorphisms via any assays as described herein.

Sample Collection from Patients or Subjects

In some embodiments, the methods further comprise: obtaining the sample from the subject. Samples used for the genotyping, can be any samples collected from patients that contain the patient's DNA such as genomic DNA. In some specific embodiment of the methods provided herein, the sample is a bodily fluid sample. In one embodiment, the sample is a tissue sample. In one embodiment, the sample is a cell sample. In one embodiment, the sample is a blood sample. In one embodiment, the sample is a bone marrow sample. In one embodiment, the sample is a plasma sample. In one embodiment, the sample is a serum sample. In one embodiment, the sample is a saliva sample. In one embodiment, the sample is a cerebrospinal fluid sample.

DNA Release from Samples

Kits and methods disclosed herein are generally suitable for analyzing a biological sample obtained from a subject. Similarly, methods disclosed herein comprises processing or analysis of a biological sample. Biological samples may be obtained through surgical biopsy or surgical resection. In some instances, a needle biopsy aspiration can be used to collect the biological sample from a subject. Biological samples may be obtained by a fluid draw, swab or fluid collection. Biological samples may be obtained through primary patient derived cell lines, or archived patient samples in the form of FFPE (Formalin fixed, paraffin embedded) samples, or fresh frozen samples. Biological samples may comprise whole blood, peripheral blood, plasma, serum, saliva, cheek swab, urine, or other bodily fluid or tissue. The sample may comprise tissue from the large or small intestine. The large intestine sample may comprise the cecum, colon (the ascending colon, the transverse colon, the descending colon, and the sigmoid colon), rectum or the anal canal. The small intestine sample may comprise the duodenum, jejunum, or the ileum. The sample may also comprise a blood sample. The sample may comprise serum. The sample may comprise tissue and blood.

DNA molecules can be released from the cells or tissues in patient's samples by various ways. For example, the DNA molecules can be released by breaking up the host cells physically, mechanically, enzymatically, chemically, or by a combination of physical, mechanical, enzymatic and chemical actions. In some embodiments, the DNA molecules can be released from the samples by subjecting the samples to a solution of cell lysis reagents. Cell lysis reagents include detergents, such as triton, SDS, Tween, NP-40, or CHAPS. In other embodiments, the DNA molecules can be released from the samples by subjecting the samples to difference in osmolarity, for example, subjecting the samples to a hypotonic solution. In other embodiments, the DNA molecules can be released from the samples by subjecting the samples to a solution of high or low pH. In certain embodiments, the DNA molecules can be released from the samples by subjecting the samples to enzyme treatment, for example, treatment by lysozyme. In some further embodiments, the DNA molecules can be released from the samples by subjecting the samples to any combinations of detergent, osmolarity pressure, high or low pH, or enzymes (e.g. lysozyme).

Alternatively, the DNA molecules can be released from the host cells by exerting physical force on the host cells. In one embodiment, the DNA molecules can be released from the host cells by directly applying force to the host cells, e.g. by using the Waring blender and the Polytron. Waring blender uses high-speed rotating blades to break up the cells and the Polytron draws tissue into a long shaft containing rotating blades. In another embodiment, the DNA molecules can be released from the host cells by applying shear stress or shear force to the host cells. Various homogenizers can be used to force the host cells through a narrow space, thereby shearing the cell membranes. In some embodiments, the DNA molecules can be released from the host cells by liquid-based homogenization. In one specific embodiment, the DNA molecules can be released from the host cells by use a Dounce homogenizer. In another specific embodiment, the DNA molecules can be released from the host cells by use a Potter-Elvehjem homogenizer. In yet another specific embodiment, the DNA molecules can be released from the host cells by use a French press. Other physical forces to release the DNA molecules from host cells include manual grinding, e.g. with a mortar and pestle. In manual grinding, host cells are often frozen, e.g. in liquid nitrogen and then crushed using a mortar and pestle, during which process the tensile strength of the cellulose and other polysaccharides of the cell wall breaks up the host cells.

Additionally, the DNA molecules can be released from the samples by subjecting the samples to freeze and thaw cycles. In some embodiments, a suspension of samples are frozen and then thawed for a number of such freeze and thaw cycles. In some embodiments, the DNA molecules can be released from the samples by applying 1, 2, 3, 4, 5, 6, 7, 8, 9, 10, 11, 12, 13, 14, 15, 16, 17, 18, 19, or 20 freeze and thaw cycles to the samples.

The above described methods for releasing the DNA molecules from the samples are not mutually exclusive. Therefore, the disclosure provides that the DNA molecules can be released from the samples by any combinations of DNA releasing methods described herein.

### DNA Purification or Enrichment

In some embodiments, the methods provided herein further comprise purifying the subject's DNA molecules before genotyping assays. In one embodiment, the methods provided herein further comprise purifying the DNA by affinity purification. In one embodiment, the methods provided herein further comprise purifying the DNA by affinity purification with spin column. In one embodiment, the methods provided herein further comprise purifying the DNA by affinity purification with a positively charged matrix in the spin column that binds to the negatively charged DNA. In one embodiment, the methods provided herein further comprise purifying the DNA by affinity purification with a silica matrix in the spin column that binds to the DNA. In one embodiment, the methods provided herein further comprise purifying the DNA by affinity purification with an affinity tag that binds to the DNA or a fragment thereof. In some embodiments, the DNA bound to the affinity purification matrix can be eluted with an elution buffer or water, thereby yielding DNA with higher purity and higher concentration.

In some embodiments, it is important to enrich or purify abnormal tissues or abnormal cells from normal tissue or cells of the biological sample. In some embodiments, the abnormal tissue or cell sample is microdissected to reduce the amount of normal tissue contamination before extraction of genomic nucleic acid or pre-RNA for use in the methods described herein. Such enrichment or purification may be accomplished according to methods, such as needle microdissection, laser microdissection, fluorescence activated cell sorting, and immunological cell sorting.

### Biomarker Detection

Nucleic acid or protein samples derived from the biological sample (e.g., tissue, fluid, cells) of a subject may be used in the methods of the inventive concepts. Analysis of the nucleic acid or protein from an individual may be performed using any of various techniques. In some instances, a genome wide association study (GWAS) is performed. In some instances, GWAS comprises use of a genotyping array, also referred to as a SNP array. In some instances, GWAS comprises sequencing. In various embodiments, assaying gene expression levels for genetic risk variants comprises northern blot, reverse transcription PCR, real-time PCR, serial analysis of gene expression (SAGE), DNA microarray, tiling array, RNA-Seq, ImmunoArray, or a combination thereof.

Determining a protein expression may be accomplished by analyzing the proteins of a biological sample from the subject. Protein expression can be detected by enzyme-linked immunosorbent assay (ELISA), immunohistochemistry, western blot, flow cytometry, fluorescence in situ hybridization (FISH), radioimmunoassays, or affinity purification. The ELISA may be a sandwich ELISA, competitive ELISA, multiple and portable ELISA.

### DNA Amplification

In some embodiments, the method provided herein comprises an DNA amplification step. The DNA amplification includes, for example, reactions comprising a forward and reverse primer, such that the primer extension products of the forward primer serve as templates for primer extension of the reverse primer, and vice versa. Amplification may be isothermal or non-isothermal. A variety of methods for amplification of target polynucleotides are available, and include without limitation, methods based on polymerase chain reaction (PCR). Conditions favorable to the amplification of target sequences by PCR can be optimized at a variety of steps in the process, and depend on characteristics of elements in the reaction, such as target type, target concentration, sequence length to be amplified, sequence of the target or one or more primers, primer length, primer concentration, polymerase used, reaction volume, ratio of one or more elements to one or more other elements, and others, some or all of which can be suitably altered. In general, PCR involves denaturation of the target to be amplified (if double stranded), hybridization of one or more primers to the target, and extension of the primers by a DNA polymerase, with the steps repeated (or “cycled”) in order to amplify the target sequence. Steps in this process can be optimized for various outcomes, such as to enhance yield, decrease the formation of spurious products, or increase or decrease specificity of primer annealing. Methods of optimization include adjustments to the type or amount of elements in the amplification reaction or to the conditions of a given step in the process, such as temperature at a particular step, duration of a particular step, or number of cycles. In some embodiments, an amplification reaction comprises at least or about 5, 10, 15, 20, 25, 30, 35, 40, 45, 50, or more cycles. In some embodiments, an amplification reaction comprises no more than 5, 10, 15, 20, 25, 35, 40, 45, 50, or more cycles. Cycles can contain any number of steps, such as 1, 2, 3, 4, 5, or more steps. Steps can comprise any temperature or gradient of temperatures, suitable for achieving the purpose of the given step, including but not limited to, 3′ end extension, primer annealing, primer extension, and strand denaturation. Steps can be of any duration, including but not limited to about or less than about 1, 5, 10, 15, 20, 25, 30, 35, 40, 45, 50, 55, 60, 70, 80,90, 100, 120, 180,240,300,360, 420, 480, 540, 600, or more seconds, including indefinitely until manually interrupted. In some embodiments, amplification is performed separately for each sample (e.g., for DNA purified from patient samples as described above). In some embodiments, amplification is performed separately for each sample (e.g., for DNA purified from patient samples as described above), but together on one PCR plate (e.g. 96 well plate wherein up to 96 PCR reactions were performed together). In some embodiments, amplification is performed before or after pooling of target polynucleotides (e.g., DNA purified from patient samples as described above) from independent samples or aliquots. Non-limiting examples of PCR amplification techniques include quantitative PCR (qPCR or real-time PCR), digital PCR, and target-specific PCR.

Non-limiting examples of polymerase enzymes for use in PCR include thermostable DNA polymerases, such as Thermus thermophilus HB8 polymerase; Thermus oshimai polymerase; Thermus scotoductus polymerase; Thermus thermophilus polymerase; Thermus aquaticus polymerase (e.g., AmpliTaq® FS or Taq (G46D; F667Y); Pyrococcus furiosus polymerase; Thermococcus sp. (strain 9° N-7) polymerase; Tsp polymerase; Phusion High-Fidelity DNA Polymerase (ThermoFisher); and mutants, variants, or derivatives thereof. Further examples of polymerase enzymes useful for some PCR reactions include, but are not limited to, DNA polymerase I, mutant DNA polymerase I, Klenow fragment, Klenow fragment (3′ to 5′ exonuclease minus), T4 DNA polymerase, mutant T4 DNA polymerase, T7 DNA polymerase, mutant T7 DNA polymerase, phi29 DNA polymerase, and mutant phi29 DNA polymerase. In some embodiments, a hot start polymerase is used. A hot start polymerase is a modified form of a DNA Polymerase that requires thermal activation. The hot start enzyme is provided in an inactive state. Upon thermal activation the modification or modifier is released, generating active enzyme. A number of hot start polymerases are available from various commercial sources, such as Applied Biosystems; Bio-Rad; ThermoFisher; New England Biolabs; Promega; QIAGEN; Roche Applied Science; Sigma-Aldrich; and the like.

In some embodiments, primer extension and amplification reactions comprise isothermal reactions. Non-limiting examples of isothermal amplification technologies are ligase chain reaction (LCR) (see e.g., U.S. Pat. Nos. 5,494,810 and 5,830,711); transcription mediated amplification (TMA) (see e.g., U.S. Pat. Nos. 5,399,491, 5,888,779, 5,705,365, 5,710,029); nucleic acid sequence-based amplification (NASBA) (see e.g., U.S. Pat. No. 5,130,238); signal mediated amplification of RNA technology (SMART) (see e.g., Wharam et al., Nucleic Acids Res. 2001, 29, e54); strand displacement amplification (SDA) (see e.g., U.S. Pat. No. 5,455,166); thermophilic SDA (see e.g., U.S. Pat. No. 5,648,211); rolling circle amplification (RCA) (see e.g., U.S. Pat. No. 5,854,033); loop-mediated isothermal amplification of DNA (LAMP) (see e.g., U.S. Pat. No. 6,410,278); helicase-dependent amplification (HDA) (see e.g., U.S. Pat. Appl. 20040058378); exponential amplification methods based on SPIA (see e.g., U.S. Pat. No. 7,094,536); and circular helicase-dependent amplification (cHDA) (e.g., U.S. Pat. Appl. 20100075384).

In an aspect, provided herein, the analysis of gene expression levels involves amplification of an individual's nucleic acid by the polymerase chain reaction (PCR), such as the methods disclosed in Mullis et al. (Eds.), The Polymerase Chain Reaction, Birkhauser, Boston, (1994)). PCR may include “quantitative” nucleic acid amplification, e.g., qPCR Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The reported nucleic acid sequence for the genes is sufficient to routinely select primers to amplify any portion of the gene. Fluorogenic quantitative PCR may also be used in aspects disclosed herein. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

A DNA sample suitable for hybridization may be obtained, e.g., by polymerase chain reaction (PCR) amplification of genomic DNA, fragments of genomic DNA, fragments of genomic DNA ligated to adaptor sequences or cloned sequences. Computer programs can be used in the design of primers with the predetermined specificity and optimal amplification properties, such as Oligo version 5.0 (National Biosciences). PCR methods are described, for example, in Innis et al., eds., 1990, PCR Protocols: A Guide to Methods And Applications, Academic Press Inc., San Diego, Calif. It will be apparent to one skilled in the art that controlled robotic systems are useful for isolating and amplifying nucleic acids and can be used.

### Determination of Genotypes

Genotypes can be determined by hybridization of probes to the amplified DNA (e.g. as described above), wherein the probes are specific for each polymorphism (e.g. each SNP) and a short sequence flanking the polymorphism. Alternatively, genotypes can be determined by adding probes to the PCR reaction mixture and having the probe hybridize with the PCR product during each cycle of the PCR amplification.

In one embodiment, genotypes (e.g. SNPs) can be determined by adding a fluorogenic probe, complementary to the target sequence (e.g. the short sequence encompassing the polymorphisms), to the PCR reaction mixture. This probe is an oligonucleotide with a reporter dye attached to the 5′ end and a quencher dye attached to the 3′ end such that the reporter and the quencher are in close proximity in the probe in a default configuration (e.g with a short hairpin structure or due to the short length of the probe). When the probe is not bound to the target or hydrolyzed by the polymerase, the quencher and the fluorophore remain in proximity to each other, separated by the length of the probe, leaving a background fluorescence. During PCR, the probe anneals specifically between the forward and reverse primer to the internal region of the PCR product encompassing the polymorphism. The polymerase then carries out the extension of the primer and replicates the template to which the probe is bound. The 5′ exonuclease activity of the polymerase cleaves the probe, releasing the reporter molecule away from the close vicinity of the quencher. The fluorescence intensity of the reporter dye increases as a result. This process repeats in every cycle and does not interfere with the accumulation of PCR product, resulting in continuous increase of the reporter fluorescence intensity. The genotypes (e.g. polymorphisms and SNPs) are determined by the fluorescence signal. The probes for the genotypes (e.g. polymorphisms and SNPs) are often 10-30 bases in length and designed to discriminate between its target and a highly related mismatch sequence. For this discrimination to be successful, the probes are designed to provide a difference in the melting temperatures of the duplex with the intended target and the duplex with highly related mismatch sequence (e.g. a high ΔTm value). The length and sequence of the probe is designed, at least in part, to optimize such ΔTm. In some embodiments, the probes are DNA molecules. In some embodiments, the probes are RNA molecules. In some embodiments, the probes are locked nucleic acids (LNA). The LNA probes provide significant differences in ΔTm, often around 20° C. for single mismatches, due to the high specificity and high affinity of the LNA probes. In some embodiments, the reporter dye is a fluorescence dye.

In some embodiments, the genotyping can be performed in a multiplexing assay. A multiplexing assay refers to an assay that can detect or determine multiple genotypes, e.g multiple polymorphisms or multiple SNPs in the sample. Multiplexing can be achieved via physical separation or multiplication of the same sample, e.g. running a 96-well plate PCR with specific PCR primer and SNP detecting probe per well, but multiple SNP detecting probes for the sample per plate, thereby detecting multiple genotypes for a sample in one 96-well PCR. Multiplexing can also be achieved by running a PCR reaction with multiple PCR primers and multiple SNP detecting probes, with each probe attached to a fluorescent dye of a unique color, thereby distinguishing the SNPs in the single reaction via unique fluorescence signal associated with each SNP. In one embodiment, the methods provide herein comprise a multiplexing PCR In another embodiment, the methods provided herein comprise a multiplexing PCR with each genotype (e.g. each polymorphism or SNP) detected in a different fluorescence signal. Other multiplexing PCR methods, such as multiplexed qPCR or multiplexed digital PCR can be used here as well. In one embodiment, the methods provided herein comprise multiplexed qPCR In another embodiment, the methods provided herein comprise multiplexed digital PCR.

Similarly, other hybridization or PCT based can also be used to detect or determining the genotypes (e.g. polymorphisms or SNPs) and are provided herein. For example, in some embodiments, the genotypes (e.g. polymorphisms or SNPs) are detected or determined via dynamic allele-specific hybridization such as described in Genome Res. 2001 January; 11(1): 152-162, molecular beacons such as described in Clin Chem Lab Med. 2003 April; 41(4):468-74, SNP microarrays as commercially available from Affymetrix.

Alternatively, the genotype (e.g. the polymorphisms or SNPs) can be detected or determined by sequencing the DNA purified from the sample as described herein or the amplified DNA described herein. In some embodiments, the methods comprise sequencing the purified DNA or the amplified DNA. In some embodiments, the methods comprise sequencing products of the amplification with a primer different from the primers used in the amplification. In some embodiments, the methods comprise sequencing the purified DNA or the amplified DNA by next generation sequencing (NGS).

A variety of sequencing methodologies are available, particularly high-throughput sequencing methodologies. Examples include, without limitation, sequencing systems manufactured by Illumina (ILLUMINA next generation sequencing, sequencing systems such as Hi Seq® and MiSeq®), Life Technologies (Ion Torrent®, SOLiD®, etc.), Roche's 454 Life Sciences systems, Pacific Biosciences systems, nanopore sequencing platforms by Oxford Nanopore Technologies, etc, which manufactures public protocols and instructions for sequencing are each hereby incorporated in their entirety by reference. In some embodiments, sequencing comprises producing reads of about or more than about 50, 75, 100, 125, 150, 175, 200, 250, 300, or more nucleotides in length. In some embodiments, sequencing comprises a sequencing by synthesis process, where individual nucleotides are identified iteratively, as they are added to the growing primer extension product. Pyrosequencing is an example of a sequence by synthesis process that identifies the incorporation of a nucleotide by assaying the resulting synthesis mixture for the presence of by-products of the sequencing reaction, namely pyrophosphate, an example description of which can be found in U.S. Pat. No. 6,210,891. According to some sequencing methodologies, the primer/template/polymerase complex is immobilized upon a substrate and the complex is contacted with labeled nucleotides. Further non-limiting examples of sequencing technologies are described in US20160304954, U.S. Pat. Nos. 7,033,764, 7,416,844, and WO2016077602. In some embodiments, the methods comprise sequencing the purified DNA or the amplified DNA by next generation sequencing (NGS)

In some cases, sequencing reactions of various types, as described herein, may comprise a variety of sample processing units. Sample processing units may include but are not limited to multiple lanes, multiple channels, multiple wells, and other methods of processing multiple sample sets substantially simultaneously. Additionally, the sample processing unit may include multiple sample chambers to facilitate processing of multiple runs simultaneously. In some embodiments, simultaneous sequencing reactions are performed using multiplex sequencing. In some embodiments, polynucleotides are sequenced to produce about or more than about 5000, 10000, 50000, 100000, 1000000, 5000000, 10000000, or more sequencing reads in parallel, such as in a single reaction or reaction vessel. Subsequent data analysis can be performed on all or part of the sequencing reactions. Where polynucleotides are associated with an index sequence, data analysis can comprise grouping sequences based on index sequence for analysis together, or comparison to sequences associated with one or more different indices.

In some embodiments, sequence analysis comprises comparison of one or more reads to a reference sequence (e.g., a control sequence, sequencing data for a reference population, or a reference genome), such as by performing an alignment. In an alignment, a base in a sequencing read alongside a non-matching base in the reference indicates a polymorphism (e.g. SNP) at that nucleoposition. Similarly, where one sequence includes a gap alongside a base in the other sequence, an insertion or deletion mutation (an “indel”) is inferred to have occurred. When it is predetermined to specify that one sequence is being aligned to one other, the alignment is sometimes called a pairwise alignment. Multiple sequence alignment generally refers to the alignment of two or more sequences, including, for example, by a series of pairwise alignments. Examples of algorithms for performing alignments include, without limitation, the Smith-Waterman (SW) algorithm, the Needleman-Wunsch (NW) algorithm, algorithms based on the Burrows-Wheeler Transform (BWT), and hash function aligners such as Novoalign (Novocraft Technologies; available at www.novocraft.com), ELAND (Illumina, San Diego, Calif.), SOAP (available at soap.genomics.org.cn), and Maq (available at maq.sourceforge.net). For example, one alignment program, which implements a BWT approach, is Burrows-Wheeler Aligner (BWA) available from the SourceForge web site maintained by Geeknet (Fairfax, Va.). An alignment program that implements a version of the Smith-Waterman algorithm is MUMmer, available from the SourceForge web site maintained by Geeknet (Fairfax, Va.). Other non-limiting examples of alignment programs include: BLAT from Kent Informatics (Santa Cruz, Calif.); SOAP2, from Beijing Genomics Institute (Beijing Conn.) or BGI Americas Corporation (Cambridge, Mass.); Bowtie; Efficient Large-Scale Alignment of Nucleotide Databases (ELAND) or the ELANDv2 component of the Consensus Assessment of Sequence and Variation (CASAVA) software (Illumina, San Diego, Calif.); RTG Investigator from Real Time Genomics, Inc. (San Francisco, Calif.); Novoalign from Novocraft (Selangor, Malaysia); Exonerate, European Bioinformatics Institute (Hinxton, UK), Clustal Omega, from University College Dublin (Dublin, Ireland); and ClustalW or ClustalX from University College Dublin (Dublin, Ireland).

Furthermore, barcode IDs can be introduced to the amplified DNA for each sample and for each SNP via the PCR primer pairs for the PCR reaction. “Barcode ID,” “barcode,” or “ID,” refers to a sequence or a series of sequences that can be used to identify, directly or indirectly through the identification information contained in the sequence or the series of the sequences. Such an ID can be a nucleic acid molecule with a given sequence, a unique fluorescent label, a unique colorimetric label, a sequence of the fluorescent labels, a sequence of the colorimetric label, or any other molecules or combination of molecules, so long as molecules or the combination of molecules used as IDs can identify or otherwise distinguish a particular target or sample from other targets or samples and be correlated with the intended target or sample. Nucleic acid molecules used as such IDs are also known as barcode sequences. Such an ID can also be a further derivative molecule that contains the information derived from but is non-identical to the original ID, so long as such derived molecules or the derived information can identify or otherwise distinguish a particular target or sample from other targets or samples and be correlated with the intended target or sample. For example, a nucleic acid ID can include both the original nucleic acid barcode sequence or the reverse complement of the original nucleic acid barcode sequence, as both can distinguish and be correlated with the intended target or sample. The barcode sequence can be any sequences, natural or non-natural, that are not present without being introduced as barcode sequences in the intended sample, the intended target, or any part of the intended sample or target, so that the barcode sequence can identify and be correlated with the sample or target. A barcode sequence can be unique to a single nucleic acid species in a population or a barcode sequence can be shared by several different nucleic acid species in a population. Each nucleic acid probe in a population can include different barcode sequences from all other nucleic acid probes in the population. Alternatively, each nucleic acid probe in a population can include different barcode sequences from some or most other nucleic acid probes in a population. For a specific example, all the amplified DNA generated from one patient sample can have the same sample barcode sequence (sample ID). For another example, all the amplified DNA generated for a target SNP can have a unique target barcode sequences (“target IDs”). Therefore, the disclosure provides that each patient sample can be identified by the patient ID and the PCR product for each SNP can be identified by a target ID, thereby providing multiplexing for multiple samples and multiple SNP detection in one reaction.

As such, in one embodiment, the methods comprising detecting multiple SNPs in a multiplexing assay by incorporating a unique target ID to each PCR primer pairs used to amplify the sequence fragment containing each SNP. In one embodiment, the methods comprising detecting multiple SNPs in a multiplexing assay by incorporating a unique sample ID to all PCR primer pairs used to amplify one patient sample. In another embodiment, the methods comprising detecting multiple SNPs in a multiplexing assay by (1) incorporating a unique target ID to each PCR primer pairs used to amplify the sequence fragment containing each SNP and (2) incorporating a unique sample ID to all PCR primer pairs used to amplify one patient sample.

The amplified DNA in the multiplexing assay methods disclosed herein can be detected by multiplexed qPCR, multiplexed digital PCR, or NGS. For example, in some embodiments, the amplified DNA in the multiplexing assay methods disclosed herein can be detected by NGS. The use of NGS to detect the amplified DNA generated by assay methods disclosed herein include some advantages. For example, by incorporating target and sample ID tags into the amplified DNA, as described herein, NGS is capable of multiplexed detection at a very large scale. For example, NGS can read a pool of 100 samples, each comprising 10 targets (e.g. 1000-plex) in a single run. This significantly reduces the per data point cost Additionally, NGS can count and aggregate the number of molecules of the same sequence, providing digital quantification at single molecule resolution. Furthermore, a wide range of error correction algorithms, such as parity check, Hamming codes (e.g. Bystrykh, PLoS ONE 7(5): e36852 (2012)), and Levenshtein codes (e.g. Buschmann, BMC Bioinformatics. 2013; 14: 272 (2013)) can be used from communication theory and applied herein to reduce false counts so that NGS based quantification can achieve high precision without repeated sequencing.

As such, provided herein are also assay methods comprising simultaneously detecting at least two SNPs in a patient sample, by simultaneously detecting the unique target IDs associated with each SNP. Also provided herein are assay methods comprising simultaneously detecting at least two SNPs in at least two samples, by simultaneously detecting the unique target IDs associated with each SNP and the unique sample IDs associated with each sample.

In some embodiments, the assay methods provided herein simultaneously detect at least two, at least three, at least four, at least five, at least six, at least seven, at least eight, at least nine, at least ten, or more SNPs in at least two, at least three, at least four, at least five, at least six, at least seven, at least eight, at least nine, at least ten, at least 15, at least 20, at least 30, at least 40, at least 50, at least 60, at least 70, at least 80, at least 90, at least 100, at least 150, at least 200, at least 250, at least 300, at least 350, at least 400, at least 450, or at least 500 samples by simultaneously detecting unique sample IDs and unique target IDs in the amplified DNA with each sample. In some embodiments, the assay methods provided herein simultaneously detect at least two, at least three, at least four, at least five, at least six, at least seven, at least eight, at least nine, at least ten, or more SNPs in a sample by detecting unique target IDs in the amplified DNA with each sample. In some embodiments, the assay methods provided herein simultaneously detect about two, about three, about four, about five, about six, about seven, about eight, about nine, about ten, or more SNPs in about two, about three, about four, about five, about six, about seven, about eight, about nine, about ten, about 15, about 20, about 30, about 40, about 50, about 60, about 70, about 80, about 90, about 100, about 150, about 200, about 250, about 300, about 350, about 400, about 450, or about 500 samples by simultaneously detecting unique sample IDs and unique target IDs in the amplified DNA with each sample. In some embodiments, the assay methods provided herein simultaneously detect about two, about three, about four, about five, about six, about seven, about eight, about nine, about ten, or more SNPs in a sample by detecting unique target IDs in the amplified DNA with each sample.

In certain embodiments, the assay methods provided herein simultaneously detect at least two, at least three, at least four, at least five, at least six, at least seven, at least eight, at least nine, at least ten, or more SNPs in a sample by detecting unique fluorescence signal associated with each SNP. In some embodiments, the assay methods provided herein simultaneously detect about two, about three, about four, about five, about six, about seven, about eight, about nine, about ten, or more SNPs in a sample by detecting unique fluorescence signal associated with each SNP.

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VIC™ to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature,” Nucleic Acids Research 28:655-661 (2000)). Minor grove binders include, but are not limited to, compounds such as dihydrocyclopyrroloindole tripeptide (DPI).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If predetermined, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are located in the center of an allele-specific oligonucleotide primer to be used in allele-specific oligonucleotide hybridization. In contrast, an allele-specific oligonucleotide primer to be used in PCR amplification contains the one or more nucleotide mismatches that distinguish between the disease-associated and other alleles at the 3′ end of the primer.

A heteroduplex mobility assay (HMA) is another assay that may be used in methods disclosed herein to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261(1993); White et al., Genomics 12:301-306 (1992)).

The technique of single strand conformational, polymorphism (SSCP) also may be used to detect the presence or absence of a SNP or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing reported alleles.

Denaturing gradient gel electrophoresis (DGGE) also may be used to detect a SNP or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP or a haplotype are useful in the methods described herein. Other approaches for determining the presence or absence of a SNP or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present methods for diagnosing or predicting susceptibility to or protection against CD in an individual may be practiced using one or any combination of the assays described above or another art-recognized genetic assay.

### Labeling

In some embodiments, a protein, polypeptide, nucleic acid, or fragment thereof is detectably labeled. In some instances, the protein, polypeptide, nucleic acid, or fragment thereof is ligated to an adaptor and the adapter is detectably labeled. The detectable label may comprise a fluorescent label, e.g., by incorporation of nucleotide analogues. Other labels suitable for use in the present methods include, but are not limited to, biotin, iminobiotin, antigens, cofactors, dinitrophenol, lipoic acid, olefinic compounds, detectable polypeptides, electron rich molecules, enzymes capable of generating a detectable signal by action upon a substrate, and radioactive isotopes.

In some instances, the detectable label is a radioactive isotope. Radioactive isotopes by way of non-limiting example, include ³²P and ¹⁴C. Fluorescent molecules suitable for the present methods include, but are not limited to, fluorescein and its derivatives, rhodamine and its derivatives, texas red, 5′carboxy-fluorescein (“FAM”), 2′, 7′-dimethoxy-4′, 5′-dichloro-6-carboxy-fluorescein (“JOE”), N, N, N′, N′-tetramethyl-6-carboxy-rhodamine (“TAMRA”), 6-carboxy-X-rhodamine (“ROX”), HEX, TET, IRD40, and IRD41.

Fluorescent molecules which are suitable for use with systems, kits and methods disclosed herein include: cyamine dyes, including but not limited to Cy2, Cy3, Cy3.5, CY5, Cy5.5, Cy7 and FLUORX; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes. Electron rich indicator molecules suitable for the present methods include, but are not limited to, ferritin, hemocyanin and colloidal gold.

Two-color fluorescence labeling and detection schemes may also be used (Shena et al., 1995, Science 270:467-470). Use of two or more labels can be useful in detecting variations due to minor differences in experimental conditions (e.g., hybridization conditions). In some embodiments of the methods, at least 5, 10, 20, or 100 dyes of different colors can be used for labeling. Such labeling can also permit analysis of multiple samples simultaneously which is encompassed by the methods.

Labeled molecules may be are contacted to a plurality of oligonucleotide probes under conditions that allow sample nucleic acids having sequences complementary to the probes to hybridize thereto (e.g., an array or chip). Depending on the type of label used, the hybridization signal may be detected using methods including, but not limited to, X-Ray film, phosphor imager, or CCD camera. When fluorescently labeled probes are used, the fluorescence emissions at each site of a transcript array may be detected by scanning confocal laser microscopy. In one embodiment, a separate scan, using the appropriate excitation line, is carried out for each of the two fluorophores used. In some instances, a laser is used that allows simultaneous specimen illumination at wavelengths specific to the two fluorophores and emissions from the two fluorophores may be analyzed simultaneously (see Shalon et al. (1996) Genome Res. 6, 639-645). In some instances, the arrays are scanned with a laser fluorescence scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al. (1996) Genome Res. 6, 639-645. Alternatively, a fiber-optic bundle can be used such as that described by Ferguson et al. (1996) Nat. Biotech. 14, 1681-1684. The resulting signals can then be analyzed to determine the expression of GPR35□ and housekeeping genes, using computer software.

In other embodiments, where genomic DNA of a subject is fragmented using restriction endonucleases and amplified before analysis, the amplification can comprise cloning regions of genomic DNA of the subject. In such methods, amplification of the DNA regions is achieved through the cloning process. For example, expression vectors can be engineered to express large quantities of particular fragments of genomic DNA of the subject (Sambrook and Russel, Molecular Cloning: A Laboratory Manual 4^{th} ed., Cold Spring Harbor Laboratory Press (Cold Spring Harbor, N Y 2012)).

In yet other embodiments, where the DNA of a subject is fragmented using restriction endonucleases and amplified before analysis, the amplification comprises expressing a nucleic acid encoding a gene, or a gene and flanking genomic regions of nucleic acids, from the subject. RNA (pre-messenger RNA) that comprises the entire transcript including introns is then isolated and used in the methods described herein to analyze and provide a genetic signature of a cancer. In certain embodiments, no amplification is required. In such embodiments, the genomic DNA, or pre-RNA, of a subject may be fragmented using restriction endonucleases or other methods. The resulting fragments may be hybridized to SNP probes. Greater quantities of DNA are required to be isolated in comparison to the quantity of DNA or pre-mRNA required where fragments are amplified. For example, where the nucleic acid of a subject is not amplified, a DNA sample of a subject for use in hybridization may be about 400 ng, 500 ng, 600 ng, 700 ng, 800 ng, 900 ng, or 1000 ng of DNA or greater. Alternatively, in other embodiments, methods are used that require very small amounts of nucleic acids for analysis, such as less than 400 ng, 300 ng, 200 ng, 100 ng, 90 ng, 85 ng, 80 ng, 75 ng, 70 ng, 65 ng, 60 ng, 55 ng, 50 ng, or less, such as is used for molecular inversion probe (MIP) assays. These techniques are particularly useful for analyzing clinical samples, such as paraffin embedded formalin-fixed material or small core needle biopsies, characterized as being readily available but generally having reduced DNA quality (e.g., small, fragmented DNA) or not providing large amounts of nucleic acids.

Once the expression levels have been determined, the resulting data can be analyzed using various algorithms, based on methods used by those skilled in the art.

The following examples are given for the purpose of illustrating various embodiments of the disclosure and are not meant to limit the present disclosure in any fashion. The present examples, along with the methods described herein are presently representative of embodiments and are not intended as limitations on the scope of the disclosure. Changes therein and other uses which are encompassed within the spirit of the disclosure as defined by the scope of the claims will occur to those skilled in the art.

### Systems

Disclosed herein, in some embodiments, is a system for treating an inflammatory disease or condition or fibrostenotic or fibrotic disease in a subject, comprising analyzing genes or gene products expressed from TNFSF15, LY86, ETS1, ARHGAP15, or SCUBE1, in a sample obtained from a subject. In some embodiments, one or more polymorphisms in Table 5 is analyzed. In some embodiments, any group of polymorphisms from Tables 6 or 7 are analyzed. The system is configured to implement the methods described in this disclosure, including but not limited to, analyzing genes or gene expression products from the genes of a subject to determine whether the subject is suitable for an anti-TL1A therapy.

In some embodiments, disclosed herein is a system for treating an inflammatory disease or condition or fibrostenotic or fibrotic disease in a subject, comprising: (a) a computer processing device, optionally connected to a computer network; and (b) a software module executed by the computer processing device to analyze genes or gene products expressed from TNFSF15, LY86, ETS1, ARHGAP15, or SCUBE1, in a sample obtained from a subject. in a sample obtained from a subject. In some embodiments, one or more polymorphisms in Table 5 is analyzed. In some embodiments, any group of polymorphisms from Tables 6 or 7 are analyzed. In some instances, the system comprises a central processing unit (CPU), memory (e.g., random access memory, flash memory), electronic storage unit, computer program, communication interface to communicate with one or more other systems, and any combination thereof. In some instances, the system is coupled to a computer network, for example, the Internet, intranet, or extranet that is in communication with the Internet, a telecommunication, or data network. In some embodiments, the system comprises a storage unit to store data and information regarding any aspect of the methods described in this disclosure. Various aspects of the system are a product or article or manufacture.

One feature of a computer program includes a sequence of instructions, executable in the digital processing device's CPU, written to perform a specified task. In some embodiments, computer readable instructions are implemented as program modules, such as functions, features, Application Programming Interfaces (APIs), data structures, and the like, that perform particular tasks or implement particular abstract data types. In some embodiments, the computer program is configured to (a) receive data corresponding to a presence or an absence of a genotype of a subject; (b) detect a presence or an absence of one or more polymorphisms from Tables 5, 6, or 7 and generate a score indicative of a risk that the subject has, or will develop a disease or disorder or respond to a therapeutic agent described herein. In some embodiments, the score is either positive or negative for the disease or disorder or response to the therapeutic agent. In some embodiments, the computer program is trained with plurality of training samples, and wherein the sample from the subject is independent from the plurality of training samples. In some embodiments, the training samples are derived from a reference population of individuals diagnosed with the disease or disorder, and a reference population of individual who are normal (e.g., not diagnosed with, and do not have, the disease or disorder). In some embodiments, a polygenic risk score (PRS) is calculated. In some embodiments, the PRS comprises a normalized weighted sum of a number of risk alleles within the genotype present in the subject with weights proportional to a beta value or odds ratio of association between the genotype with the disease or condition. To the extent an absence of a genotype is detected, the systems disclosed herein further comprises utilize data corresponding to a presence or an absence of a surrogate genotype to calculate the PRS. In some embodiments, a surrogate genotype is selected if it is linkage disequilibrium (LD) with the absence genotype, as determined by an r2 value of at least about, 0.8, about 0.85, about 0.90, about 0.95, or about 1.0.

The functionality of the computer readable instructions are combined or distributed as to achieve in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, a web application may utilize one or more software frameworks and one or more database systems. A web application, for example, is created upon a software framework such as Microsoft® .NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, my SQL™, and Oracle®. A web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques using hardware, languages, and development environments. Mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, Airplay SDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that may be run as an independent computer process, not an add-on to an existing process, e.g., not a plug-in. Standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation may be often performed, at least in part, to create an executable program. In some instances, a computer program includes one or more executable complied applications.

### Web Browser Plug-In

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, several plug-in frameworks are available that enable development of plug-ins in various programming languages, including, by way of non-limiting examples, C++, Delphi, Java™, PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called microbrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules may be created by techniques using machines, software, and languages. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprises a web application, a mobile application, or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The medium, method, and system disclosed herein comprise one or more databases, or use of the same. In view of the disclosure provided herein, many databases are suitable for storage and retrieval of geologic profile, operator activities, division of interest, or contact information of royalty owners. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

### Data Transmission

The subject matter described herein, are configured to be performed in one or more facilities at one or more locations. Facility locations are not limited by country and include any country or territory. In some instances, one or more steps of a method herein are performed in a different country than another step of the method. In some instances, one or more steps for obtaining a sample are performed in a different country than one or more steps for analyzing a genotype of a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a sample of a subject and any article or product disclosed herein as an article or product. Data includes, but is not limited to, information regarding genotype and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as cellular material, performed in one facility in a particular location and corresponding data transmitted to another location, or directly to an individual, such as data related to the diagnosis, prognosis, responsiveness to therapy, or the like, in the same or different location or country.

### EXAMPLES

Patients with IBD were recruited at the Cedars-Sinai Inflammatory Bowel Disease Centers. The diagnosis of each patient was based on standard endoscopic, histologic, and radiographic features. Blood samples were collected from patients at the time of enrollment Blood samples were also collected from individuals without IBD. Genotyping was performed at Cedars-Sinai Medical Center using Illumina whole-genome arrays per manufacturer's protocol (Illumina, San Diego, CA) on all samples collected. A stringent quality control (QC) procedure was applied to the genome-wide association (GWAS).

### Example 1

Polymorphisms Associated with Crohn's Disease and a Time to First Surgery

Time to first surgery data from patients with Crohn's disease (CD) who underwent a first small bowel resection that were recruited at the Cedars-Sinai Inflammatory Bowel Disease Centers was used (n˜1090). The diagnosis of each patient was based on standard endoscopic, histologic, and radiographic features. Patients were selected based on being diagnosed with CD and having undergone a second small bowel resection for disease. All Patients were genotyped either by Illumina ImmunoArray or polymerase chain reaction (PCR) under standard hybridization conditions. A survival analysis (e.g., Cox Proportional-Hazards model) was performed to identify the polymorphisms in Table 5 in association with a time to first surgery, with rs201292440 being the causal polymorphism (“Signal 1”). Signal 1 was selected using the methods and materials described in Huang, H. Fine-Mapping Inflammatory Bowel Disease Loci to Single Variant Resolution, Nature, Vol. 547, No. 7662 (Jul. 13, 2017), pp. 173-178. Table 5 shows polymorphisms in linkage disequilibrium with Signal 1 as defined by an r² value of at least 0.80, or a D′ value of at least 0.90, that were significantly correlated with a time to first surgery in patients with CD. “Time to first surgery” was defined as time from diagnosis to a first surgery. These polymorphisms are considered predictive of a faster progression to a first surgery as compared to an individual diagnosed with CD who does not carry the polymorphism.

TABLE 5
Polymorphisms associated with a Time to First Surgery

| Gene | dbSNP | p_value | Minor Allele | Risk Allele |
|---|---|---|---|---|
| TNFSF15 | rs80271384 | 1.17E−04 | T | T |
| TNFSF15 | rs10982413 | 8.82E−03 | A | A |
| TNFSF15 | rs10982412 | 1.98E−02 | A | A |
| TNFSF15 | rs11792988 | 2.08E−02 | A | N/A |
| TNFSF15 | rs17292046 | 2.69E−02 | C | C |
| TNFSF15 | rs1322063 | 3.66E−02 | A | A |
| ARHGAP 15 | rs6757588 | 4.31E−06 | A | G |
| SCUBE | rs6003160 | 6.55E−05 | G | G |

### Example 2

Polymorphisms Associated with a Second Surgery

Time to second surgery data from patients with Crohn's disease (CD) who underwent a second small bowel resection that were recruited at the Cedars-Sinai Inflammatory Bowel Disease Centers was used (n=181). The diagnosis of each patient was based on standard endoscopic, histologic, and radiographic features. Patients were selected based on being diagnosed with CD and having undergone a second small bowel resection for disease. All Patients were genotyped either by Illumina ImmunoArray or polymerase chain reaction (PCR) under standard hybridization conditions. A survival analysis (e.g., Cox Proportional-Hazards model) was performed to identify the polymorphisms in Table 6 in association with a time to second surgery, with rs201292440 being the causal polymorphism (“Signal 1”). Signal 1 was selected using the methods and materials described in Huang, H. Fine-Mapping Inflammatory Bowel Disease Loci to Single Variant Resolution, Nature, Vol. 547, No. 7662 (Jul. 13, 2017), pp. 173-178. Table 6 shows polymorphisms in linkage disequilibrium with Signal 1 as defined by an r² value of at least 0.80, or a D′ value of at least 0.90, that were significantly correlated with a time to second surgery in patients with CD. “Time to second surgery” refers to time from first to second surgery. These polymorphisms are considered predictive of a faster progression to a second surgery as compared to an individual diagnosed with CD who does not carry the polymorphism.

TABLE 6
Polymorphisms associated with time to Second Surgery

| Gene | dbSNP | p_value | Minor Allele | Risk Allele |
|---|---|---|---|---|
| TNFSF15 | rs6478108 | 1.63E−03 | G | G |
| TNFSF15 | rs7869487 | 3.35E−03 | G | G |
| TNFSF15 | rs6478109 | 4.62E−03 | A | A |
| TNFSF15 | rs7848647 | 4.62E−03 | A | A |
| TNFSF15 | rs4366152 | 5.73E−03 | A | A |
| TNFSF15 | rs1322063 | 7.23E−03 | A | A |
| TNFSF15 | rs722126 | 8.99E−03 | C | C |
| TNFSF15 | rs1853187 | 1.12E−02 | C | C |
| TNFSF15 | rs4979464 | 1.41E−02 | A | A |
| LY86 | rs6921610 | 1.91E−04 | G | G |

### Example 3

Small Bowel Expression Quantitative Trait Loci Mapping (eQTL)

Patients with Crohn's disease (CD) who underwent small bowel resection were recruited at the Cedars-Sinai Inflammatory Bowel Disease Centers. The diagnosis of each patient was based on standard endoscopic, histologic, and radiographic features. Patients were selected based on being diagnosed with CD and having undergone small bowel resection for disease. Tissue biopsy samples were collected from uninvolved tissue sections taken from small bowel resection after surgery. Expression Quantitative Trait Loci Mapping (eQTL) was performed on these samples. Polymorphisms listed in Table 7 show a strong associated with increase or decrease in messenger RNA (mRNA) expression in the small bowel section of the intestine. A negative eQTL beta value indicates a decrease in expression of the “cis_eGene” provided in the third column. While a positive eQTL beta value indicates an increase in expression of the gene.

TABLE 7
eQTL of mRNA expression in Ileal Tissue of the Small Bowel(n = 139)

| | | | | | Minor | Risk |
|---|---|---|---|---|---|---|
| SNP locus | dbSNP | cis_eGENE | eqtl_beta | eqtl_p | Allele | Allele |
| TNFSF15 | rs80271384 | TNFSF15 | −0.0854745 | 1.60E−02 | T | T |
| TNFSF15 | rs10982413 | TNFSF15 | −0.0889247 | 2.18E−02 | A | A |
| TNFSF15 | rs10982412 | TNFSF15 | −0.0911504 | 1.81E−02 | A | A |
| TNFSF15 | rs11792988 | TNFSF15 | 0.10633331 | 1.86E−02 | A | N/A |
| TNFSF15 | rs17292046 | TNFSF15 | −0.0911504 | 1.81E−02 | C | C |
| TNFSF15 | rs1322063 | TNFSF15 | 0.36098178 | 6.10E−03 | A | A |
| ETS1 | rs10790957 | ETS1 | −0.093 | 3.22E−02 | G | A |
| TNFSF15 | rs6478108 | TNFSF15 | 0.18995528 | 2.29E−02 | G | G |
| TNFSF15 | rs7869487 | TNFSF15 | 0.27823738 | 1.58E−03 | G | G |
| TNFSF15 | rs6478109 | TNFSF15 | 0.21416991 | 1.06E−02 | A | A |
| TNFSF15 | rs7848647 | TNFSF15 | 0.20302823 | 1.78E−02 | A | A |
| TNFSF15 | rs4366152 | TNFSF15 | 0.17123624 | 4.50E−02 | A | A |
| TNFSF15 | rs1322063 | TNFSF15 | −0.2115226 | 6.10E−03 | A | A |
| TNFSF15 | rs722126 | TNFSF15 | 0.20684207 | 1.30E−02 | C | C |
| TNFSF15 | rs1853187 | TNFSF15 | 0.1773396 | 2.98E−02 | C | C |
| TNFSF15 | rs4979464 | TNFSF15 | 0.1773396 | 2.98E−02 | A | A |

### Example 4

Polymorphisms Associated with Increased TL1A Fold-Change

99 patients were recruited at the Cedars-Sinai Inflammatory Bowel Disease Centers. All patients were genotyped for a risk TNFSF15 genotype (heterozygous risk or homozygous risk) either by Illumina ImmunoArray or polymerase chain reaction (PCR) under standard hybridization conditions. The TNFSF15 genotypes include heterozygous (AG) and homozygous (GG) at nucleopositon(s) 501 within rs6478109, which served as the causal polymorphism (“Signal 1”). Notably, however, any polymorphism at the TNFSF15 gene locus in linkage disequilibrium with Signal 1 can be used. Blood samples were collected from the patients, and peripheral blood mononuclear cells (PBMCs) were isolated from the blood samples. The PMBCs were stimulated in vitro with immune complex. Supernatants were collected from unstimulated samples and from stimulated samples at 6, 24, and 72 hours. Soluble TL1A protein in the supernatants was quantified using a plate-based ELISA using and monoclonal antibodies at all time points. Fold-change in TL1A was defined as TL1A levels in the supernatant at 24 hours divided by the TL1A levels in the supernatant at 6 hours.

Samples were collected from patients wherein an increased fold-change in TL1A was detected using the protocols above. Samples were collected from patients wherein an increase fold-change in TL1A, and the heterozygous TNFSF15 risk genotype, were detected using the protocols above. Samples were collected from patients wherein an increase fold-change in TL1A, and the homozygous TNFSF15 risk genotype, were detected using the protocols above. All samples collected were again genotyped using Illumina ImmunoArray. Genetic associations were performed using linear model between TL fold-change levels and single nucleotide polymorphisms (SNPS) (Table 8) or logistic model between TL1A fold-change high/low and SNPs (Table 10) with minor allele-frequency >0.01, less than 2% missingness in samples and using first two principal components in genotype data as covariates. Genetic associations were performed using linear model between TL1A fold-change levels and the TNFSF15 risk genotypes, and single nucleotide polymorphisms (SNPS) (Tables 8 and 9) or logistic model between TL1A fold-change high/low and the TNFSF15 risk genotypes and SNPs (Tables 12 and 13) with minor allele-frequency >0.01, less than 2% missingness in samples and using first two principal components in genotype data as covariates. The TNFSF15 risk genotypes included expression of the heterozygous risk polymorphism rs6478109 (AG)(“Signal One Carrier”), or homozygous polymorphism rs6478109 (“GG)(“Signal One Risk”).

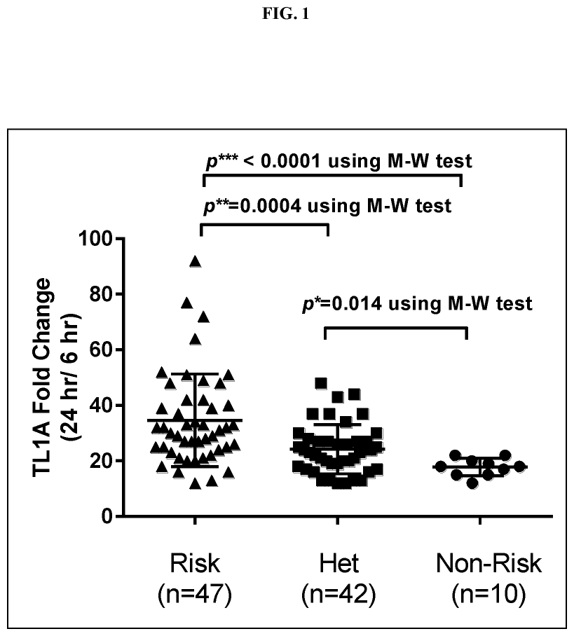

In all samples (n=98) the TNFSF15 risk genotypes resulted in higher TL1A fold-change as compared to TL1A fold-change in non-risk subjects, with homozygous risk genotype resulting in the highest TL1A fold-change (FIG. 1) In all the samples (n=98), polymorphisms at nucleobase 501 within rs6757588 (SEQ ID NO: 35) of the gene locus ARHGAP15, rs10790957 (SEQ ID NO: 34) at the ETS1 gene locus, rs6921610 (SEQ ID NO: 33) at the LY86 gene locus, and rs6003160 (SEQ ID NO: 36) at the SCUBE1 gene locus were strongly associated (ETS1 P=8.04×10⁻⁵; LY86 P=1.91E⁻⁴; SCUBE1 P=6.55×10⁻⁵; ARHGAP15 P=4.31×10⁻⁶) with increased TL1A fold-change. In all the samples (n=98), a polymorphism at nucleobase 700 within rs6921610 (SEQ ID NO: 33) at the LY86 gene locus was associated with high and low TL1A fold-change production with less 25 TL1A fold-change defined as low (LY86 P=1.91×10⁻⁴). In samples (n=88) collected from patients with the heterozygous TNFSF15 risk genotype, polymorphisms at nucleobase 501 within rs6757588 (SEQ ID NO: 35) of the gene locus ARHGAP15, rs10790957 (SEQ ID NO: 34) at the ETS1 gene locus, rs6921610 (SEQ ID NO: 33) at the LY86 gene locus, and rs6003160 (SEQ ID NO: 36) at the SCUBE1 gene locus were strongly associated (ETS1 P=8.04×10⁻⁵; LY86 P=1.91E⁻⁴; SCUBE1P=6.55×10⁻⁵; ARHGAP15 P=4.31×10⁻⁶). In samples (n=47) collected from patients with the homozygous TNFSF15 risk genotype, polymorphisms at nucleobase 501 within rs6757588 (SEQ ID NO: 35) of the gene locus ARHGAP15, rs10790957 (SEQ ID NO: 34) at the ETS1 gene locus, rs6921610 (SEQ ID NO: 33) at the LY86 gene locus, and rs6003160 (SEQ ID NO: 36) at the SCUBE1 gene locus were strongly associated (ETS1 P=8.04×10⁻⁵; LY86 P=1.91E⁻⁴; SCUBE1 P=6.55×10⁻⁵; ARHGAP15 P=4.31×10⁻⁶).

Enrichment of increased TL1A fold change was studied in samples collected from patients expressing the TNFSF15 risk genotypes and the polymorphisms associated with an increase in TL1A fold-change above using a TL1A enrichment analysis. A TL1A enrichment analysis indicates which of the polymorphisms above in combination with a TNFSF15 risk genotype show the highest increases of TL fold change, as compared to the increase in TL1A fold-change observed in samples from patients expressing the TNFSF15 risk genotype alone. These combinations are useful for identifying patients uniquely suitable for treatment with an inhibitor of TL1A or characterizing, predicting, or diagnosing a disease associated with elevated levels of TL1A, without a need to measure the TL1A levels, themselves, in the patient sample. A statistical significant amount of an increase in TL1A fold-change is above the mean (+/−the standard deviation) of TL1A fold-change level associated with TNFSF15 non-risk population (e.g., non-carriers of either TNFSF15 risk genotypes). The mean comprised about 25-fold change.

In samples wherein the homozygous risk rs6478109 polymorphism was detected (homozygous TNFSF15 genotype (GG) (n=47)), polymorphisms at rs10790957 (SEQ ID NO: 34) at the ETS1 gene locus, rs6921610 (SEQ ID NO: 33) at the LY86 gene locus, and rs6003160 (SEQ ID NO: 36) at the SCUBE1 gene locus (ETS1 P=8.04×10⁻⁵; LY86 P=1.91E⁻⁴; SCUBE1 P=6.55×10⁻⁵), enriched the homozygous risk rs6478109 genotype risk samples, with the majority of samples in each analysis above the mean (+/−standard deviation) and TL1A fold change levels reaching 95 and higher. The observed increase in TL1A fold-change was higher when the homozygous TNFSF15 genotype in combination with one or more of the polymorphisms at rs10790957 (SEQ ID NO: 34) and the ETS1 gene locus, rs6921610 (SEQ ID NO: 33) at the LY86 gene locus, and rs6003160 (SEQ ID NO: 36) at the SCUBE1 gene locus, than the fold-change observed when the homozygous TNFSF15 genotype is detected alone, with the majority of samples below the mean (+/−standard deviation) and maximum fold-change of about 40-fold.

In samples wherein the heterozygous risk TNFSF15 genotype (AG) was detected, a polymorphism at nucleobase 501 within rs6757588 (SEQ ID NO: 35) of the gene locus ARHGAP15 carrying minor allele risk genotype enriched the heterozygous TNFSF15 genotype (AG) risk samples with the majority of samples above the mean (+/−standard deviation), ranging from 25 to 95-fold increase in TL1A fold-change level. The observed increase in TL1A fold-change was higher when the heterozygous TNFSF15 genotype in combination with the polymorphism at nucleobase 501 within rs6757588 (SEQ ID NO: 35) of the gene locus ARHGAP15, than the fold-change observed when the heterozygous TNFSF15 genotype is detected alone, with more samples below the mean (+/−standard deviation).

In contrast, samples wherein the homozygous risk TNFSF15 genotype was detected did not show a statistically significant level of TL1A fold-change when expressed in combination with the polymorphism at nucleobase 501 within rs6757588 (SEQ ID NO: 35) of the gene locus ARHGAP15. Similarly, samples wherein the heterozygous risk TNFSF15 genotype was detected did not show a statistically significant TL1A fold-change when expressed in combination with the polymorphisms at nucleobase 700 within rs6921610 (SEQ ID NO: 33) at the LY86 gene locus, rs10790957 (SEQ ID NO: 34) and the ETS1 gene locus, and rs6003160 (SEQ ID NO: 36) at the SCUBE1 gene locus with significance seen in homozygous risk TNFSF15. Thus, without wishing to be bound by any particular theory, these results are highly suggestive that the TNFSF15 risk genotype (e.g., homozygous or heterozygous) heavily influences which of the disclosed polymorphisms, when expressed in combination with the particular TNFSF15 risk genotype, are indicative of an increase in TL1A fold-change. Further, the TNFSF15 risk genotype may not be confined to the rs6478109 polymorphism, as any polymorphism at the TNFSF15 gene locus in linkage disequilibrium with the rs6478109 polymorphism can be expected to yield similar results. As such, any of the combinations of polymorphisms in Tables 3 and 4 may be used to predict increased TL1A fold-change in a subject for use in treating or characterizing an inflammatory disease or condition or fibrotic or fibrostenotic disease disclosed herein.

TABLE 8
Polymorphisms associated with TL1A fold-change (linear model)

| | | Minor | | | | |
|---|---|---|---|---|---|---|
| Polymorphism | Illumina_id | Allele (A1) | BETA | P | Gene | MAF |
| rs116347760 | imm_1_114002774 | T | 45.69 | 5.46E−09 | MAGI3 | 0.01597 |
| rs115611397 | 1kg_8_79548346 | A | 50.35 | 1.86E−07 | LOC102724874, PKIA | 0.01008 |
| rs74395031 | imm_1_13740022 | G | 35.68 | 2.09E−07 | MAGI3 | 0.01702 |
| rs11912198 | rs11912198 | A | 24.75 | 7.53E−07 | ZNRF3 | 0.01691 |
| rs16986990 | rs16986990 | G | 24.75 | 7.53E−07 | ZNRF3 | 0.01849 |
| rs4823000 | rs4823000 | G | 24.75 | 7.53E−07 | ZNRF3 | 0.0214 |
| rs8137391 | rs8137391 | A | 24.75 | 7.53E−07 | ZNRF3-AS1 | 0.01848 |
| rs28550609 | 1kg_8_79506636 | A | 22.12 | 1.10E−06 | LOC102724874, PKIA | 0.07173 |
| rs4145315 | 1kg_8_79562307 | A | 22.12 | 1.10E−06 | LOC102724874, PKIA | 0.07256 |
| rs116297428 | imm_2_162683961 | A | 29.66 | 1.91E−06 | LOC101929532 | 0.01535 |
| rs13403657 | rs13403657 | G | 32.48 | 2.79E−06 | SNED1 | 0.01724 |
| rs7812931 | rs7812931 | A | 19.59 | 3.31E−06 | ZHX2, DERL1 | 0.06666 |
| rs34209542 | imm_1_114174047 | G | 32.21 | 3.84E−06 | AP4B1-AS1 | 0.02318 |
| rs115870915 | imm_1_113935553 | A | 32.21 | 3.84E−06 | MAGI3 | 0.02177 |
| rs33996649 | imm_1_114196212 | A | 32.21 | 3.84E−06 | PTPN22 | 0.02177 |
| rs73688944 | 1kg_8_79493024 | G | 24.74 | 3.85E−06 | LOC102724874, PKIA | 0.03649 |
| rs61394970 | 1kg_8_79494228 | G | 24.74 | 3.85E−06 | LOC102724874, PKIA | 0.03644 |
| rs1864577 | 1kg_8_79411977 | A | 23.18 | 4.00E−06 | LOC102724874, PKIA | 0.03639 |
| rs77128194 | imm_1_113741794 | A | 23.13 | 4.24E−06 | MAGI3 | 0.03049 |
| rs76975167 | imm_1_113754608 | A | 23.13 | 4.24E−06 | MAGI3 | 0.03059 |
| rs75948156 | imm_1_113767166 | A | 23.13 | 4.24E−06 | MAGI3 | 0.03054 |
| rs74431747 | imm_1_113814325 | G | 23.13 | 4.24E−06 | MAGI3 | 0.03085 |
| rs6757588 | rs6757588 | G | 9.357 | 4.31E−06 | ARHGAP15 | 0.3473 |
| rs116767299 | imm_2_60785645 | A | 63.12 | 4.45E−06 | BCL11A, PAPOLG | 0.01545 |
| rs183396336 | imm_1_117099551 | A | 63.12 | 4.45E−06 | CD2 | 0.01034 |
| rs72832303 | ccc-6-20826759-A-G | G | 131.56 | 4.45E−06 | CDKAL1 | 0.02041 |
| rs76824122 | imm_2_204472857 | A | 63.12 | 4.45E−06 | CTLA4, ICOS | 0.01676 |
| rs117542910 | imm_17_35391423 | G | 63.12 | 4.45E−06 | PSMD3 | 0.0142 |
| rs77411382 | seq-VH-424 | A | 63.12 | 4.45E−06 | RGS21, RGS1 | 0.01697 |
| rs11847179 | rs11847179 | A | 63.12 | 4.45E−06 | TTC7B | 0.01352 |
| rs12591019 | rs12591019 | A | 63.12 | 4.45E−06 | TTC7B | 0.0142 |
| rs17126980 | rs17126980 | A | 63.12 | 4.45E−06 | TTC7B | 0.01378 |
| rs17126982 | rs17126982 | A | 63.12 | 4.45E−06 | TTC7B | 0.01289 |
| rs1998188 | rs1998188 | A | 63.12 | 4.45E−06 | TTC7B | 0.01354 |
| rs2401911 | rs2401911 | G | 63.12 | 4.45E−06 | TTC7B | 0.01425 |
| rs4900059 | rs4900059 | A | 63.12 | 4.45E−06 | TTC7B | 0.01446 |
| rs4904723 | rs4904723 | G | 63.12 | 4.45E−06 | TTC7B | 0.02013 |
| rs6575143 | rs6575143 | C | 63.12 | 4.45E−06 | TTC7B | 0.01357 |
| rs6575144 | rs6575144 | G | 63.12 | 4.45E−06 | TTC7B | 0.01357 |
| rs8004183 | rs8004183 | A | 63.12 | 4.45E−06 | TTC7B | 0.01357 |
| rs8019797 | rs8019797 | C | 63.12 | 4.45E−06 | TTC7B | 0.01357 |
| rs115537678 | imm_5_150351522 | A | 63.12 | 4.45E−06 | ZNF300P1, GPX3 | 0.01122 |
| rs6478109 | imm_9_116608587 | A | −9.551 | 6.47E−06 | TNFSF15 | 0.2995 |
| rs7848647 | imm_9_116608867 | A | −9.551 | 6.47E−06 | TNFSF15 | 0.2978 |
| rs77984256 | imm_14_68259573 | A | 26.11 | 8.43E−06 | RAD51B, ZFP36L1 | 0.01665 |
| rs12982003 | imm_19_51997600 | C | 27.86 | 1.45E−05 | SLC1A5, SNAR-E | 0.04808 |
| rs6708276 | rs6708276 | G | 8.757 | 1.48E−05 | ARHGAP15 | 0.3447 |
| rs76709465 | imm_5_132158742 | C | 24.33 | 1.87E−05 | SEPT8, SOWAHA | 0.02516 |
| rs77770153 | 1kg_17_29603462 | A | 18.12 | 1.89E−05 | LOC101927239, CCL2 | 0.06682 |
| rs10169606 | rs10169606 | G | 8.115 | 2.01E−05 | ARHGAP15 | 0.3662 |
| rs115984727 | imm_5_132143609 | A | 24.33 | 2.08E−05 | SEPT8, SOWAHA | 0.02534 |
| rs8009181 | rs8009181 | A | 20.9 | 2.08E−05 | MAPK1IP1L, LGALS3 | 0.03743 |
| rs201292440 | 9-116611115-GAA- | D | −9.02 | 2.09E−05 | TNFSF15_TNFSF8 | 0.2695 |
| | INSERTION | | | | | |
| rs62437166 | imm_6_127308860 | A | 34.23 | 2.09E−05 | MIR588, RSPO3 | 0.01989 |
| rs1944961 | rs1944961 | A | 19.106 | 2.82E−05 | TENM4, LOC101928944 | 0.2136 |
| rs6928830 | rs6928830 | G | 9.862 | 3.32E−05 | ME1, PRSS35 | 0.1732 |
| rs12035823 | rs12035823 | G | 26.07 | 3.64E−05 | OLFM3, COL11A1 | 0.04886 |
| rs7869487 | imm_9_116620735 | G | −8.824 | 3.68E−05 | TNFSF15, TNFSF8 | 0.2841 |
| rs76655944 | imm_1_181829847 | A | 41 | 3.69E−05 | NCF2, ARPC5 | 0.01143 |
| rs4366152 | imm_9_116604696 | A | −8.559 | 3.85E−05 | TNFSF15 | 0.2982 |
| rs11465283 | rs11465283 | A | 12.24 | 3.95E−05 | ADAM19 | 0.1133 |
| rs74675346 | imm_19_10343638 | A | 24.41 | 4.14E−05 | TYK2 | 0.02678 |
| rs7677400 | rs7677400 | G | 16.09 | 4.54E−05 | MAPK10 | 0.1008 |
| rs10189240 | rs10189240 | G | 7.954 | 4.59E−05 | ARHGAP15 | 0.3637 |
| rs2048957 | rs2048957 | A | 8.063 | 4.72E−05 | ARHGAP15 | 0.3606 |
| rs55720245 | imm_9_35368401 | A | 33.09 | 5.25E−05 | UNC13B | 0.01561 |
| rs72772074 | mm_5_96024270 | G | 10.88 | 5.76E−05 | CAST | 0.1342 |
| rs117889858 | imm_16_67072555 | A | 28.3 | 5.99E−05 | SMPD3, ZFP90 | 0.0213 |
| rs12539781 | rs12539781 | A | 9.205 | 6.32E−05 | LIMK1, EIF4H | 0.2135 |
| rs12137209 | seq-rs12137209 | A | 32.37 | 6.72E−05 | ATP6V1G3, PTPRC | 0.02135 |
| rs12118482 | seq-rs12118482 | G | 32.37 | 6.72E−05 | PTPRC | 0.02 |
| rs4910068 | rs4910068 | G | 8.855 | 7.54E−05 | ST5 | 0.2834 |
| rs114797146 | imm_2_99955021 | A | 39.36 | 7.92E−05 | AFF3 | 0.01655 |
| rs76990532 | imm_2_99961776 | A | 39.36 | 7.92E−05 | AFF3 | 0.01498 |
| rs12722547 | imm_10_6112099 | C | 38.9 | 8.01E−05 | IL2RA | 0.01383 |
| rs12722502 | imm_10_6133145 | A | 38.9 | 8.01E−05 | IL2RA | 0.01383 |
| rs17086512 | imm_5_96040813 | G | 9.449 | 8.41E−05 | CAST | 0.2483 |
| rs6478108 | imm_9_116598524 | G | −8.19 | 8.44E−05 | TNFSF15 | 0.3126 |
| rs2141102 | rs2141102 | A | 9.591 | 8.79E−05 | NEFL, DOCK5 | 0.2772 |
| rs7002363 | rs7002363 | G | 9.591 | 8.79E−05 | NEFL, DOCK5 | 0.275 |
| rs12465492 | rs12465492 | A | 7.641 | 9.31E−05 | ARHGAP15 | 0.365 |
| rs747024 | rs747024 | A | 10.75 | 9.44E−05 | HERC4 | 0.1289 |
| rs4303275 | rs4303275 | A | 8.581 | 9.69E−05 | TRHDE | 0.2789 |
| rs13187079 | imm_5_96043366 | G | 9.609 | 9.83E−05 | CAST | 0.2479 |
| rs3729703 | rs3729703 | C | 38.74 | 9.99E−05 | MEF2C | 0.01618 |
| rs1944959 | rs1944959 | A | 8.253 | 1.00E−04 | TENM4, LOC101928944 | 0.2259 |
| rs17645980 | imm_5_55460497 | A | 9.854 | 1.03E−04 | ANKRD55 | 0.2127 |
| rs10461422 | imm_5_55468005 | C | 19.854 | 1.03E−04 | ANKRD55 | 0.2104 |
| rs17031888 | imm_1_114163459 | G | 24.79 | 1.08E−04 | AP4B1-AS1 | 0.03863 |
| rs17031955 | imm_1_114212503 | A | 24.79 | 1.08E−04 | AP4B1-AS1 | 0.03644 |
| rs442995 | rs442995 | C | 9.096 | 1.13E−04 | MIR99AHG, LINC01549 | 0.232 |
| rs10760109 | imm_9_122437397 | A | 22.64 | 1.29E−04 | MEGF9 | 0.02328 |
| rs1886338 | imm_9_122451373 | G | 22.64 | 1.29E−04 | MEGF9 | 0.02325 |
| rs275217 | rs275217 | G | 13.34 | 1.33E−04 | C15orf53, C15orf54 | 0.05848 |
| rs7713991 | rs7713991 | A | 9.375 | 1.35E−04 | LOC401177, CDH18 | 0.1978 |
| rs17720798 | imm_6_127396930 | A | 20.64 | 1.49E−04 | MIR588, RSPO3 | 0.05967 |
| rs3131296 | rs3131296 | A | 12.55 | 1.79E−04 | NOTCH4 | 0.121 |
| rs3132956 | rs3132956 | A | 12.55 | 1.79E−04 | NOTCH4 | 0.1212 |
| rs3134796 | rs3134796 | G | 12.55 | 1.79E−04 | NOTCH4 | 0.1218 |
| rs3134942 | rs3134942 | A | 12.55 | 1.79E−04 | NOTCH4 | 0.121 |
| rs1169293 | rs1169293 | G | 14.25 | 1.80E−04 | HNF1A | 0.07027 |
| rs115443294 | imm_3_49061321 | G | 37.39 | 1.86E−04 | QRICH1 | 0.009397 |
| rs79149734 | imm_3_49977545 | A | 37.39 | 1.86E−04 | RBM6 | 0.009608 |
| rs116759321 | imm_5_40662246 | A | 27.03 | 1.92E−04 | LINC00603, PTGER4 | 0.02924 |
| rs1277016 | rs1277016 | G | 9.371 | 1.98E−04 | STXBP3 | 0.2562 |
| rs16924888 | rs16924888 | A | 10.18 | 2.07E−04 | DNAJC12 | 0.131 |
| rs17456400 | rs17456400 | C | 10.18 | 2.07E−04 | HERC4, MYPN | 0.1342 |
| rs17390873 | rs17390873 | A | 11.74 | 2.08E−04 | ATG4C, LINC00466 | 0.1236 |
| rs7895833 | rs7895833 | G | 9.384 | 2.13E−04 | DNAJC12, SIRT1 | 0.1969 |
| rs2229136 | rs2229136 | G | 20.2 | 2.26E−04 | ALOX5 | 0.05768 |
| rs60835488 | imm_14_68276435 | A | 15.14 | 2.42E−04 | RAD51B, ZFP36L1 | 0.05737 |
| rs73277289 | imm_14_68277780 | G | 15.14 | 2.42E−04 | RAD51B, ZFP36L1 | 0.0581 |
| rs7647337 | rs7647337 | G | 9.516 | 2.50E−04 | RPN1 | 0.2264 |
| rs4648892 | rs4648892 | G | 8.493 | 2.53E−04 | TCEA3 | 0.2663 |
| rs4806768 | seq-rs4806768 | A | 7.235 | 2.62E−04 | LAIR2 | 0.4648 |
| rs1836767 | rs1836767 | G | 11.97 | 2.66E−04 | PLD5 | 0.07658 |
| rs878983 | rs878983 | A | 8.624 | 2.72E−04 | LAPTM4A, SDC1 | 0.2088 |
| rs75326394 | imm_5_141398210 | A | 36.41 | 2.72E−04 | GNPDA1, NDFIP1 | 0.01566 |
| • | vh_11_124129360 | A | 15.74 | 2.75E−04 | ESAM | 0.08091 |
| rs11779459 | rs11779459 | A | 7.535 | 2.77E−04 | ZHX2 | 0.3691 |
| rs117324436 | imm_9_4995771 | G | 12.41 | 2.89E−04 | JAK2 | 0.08959 |
| rs76923469 | imm_3_161148501 | A | 29.59 | 2.89E−04 | IL12A-AS1 | 0.02824 |
| rs77908676 | imm_3_161172814 | G | 29.59 | 2.89E−04 | IL12A-AS1 | 0.0285 |
| rs2528691 | rs2528691 | G | 7.806 | 3.05E−04 | IMMP2L, DOCK4 | 0.4921 |
| rs35211634 | vh_11_59369435 | G | 21.14 | 3.05E−04 | GIF | 0.04339 |
| rs74398490 | vh_11_59237695 | A | 21.14 | 3.05E−04 | OR10V1 | 0.03691 |
| rs1466085 | rs1466085 | A | 15.47 | 3.39E−04 | FRC, LINC00885 | 0.07366 |
| rs16897813 | rs16897813 | G | 12.54 | 3.60E−04 | ZHX2 | 0.1046 |
| rs17086609 | rs17086609 | G | 7.07 | 3.64E−04 | FLT1 | 0.3457 |
| rs1984775 | rs1984775 | G | 8.031 | 3.65E−04 | NRIP3, SCUBE2 | 0.2777 |
| rs55741542 | imm_4_123607966 | G | 35.4 | 3.68E−04 | IL2, IL21 | 0.01472 |
| rs77415229 | imm_4_123656789 | A | 35.4 | 3.68E−04 | IL2, IL21 | 0.01425 |
| rs56668170 | imm_10_35666576 | A | 115.57 | 3.92E−04 | CCNY | 0.06234 |
| rs2724011 | 1kg_7_37365041 | A | 7.339 | 3.99E−04 | ELMO1 | 0.2354 |
| rs62437245 | imm_6_127438329 | A | 14.68 | 4.08E−04 | MIR588, RSPO3 | 0.09294 |
| rs180782 | rs180782 | G | 7.62 | 4.29E−04 | YY1P2, LRP1B | 0.2339 |
| rs7045305 | rs7045305 | A | 9.207 | 4.30E−04 | ANKRD19P | 0.173 |
| rs13154564 | imm_5_158472008 | G | 22.74 | 4.34E−04 | LOC101927740 | 0.01952 |
| rs4320976 | imm_11_75765492 | A | 9.434 | 4.36E−04 | PRKRIR | 0.1918 |
| rs7948288 | imm_11_75768491 | A | 19.434 | 4.36E−04 | PRKRIR | 0.1935 |
| rs113018253 | imm_10_64049686 | G | 34.91 | 4.47E−04 | ZNF365 | 0.0142 |
| rs1432295 | imm_2_60920170 | G | 8.36 | 4.49E−04 | PAPOLG, LINC01185 | 0.3779 |
| rs1407308 | imm_9_116610044 | A | −7.408 | 4.55E−04 | TNFSF15, TNFSF8 | 0.4745 |
| rs6832887 | rs6832887 | A | 13.77 | 4.71E−04 | SLC4A4 | 0.08502 |
| rs2644898 | rs2644898 | G | 6.968 | 4.78E−04 | MIA-RAB4B, RAB4B-EGLN2 | 0.2608 |
| rs62362364 | imm_5_55477328 | G | 8.843 | 4.85E−04 | ANKRD55 | 0.2034 |
| rs181985936 | 1kg_5_173384682 | A | 35.06 | 4.87E−04 | C5orf47, HMP19 | 0.01409 |
| rs115910131 | imm_5_6075852 | G | 35.06 | 4.87E−04 | CAST | 0.01624 |
| rs61839083 | ccc-10-6492013-G-A | A | 35.06 | 4.87E−04 | LOC399715, PRKCQ | 0.01227 |
| rs8028957 | rs8028957 | G | 48.74 | 5.12E−04 | ASB7, ALDH1A3 | 0.009919 |
| rs80146815 | imm_10_35911006 | G | 48.74 | 5.12E−04 | CCNY, GJD4 | 0.02271 |
| rs73102465 | imm_12_56535184 | T | 48.74 | 5.12E−04 | CTDSP2, LOC100506844 | 0.03884 |
| rs16976362 | rs16976362 | A | 48.74 | 5.12E−04 | HS3ST4, C16orf82 | 0.01122 |
| rs12748226 | 1kg_1_22578368 | A | 48.74 | 5.12E−04 | MIR4418, ZBTB40 | 0.01263 |
| rs7324708 | rs7324708 | A | 8.786 | 5.18E−04 | KLF12 | 0.213 |
| rs6003160 | rs6003160 | G | 8.373 | 5.19E−04 | SCUBE1 | 0.295 |
| rs7179025 | rs7179025 | G | −8.878 | 5.23E−04 | SLC27A2 | 0.1883 |
| rs17268037 | rs17268037 | C | 9.361 | 5.44E−04 | GPR15, CPOX | 0.1582 |
| rs911887 | rs911887 | G | −7.106 | 5.49E−04 | SFTPD | 0.3975 |
| rs74998771 | imm_16_30864143 | A | 18.94 | 5.50E−04 | FBXL19 | 0.03059 |
| rs9434618 | imm_1_8123165 | A | 20.38 | 5.70E−04 | ERRFI1, LOC 102724539 | 0.04333 |
| rs2302179 | rs2302179 | A | 8.633 | 5.78E−04 | CTNND2 | 0.2285 |
| rs4262006 | rs4262006 | G | 11.19 | 5.90E−04 | QRFPR, ANXA5 | 0.1405 |
| rs139955747 | imm_7_107378998 | C | 34.21 | 5.96E−04 | LAMB1 | 0.01263 |
| rs36027286 | imm_2_204351502 | G | 34.49 | 5.99E−04 | CD28, CTLA4 | 0.01958 |
| rs113656426 | 1kg_5_173455093 | A | 34.49 | 5.99E−04 | HMP19 | 0.01054 |
| rs71427708 | imm_2_204201527 | C | 34.49 | 5.99E−04 | RAPH1, CD28 | 0.0201 |
| rs11965547 | rs11965547 | A | 14.72 | 6.04E−04 | SLC44A4 | 0.07616 |
| rs11761905 | rs11761905 | A | 8.459 | 6.10E−04 | JAZF1 | 0.2309 |
| rs74674305 | imm_1_113680743 | A | 18.75 | 6.30E−04 | LOC643441, MAGI3 | 0.03023 |
| rs3101943 | rs3101943 | A | 28.33 | 6.35E−04 | HLA-DMB | 0.01049 |
| rs6920606 | rs6920606 | A | 6.951 | 6.51E−04 | HLA-DOA, HLA-DPA1 | 0.4959 |
| rs180473 | rs180473 | A | 6.438 | 6.81E−04 | EPB41L4A- | 0.3998 |
| | | | | | AS2, LOC102467214 | |
| rs1590345 | imm_9_123025169 | A | 17.53 | 6.82E−04 | RAB14, GSN | 0.04724 |
| rs10985184 | imm_9_123027492 | A | 17.53 | 6.82E−04 | RAB14, GSN | 0.04709 |
| rs62547034 | imm_9_34692588 | A | 17.57 | 6.88E−04 | CCL19, CCL21 | 0.02851 |
| rs1761455 | seq-rs1761455 | G | 8.283 | 6.90E−04 | LILRA3, LILRA5 | 0.2835 |
| rs404032 | seq-rs404032 | C | 8.283 | 6.90E−04 | LILRA3, LILRA5 | 0.2834 |
| rs414135 | seq-rs414135 | A | 8.283 | 6.90E−04 | LILRA3, LILRA5 | 0.2833 |
| rs651279 | seq-rs651279 | G | 8.283 | 6.90E−04 | LILRA3, LILRA5 | 0.2841 |
| rs759819 | seq-rs759819 | G | 8.283 | 6.90E−04 | LILRA3, LILRA5 | 0.2835 |
| rs7030574 | imm_9_116607870 | C | −7.127 | 7.26E−04 | TNFSF15 | 0.4808 |
| rs10114470 | imm_9_116587593 | A | −7.323 | 7.29E−04 | TNFSF15 | 0.302 |
| rs10976810 | rs10976810 | G | 9.369 | 7.68E−04 | TMEM261, PTPRD | 0.1917 |
| rs118077986 | 1kg_14_34839774 | A | 33.84 | 7.76E−04 | PSMA6 | 0.02109 |
| rs4787451 | imm_16_28229838 | A | 33.84 | 7.76E−04 | SBK1 | 0.01322 |
| rs12444319 | imm_16_28245764 | A | 33.84 | .76E−04 | SBK1, NPIPB6 | 0.01331 |
| rs2680344 | rs2680344 | G | −8.664 | 7.88E−04 | HCN4 | 0.2237 |
| • | vh_10_1058639 | A | 15.86 | 8.06E−04 | IDI2 | 0.07376 |
| rs6936620 | rs6936620 | A | 7.782 | 8.17E−04 | HLA-DOA, HLA-DPA1 | 0.3609 |
| rs27991 | imm_5_96083069 | A | 8.358 | 8.21E−04 | CAST | 0.1854 |
| rs4979464 | imm_9_116641968 | A | −7.165 | 8.24E−04 | TNFSF15, TNFSF8 | 0.3041 |
| rs116623623 | imm_1_199275217 | G | 33.73 | 8.37E−04 | CACNAIS | 0.01712 |
| rs78651839 | seq-VH-1536 | A | 33.73 | 8.37E−04 | IL12A-AS1 | 0.01383 |
| rs140226558 | imm_10_61671881 | A | 23.78 | 8.84E−04 | ANK3 | 0.02563 |
| rs26517 | imm_5_96081760 | A | 8.6 | 8.91E−04 | CAST | 0.1829 |
| rs79664017 | imm_9_122925976 | C | 17.3 | 8.92E−04 | CNTRL | 0.02732 |
| rs75010357 | imm_9_122974826 | G | 17.3 | 8.92E−04 | CNTRL | 0.02741 |
| rs115282331 | imm_9_122998531 | A | 17.3 | 8.92E−04 | RAB14 | 0.02736 |
| rs76887590 | imm_14_68364326 | A | 20.15 | 8.95E−04 | ZFP36L1, ACTN1 | 0.02328 |
| rs2474759 | imm_10_35913080 | G | 15.92 | 9.26E−04 | CCNY, GJD4 | 0.06249 |
| rs2506110 | imm_10_35913395 | C | 15.92 | 9.26E−04 | CCNY, GJD4 | 0.06249 |
| rs2474758 | imm_10_3591346 | A | 15.92 | 9.26E−04 | CCNY, GJD4 | 0.06249 |
| rs2474757 | imm_10_35913499 | C | 15.92 | 9.26E−04 | CCNY, GJD4 | 0.0625 |
| rs2506109 | imm_10_35913651 | G | 15.92 | 9.26E−04 | CCNY, GJD4 | 0.0625 |
| rs2474756 | imm_10_35914385 | A | 15.92 | 9.26E−04 | CCNY, GJD4 | 0.06244 |
| rs2506108 | imm_10_35914645 | G | 15.92 | 9.26E−04 | CCNY, GJD4 | 0.06249 |
| rs2506107 | imm_10_35914668 | A | 15.92 | 9.26E−04 | CCNY, GJD4 | 0.06249 |
| rs2474755 | imm_10_35914901 | G | 15.92 | 9.26E−04 | CCNY, GJD4 | 0.06249 |
| rs1862082 | imm_10_35915361 | A | 15.92 | 9.26E−04 | CCNY, GJD4 | 0.06239 |
| rs2474754 | imm_10_35915737 | G | 15.92 | 9.26E−04 | CCNY, GJD4 | 0.06243 |
| rs2245348 | imm_10_35916724 | C | 15.92 | 9.26E−04 | CCNY, GJD4 | 0.06223 |
| rs1064524 | imm_16_30400324 | A | 23.92 | 9.71E−04 | ITGAL | 0.04093 |
| rs16966547 | rs16966547 | G | 12.12 | 9.75E−04 | MAPRE2, ZNF397 | 0.07 |
| rs8093515 | rs8093515 | G | 12.12 | 9.75E−04 | MAPRE2, ZNF397 | 0.06729 |
| rs12666501 | 1kg_7_37392319 | A | 7.348 | 9.82E−04 | ELMO1 | 0.3327 |
| rs12532031 | 1kg_7_37393277 | A | 7.348 | 9.82E−04 | ELMO1 | 0.3328 |
| rs75779749 | imm_2_191691363 | A | 18.17 | 9.82E−04 | STAT4 | 0.0249 |
| rs3810936 | imm_9_116592706 | A | −7.363 | 9.91E−04 | TNFSF15 | 0.3013 |

TABLE 9
Polymorphisms associated with TL1A fold-change and Signal One Carrier (linear model)

| | | Minor | | | | |
|---|---|---|---|---|---|---|
| Polymorphism | Illumina_id | Allele (A1) | BETA | P | Gene | MAF |
| rs116347760 | imm_1_114002774 | T | 44.04 | 4.15E−08 | MAGI3 | 0.01597 |
| rs77984256 | imm_14_68259573 | A | 33.2 | 2.42E−07 | RAD51B, ZFP36L1 | 0.01665 |
| rs74431747 | imm_1_113814325 | G | 26.72 | 6.92E−07 | MAGI3 | 0.03085 |
| rs75948156 | imm_1_113767166 | A | 26.72 | 6.92E−07 | MAGI3 | 0.03054 |
| rs76975167 | imm_1_113754608 | A | 26.72 | 6.92E−07 | MAGI3 | 0.03059 |
| rs77128194 | imm_1_113741794 | A | 26.72 | 6.92E−07 | MAGI3 | 0.03049 |
| rs115611397 | 1kg_8_79548346 | A | 48.65 | 8.28E−07 | LOC102724874, PKIA | 0.01008 |
| rs74395031 | imm_1_113740022 | G | 34.23 | 1.15E−06 | MAGI3 | 0.01702 |
| rs76709465 | imm_5_132158742 | C | 29.47 | 1.98E−06 | SEPT8, SOWAHA | 0.02516 |
| rs115984727 | imm_5_132143609 | A | 29.45 | 2.30E−06 | SEPT8, SOWAHA | 0.02534 |
| rs7812931 | rs7812931 | A | 21.05 | 2.33E−06 | ZHX2,DERLI | 0.06666 |
| rs11912198 | rs11912198 | A | 23.78 | 3.41E−06 | ZNRF3 | 0.01691 |
| rs16986990 | rs16986990 | G | 23.78 | 3.41E−06 | ZNRF3 | 0.01849 |
| rs4823000 | rs4823000 | G | 23.78 | 3.41E−06 | ZNRF3 | 0.0214 |
| rs8137391 | rs8137391 | A | 23.78 | 3.41E−06 | ZNRF3-AS1 | 0.01848 |
| rs7677400 | rs7677400 | G | 19.52 | 5.05E−06 | MAPK10 | 0.1008 |
| rs11600746 | imm_11_127851599 | G | 17.64 | 6.49E−06 | ETS1 | 0.1551 |
| rs11600915 | imm_11_127846698 | G | 17.64 | 6.49E−06 | ETS1 | 0.1542 |
| rs11606640 | imm_11_127840459 | A | 17.64 | 6.49E−06 | ETS1 | 0.1531 |
| rs12294634 | imm_11_127848372 | A | 17.64 | 6.49E−06 | ETS1 | 0.154 |
| rs61909068 | imm_11_127848167 | G | 17.64 | 6.49E−06 | ETS1 | 0.1544 |
| rs61909072 | imm_11_127855281 | A | 17.64 | 6.49E−06 | ETS1 | 0.1554 |
| rs73029052 | imm_11_127844385 | A | 17.64 | 6.49E−06 | ETS1 | 0.1539 |
| rs73029062 | imm_11_127849992 | G | 17.64 | 6.49E−06 | ETS1 | 0.1542 |
| rs116297428 | imm_2_162683961 | A | 28.63 | 6.67E−06 | LOC101929532 | 0.01535 |
| rs116352370 | 1kg_2_241302416 | T | 40.99 | 6.82E−06 | KIFIA | 0.06014 |
| rs28550609 | 1kg_8_79506636 | A | 20.79 | 9.39E−06 | LOC102724874, PKIA | 0.07173 |
| rs4145315 | 1kg_8_79562307 | A | 20.79 | 9.39E−06 | LOC102724874, PKIA | 0.07256 |
| rs13403657 | rs13403657 | G | 31.25 | 1.02E−05 | SNED1 | 0.01724 |
| rs115537678 | imm_5_150351522 | A | 61.41 | 1.17E−05 | ZNF300P1, GPX3 | 0.01122 |
| rs116767299 | imm_2_60785645 | A | 61.41 | 1.17E−05 | BCL11A, PAPOLG | 0.01545 |
| rs117542910 | imm_17_35391423 | G | 61.41 | 1.17E−05 | PSMD3 | 0.0142 |
| rs11847179 | rs11847179 | A | 61.41 | 1.17E−05 | TTC7B | 0.01352 |
| rs12591019 | rs12591019 | A | 61.41 | 1.17E−05 | TTC7B | 0.0142 |
| rs17126980 | rs17126980 | A | 61.41 | 1.17E−05 | TTC7B | 0.01378 |
| rs17126982 | rs17126982 | A | 61.41 | 1.17E−05 | TTC7B | 0.01289 |
| rs183396336 | imm_1_117099551 | A | 61.41 | 1.17E−05 | CD2 | 0.01034 |
| rs1998188 | rs1998188 | A | 61.41 | 1.17E−05 | TTC7B | 0.01354 |
| rs2401911 | rs2401911 | G | 61.41 | 1.17E−05 | TTC7B | 0.01425 |
| rs4496303 | imm_2_169021220 | A | 61.41 | 1.17E−05 | CERS6 | 0.01216 |
| rs4900059 | rs4900059 | A | 61.41 | 1.17E−05 | TTC7B | 0.01446 |
| rs4904723 | rs4904723 | G | 61.41 | 1.17E−05 | TTC7B | 0.02013 |
| rs6575143 | rs6575143 | C | 61.41 | 1.17E−05 | TTC7B | 0.01357 |
| rs6575144 | rs6575144 | G | 61.41 | 1.17E−05 | TTC7B | 0.01357 |
| rs72832303 | ccc-6-20826759-A-G | G | 30.7 | 1.17E−05 | CDKAL1 | 0.02041 |
| rs76824122 | imm_2_204472857 | A | 61.41 | 1.17E−05 | CTLA4, ICOS | 0.01676 |
| rs77411382 | seq-VH-424 | A | 61.41 | 1.17E−05 | RGS21, RGS1 | 0.01697 |
| rs78498467 | imm_2_181832217 | C | 61.41 | 1.17E−05 | LOC101927156 | 0.01624 |
| rs8004183 | rs8004183 | A | 61.41 | 1.17E−05 | TTC7B | 0.01357 |
| rs8019797 | rs8019797 | C | 61.41 | 1.17E−05 | TTC7B | 0.01357 |
| rs6757588 | rs6757588 | G | 9.376 | 1.29E−05 | ARHGAP15 | 0.3473 |
| rs76887590 | imm_14_68364326 | A | 43.09 | 1.49E−05 | ZFP36L1, ACTN1 | 0.02328 |
| rs7713991 | rs7713991 | A | 15.8 | 1.66E−05 | LOC401177, CDH18 | 0.1978 |
| rs115870915 | imm_1_113935553 | A | 30.75 | 1.67E−05 | MAGI3 | 0.02177 |
| rs33996649 | imm_1_114196212 | A | 30.75 | 1.67E−05 | PTPN22 | 0.02177 |
| rs34209542 | imm_1_114174047 | G | 30.75 | 1.67E−05 | AP4B1-AS1 | 0.02318 |
| rs12637133 | 1kg_3_18730712 | A | 42.46 | 1.89E−05 | SATB1-AS1, KCNH8 | 0.02897 |
| rs61394970 | 1kg_8_79494228 | G | 23.37 | 2.21E−05 | LOC102724874, PKIA | 0.03644 |
| rs73688944 | 1kg_8_79493024 | G | 23.37 | 2.21E−05 | LOC102724874, PKIA | 0.03649 |
| rs1864577 | 1kg_8_79411977 | A | 21.91 | 2.22E−05 | LOC102724874, PKIA | 0.03639 |
| rs60835488 | imm_14_68276435 | A | 18.98 | 2.39E−05 | RAD51B, ZFP36L1 | 0.05737 |
| rs73277289 | imm_14_68277780 | G | 18.98 | 2.39E−05 | RAD51B, ZFP36L1 | 0.0581 |
| rs115870915 | imm_1_113935553 | A | 40.5 | 2.67E−05 | MAGI3 | 0.02177 |
| rs116347760 | imm_1_114002774 | T | 40.5 | 2.67E−05 | MAGI3 | 0.01597 |
| rs17031888 | imm_1_114163459 | G | 40.5 | 2.67E−05 | AP4B1-AS1 | 0.03863 |
| rs17031955 | imm_1_114212503 | A | 40.5 | 2.67E−05 | AP4B1-AS1 | 0.03644 |
| rs33996649 | imm_1_114196212 | A | 40.5 | 2.67E−05 | PTPN22 | 0.02177 |
| rs34209542 | imm_1_114174047 | G | 40.5 | 2.67E−05 | AP4B1-AS1 | 0.02318 |
| rs74334220 | imm_9_138275616 | G | 46.29 | 2.74E−05 | QSOX2 | 0.02261 |
| rs2229136 | rs2229136 | G | 38.87 | 2.83E−05 | ALOX5 | 0.05768 |
| rs146848541 | rs12013474 | A | 28.37 | 3.16E−05 | FMR1_FMRINB | 0.06752 |
| rs146850466 | rs5904818 | A | 28.37 | 3.16E−05 | FMR1_FMRINB | 0.06703 |
| rs7713991 | rs7713991 | A | 10.99 | 3.67E−05 | LOC401177, CDH18 | 0.1978 |
| rs11465283 | rs11465283 | A | 12.97 | 3.91E−05 | ADAM19 | 0.1133 |
| rs7812931 | rs7812931 | A | 24.86 | 3.96E−05 | ZHX2, DERLI | 0.06666 |
| rs74998771 | imm_16_30864143 | A | 24.24 | 4.39E−05 | FBXL19 | 0.03059 |
| rs6708276 | rs6708276 | G | 8.699 | 4.72E−05 | ARHGAP15 | 0.3447 |
| rs74675346 | imm_19_10343638 | A | 39.12 | 5.34E−05 | TYK2 | 0.02678 |
| rs1944961 | rs1944961 | A | 9.197 | 5.39E−05 | TENM4, LOC101928944 | 0.2136 |
| rs191204 | imm_5_55463560 | A | 13.27 | 5.85E−05 | ANKRD55 | 0.4793 |
| rs8009181 | rs8009181 | A | 20.15 | 6.04E−05 | MAPK1IP1L, LGALS3 | 0.03743 |
| rs117413168 | imm_6_126801166 | A | 32.89 | 6.24E−05 | CENPW, MIR588 | 0.01342 |
| rs62437166 | imm_6_127308860 | A | 32.89 | 6.24E−05 | MIR588, RSPO3 | 0.01989 |
| rs74885500 | imm_6_126728598 | A | 32.89 | 6.24E−05 | CENPW, NONE | 0.01796 |
| rs6003160 | rs6003160 | G | 16.03 | 6.55E−05 | SCUBE1 | 0.295 |
| rs78103074 | imm_1_171126786 | A | 34.5 | 6.74E−05 | FASLG, TNFSF18 | 0.05048 |
| rs10461422 | imm_5_55468005 | C | 14.9 | 7.11E−05 | ANKRD55 | 0.2104 |
| rs17645980 | imm_5_55460497 | A | 14.9 | 7.11E−05 | ANKRD55 | 0.2127 |
| rs2940520 | rs2940520 | G | 30.43 | 7.12E−05 | UNC5A | 0.05085 |
| rs10461422 | imm_5_55468005 | C | 10.63 | 7.36E−05 | ANKRD55 | 0.2104 |
| rs17645980 | imm_5_55460497 | A | 10.63 | 7.36E−05 | ANKRD55 | 0.2127 |
| rs56086356 | imm_11_127881686 | C | 13.9 | 7.65E−05 | ETS1 | 0.1774 |
| rs6003160 | rs6003160 | G | 10.12 | 7.69E−05 | SCUBE1 | 0.295 |
| rs12982003 | imm_19_51997600 | C | 26.17 | 7.89E−05 | SLC1A5, SNAR-E | 0.04808 |
| rs10790957 | imm_11_127860440 | G | 14.61 | 8.04E−05 | ETS1 | 0.4149 |
| rs878983 | rs878983 | A | 10.15 | 8.05E−05 | LAPTM4A, SDC1 | 0.2088 |
| rs2229136 | rs2229136 | G | 23.33 | 8.74E−05 | ALOX5 | 0.05768 |
| rs10169606 | rs10169606 | G | 7.908 | 8.75E−05 | ARHGAP15 | 0.3662 |
| rs77984256 | imm_14_68259573 | A | 30.12 | 8.78E−05 | RAD51B, ZFP36L1 | 0.01665 |
| rs61732805 | imm_1_153675260 | A | 28.87 | 9.00E−05 | ASH1L | 0.013 |
| rs6056048 | rs6056048 | A | 12.87 | 9.11E−05 | PLCB1 | 0.3511 |
| rs2888456 | rs2888456 | G | 8.787 | 9.33E−05 | LOC101928327, DIRC3-AS1 | 0.4371 |
| rs2940520 | rs2940520 | G | 21.74 | 9.44E−05 | UNC5A | 0.05085 |
| rs747024 | rs747024 | A | 16.11 | 9.68E−05 | HERC4 | 0.1289 |
| rs4291387 | rs4291387 | A | 13.12 | 9.97E−05 | LOC158435 | 0.3516 |
| rs77770153 | 1kg_17_29603462 | A | 16.96 | 1.02E−04 | LOC101927239, CCL2 | 0.06682 |
| rs6928830 | rs6928830 | G | 13.44 | 1.10E−04 | ME1, PRSS35 | 0.1732 |
| rs5992462 | rs5992462 | G | 25.44 | 1.19E−04 | LINC00895, SEPT5 | 0.07632 |
| rs8137838 | rs8137838 | C | 25.44 | 1.19E−04 | LINC00895, SEPT5 | 0.07605 |
| rs12035823 | rs12035823 | G | 24.83 | 1.21E−04 | OLFM3, COL11A1 | 0.04886 |
| rs6928830 | rs6928830 | G | 9.647 | 1.21E−04 | ME1, PRSS35 | 0.1732 |
| rs76655944 | imm_1_181829847 | A | 39.07 | 1.23E−04 | NCF2, ARPC5 | 0.01143 |
| rs17491714 | imm_12_56688746 | G | 33.42 | 1.25E−04 | XRCC6BP1, LOC101927653 | 0.04333 |
| rs17086512 | imm_5_96040813 | G | 9.736 | 1.26E−04 | CAST | 0.2483 |
| rs275217 | rs275217 | G | 13.96 | 1.27E−04 | C15orf53, C15orf54 | 0.05848 |
| rs13187079 | imm_5_96043366 | G | 9.976 | 1.31E−04 | CAST | 0.2479 |
| rs4320976 | imm_11_75765492 | A | 15.4 | 1.33E−04 | PRKRIR | 0.1918 |
| rs7948288 | imm_11_75768491 | A | 15.4 | 1.33E−04 | PRKRIR | 0.1935 |
| rs4910068 | rs4910068 | G | 13.68 | 1.34E−04 | ST5 | 0.2834 |
| rs2680344 | rs2680344 | G | −10.86 | 1.40E−04 | HCN4 | 0.2237 |
| rs113514774 | ccc-12-56505633-G-A | A | 44.39 | 1.42E−04 | CTDSP2 | 0.02427 |
| rs115611397 | 1kg_8_79548346 | A | 44.39 | 1.42E−04 | LOC102724874, PKIA | 0.01008 |
| rs116432609 | imm_5_150340428 | G | 44.39 | 1.42E−04 | ZNF300P1, GPX3 | 0.02563 |
| rs118001674 | imm_20_42669344 | A | 44.39 | 1.42E−04 | PKIG | 0.04463 |
| rs76540957 | imm_20_42679347 | G | 44.39 | 1.42E−04 | PKIG | 0.04516 |
| rs4303275 | rs4303275 | A | 8.86 | 1.52E−04 | TRHDE | 0.2789 |
| rs62362364 | imm_5_55477328 | G | 10.1 | 1.53E−04 | ANKRD55 | 0.2034 |
| rs10189240 | rs10189240 | G | 7.777 | 1.63E−04 | ARHGAP15 | 0.3637 |
| rs2048957 | rs2048957 | A | 7.904 | 1.63E−04 | ARHGAP15 | 0.3606 |
| rs74674305 | imm_1_113680743 | A | 22.43 | 1.63E−04 | LOC643441, MAGI3 | 0.03023 |
| rs12722502 | imm_10_6133145 | A | 37.74 | 1.73E−04 | IL2RA | 0.01383 |
| rs12722547 | imm_10_6112099 | C | 37.74 | 1.73E−04 | IL2RA | 0.01383 |
| rs76887590 | imm_14_68364326 | A | 25.04 | 1.75E−04 | ZFP36L1, ACTN1 | 0.02328 |
| rs878983 | rs878983 | A | 16.29 | 1.78E−04 | LAPTM4A, SDC1 | 0.2088 |
| rs10490444 | rs10490444 | G | −8.452 | 1.81E−04 | LOC101928327, DIRC3-AS1 | 0.438 |
| rs12118482 | seq-rs12118482 | G | 31.01 | 1.85E−04 | PTPRC | 0.02 |
| rs12137209 | seq-rs12137209 | A | 31.01 | 1.85E−04 | ATP6V1G3, PTPRC | 0.02135 |
| rs55720245 | imm_9_35368401 | A | 31.34 | 1.88E−04 | UNC13B | 0.01561 |
| rs34279840 | ccc-21-44452087-C-T | A | 24.25 | 1.89E−04 | C21orf33, ICOSLG | 0.02819 |
| rs16966547 | rs16966547 | G | 14.79 | 1.91E−04 | MAPRE2, ZNF397 | 0.07 |
| rs8093515 | rs8093515 | G | 14.79 | 1.91E−04 | MAPRE2, ZNF397 | 0.06729 |
| rs118001674 | imm_20_42669344 | A | 31.38 | 1.95E−04 | PKIG | 0.04463 |
| rs76540957 | imm_20_42679347 | G | 31.38 | 1.95E−04 | PKIG | 0.04516 |
| rs117889858 | imm_16_67072555 | A | 26.88 | 1.96E−04 | SMPD3, ZFP90 | 0.0213 |
| rs1277016 | rs1277016 | G | 9.905 | 2.00E−04 | STXBP3 | 0.2562 |
| rs76709465 | imm_5_132158742 | C | 26.77 | 2.03E−04 | SEPT8, SOWAHA | 0.02516 |
| rs11912198 | rs11912198 | A | 23.99 | 2.08E−04 | ZNRF3 | 0.01691 |
| rs16986990 | rs16986990 | G | 23.99 | 2.08E−04 | ZNRF3 | 0.01849 |
| rs4823000 | rs4823000 | G | 23.99 | 2.08E−04 | ZNRF3 | 0.0214 |
| rs8137391 | rs8137391 | A | 23.99 | 2.08E−04 | ZNRF3-AS1 | 0.01848 |
| rs180473 | rs180473 | A | 7.385 | 2.12E−04 | EPB41L4A- | 0.3998 |
| | | | | | AS2, LOC102467214 | |
| rs10823062 | rs10823062 | A | 15.01 | 2.21E−04 | CTNNA3 | 0.2119 |
| rs2394411 | rs2394411 | A | 15.01 | 2.21E−04 | CTNNA3 | 0.2118 |
| rs117889858 | imm_16_67072555 | A | 34.84 | 2.23E−04 | SMPD3, ZFP90 | 0.0213 |
| rs73003218 | imm_11_118155373 | A | 23.7 | 2.25E−04 | DDX6 | 0.01665 |
| rs74675346 | imm_19_10343638 | A | 22.7 | 2.29E−04 | TYK2 | 0.02678 |
| rs117079792 | 1kg_8_79665101 | A | 35.03 | 2.29E−04 | PKIA | 0.04991 |
| rs117927932 | 1kg_8_79702265 | G | 35.03 | 2.29E−04 | PKIA, ZC2HC1A | 0.03905 |
| rs118136953 | 1kg_8_79710777 | A | 35.03 | 2.29E−04 | PKIA, ZC2HC1A | 0.0392 |
| rs118149281 | 1kg_8_79689940 | A | 35.03 | 2.29E−04 | PKIA, ZC2HC1A | 0.0389 |
| rs16905875 | 1kg_8_79714122 | A | 35.03 | 2.29E−04 | PKIA, ZC2HC1A | 0.04041 |
| rs74696769 | 1kg_8_79694906 | A | 35.03 | 2.29E−04 | PKIA, ZC2HC1A | 0.03881 |
| rs74761562 | 1kg_8_79662196 | G | 35.03 | 2.29E−04 | PKIA | 0.03649 |
| rs75488794 | 1kg_8_79664297 | G | 35.03 | 2.29E−04 | PKIA | 0.05006 |
| rs75878904 | 1kg_8_79674210 | G | 35.03 | 2.29E−04 | PKIA | 0.05013 |
| rs75901112 | 1kg_8_79696293 | A | 35.03 | 2.29E−04 | PKIA, ZC2HC1A | 0.039 |
| rs76195974 | 1kg_8_79708007 | A | 35.03 | 2.29E−04 | PKIA, ZC2HC1A | 0.03921 |
| rs76483342 | 1kg_8_79683854 | A | 35.03 | 2.29E−04 | PKIA, ZC2HC1A | 0.039 |
| rs77650073 | 1kg_8_79712780 | G | 35.03 | 2.29E−04 | PKIA, ZC2HC1A | 0.0392 |
| rs78100278 | 1kg_8_79681331 | G | 35.03 | 2.29E−04 | PKIA, ZC2HC1A | 0.0391 |
| rs78767737 | 1kg_8_79687223 | G | 35.03 | 2.29E−04 | PKIA, ZC2HC1A | 0.039 |
| rs79641310 | 1kg_8_79704013 | A | 35.03 | 2.29E−04 | PKIA, ZC2HC1A | 0.0392 |
| rs114797146 | imm_2_99955021 | A | 37.55 | 2.34E−04 | AFF3 | 0.01655 |
| rs76990532 | imm_2_99961776 | A | 37.55 | 2.34E−04 | AFF3 | 0.01498 |
| rs1944959 | rs1944959 | A | 8.191 | 2.38E−04 | TENM4, LOC101928944 | 0.2259 |
| rs115984727 | imm_5_132143609 | A | 26.69 | 2.43E−04 | SEPT8, SOWAHA | 0.02534 |
| rs607660 | rs607660 | G | 10.92 | 2.55E−04 | CTAGE1, LOC101927571 | 0.4541 |
| rs2141102 | rs2141102 | A | 9.493 | 2.71E−04 | NEFL, DOCK5 | 0.2772 |
| rs7002363 | rs7002363 | G | 9.493 | 2.71E−04 | NEFL, DOCK5 | 0.275 |
| rs1277016 | rs1277016 | G | 13.94 | 2.72E−04 | STXBP3 | 0.2562 |
| rs6462484 | rs6462484 | A | −8.754 | 2.73E−04 | BBS9 | 0.3993 |
| rs7895833 | rs7895833 | G | 13.9 | 2.75E−04 | DNAJC12, SIRT1 | 0.1969 |
| rs7005778 | rs7005778 | A | 7.87 | 2.76E−04 | FBXO32, KLHL38 | 0.3173 |
| rs3729703 | rs3729703 | C | 37.01 | 2.79E−04 | MEF2C | 0.01618 |
| rs8036951 | rs8036951 | G | 15.78 | 2.83E−04 | FAM189A1 | 0.2253 |
| rs12539781 | rs12539781 | A | 8.854 | 2.86E−04 | LIMK1, EIF4H | 0.2135 |
| rs4648892 | rs4648892 | G | 8.833 | 2.88E−04 | TCEA3 | 0.2663 |
| rs76321080 | imm_14_68299875 | A | 23.3 | 2.91E−04 | RAD51B, ZFP36L1 | 0.05847 |
| rs13208357 | rs13208357 | A | 10.4 | 2.92E−04 | EPHA7, TSG1 | 0.1341 |
| rs114979698 | imm_1_195931930 | G | 27.46 | 2.96E−04 | DENND1B | 0.0367 |
| rs74792569 | imm_1_195719456 | A | 27.46 | 2.96E−04 | CRB1, DENND1B | 0.03247 |
| rs75622950 | imm_1_196060621 | A | 27.46 | 2.96E−04 | DENND1B, C1orf53 | 0.03858 |
| rs17783485 | rs17783485 | G | 36.45 | 2.97E−04 | LAMA2 | 0.03936 |
| rs1864577 | 1kg_8_79411977 | A | 25.35 | 2.97E−04 | LOC102724874, PKIA | 0.03639 |
| rs61394970 | 1kg_8_79494228 | G | 25.35 | 2.97E−04 | LOC102724874, PKIA | 0.03644 |
| rs73688944 | 1kg_8_79493024 | G | 25.35 | 2.97E−04 | LOC102724874, PKIA | 0.03649 |
| rs11779459 | rs11779459 | A | 12.32 | 2.98E−04 | ZHX2 | 0.3691 |
| rs6063456 | imm_20_48047586 | C | 12.75 | 3.03E−04 | SNAI1, TRERNA1 | 0.3958 |
| rs6125855 | imm_20_48057194 | A | 12.75 | 3.03E−04 | SNAI1, TRERNA1 | 0.3939 |
| rs6125864 | imm_20_48066512 | A | 12.75 | 3.03E−04 | SNAI1, TRERNA1 | 0.3962 |
| rs3920079 | rs3920079 | A | 13.66 | 3.08E−04 | CTNNA3 | 0.3193 |
| rs11769844 | rs11769844 | A | 13.98 | 3.09E−04 | STRA8 | 0.219 |
| rs11761905 | rs11761905 | A | 9.377 | 3.14E−04 | JAZF1 | 0.2309 |
| rs72802340 | imm_16_73790029 | A | 20.07 | 3.22E−04 | ZFP1, CTRB2 | 0.03691 |
| rs180782 | rs180782 | G | 8.124 | 3.22E−04 | YY1P2, LRP1B | 0.2339 |
| rs17779592 | imm_17_23020853 | A | 34.32 | 3.25E−04 | LGALS9, NOS2 | 0.03749 |
| rs12465492 | rs12465492 | A | 7.432 | 3.32E−04 | ARHGAP15 | 0.365 |
| rs114442346 | imm_1_154121831 | G | 35.57 | 3.56E−04 | SYT11 | 0.02433 |
| rs116547449 | imm_1_154148459 | A | 35.57 | 3.56E−04 | RIT1; KIAA0907 | 0.02444 |
| rs61732805 | imm_1_153675260 | A | 35.57 | 3.56E−04 | ASH1L | 0.013 |
| rs78029196 | imm_1_154247058 | C | 35.57 | 3.56E−04 | SSR2 | 0.01467 |
| rs236768 | imm_4_103295006 | G | 9.714 | 3.56E−04 | BANK1, SLC39A8 | 0.193 |
| rs6102912 | rs6102912 | G | −8.1 | 3.61E−04 | PTPRT | 0.4443 |
| rs6124476 | rs6124476 | G | −8.1 | 3.61E−04 | PTPRT | 0.4476 |
| rs6130169 | rs6130169 | A | −8.1 | 3.61E−04 | PTPRT | 0.4475 |
| rs4910068 | rs4910068 | G | 8.401 | 3.63E−04 | ST5 | 0.2834 |
| rs2528691 | rs2528691 | G | 8.325 | 3.67E−04 | IMMP2L, DOCK4 | 0.4921 |
| rs12792040 | vh_11_124129360 | A | 16.57 | 3.71E−04 | ESAM | 0.08091 |
| rs6435959 | rs6435959 | A | −8.015 | 3.74E−04 | LOC101928327, DIRC3-AS1 | 0.3867 |
| rs8036951 | rs8036951 | G | 9.919 | 3.83E−04 | FAM189A1 | 0.2253 |
| rs6478109 | imm_9_116608587 | A | −10.73 | 3.86E−04 | TNFSF15 | 0.2995 |
| rs7848647 | imm_9_116608867 | A | −10.73 | 3.86E−04 | TNFSF15 | 0.2978 |
| rs16924888 | rs16924888 | A | 14.93 | 3.89E−04 | DNAJC12 | 10.131 |
| rs17456400 | rs17456400 | C | 14.93 | 3.89E−04 | HERC4, MYPN | 0.1342 |
| rs2284665 | s2284665 | A | 17.05 | 3.90E−04 | HTRA1 | 0.2158 |
| rs77498465 | imm_2_162718729 | A | 33.82 | 3.90E−04 | LOC101929532 | 0.04647 |
| rs34279840 | ccc-21-44452087-C-T | A | 30.31 | 3.92E−04 | C21orf33, ICOSLG | 0.02819 |
| rs747024 | rs747024 | A | 10.26 | 3.94E−04 | HERC4 | 0.1289 |
| rs74395031 | imm_1_113740022 | G | 30 | 3.94E−04 | MAGI3 | 0.01702 |
| rs16834177 | seq-rs16834177 | G | 15.18 | 4.03E−04 | RGS21 | 0.07836 |
| rs4806768 | seq-rs4806768 | A | 7.587 | 4.03E−04 | LAIR2 | 0.4648 |
| rs1113283 | imm_17_23131525 | A | 14.41 | 4.05E−04 | NOS2 | 0.2454 |
| rs10760109 | imm_9_122437397 | A | 35.25 | 4.10E−04 | MEGF9 | 0.02328 |
| rs1886338 | imm_9_122451373 | G | 35.25 | 4.10E−04 | MEGF9 | 0.02325 |
| rs17031888 | imm_1_114163459 | G | 23.22 | 4.16E−04 | AP4B1-AS1 | 0.03863 |
| rs17031955 | imm_1_114212503 | A | 23.22 | 4.16E−04 | AP4B1-AS1 | 0.03644 |
| rs12428125 | rs12428125 | A | 46.06 | 4.16E−04 | BASP1P1, SGCG | 0.0427 |
| rs2287773 | rs2287773 | A | 46.06 | 4.16E−04 | SPINK5 | 0.02005 |
| rs74334220 | imm_9_138275616 | G | 46.06 | 4.16E−04 | QSOX2 | 0.02261 |
| rs13086717 | imm_3_46114503 | G | 9.223 | 4.18E−04 | XCR1, CCR1 | 0.1898 |
| rs17720798 | imm_6_127396930 | A | 19.57 | 4.42E−04 | MIR588, RSPO3 | 0.05967 |
| rs79033062 | imm_1_190803780 | A | 35.47 | 4.43E−04 | RGS21, RGS1 | 0.01289 |
| rs79454488 | seq-VH-748 | G | 35.47 | 4.43E−04 | RGS1 | 0.0131 |
| rs2724011 | 1kg_7_37365041 | A | 7.787 | 4.47E−04 | ELMO1 | 0.2354 |
| rs1836767 | rs1836767 | G | 12.04 | 4.53E−04 | PLD5 | 0.07658 |
| rs191204 | imm_5_55463560 | A | 7.904 | 4.53E−04 | ANKRD55 | 0.4793 |
| rs4291387 | rs4291387 | A | 7.729 | 4.58E−04 | LOC158435 | 0.3516 |
| rs2104517 | rs2104517 | A | 26.54 | 4.58E−04 | MIR548F5 | 0.05011 |
| rs6125877 | rs6125877 | C | 13.22 | 4.62E−04 | TRERNA1 | 0.4207 |
| rs9897780 | rs9897780 | A | 19.43 | 4.73E−04 | MYH10, CCDC42 | 0.1904 |
| rs4606022 | imm_8_11392342 | G | 12.04 | 4.84E−04 | BLK | 0.3851 |
| rs16949 | imm_17_23148826 | G | 14.17 | 4.93E−04 | NOS2 | 0.2491 |
| rs3794766 | imm_17_23146048 | A | 14.17 | 4.93E−04 | NOS2 | 0.2482 |
| rs4796080 | imm_17_23146864 | G | 14.17 | 4.93E−04 | NOS2 | 0.2482 |

TABLE 10
Polymorphisms associated with high-low TL1A fold-change (logistic model)

| | | Minor | | | | |
|---|---|---|---|---|---|---|
| Polymorphism | Illumina_id | Allele (A1) | OR | P | Gene.refGene | MAF |
| rs6737109 | rs6737109 | G | 0.1922 | 4.77E−05 | LOC102723362, KLHL29 | 0.406 |
| rs6478109 | imm_9_116608587 | A | 0.1922 | 5.03E−05 | TNFSF15 | 0.2995 |
| rs7848647 | imm_9_116608867 | A | 0.1922 | 5.03E−05 | TNFSF15 | 0.2978 |
| rs201292440 | 9-116611115-GAA- | D | 0.2233 | 1.44E−04 | TNFSF15_TNFSF8 | 0.2695 |
| | INSERTION | | | | | |
| rs1407308 | imm_9_116610044 | A | 0.2222 | 1.52E−04 | TNFSF15, TNFSF8 | 0.4745 |
| rs3851519 | rs3851519 | A | 4.027 | 1.71E−04 | LY86, RREB1 | 0.3995 |
| rs6921610 | rs6921610 | G | 3.803 | 1.91E−04 | LY86, RREB1 | 0.4637 |
| rs11793394 | imm_9_116611852 | G | 0.2279 | 2.07E−04 | TNFSF15, TNFSF8 | 0.4756 |
| rs4979466 | imm_9_116669530 | A | 0.2563 | 2.15E−04 | TNFSF15, TNFSF8 | 0.4222 |
| rs4979467 | imm_9_116669864 | G | 0.2563 | 2.15E−04 | TNFSF15, TNFSF8 | 0.4225 |
| rs7043505 | imm_9_116668349 | G | 0.2563 | 2.15E−04 | TNFSF15, TNFSF8 | 0.4224 |
| rs7869487 | imm_9_116620735 | G | 0.2431 | 2.39E−04 | TNFSF15, TNFSF8 | 0.2841 |
| rs17219926 | imm_9_116619674 | A | 0.2329 | 2.79E−04 | TNFSF15, TNFSF8 | 0.472 |
| rs4979469 | imm_9_116680242 | G | 0.2584 | 2.85E−04 | TNFSF15, TNFSF8 | 0.4004 |
| rs7863183 | imm_9_116682239 | A | 0.2584 | 2.85E−04 | TNFSF15, TNFSF8 | 0.3985 |
| rs2857201 | rs2857201 | C | 0.189 | 3.13E−04 | HLA-DQB2, HLA-DOB | 0.2835 |
| rs1842399 | rs1842399 | C | 0.189 | 3.13E−04 | HLA-DQB2, HLA-DOB | 0.2834 |
| rs2621390 | rs2621390 | G | 0.189 | 3.13E−04 | HLA-DQB2, HLA-DOB | 0.2839 |
| rs2621391 | rs2621391 | G | 0.189 | 3.13E−04 | HLA-DQB2, HLA-DOB | 0.2839 |
| rs2621393 | rs2621393 | G | 0.189 | 3.13E−04 | HLA-DQB2, HLA-DOB | 0.2834 |
| rs2857205 | rs2857205 | A | 0.189 | 3.13E−04 | HLA-DQB2, HLA-DOB | 0.2836 |
| rs12913742 | rs12913742 | G | 3.367 | 3.23E−04 | RGMA, LOC101927153 | 0.4576 |
| rs10509690 | rs10509690 | A | 0.2814 | 3.59E−04 | SORBS1 | 0.2369 |
| rs4366152 | imm_9_116604696 | A | 0.2679 | 3.81E−04 | TNFSF15 | 0.2982 |
| rs7030574 | imm_9_116607870 | C | 0.2671 | 4.45E−04 | TNFSF15 | 0.4808 |
| rs1233651 | 1kg_17_29663474 | G | 0.192 | 4.46E−04 | CCL11, CCL8 | 0.1864 |
| rs1233651 | rs1233651 | G | 0.192 | 4.46E−04 | CCL11, CCL8 | 0.1864 |
| rs2215185 | 1kg_17_29658015 | G | 0.192 | 4.46E−04 | CCL11, CCL8 | 0.1868 |
| rs885691 | 1kg_17_29665338 | A | 0.192 | 4.46E−04 | CCL11, CCL8 | 0.1863 |
| rs3125037 | rs3125037 | G | 0.2583 | 6.09E−04 | ZMYND11 | 0.2784 |
| rs17390873 | rs17390873 | A | 6.16 | 6.84E−04 | ATG4C, LINC00466 | 0.1236 |
| rs10169606 | rs10169606 | G | 2.938 | 7.19E−04 | ARHGAP15 | 0.3662 |
| rs9375487 | imm_6_127438933 | G | 3.011 | 7.32E−04 | MIR588, RSPO3 | 0.4033 |
| rs2724011 | 1kg_7_37365041 | A | 3.459 | 7.41E−04 | ELMO1 | 0.2354 |
| rs2621421 | rs2621421 | C | 0.2467 | 7.47E−04 | HLA-DQB2, HLA-DOB | 0.3388 |
| rs62056381 | 1kg_17_29699681 | A | 0.2065 | 7.70E−04 | CCL8, CCL13 | 0.1897 |
| rs17461863 | rs17461863 | A | 0.3362 | 8.36E−04 | GABRB1 | 0.4427 |
| rs7677890 | rs7677890 | A | 0.3362 | 8.36E−04 | GABRB1 | 0.4432 |
| rs2913784 | rs2913784 | A | 3.526 | 8.52E−04 | COL23A1 | 0.3284 |
| rs2516470 | rs2516470 | C | 0.2824 | 8.60E−04 | MICA, HCP5 | 0.3161 |
| rs7164805 | rs7164805 | A | 0.2961 | 8.67E−04 | BCL2A1, ZFAND6 | 0.4474 |
| rs748569 | imm_2_61710681 | C | 5.115 | 8.96E−04 | XPO1, FAM161A | 0.1911 |
| rs4979464 | imm_9_116641968 | A | 0.3035 | 9.34E−04 | TNFSF15, TNFSF8 | 0.3041 |
| rs2700990 | 1kg_7_37349302 | A | 3.187 | 9.86E−04 | ELMO1 | 0.2521 |
| rs3128941 | rs3128941 | G | 3.03 | 1.01E−03 | HLA-DOA, HLA-DPA1 | 0.4577 |
| rs1936811 | imm_6_127425553 | T | 2.93 | 1.03E−03 | MIR588, RSPO3 | 0.4041 |
| rs1936812 | imm_6_127432378 | G | 2.93 | 1.03E−03 | MIR588, RSPO3 | 0.4025 |
| rs1936814 | imm_6_127434157 | A | 2.93 | 4.77E−05 | MIR588, RSPO3 | 0.4028 |
| rs9372856 | imm_6_127430145 | C | 2.93 | 5.03E−05 | MIR588, RSPO3 | 0.4038 |
| rs9401938 | imm_6_127432412 | A | 2.93 | 5.03E−05 | MIR588, RSPO3 | 0.4024 |
| rs972275 | imm_6_127433537 | G | 2.93 | 1.44E−04 | MIR588, RSPO3 | 0.4024 |
| rs7404848 | rs7404848 | A | 0.2024 | 1.52E−04 | CDYL2 | 0.2421 |
| rs3099840 | rs3099840 | G | 4.297 | 1.71E−04 | HCP5 | 0.2055 |
| rs3094228 | rs3094228 | G | 4.297 | 1.91E−04 | MICA, HCP5 | 0.2056 |
| rs722126 | imm_9_116632599 | C | 0.3043 | 2.07E−04 | TNFSF15, TNFSF8 | 0.2683 |
| rs4798791 | rs4798791 | A | 3.092 | 2.15E−04 | ANKRD12 | 0.3775 |
| rs2280728 | rs2280728 | C | 2.735 | 2.15E−04 | CASC23 | 0.4916 |
| rs683028 | rs683028 | G | 3.357 | 2.15E−04 | DKFZp686K1684, | 0.4055 |
| | | | | | LOC100506675 | |
| rs79517864 | imm_6_127433740 | G | 0.09062 | 2.39E−04 | MIR588, RSPO3 | 0.06479 |
| rs2067577 | rs2067577 | C | 0.278 | 2.79E−04 | HLA-DQB2, HLA-DOB | 0.3311 |
| rs2157079 | rs2157079 | A | 0.278 | 2.85E−04 | HLA-DQB2, HLA-DOB | 0.3308 |
| rs1837 | imm_9_122658050 | A | 3.426 | 2.85E−04 | PHF19 | 0.2603 |
| rs2717954 | 1kg_7_37361898 | G | 2.802 | 3.13E−04 | ELMO1 | 0.2877 |
| rs1761455 | seq-rs1761455 | G | 4.025 | 3.13E−04 | LILRA3, LILRA5 | 0.2835 |
| rs404032 | seq-rs404032 | C | 4.025 | 3.13E−04 | LILRA3, LILRA5 | 0.2834 |
| rs414135 | seq-rs414135 | A | 4.025 | 3.13E−04 | LILRA3, LILRA5 | 0.2833 |
| rs651279 | seq-rs651279 | G | 4.025 | 3.13E−04 | LILRA3, LILRA5 | 0.2841 |
| rs759819 | seq-rs759819 | G | 4.025 | 3.13E−04 | LILRA3, LILRA5 | 0.2835 |
| rs1003533 | imm_5_131783550 | A | 0.2496 | 3.23E−04 | C5orf56 | 0.2059 |
| rs10900807 | imm_5_131785379 | C | 0.2496 | 3.59E−04 | C5orf56 | 0.2041 |
| rs1981524 | imm_5_131784405 | A | 0.2496 | 3.81E−04 | C5orf56 | 0.2057 |
| rs2745358 | imm_6_127433163 | G | 2.695 | 4.45E−04 | MIR588, RSPO3 | 0.4553 |
| rs2548278 | rs2548278 | A | 3.175 | 4.46E−04 | ST8SIA4 | 0.3496 |
| rs2548276 | rs2548276 | A | 3.175 | 4.46E−04 | ST8SIA4 | 0.3498 |
| rs7180547 | rs7180547 | G | 2.908 | 4.46E−04 | RORA | 0.3919 |
| rs1853187 | imm_9_116636173 | C | 0.3233 | 4.46E−04 | TNFSF15, TNFSF8 | 0.3049 |
| rs77130822 | imm_4_123232824 | G | 0.2415 | 6.09E−04 | TRPC3, KIAA1109 | 0.2005 |
| rs945855 | rs945855 | A | 0.3599 | 6.84E−04 | LINC01526, IBTK | 10.428 |
| rs6447550 | rs6447550 | A | 0.3539 | 7.19E−04 | GABRB1 | 0.4839 |
| rs6902885 | imm_6_127422175 | A | 2.776 | 7.32E−04 | MIR588, RSPO3 | 0.4009 |
| rs7743393 | imm_6_127437908 | A | 2.776 | 7.41E−04 | MIR588, RSPO3 | 0.3998 |
| rs9321069 | imm_6_127434670 | A | 2.776 | 7.47E−04 | MIR588, RSPO3 | 0.3996 |
| rs9388546 | imm_6_127432542 | C | 2.776 | 7.70E−04 | MIR588, RSPO3 | 0.4 |
| rs17006627 | imm_2_61243113 | G | 4.546 | 8.36E−04 | C2orf74 | 0.1807 |
| rs59197404 | imm_2_61707640 | G | 4.628 | 8.36E−04 | XPO1, FAM161A | 0.1898 |
| rs6740218 | imm_2_61712593 | A | 4.628 | 8.52E−04 | XPO1, FAM161A | 0.1885 |
| rs748570 | imm_2_61711025 | G | 4.628 | 8.60E−04 | XPO1, FAM161A | 0.1893 |
| rs748571 | imm_2_61711589 | G | 4.628 | 8.67E−04 | XPO1, FAM161A | 0.1893 |
| rs7590132 | imm_2_61713189 | A | 4.628 | 8.96E−04 | XPO1, FAM161A | 0.1886 |
| rs1761456 | seq-rs1761456 | A | 3.944 | 9.34E−04 | LILRA3, LILRA5 | 0.2703 |
| rs2680344 | rs2680344 | G | 0.2499 | 9.86E−04 | HCN4 | 0.2237 |
| rs7179025 | rs7179025 | G | 0.2315 | 1.01E−03 | SLC27A2 | 0.1883 |
| rs11544238 | imm_12_56156422 | A | 3.154 | 1.03E−03 | ARHGAP9 | 0.3652 |
| rs4806768 | seq-rs4806768 | A | 2.783 | 1.03E−03 | LAIR2 | 0.4648 |
| rs914842 | imm_9_122658792 | A | 3.557 | 1.03E−03 | PHF19 | 0.226 |
| rs3131296 | rs3131296 | A | 8.203 | 1.03E−03 | NOTCH4 | 0.121 |
| rs3132956 | rs3132956 | A | 8.203 | 1.03E−03 | NOTCH4 | 0.1212 |
| rs3134796 | rs3134796 | G | 8.203 | 1.03E−03 | NOTCH4 | 0.1218 |
| rs3134942 | rs3134942 | A | 8.203 | 1.12E−03 | NOTCH4 | 0.121 |
| rs2228224 | imm_12_56151588 | G | 3.048 | 1.12E−03 | GLI1 | 0.3718 |
| rs75424572 | imm_6_127405932 | C | 0.1127 | 1.12E−03 | MIR588, RSPO3 | 0.06444 |
| rs6708276 | rs6708276 | G | 2.823 | 1.14E−03 | ARHGAP15 | 0.3447 |
| rs86567 | rs86567 | C | 3.175 | 1.19E−03 | HLA-DOA | 0.3778 |
| rs16863769 | rs16863769 | G | 0.2988 | 1.22E−03 | MTX2, MIR1246 | 0.244 |
| rs3130615 | rs3130615 | G | 0.2311 | 1.24E−03 | MICB | 0.2195 |
| rs3130573 | rs3130573 | G | 3.355 | 1.24E−03 | PSORS1C1, PSORS1C2 | 0.3434 |
| rs4303275 | rs4303275 | A | 3.091 | 1.33E−03 | TRHDE | 0.2789 |
| rs78698613 | imm_6_127382349 | A | 0.1175 | 1.33E−03 | MIR588, RSPO3 | 0.07251 |
| rs6757588 | rs6757588 | G | 2.836 | 1.33E−03 | ARHGAP15 | 0.3473 |
| rs4694846 | rs4694846 | G | 2.791 | 1.34E−03 | GABRB1 | 0.4309 |
| rs2544913 | rs2544913 | A | 3.029 | 1.44E−03 | ST8SIA4 | 0.3524 |
| rs2621332 | rs2621332 | G | 0.301 | 1.44E−03 | HLA-DOB | 0.3311 |
| rs2857114 | rs2857114 | G | 0.301 | 1.44E−03 | HLA-DOB | 0.3415 |
| rs2199870 | rs2199870 | G | 0.301 | 1.44E−03 | HLA-DQB2, HLA-DOB | 0.3312 |
| rs2621336 | rs2621336 | G | 0.301 | 1.44E−03 | HLA-DQB2, HLA-DOB | 0.3311 |
| rs2857130 | rs2857130 | A | 0.301 | 1.47E−03 | HLA-DQB2, HLA-DOB | 0.3311 |
| rs117324436 | imm_9_4995771 | G | 11.28 | 1.47E−03 | JAK2 | 0.08959 |
| rs10189240 | rs10189240 | G | 2.628 | 1.47E−03 | ARHGAP15 | 0.3637 |
| rs739456 | rs739456 | A | 0.2616 | 1.48E−03 | LOC285692 | 0.1975 |
| rs3132468 | rs3132468 | G | 0.2363 | 1.48E−03 | MICB | 0.2195 |
| rs911887 | rs911887 | G | 0.3593 | 1.48E−03 | SFTPD | 0.3975 |
| rs4684448 | rs4684448 | G | 0.3297 | 1.49E−03 | ITPR1, BHLHE40-AS1 | 0.4267 |
| rs11690566 | rs11690566 | A | 0.291 | 1.52E−03 | FAM136A, TGFA | 0.2698 |
| rs13147245 | imm_4_123742806 | A | 2.871 | 1.52E−03 | IL2, IL21 | 0.4048 |
| rs6820791 | imm_4_123741233 | A | 2.871 | 1.55E−03 | IL2, IL21 | 0.4047 |
| rs6820964 | imm_4_123741173 | A | 2.871 | 1.56E−03 | IL2, IL21 | 0.4048 |
| rs6826110 | imm_4_123741689 | G | 2.871 | 1.60E−03 | IL2, IL21 | 0.4048 |
| rs7669697 | imm_4_123741889 | T | 2.871 | 1.60E−03 | IL2, IL21 | 0.4045 |
| rs7670387 | seq-rs7670387 | C | 2.871 | 1.60E−03 | IL2, IL21 | 0.4046 |
| rs975403 | imm_4_123741090 | A | 2.871 | 1.60E−03 | IL2, IL21 | 0.4048 |
| rs975405 | imm_4_123740630 | G | 2.871 | 1.61E−03 | IL2, IL21 | 0.4049 |
| rs976183 | imm_4_123742180 | G | 2.871 | 1.61E−03 | IL2, IL21 | 0.4049 |
| rs976184 | imm_4_123742121 | G | 2.871 | 1.61E−03 | IL2, IL21 | 0.4048 |
| rs4606022 | imm_8_11392342 | G | 2.794 | 1.61E−03 | BLK | 0.3851 |
| rs2700986 | 1kg_7_37356329 | A | 3.11 | 1.61E−03 | ELMO1 | 0.2047 |
| rs2724018 | 1kg_7_37358537 | A | 3.11 | 1.61E−03 | ELMO1 | 0.2044 |
| rs6920606 | rs6920606 | A | 2.739 | 1.64E−03 | HLA-DOA, HLA-DPA1 | 0.4959 |
| rs11610401 | imm_12_66773584 | T | 0.3548 | 1.65E−03 | IFNG-AS1, IFNG | 0.3964 |
| rs7304878 | imm_12_66772251 | G | 0.3548 | 1.65E−03 | IFNG-AS1, IFNG | 0.3953 |
| rs11224827 | rs11224827 | A | 4.492 | 1.67E−03 | TRPC6 | 0.1086 |
| rs1457020 | rs1457020 | A | 0.286 | 1.68E−03 | LINC01467, NONE | 0.2842 |
| rs17771891 | imm_5_131772101 | A | 0.2819 | 1.72E−03 | SLC22A5, C5orf56 | 0.2052 |
| rs26519 | imm_5_96175859 | A | 0.1557 | 1.81E−03 | ERAP1 | 0.08176 |
| rs10188460 | imm_2_61712172 | A | 4.285 | 1.81E−03 | XPO1, FAM161A | 0.1738 |
| rs12541603 | rs12541603 | G | 2.97 | 1.81E−03 | LINC00824 | 0.4047 |
| rs17650496 | imm_6_127312457 | G | 0.08501 | 1.81E−03 | MIR588, RSPO3 | 0.07131 |
| rs728294 | rs728294 | A | 2.587 | 1.82E−03 | GABRB1 | 0.4624 |
| rs16927618 | rs16927618 | G | 0.2683 | 1.84E−03 | PAMR1 | 0.2355 |
| rs16927625 | rs16927625 | G | 0.2683 | 1.89E−03 | PAMR1 | 0.2371 |
| rs2621383 | rs2621383 | C | 0.3288 | 1.93E−03 | HLA-DQB2, HLA-DOB | 0.3567 |
| rs2621384 | rs2621384 | G | 0.3288 | 1.96E−03 | HLA-DQB2, HLA-DOB | 0.3601 |
| rs2621387 | rs2621387 | C | 0.3288 | 1.99E−03 | HLA-DQB2, HLA-DOB | 0.3603 |
| rs2621408 | rs2621408 | G | 0.3288 | 2.00E−03 | HLA-DQB2, HLA-DOB | 0.3529 |
| rs1930952 | imm_6_127275973 | A | 2.676 | 2.00E−03 | MIR588, RSPO3 | 0.4573 |
| rs2027033 | imm_6_127262945 | G | 2.676 | 2.08E−03 | MIR588, RSPO3 | 0.4579 |
| rs4895819 | imm_6_127266989 | A | 2.676 | 2.14E−03 | MIR588, RSPO3 | 0.4575 |
| rs9375478 | imm_6_127274638 | G | 2.676 | 2.14E−03 | MIR588, RSPO3 | 0.4577 |
| rs9388538 | imm_6_127271081 | G | 2.676 | 2.16E−03 | MIR588, RSPO3 | 0.4578 |
| rs6920701 | rs6920701 | G | 0.2797 | 2.18E−03 | MAS1, IGF2R | 0.2233 |
| rs9973057 | 1kg_18_41078925 | G | 3.438 | 2.18E−03 | SLC14A2 | 0.2024 |
| rs10986432 | rs10986432 | G | 0.283 | 2.18E−03 | OLFML2A | 0.1875 |
| rs9444259 | rs9444259 | G | 2.628 | 2.18E−03 | TBX18, NT5E | 0.3339 |
| rs11082436 | 1kg_18_41083040 | G | 3.374 | 2.18E−03 | SLC14A2 | 0.1949 |
| rs7607342 | rs7607342 | A | 2.647 | 2.20E−03 | MIR4431, ASB3 | 0.4733 |
| rs3763341 | rs3763341 | A | 0.2735 | 2.29E−03 | HLA-DOA, HLA-DPA1 | 0.1397 |
| rs3129887 | rs3129887 | A | 5.07 | 2.29E−03 | HLA-DRA | 0.1628 |
| rs11177049 | imm_12_66784143 | G | 0.3644 | 2.33E−03 | IFNG-AS1, IFNG | 0.3964 |
| rs11177050 | imm_12_66784252 | G | 0.3644 | 2.37E−03 | IFNG-AS1, IFNG | 0.3963 |
| rs6478108 | imm_9_116598524 | G | 0.3618 | 2.38E−03 | TNFSF15 | 0.3126 |
| rs2235686 | rs2235686 | A | 0.214 | 2.45E−03 | CBX7 | 0.1383 |
| rs2246638 | rs2246638 | A | 0.2648 | 2.51E−03 | HCG9, ZNRD1-AS1 | 0.2072 |
| rs10438808 | 1kg_17_29642134 | A | 0.276 | 2.51E−03 | CCL11, CCL8 | 0.1883 |
| rs4795903 | 1kg_17_29642880 | A | 0.276 | 2.51E−03 | CCL11, CCL8 | 0.1885 |
| rs4795895 | 1kg_17_29635559 | A | 0.276 | 2.51E−03 | CCL7, CCL11 | 0.1878 |
| rs6505403 | 1kg_17_29627078 | G | 0.276 | 2.51E−03 | CCL7, CCL11 | 0.187 |
| rs201017 | rs201017 | G | 0.3164 | 2.51E−03 | LY86, RREB1 | 0.256 |
| rs2048957 | rs2048957 | A | 2.562 | 2.51E−03 | ARHGAP15 | 0.3606 |
| rs7774158 | rs7774158 | A | 0.389 | 2.51E−03 | HLA-DOA, HLA-DPA1 | 0.4 |
| rs11773945 | rs11773945 | A | 10.46 | 2.51E−03 | LINC00824 | 0.0876 |
| rs16903001 | rs16903001 | A | 10.46 | 2.51E−03 | LINC00824 | 0.08567 |
| rs132001 | rs132001 | A | 3.254 | 2.52E−03 | PHF21B, NUP50-AS1 | 0.1615 |
| rs17518038 | imm_4_123212950 | G | 0.2809 | 2.53E−03 | TRPC3, KIAA1109 | 0.212 |
| rs10256927 | rs10256927 | A | 0.3113 | 2.53E−03 | LOC101928283, GRM8 | 0.2437 |
| rs1512973 | imm_4_123725506 | A | 2.814 | 2.57E−03 | IL2, IL21 | 0.3311 |
| rs2175679 | imm_4_123743075 | A | 2.814 | 2.59E−03 | IL2, IL21 | 0.3311 |
| rs6835457 | imm_4_123730576 | G | 2.814 | 2.59E−03 | IL2, IL21 | 0.3309 |
| rs6819371 | imm_4_123770482 | A | 2.814 | 2.66E−03 | IL21-AS1 | 0.3346 |
| • | rs2280964 | A | 3.966 | 2.68E−03 | CXCR3 | 0.2505 |
| rs2767329 | seq-rs2767329 | A | 0.3017 | 2.71E−03 | CD2, PTGFRN | 0.167 |
| rs1938341 | rs1938341 | A | 0.3919 | 2.71E−03 | PLD5, LINC01347 | 10.46 |
| rs2528691 | rs2528691 | G | 2.784 | 2.74E−03 | IMMP2L, DOCK4 | 0.4921 |
| rs4255613 | imm_12_66784937 | C | 0.3667 | 2.74E−03 | IFNG-AS1, IFNG | 0.4026 |
| rs259942 | 1kg_6_30123146 | A | 0.2518 | 2.76E−03 | ZNRD1-AS1 | 0.1749 |
| rs259942 | rs259942 | A | 0.2518 | 2.76E−03 | ZNRD1-AS1 | 0.1749 |
| rs4771332 | 1kg_13_98868458 | A | 0.3819 | 2.76E−03 | MIR548AN, LINC01232 | 0.2977 |
| rs9388541 | imm_6_127322167 | G | 2.65 | 2.76E−03 | MIR588, RSPO3 | 0.4065 |
| rs987763 | imm_6_127323240 | A | 2.65 | 2.78E−03 | MIR588, RSPO3 | 0.406 |
| rs8081687 | rs8081687 | A | 0.3316 | 2.78E−03 | ABR, BHLHA9 | 0.3198 |
| rs2228225 | imm_12_56145698 | G | 2.817 | 2.78E−03 | GLI1 | 0.3756 |
| rs2292657 | imm_12_56146199 | G | 2.817 | 2.78E−03 | GLI1 | 0.3758 |
| rs3817475 | imm_12_56144681 | A | 2.817 | 2.80E−03 | GLI1 | 0.3752 |
| rs17806015 | imm_12_9796538 | G | 4.586 | 2.80E−03 | CD69 | 0.1699 |
| rs3176793 | imm_12_9801987 | A | 4.586 | 2.80E−03 | CD69 | 0.1695 |
| rs4763299 | imm_12_9795716 | A | 4.586 | 2.80E−03 | CD69 | 0.1698 |
| rs10887816 | imm_10_90168800 | G | 17.218 | 2.80E−03 | RNLS | 0.07587 |
| rs1434254 | rs1434254 | G | 0.3472 | 2.82E−03 | PTPRD | 0.4741 |
| rs3131631 | rs3131631 | G | 0.2548 | 2.87E−03 | MICB, MCCD1 | 0.1989 |
| rs1437950 | rs1437950 | G | 0.3627 | 2.91E−03 | LOC101929231, RND3 | 0.3071 |
| rs331122 | rs331122 | A | 0.3627 | 2.98E−03 | LOC101929231, RND3 | 0.2974 |
| rs859641 | imm_1_170973027 | A | 2.516 | 3.00E−03 | FASLG, TNFSF18 | 0.437 |
| rs13420455 | rs13420455 | A | 0.3122 | 3.05E−03 | FAM136A, TGFA | 0.267 |
| rs7669958 | rs7669958 | A | 2.522 | 3.07E−03 | GABRB1 | 0.3795 |
| rs1900493 | rs1900493 | A | 2.571 | 3.08E−03 | PCDH15, MTRNR2L5 | 0.4954 |
| rs3130637 | rs3130637 | A | 0.3012 | 3.08E−03 | MICB, MCCD1 | 0.2232 |
| rs2621331 | rs2621331 | G | 0.3394 | 3.08E−03 | HLA-DOB | 0.3557 |
| rs9892880 | rs9892880 | A | 0.3093 | 3.10E−03 | NXN | 0.2527 |
| rs2163625 | rs2163625 | G | 2.467 | 3.15E−03 | TMEM9B | 0.4115 |
| rs595158 | rs595158 | A | 2.474 | 3.16E−03 | VPS37C | 0.4987 |
| rs62385693 | imm_5_131801573 | G | 0.2991 | 3.18E−03 | C5orf56 | 0.2068 |
| rs2241392 | rs2241392 | G | 0.357 | 3.18E−03 | C3 | 0.3681 |
| rs1999805 | rs1999805 | G | 2.621 | 3.18E−03 | ESR1 | 0.4465 |
| rs10131232 | rs10131232 | A | 0.314 | 3.18E−03 | GCH1 | 0.2987 |
| rs4317621 | rs4317621 | A | 2.477 | 3.21E−03 | ANK1 | 0.432 |
| rs2245916 | rs2245916 | A | 3.579 | 3.25E−03 | CNTNAP2 | 0.1706 |
| rs1005048 | imm_12_66786506 | A | 0.3764 | 3.29E−03 | IFNG-AS1, IFNG | 0.4023 |
| rs11177053 | imm_12_66785504 | G | 0.3764 | 3.30E−03 | IFNG-AS1, IFNG | 0.4024 |
| rs1558744 | imm_12_66790859 | A | 0.3764 | 3.30E−03 | IFNG-AS1, IFNG | 0.4023 |
| rs2111057 | imm_12_66787546 | C | 0.3764 | 3.33E−03 | IFNG-AS1, IFNG | 0.4024 |
| rs2870955 | imm_12_66788592 | A | 0.3764 | 3.34E−03 | IFNG-AS1, IFNG | 0.4023 |
| rs7133171 | imm_12_66789421 | G | 0.3764 | 3.34E−03 | IFNG-AS1, IFNG | 0.4024 |
| rs7137158 | imm_12_66790187 | G | 0.3764 | 3.43E−03 | IFNG-AS1, IFNG | 0.4023 |
| rs722748 | imm_12_66786791 | A | 0.3764 | 3.43E−03 | IFNG-AS1, IFNG | 0.4024 |
| rs722749 | imm_12_66786905 | G | 0.3764 | 3.43E−03 | IFNG-AS1, IFNG | 0.4024 |
| rs7301797 | imm_12_66789157 | G | 0.3764 | 3.43E−03 | IFNG-AS1, IFNG | 0.4023 |
| rs7306440 | imm_12_66790296 | G | 0.3764 | 3.47E−03 | IFNG-AS1, IFNG | 0.4023 |
| rs2239525 | rs2239525 | G | 0.3178 | 3.56E−03 | ATP6V1G2-DDX39B | 0.235 |
| rs2239526 | rs2239526 | G | 0.3178 | 3.59E−03 | ATP6V1G2-DDX39B | 0.2349 |
| rs2239528 | rs2239528 | A | 0.3178 | 3.64E−03 | DDX39B-AS1 | 0.2349 |
| rs2523504 | rs2523504 | A | 0.3178 | 3.66E−03 | DDX39B-AS1 | 0.235 |
| rs7248930 | rs7248930 | C | 2.644 | 3.70E−03 | BTBD2 | 0.4057 |
| rs11737439 | rs11737439 | A | 0.3234 | 3.70E−03 | MIR1255B1, MIR4801 | 0.2847 |
| rs4573488 | 1kg_1_22610470 | A | 0.1509 | 3.72E−03 | MIR4418, ZBTB40 | 0.1101 |
| rs56411893 | imm_3_48744859 | G | 3.352 | 3.72E−03 | IP6K2, PRKAR2A | 0.157 |
| rs12191230 | rs12191230 | A | 0.3679 | 3.72E−03 | BRD2, HLA-DOA | 0.2748 |
| rs12727925 | rs12727925 | A | 0.1001 | 3.74E−03 | RNF186 | 0.08535 |
| rs17730380 | rs17730380 | A | 0.3596 | 3.77E−03 | PTPN14 | 0.2934 |
| rs212664 | rs212664 | C | 2.92 | 3.77E−03 | HDAC9 | 0.2977 |
| rs7759927 | rs7759927 | C | 2.413 | 3.77E−03 | MEI4, IRAK1BP1 | 0.4074 |
| rs2004317 | imm_2_61711469 | A | 3.886 | 3.82E−03 | XPO1, FAM161A | 0.1733 |
| rs10751118 | seq-rs10751118 | C | 2.575 | 3.82E−03 | KRTAP5-11 | 0.3802 |
| rs1016988 | imm_5_131772473 | G | 0.3145 | 3.82E−03 | SLC22A5, C5orf56 | 0.2064 |
| rs7704457 | imm_5_131772689 | G | 0.3145 | 3.85E−03 | SLC22A5, C5orf56 | 0.2067 |
| rs6833591 | imm_4_123765732 | G | 2.696 | 3.85E−03 | IL21-AS1 | 0.3364 |
| rs496547 | imm_11_118081673 | T | 2.513 | 3.86E−03 | TREH, DDX6 | 0.3581 |
| rs2101598 | rs2101598 | G | 2.439 | 3.87E−03 | LOC101928858, | 0.3626 |
| | | | | | LOC102467655 | |
| rs6872437 | rs6872437 | A | 2.439 | 3.87E−03 | LOC101928858, | 0.3629 |
| | | | | | LOC102467655 | |
| rs1425806 | 1kg_11_34992974 | G | 0.3829 | 3.95E−03 | PDHX, LOC100507144 | 0.3017 |
| rs1922240 | rs1922240 | G | 2.64 | 3.96E−03 | ABCB1 | 0.3309 |
| rs1860598 | rs1860598 | G | 2.612 | 4.00E−03 | FAM184B | 0.4222 |
| rs7559601 | rs7559601 | A | 2.484 | 4.04E−03 | LINC01317 | 0.3652 |
| rs859623 | imm_1_170949524 | A | 2.507 | 4.11E−03 | FASLG, TNFSF18 | 0.4316 |
| rs859673 | imm_1_170947088 | A | 2.507 | 4.13E−03 | FASLG, TNFSF18 | 0.4321 |
| rs57857640 | imm_4_123203360 | G | 0.3768 | 4.16E−03 | TRPC3, KIAA1109 | 0.4077 |
| rs12454802 | rs12454802 | G | 2.574 | 4.16E−03 | NETO1 | 0.4085 |
| rs704847 | imm_1_170995554 | C | 2.362 | 4.17E−03 | FASLG, TNFSF18 | 0.3957 |
| rs1996077 | imm_4_123729236 | A | 2.668 | 4.22E−03 | IL2, IL21 | 0.3343 |
| rs12465492 | rs12465492 | A | 2.379 | 4.23E−03 | ARHGAP15 | 0.365 |
| rs1051336 | rs1051336 | A | 4.976 | 4.29E−03 | HLA-DRA | 0.1581 |
| rs1041885 | rs1041885 | A | 4.976 | 4.32E−03 | HLA-DRA | 0.1581 |
| rs2239805 | rs2239805 | C | 4.976 | 4.34E−03 | HLA-DRA | 0.1563 |
| rs2239806 | rs2239806 | A | 4.976 | 4.34E−03 | HLA-DRA | 0.1581 |
| rs78664442 | imm_3_161187500 | A | 0.113 | 4.35E−03 | IL12A-AS1 | 0.0612 |
| rs196595 | rs196595 | G | 0.3776 | 4.35E−03 | EEPD1 | 0.3425 |
| rs196600 | rs196600 | G | 0.3776 | 4.35E−03 | EEPD1 | 0.3419 |
| rs74298291 | imm_6_106730592 | A | 0.1514 | 4.35E−03 | PRDM1, ATG5 | 0.1109 |
| rs9486298 | imm_6_106725478 | A | 0.1514 | 4.35E−03 | PRDM1, ATG5 | 0.1109 |
| rs2316184 | rs2316184 | G | 0.2828 | 4.35E−03 | CDYL2 | 0.2381 |
| rs10876986 | imm_12_56142934 | G | 2.8 | 4.35E−03 | GLI1 | 0.3706 |
| rs3825077 | imm_12_56142281 | G | 2.8 | 4.35E−03 | GLI1 | 0.3709 |
| rs7024944 | imm_9_4301574 | G | 0.37 | 4.35E−03 | GLIS3, SLC1A1 | 0.2811 |
| rs2700982 | 1kg_7_37361345 | G | 2.445 | 4.35E−03 | ELMO1 | 0.4571 |
| rs2700983 | 1kg_7_37360904 | C | 2.445 | 4.35E−03 | ELMO1 | 0.4571 |
| rs2277315 | imm_12_56155849 | A | 2.697 | 4.36E−03 | ARHGAP9 | 0.3106 |
| rs2277318 | imm_12_56155714 | A | 2.697 | 4.36E−03 | ARHGAP9 | 0.3105 |
| rs10783828 | imm_12_56147751 | A | 2.697 | 4.36E−03 | GLI1 | 0.31 |
| rs4760259 | imm_12_56147093 | A | 2.697 | 4.36E−03 | GLI1 | 0.3099 |
| rs1529028 | rs1529028 | A | 0.1638 | 4.36E−03 | GBE1, NONE | 0.1035 |
| rs1570452 | 1kg_13_98867496 | G | 0.4133 | 4.37E−03 | MIR548AN, LINC01232 | 0.3054 |
| rs16899792 | imm_6_167353485 | G | 5.561 | 4.42E−03 | FGFR1OP | 0.06949 |
| rs431159 | imm_6_167329832 | A | 5.561 | 4.54E−03 | RNASET2, MIR3939 | 0.07032 |
| rs10946197 | imm_6_167268406 | A | 0.3643 | 4.55E−03 | RNASET2 | 0.2716 |
| rs7370700 | imm_2_185898466 | A | 2.76 | 4.59E−03 | ZNF804A, LOC101927196 | 0.2517 |
| rs2245545 | rs2245545 | C | 2.502 | 4.60E−03 | BMS1P21, SFTPD | 0.4648 |
| rs1343658 | imm_6_127304968 | G | 2.393 | 4.67E−03 | MIR588, RSPO3 | 0.4628 |
| rs6907995 | imm_6_127304781 | G | 2.393 | 4.67E−03 | MIR588, RSPO3 | 0.4624 |
| rs9385412 | imm_6_127303760 | G | 2.393 | 4.72E−03 | MIR588, RSPO3 | 0.4625 |
| rs6006421 | imm_22_28985692 | A | 3.447 | 4.83E−03 | LIF, OSM | 0.1798 |
| rs62011167 | imm_15_77049780 | G | 0.2604 | 4.84E−03 | RASGRF1 | 0.1746 |
| rs504215 | imm_19_53964296 | A | 2.834 | 4.84E−03 | FGF21, BCAT2 | 0.3304 |
| rs4919234 | rs4919234 | A | 2.487 | 4.85E−03 | HPSE2 | 0.3027 |
| rs7030473 | rs7030473 | A | 2.509 | 4.87E−03 | RGS3, ZNF618 | 0.3209 |
| rs10878749 | imm_12_66793406 | T | 0.3848 | 4.87E−03 | IFNG-AS1, IFNG | 0.401 |
| rs11177059 | imm_12_66793735 | A | 0.3848 | 4.87E−03 | IFNG-AS1, IFNG | 0.4003 |
| rs10114470 | imm_9_116587593 | A | 0.3837 | 4.89E−03 | TNFSF15 | 0.302 |
| 50404601310A0 | 5-40460131-A- | D | 3.777 | 4.92E−03 | LOC285634_LOC100127944 | 0.1298 |
| DELETION | DELETION | | | | | |
| rs140935661 | imm_5_40408209 | A | 3.777 | 4.97E−03 | LINC00603, PTGER4 | 0.1273 |
| rs10512737 | imm_5_40445800 | A | 3.777 | 4.98E−03 | LINC00603, PTGER4 | 0.1298 |
| rs1124233 | imm_5_40425044 | A | 3.777 | 5.00E−03 | LINC00603, PTGER4 | 0.1272 |
| rs11739261 | imm_5_40446496 | A | 3.777 | 5.00E−03 | LINC00603, PTGER4 | 0.1298 |
| rs11739725 | imm_5_40459216 | G | 3.777 | 5.04E−03 | LINC00603, PTGER4 | 0.1299 |
| rs11749040 | imm_5_40432182 | A | 3.777 | 5.06E−03 | LINC00603, PTGER4 | 0.1271 |
| rs12187530 | imm_5_40425609 | A | 3.777 | 5.16E−03 | LINC00603, PTGER4 | 0.1271 |
| rs1373693 | imm_5_40466932 | G | 3.777 | 5.17E−03 | LINC00603, PTGER4 | 0.1299 |
| rs1373694 | imm_5_40438950 | A | 3.777 | 5.17E−03 | LINC00603, PTGER4 | 0.1271 |
| rs17227583 | imm_5_40413623 | G | 3.777 | 5.23E−03 | LINC00603, PTGER4 | 0.1273 |
| rs17234657 | imm_5_40437266 | C | 3.777 | 5.23E−03 | LINC00603, PTGER4 | 0.1271 |
| rs17235132 | imm_5_40448114 | G | 3.777 | 5.23E−03 | LINC00603, PTGER4 | 0.1299 |
| rs17826145 | imm_5_40433947 | A | 3.777 | 5.23E−03 | LINC00603, PTGER4 | 0.127 |
| rs2371685 | imm_5_40427983 | T | 3.777 | 5.25E−03 | LINC00603, PTGER4 | 0.1271 |
| rs4613763 | imm_5_40428485 | G | 3.777 | 5.27E−03 | LINC00603, PTGER4 | 0.1271 |
| rs55782190 | imm_5_40449187 | G | 3.777 | 5.27E−03 | LINC00603, PTGER4 | 0.1299 |
| rs56244034 | imm_5_40411916 | A | 3.777 | 5.29E−03 | LINC00603, PTGER4 | 0.1272 |
| rs56309786 | imm_5_40468984 | A | 3.777 | 5.29E−03 | LINC00603, PTGER4 | 0.1298 |
| rs6879283 | imm_5_40437990 | G | 3.777 | 5.30E−03 | LINC00603, PTGER4 | 0.1271 |
| rs6883975 | imm_5_40438434 | A | 3.777 | 5.32E−03 | LINC00603, PTGER4 | 0.1271 |
| rs6889364 | imm_5_40383226 | A | 3.777 | 5.32E−03 | LINC00603, PTGER4 | 0.1274 |
| rs73090828 | imm_5_40473854 | A | 3.777 | 5.36E−03 | LINC00603, PTGER4 | 0.1299 |
| rs73099728 | imm_5_40368755 | G | 3.777 | 5.36E−03 | LINC00603, PTGER4 | 0.1275 |
| rs73099741 | imm_5_40382448 | A | 3.777 | 5.36E−03 | LINC00603, PTGER4 | 0.1274 |
| rs7734434 | imm_5_40472455 | A | 3.777 | 5.38E−03 | LINC00603, PTGER4 | 0.1297 |
| rs895123 | imm_5_40419818 | G | 3.777 | 5.38E−03 | LINC00603, PTGER4 | 0.1272 |
| rs2187685 | rs2187685 | A | 0.3808 | 5.38E−03 | HLA-DQB2, HLA-DOB | 0.4072 |
| rs2621377 | rs2621377 | G | 0.3808 | 5.38E−03 | HLA-DQB2, HLA-DOB | 0.4072 |
| rs2621379 | rs2621379 | G | 0.3808 | 5.38E−03 | HLA-DQB2, HLA-DOB | 0.4072 |
| rs4796221 | rs4796221 | A | 0.3924 | 5.40E−03 | TBC1D3B, ZNHIT3 | 0.4476 |
| rs62578666 | imm_9_116561068 | A | 6.038 | 5.41E−03 | LOC100505478, TNFSF15 | 0.08274 |
| rs11870190 | rs11870190 | G | 0.3315 | 5.41E−03 | NXN | 0.2383 |
| rs225100 | imm_1_7989501 | A | 2.366 | 5.49E−03 | PARK7, ERRFI1 | 0.432 |
| rs10928195 | rs10928195 | C | 3.984 | 5.51E−03 | ARHGAP15 | 0.1343 |
| rs1863270 | rs1863270 | C | 2.471 | 5.55E−03 | RORA | 0.3048 |
| rs10055349 | imm_5_40477475 | A | 2.83 | 5.61E−03 | LINC00603, PTGER4 | 0.2207 |
| rs1445002 | imm_5_40355634 | A | 4.05 | 5.61E−03 | LINC00603, PTGER4 | 0.1243 |
| rs1056567 | imm_9_122671866 | A | 2.662 | 5.61E−03 | PHF19 | 0.3041 |
| rs3933326 | imm_9_122673769 | A | 2.662 | 5.64E−03 | PHF19 | 0.3046 |
| rs4836833 | imm_9_122672650 | G | 2.662 | 5.67E−03 | PHF19 | 0.3059 |
| rs616340 | rs616340 | A | 2.453 | 5.68E−03 | CD5 | 0.3743 |
| rs11004384 | rs11004384 | C | 2.926 | 5.69E−03 | PCDH15 | 0.2936 |
| rs7210639 | 1kg_17_29612741 | G | 0.2957 | 5.70E−03 | CCL2, CCL7 | 0.1923 |
| rs11739622 | imm_5_131897867 | A | 2.915 | 5.72E−03 | IRF1, IL5 | 0.1899 |
| rs1848186 | imm_1_25155443 | C | 0.4061 | 5.72E−03 | RUNX3 | 0.3846 |
| rs10516615 | imm_4_123194057 | G | 3.922 | 5.73E−03 | TRPC3, KIAA1109 | 0.1309 |
| rs6932387 | rs6932387 | A | 2.325 | 5.75E−03 | MIR7641-2, KU-MEL-3 | 0.3134 |
| rs9353048 | rs9353048 | C | 0.4201 | 5.75E−03 | LINC01526, IBTK | 0.3876 |
| rs11664603 | 1kg_18_41082724 | G | 2.9 | 5.75E−03 | SLC14A2 | 0.2203 |
| rs72772074 | imm_5_96024270 | G | 3.503 | 5.75E−03 | CAST | 0.1342 |
| rs8191663 | rs8191663 | A | 2.626 | 5.75E−03 | NEIL2 | 0.2424 |
| rs225119 | imm_1_7966948 | A | 2.35 | 5.75E−03 | PARK7 | 0.4294 |
| rs7305123 | rs7305123 | G | 0.3264 | 5.75E−03 | LOC100507195, RAP1B | 0.1948 |
| rs2078610 | rs2078610 | C | 2.363 | 5.75E−03 | GABRB1 | 0.4154 |
| rs2426741 | rs2426741 | A | 2.88 | 5.75E−03 | RBM38, CTCFL | 0.2478 |
| rs637174 | imm_19_53958748 | A | 2.778 | 5.75E−03 | FGF21, BCAT2 | 0.3205 |
| rs1024610 | 1kg_17_29604344 | A | 0.3136 | 5.75E−03 | LOC101927239, CCL2 | 0.1982 |
| rs1983608 | rs1983608 | G | 0.3258 | 5.75E−03 | PRDM2, KAZN | 0.3428 |
| rs10174088 | rs10174088 | G | 2.302 | 5.75E−03 | YY1P2, LRP1B | 0.4751 |
| rs13281279 | rs13281279 | A | 2.537 | 5.75E−03 | LINC00824 | 0.4161 |
| rs9807677 | 1kg_18_41082119 | A | 3.065 | 5.75E−03 | SLC14A2 | 0.1927 |
| rs9466072 | rs9466072 | C | 2.618 | 5.75E−03 | CDKAL1, LINC00581 | 0.395 |
| rs164732 | rs164732 | A | 0.4004 | 5.75E−03 | KYNU | 0.4581 |
| rs7839434 | imm_8_11363051 | G | 2.855 | 5.75E−03 | FAM167A, BLK | 0.2173 |
| rs12468414 | rs12468414 | G | 0.2938 | 5.75E−03 | XPO1, FAM161A | 0.1925 |
| rs898892 | rs898892 | C | 2.531 | 5.75E−03 | HPSE2 | 0.294 |
| rs12152961 | rs12152961 | A | 2.851 | 5.75E−03 | LINC01470, GRIA1 | 0.2004 |
| rs67946532 | seq-t1d-19-59704204-T-C | G | 2.43 | 5.75E−03 | CDC42EP5, LAIR2 | 0.4368 |
| rs2239186 | rs2239186 | G | 0.2909 | 5.75E−03 | VDR | 0.1897 |
| rs12908584 | rs12908584 | C | 2.282 | 5.75E−03 | LINC01584 | 0.3552 |
| rs17659542 | rs17659542 | A | 0.2584 | 5.75E−03 | TRPS1 | 0.1445 |
| rs2383135 | rs2383135 | C | 0.3038 | 5.75E−03 | SLC24A2, MLLT3 | 0.2111 |
| rs2122001 | 1kg_5_173230976 | G | 5.281 | 5.75E−03 | LINC01485, CPEB4 | 0.08196 |
| rs1539234 | rs1539234 | G | 0.4033 | 5.76E−03 | PFKFB3 | 0.3984 |
| rs4945744 | imm_6_106720616 | A | 0.3312 | 5.76E−03 | PRDM1, ATG5 | 0.2513 |
| rs4946730 | imm_6_106719784 | A | 0.3312 | 5.76E−03 | PRDM1, ATG5 | 0.2535 |
| rs4946731 | imm_6_106720617 | C | 0.3312 | 5.76E−03 | PRDM1, ATG5 | 0.2513 |
| rs7748394 | imm_6_106732576 | G | 0.3312 | 5.76E−03 | PRDM1, ATG5 | 0.2538 |
| rs11679301 | imm_2_185855392 | G | 2.731 | 5.76E−03 | ZNF804A, LOC 101927196 | 0.2495 |
| rs17712328 | imm_2_185817565 | A | 2.731 | 5.79E−03 | ZNF804A, LOC101927196 | 0.2488 |
| rs2194476 | imm_2_185811060 | A | 2.731 | 5.80E−03 | ZNF804A, LOC101927196 | 0.2508 |
| rs62200005 | imm_2_185836565 | G | 2.731 | 5.81E−03 | ZNF804A, LOC101927196 | 0.2492 |
| rs10283808 | imm_9_34932073 | A | 3.105 | 5.81E−03 | FAM205C, PHF24 | 0.1917 |
| rs12002089 | imm_9_34917690 | A | 3.105 | 5.85E−03 | FAM205C, PHF24 | 0.1964 |
| rs6476470 | imm_9_34919071 | G | 3.105 | 5.91E−03 | FAM205C, PHF24 | 0.1964 |
| rs7033016 | imm_9_34901879 | G | 3.105 | 5.91E−03 | FAM205C, PHF24 | 0.1978 |
| rs7040756 | imm_9_34919667 | T | 3.105 | 5.91E−03 | FAM205C, PHF24 | 0.1964 |
| rs7041922 | imm_9_34928198 | G | 3.105 | 5.92E−03 | FAM205C, PHF24 | 0.1958 |
| rs73495567 | imm_9_34920450 | G | 3.105 | 5.95E−03 | FAM205C, PHF24 | 0.1963 |
| rs5766248 | rs5766248 | A | 2.74 | 5.95E−03 | PHF21B | 0.1716 |
| rs172811 | imm_1_7962536 | A | 2.269 | 5.96E−03 | PARK7 | 0.4275 |
| rs225092 | imm_1_7958662 | G | 2.269 | 6.01E−03 | PARK7 | 0.4282 |
| rs226242 | imm_1_7956055 | G | 2.269 | 6.07E−03 | PARK7 | 0.4283 |
| rs1014054 | imm_6_127353340 | G | 2.381 | 6.08E−03 | MIR588, RSPO3 | 0.4604 |
| rs12176348 | imm_6_127373235 | G | 2.381 | 6.08E−03 | MIR588, RSPO3 | 0.4614 |
| rs17572870 | imm_6_127366607 | A | 2.381 | 6.08E−03 | MIR588, RSPO3 | 0.4499 |
| rs1930941 | imm_6_127358536 | A | 2.381 | 6.14E−03 | MIR588, RSPO3 | 0.4489 |
| rs34303228 | imm_6_127364411 | G | 2.381 | 6.18E−03 | MIR588, RSPO3 | 0.4609 |
| rs4897200 | imm_6_127369187 | A | 2.381 | 6.20E−03 | MIR588, RSPO3 | 0.4609 |
| rs6913010 | imm_6_127369036 | G | 2.381 | 6.23E−03 | MIR588, RSPO3 | 10.45 |
| rs6929547 | imm_6_127354066 | A | 2.381 | 6.25E−03 | MIR588, RSPO3 | 0.4491 |
| rs7756698 | imm_6_127357269 | G | 2.381 | 6.27E−03 | MIR588, RSPO3 | 0.4607 |
| rs9388543 | imm_6_127369989 | G | 2.381 | 6.28E−03 | MIR588, RSPO3 | 0.4507 |
| rs9401934 | imm_6_127371660 | A | 2.381 | 6.32E−03 | MIR588, RSPO3 | 0.4505 |
| rs997112 | imm_6_127363178 | G | 2.381 | 6.34E−03 | MIR588, RSPO3 | 0.4499 |
| rs17537576 | rs17537576 | C | 0.364 | 6.44E−03 | SORBS1 | 0.1587 |
| rs45515895 | imm_4_123404277 | A | 0.3404 | 6.44E−03 | KIAA1109 | 0.2108 |
| rs72687036 | imm_4_123377591 | G | 0.3404 | 6.46E−03 | KIAA1109 | 0.211 |
| rs11702189 | imm_21_44511885 | A | 0.324 | 6.49E−03 | DNMT3L, AIRE | 0.2265 |
| rs2143461 | rs2143461 | A | 5.17 | 6.49E−03 | C6orf10 | 0.1495 |
| rs3129924 | rs3129924 | A | 5.17 | 6.56E−03 | C6orf10 | 0.1495 |
| rs3129939 | rs3129939 | G | 5.17 | 6.56E−03 | C6orf10 | 0.1495 |
| rs10993 | imm_19_43435431 | A | 0.1181 | 6.58E−03 | PPP1R14A | 0.04589 |
| rs17762453 | imm_2_185776058 | G | 2.742 | 6.62E−03 | ZNF804A, LOC101927196 | 0.2476 |
| rs2824115 | rs2824115 | A | 2.534 | 6.63E−03 | MIR99AHG, LINC01549 | 0.3067 |
| rs12361165 | rs12361165 | C | 8.517 | 6.70E−03 | STK33 | 0.09326 |
| rs9933766 | rs9933766 | A | 3.808 | 6.72E−03 | MIR5093, GSE1 | 0.1478 |
| rs61818748 | vh_1_156635016 | A | 0.1163 | 6.73E−03 | OR10T2 | 0.08112 |
| rs4489574 | rs4489574 | A | 0.3742 | 6.73E−03 | FCER1G | 0.3294 |
| rs149598 | imm_5_96195447 | A | 0.1939 | 6.73E−03 | ERAP1, ERAP2 | 0.07408 |
| rs249959 | imm_5_96190602 | A | 0.1939 | 6.83E−03 | ERAP1, ERAP2 | 0.07402 |
| rs34733 | imm_5_96187950 | A | 0.1939 | 6.86E−03 | ERAP1, ERAP2 | 0.07413 |
| rs34734 | imm_5_96191025 | A | 0.1939 | 6.86E−03 | ERAP1, ERAP2 | 0.07414 |
| rs34736 | imm_5_96193646 | A | 0.1939 | 6.86E−03 | ERAP1, ERAP2 | 0.07408 |
| rs647031 | imm_5_96184512 | A | 0.1939 | 6.86E−03 | ERAP1, ERAP2 | 0.07418 |
| 10061600650G0 | 10-6160065-G- | I | 6.077 | 6.88E−03 | IL2RA_RBM17 | 0.09007 |
| DELETION | DELETION | | | | | |
| rs4246905 | imm_9_116593070 | A | 0.3799 | 6.88E−03 | TNFSF15 | 0.2752 |
| rs2788478 | rs2788478 | G | 2.361 | 6.88E−03 | WDR60 | 0.3595 |
| rs76295456 | imm_2_204238769 | A | 4.538 | 6.88E−03 | RAPH1, CD28 | 0.09078 |
| rs213230 | rs213230 | G | 0.3982 | 6.93E−03 | ZKSCAN3 | 0.2742 |
| rs10468612 | rs10468612 | A | 2.525 | 6.93E−03 | MRM1, LHX1 | 0.3348 |
| rs1044193 | vh_9_137971388 | G | 3.807 | 6.93E−03 | UBAC1 | 0.125 |
| rs11129012 | rs11129012 | A | 0.3324 | 6.93E−03 | ZNF385D | 0.2245 |
| rs1931737 | rs1931737 | A | 0.3583 | 6.93E−03 | DOCK1, NPS | 0.3416 |
| rs1444291 | rs1444291 | G | 2.806 | 6.93E−03 | LINC01584 | 0.2576 |
| rs17444900 | rs17444900 | G | 3.669 | 6.93E−03 | LIMCH1, PHOX2B | 0.1318 |
| rs10872310 | imm_6_127318574 | A | 2.332 | 6.94E−03 | MIR588, RSPO3 | 0.4627 |
| rs13204542 | imm_6_127324441 | A | 2.332 | 6.97E−03 | MIR588, RSPO3 | 0.4628 |
| rs17572416 | imm_6_127332487 | G | 2.332 | 6.97E−03 | MIR588, RSPO3 | 0.4517 |
| rs1930940 | imm_6_127345598 | A | 2.332 | 6.97E−03 | MIR588, RSPO3 | 0.4516 |
| rs1930958 | imm_6_127326261 | A | 2.332 | 6.99E−03 | MIR588, RSPO3 | 0.4629 |
| rs1930959 | imm_6_127326298 | G | 2.332 | 6.99E−03 | MIR588, RSPO3 | 0.4517 |
| rs4897197 | imm_6_127325477 | A | 2.332 | 6.99E−03 | MIR588, RSPO3 | 0.4512 |
| rs4897198 | imm_6_127330782 | A | 2.332 | 6.99E−03 | MIR588, RSPO3 | 0.4517 |
| rs4897199 | imm_6_127330861 | A | 2.332 | 6.99E−03 | MIR588, RSPO3 | 0.4516 |
| rs6906261 | imm_6_127316974 | A | 2.332 | 6.99E−03 | MIR588, RSPO3 | 0.4626 |
| rs7751138 | imm_6_127328488 | A | 2.332 | 6.99E−03 | MIR588, RSPO3 | 0.4519 |
| rs9372855 | imm_6_127336190 | G | 2.332 | 6.99E−03 | MIR588, RSPO3 | 0.4517 |
| rs9398828 | imm_6_127336335 | G | 2.332 | 6.99E−03 | MIR588, RSPO3 | 0.4523 |
| rs9401924 | imm_6_127327787 | C | 2.332 | 6.99E−03 | MIR588, RSPO3 | 0.463 |
| rs9401929 | imm_6_127342073 | A | 2.332 | 6.99E−03 | MIR 588, RSPO3 | 0.4624 |
| rs193807 | rs193807 | A | 0.403 | 6.99E−03 | CDHR3 | 0.4847 |
| rs11931074 | rs11931074 | A | 7.994 | 7.01E−03 | GPRIN3, SNCA | 0.08535 |
| rs61032876 | chr4:90860426 | A | 7.994 | 7.09E−03 | GPRIN3, SNCA | 0.08535 |
| rs7681312 | rs7681312 | G | 7.994 | 7.09E−03 | GPRIN3, SNCA | 0.0854 |
| rs7681815 | rs7681815 | G | 7.994 | 7.17E−03 | GPRIN3, SNCA | 0.08537 |
| rs3822086 | rs3822086 | A | 7.994 | 7.19E−03 | SNCA | 0.08504 |
| rs3857059 | rs3857059 | G | 7.994 | 7.19E−03 | SNCA | 0.08479 |
| rs17624462 | rs17624462 | G | 5.096 | 7.19E−03 | ITGBL1 | 0.1023 |
| rs7726182 | imm_5_35850767 | C | 0.1305 | 7.22E−03 | SPEF2 | 0.09748 |
| rs1483242 | imm_2_185752872 | A | 2.197 | 7.23E−03 | ZNF804A, LOC101927196 | 0.4653 |
| rs62199977 | imm_2_185763910 | G | 2.197 | 7.23E−03 | ZNF804A, LOC101927196 | 0.4666 |
| rs6724681 | imm_2_185758211 | A | 2.197 | 7.23E−03 | ZNF804A, LOC101927196 | 0.465 |
| rs6872249 | rs6872249 | A | 2.292 | 7.25E−03 | LOC101928858, | 0.3619 |
| | | | | | LOC102467655 | |
| rs4823779 | rs4823779 | G | 0.2722 | 7.28E−03 | MIR3201, FAM19A5 | 0.112 |
| rs4823780 | rs4823780 | A | 0.2722 | 7.31E−03 | MIR3201, FAM19A5 | 0.1123 |
| rs45610037 | imm_4_123622458 | A | 0.3507 | 7.33E−03 | IL2, IL21 | 0.2199 |
| rs10903116 | imm_1_25155749 | G | 0.4143 | 7.33E−03 | RUNX3 | 0.3825 |
| rs10903117 | imm_1_25156179 | G | 0.4143 | 7.33E−03 | RUNX3 | 0.3825 |
| rs11249207 | imm_1_25155656 | G | 0.4143 | 7.33E−03 | RUNX3 | 0.3822 |
| rs11580845 | imm_1_25155943 | C | 0.4143 | 7.33E−03 | RUNX3 | 0.3823 |
| rs12031692 | imm_1_25155861 | A | 0.4143 | 7.33E−03 | RUNX3 | 0.382 |
| rs4288539 | imm_1_25155580 | G | 0.4143 | 7.34E−03 | RUNX3 | 0.3825 |
| rs6600245 | imm_1_25157265 | A | 0.4143 | 7.35E−03 | RUNX3 | 0.3809 |
| rs8076157 | rs8076157 | A | 0.4309 | 7.37E−03 | CYB561, ACE | 0.3006 |
| rs2241393 | rs2241393 | G | 0.42 | 7.38E−03 | C3 | 0.3737 |
| rs1015976 | rs1015976 | A | 2.356 | 7.43E−03 | MAN2A1 | 0.3583 |
| rs16897813 | rs16897813 | G | 4.639 | 7.50E−03 | ZHX2 | 0.1046 |
| rs17086609 | rs17086609 | G | 2.292 | 7.51E−03 | FLT1 | 0.3457 |
| rs7618618 | imm_3_45938501 | C | 0.3438 | 7.53E−03 | FYCO1 | 0.2331 |
| rs17806523 | imm_8_11443584 | A | 3.233 | 7.57E−03 | BLK | 0.1815 |
| rs35354254 | imm_6_127303201 | G | 2.322 | 7.66E−03 | MIR588, RSPO3 | 0.452 |
| rs9398824 | imm_6_127304193 | A | 2.322 | 7.67E−03 | MIR588, RSPO3 | 0.4509 |
| rs4648888 | imm_1_25158738 | G | 0.4151 | 7.68E−03 | RUNX3 | 0.3864 |
| rs768257 | rs768257 | A | 2.529 | 7.68E−03 | RBM19, TBX5 | 0.4466 |
| rs57770060 | seq-rs57770060 | A | 2.303 | 7.68E−03 | PFKFB3 | 0.4166 |
| rs12607033 | rs12607033 | C | 2.556 | 7.68E−03 | VAPA, LINC01254 | 0.3631 |
| rs3782125 | imm_12_56143267 | G | 2.629 | 7.68E−03 | GLI1 | 0.3627 |
| rs228651 | imm_1_7833686 | A | 2.284 | 7.68E−03 | UTS2 | 0.3905 |
| rs12342902 | rs12342902 | A | 3.878 | 7.68E−03 | NTRK2 | 0.1383 |
| rs1915279 | rs1915279 | A | 0.3427 | 7.68E−03 | LINC01139, CHRM3 | 0.2272 |
| rs2113378 | 1kg_2_207039068 | G | 0.2984 | 7.68E−03 | ADAM23 | 0.1687 |
| rs12474299 | rs12474299 | C | 0.3257 | 7.68E−03 | LINC01107, TWIST2 | 0.1619 |
| rs4567718 | rs4567718 | G | 0.3828 | 7.68E−03 | MIR8065, RBFOX1 | 0.2786 |
| rs3115962 | imm_2_204264951 | A | 4.497 | 7.68E−03 | RAPH1, CD28 | 0.0913 |
| rs3115968 | imm_2_204245415 | G | 4.497 | 7.68E−03 | RAPH1, CD28 | 0.09125 |
| rs3116498 | imm_2_204246959 | A | 4.497 | 7.68E−03 | RAPH1, CD28 | 0.09126 |
| rs11098666 | imm_4_123727364 | A | 0.3966 | 7.68E−03 | IL2, IL21 | 0.3113 |
| rs6814458 | rs6814458 | G | 0.3966 | 7.69E−03 | IL2, IL21 | 0.3095 |
| rs6829845 | imm_4_123730216 | A | 0.3966 | 7.70E−03 | IL2, IL21 | 0.3115 |
| rs75039958 | imm_4_123727623 | G | 0.3966 | 7.70E−03 | IL2, IL21 | 0.3117 |
| rs7676523 | imm_4_123742729 | G | 0.3966 | 7.70E−03 | IL2, IL21 | 0.3093 |
| rs7676741 | imm_4_123742891 | G | 0.3966 | 7.70E−03 | IL2, IL21 | 0.3094 |
| rs78863329 | imm_4_123727620 | A | 0.3966 | 7.70E−03 | IL2, IL21 | 0.3117 |
| rs2221903 | imm_4_123758362 | G | 0.3966 | 7.70E−03 | IL21 | 0.3076 |
| rs4833837 | imm_4_123756413 | G | 0.3966 | 7.74E−03 | IL21 | 0.3074 |
| rs1353280 | rs1353280 | G | 0.3698 | 7.75E−03 | UGT2B28, UGT2B4 | 0.3291 |
| rs1439876 | rs1439876 | A | 0.4074 | 7.76E−03 | KYNU | 0.4486 |
| rs1181390 | imm_2_204280922 | A | 2.393 | 7.76E−03 | CD28 | 0.2142 |
| rs6441996 | imm_3_46480270 | G | 0.3706 | 7.76E−03 | LTF | 0.2595 |
| rs7175099 | rs7175099 | A | 2.546 | 7.77E−03 | LOC101927286 | 0.3224 |
| rs1877536 | rs1877536 | G | 0.2019 | 7.78E−03 | TMEM192, KLHL2 | 0.1254 |
| rs9653015 | 1kg_18_41075160 | A | 2.777 | 7.78E−03 | SLC14A2 | 0.1932 |
| rs1800629 | rs1800629 | A | 4.615 | 7.78E−03 | TNF; LTA | 0.1558 |
| rs10918931 | rs10918931 | A | 2.276 | 7.79E−03 | XCL1, DPT | 0.3795 |
| rs2723980 | 1kg_7_37331947 | A | 2.694 | 7.79E−03 | ELMO1 | 0.1898 |
| rs3870336 | imm_3_49532861 | A | 4.692 | 7.79E−03 | DAG1 | 0.0834 |
| rs6862868 | rs6862868 | A | 0.3749 | 7.79E−03 | WWC1 | 0.4007 |
| rs1444300 | rs1444300 | A | 2.538 | 7.79E−03 | LINC01584 | 0.2677 |
| rs1845931 | rs1845931 | A | 2.538 | 7.79E−03 | LINC01584 | 0.2688 |
| rs10518402 | seq-rs10518402 | G | 3.227 | 7.79E−03 | IL21-AS1 | 0.1839 |
| rs6840978 | imm_4_123774157 | A | 3.227 | 7.79E−03 | IL21-AS1 | 0.1835 |
| rs6936620 | rs6936620 | A | 2.732 | 7.80E−03 | HLA-DOA, HLA-DPA1 | 0.3609 |
| rs226249 | imm_1_7944365 | A | 2.236 | 7.80E−03 | PARK7 | 0.4334 |
| rs226251 | imm_1_7947277 | A | 2.236 | 7.81E−03 | PARK7 | 0.4337 |
| rs226253 | imm_1_7950293 | A | 2.236 | 7.84E−03 | PARK7 | 0.4339 |
| rs6835929 | rs6835929 | G | 5.032 | 7.84E−03 | ELOVL6 | 0.1291 |
| rs1102707 | imm_1_170966032 | G | 2.29 | 7.87E−03 | FASLG, TNFSF18 | 0.4535 |
| rs859633 | imm_1_170979047 | G | 2.29 | 7.90E−03 | FASLG, TNFSF18 | 0.4538 |
| rs859634 | imm_1_170978848 | G | 2.29 | 7.90E−03 | FASLG, TNFSF18 | 0.455 |
| rs859637 | imm_1_170977623 | A | 2.29 | 7.93E−03 | FASLG, TNFSF18 | 0.453 |
| rs859639 | imm_1_170976789 | A | 2.29 | 7.94E−03 | FASLG, TNFSF18 | 0.4551 |
| rs4908678 | imm_1_7661837 | G | 2.23 | 7.97E−03 | CAMTA1 | 0.3648 |
| rs7708673 | rs7708673 | G | 2.394 | 7.98E−03 | LOC101928858, | 0.25 |
| | | | | | LOC102467655 | |
| rs11624462 | 1kg_14_34741437 | C | 2.863 | 8.00E−03 | KIAA0391 | 0.2311 |
| rs61989546 | 1kg_14_34754403 | G | 2.863 | 8.04E−03 | KIAA0391 | 0.2358 |
| rs61989547 | 1kg_14_34761831 | G | 2.863 | 8.06E−03 | KIAA0391 | 0.2344 |
| rs6562463 | rs6562463 | T | 0.4172 | 8.07E−03 | PCDH9 | 0.4401 |
| rs59366011 | 1kg_2_206983501 | A | 0.3829 | 8.08E−03 | ZDBF2, ADAM23 | 0.2494 |
| rs7180888 | 15_95102199 | A | 0.4583 | 8.11E−03 | NR2F2, SPATA8-AS1 | 0.4605 |
| rs1607785 | rs1607785 | G | 0.4148 | 8.13E−03 | E2F7, NAV3 | 0.3327 |
| rs6684369 | rs6684369 | G | 4.087 | 8.15E−03 | PLXNA2, MIR205HG | 0.1467 |
| rs10040272 | imm_5_131872479 | G | 0.3159 | 8.15E−03 | IRF1, IL5 | 0.1819 |
| rs17690122 | imm_5_131895734 | G | 0.3159 | 8.15E−03 | IRF1, IL5 | 0.1787 |
| rs2548991 | imm_5_131889930 | A | 0.3159 | 8.15E−03 | IRF1, IL5 | 0.1819 |
| rs2706390 | imm_5_131870179 | A | 0.3159 | 8.15E−03 | IRF1, IL5 | 0.1809 |
| rs2706391 | imm_5_131871205 | G | 0.3159 | 8.15E−03 | IRF1, IL5 | 0.1791 |
| rs4705863 | imm_5_131870120 | C | 0.3159 | 8.15E−03 | IRF1, IL5 | 0.1817 |
| rs4705864 | imm_5_131870226 | C | 0.3159 | 8.15E−03 | IRF1, IL5 | 0.1816 |
| rs72797340 | imm_5_131895464 | A | 0.3159 | 8.15E−03 | IRF1, IL5 | 0.1784 |
| rs7736328 | imm_5_131868295 | G | 0.3159 | 8.15E−03 | IRF1, IL5 | 0.1819 |
| rs1044429 | rs1044429 | A | 0.3518 | 8.15E−03 | HLA-DOA | 0.1628 |
| rs592625 | rs592625 | G | 0.3518 | 8.15E−03 | HLA-DOA | 0.1785 |
| rs59179941 | seq-rs59179941 | A | 0.3236 | 8.16E−03 | LAIR2, KIR3DX1 | 0.2432 |
| rs1821393 | rs1821393 | A | 2.616 | 8.21E−03 | LINC01060 | 0.3917 |
| rs4863354 | rs4863354 | A | 2.616 | 8.23E−03 | LINC01060 | 0.3919 |
| rs6481157 | rs6481157 | A | 2.243 | 8.24E−03 | PCDH15, MTRNR2L5 | 0.4866 |
| rs370812 | imm_1_7998481 | G | 2.222 | 8.24E−03 | ERRFI1 | 0.4349 |
| rs371452 | imm_1_8006638 | G | 2.222 | 8.27E−03 | ERRFI1 | 0.4343 |
| rs400736 | imm_1_8000896 | A | 2.222 | 8.34E−03 | ERRFI1 | 0.445 |
| rs408320 | imm_1_8007915 | A | 2.222 | 8.35E−03 | ERRFI1 | 0.4336 |
| rs442862 | imm_1_8002081 | A | 2.222 | 8.35E−03 | ERRFI1 | 0.4337 |
| rs7249320 | rs7249320 | A | 0.363 | 8.39E−03 | FCER2 | 0.2447 |
| rs7249360 | rs7249360 | A | 0.363 | 8.51E−03 | FCER2 | 0.2438 |
| rs3790093 | rs3790093 | A | 2.466 | 8.53E−03 | GNAO1 | 0.3215 |
| rs61649748 | imm_8_11368574 | G | 2.715 | 8.56E−03 | FAM167A, BLK | 0.2212 |
| rs4703134 | rs4703134 | A | 2.41 | 8.56E−03 | ST8SIA4, SLCO4C1 | 0.3773 |
| rs59491394 | seq-rs59491394 | A | 2.707 | 8.58E−03 | FCAR | 0.2653 |
| rs17673852 | rs17673852 | G | 4.496 | 8.58E−03 | BMP6 | 0.08145 |
| rs927392 | imm_6_167281297 | A | 5.061 | 8.61E−03 | RNASET2 | 0.06645 |
| rs6941553 | rs6941553 | G | 2.321 | 8.63E−03 | MAP3K5 | 0.4758 |
| rs9285484 | rs9285484 | G | 2.321 | 8.63E−03 | MAP3K5 | 0.4786 |
| rs9483945 | rs9483945 | A | 2.321 | 8.63E−03 | MAP3K5 | 0.4768 |
| rs11059985 | rs11059985 | A | 2.918 | 8.64E−03 | GLT1D1 | 0.2692 |
| rs3129716 | rs3129716 | G | 16.58 | 8.64E−03 | HLA-DQB1, HLA-DQA2 | 0.1074 |
| rs1619379 | rs1619379 | A | 0.4437 | 8.64E−03 | LOC554223, HLA-G | 0.4433 |
| rs3810936 | imm_9_116592706 | A | 0.3961 | 8.64E−03 | TNFSF15 | 0.3013 |
| rs743562 | imm_5_131900282 | A | 0.4346 | 8.64E−03 | IRF1, IL5 | 0.4294 |
| rs1122730 | rs1122730 | A | 2.279 | 8.64E−03 | KIAA1462, LOC101929279 | 0.3687 |
| rs12946454 | rs12946454 | T | 0.3793 | 8.68E−03 | PLCD3 | 0.261 |
| rs4340374 | 1kg_17_29580545 | G | 0.3146 | 8.72E−03 | LOC101927239, CCL2 | 0.1849 |
| rs758294 | 1kg_17_29589233 | C | 0.3146 | 8.73E−03 | LOC101927239, CCL2 | 0.1839 |
| rs7634822 | imm_3_46149940 | C | 0.3408 | 8.73E−03 | XCR1, CCR1 | 0.21 |
| rs4978557 | rs4978557 | A | 2.355 | 8.73E−03 | RGS3, ZNF618 | 0.4251 |
| rs7741317 | rs7741317 | C | 5.902 | 8.73E−03 | PPP1R14C | 0.07141 |
| rs6806583 | rs6806583 | G | 0.1728 | 9.47E−03 | TNIK | 0.1119 |
| rs1424534 | imm_2_185885354 | G | 0.4405 | 9.48E−03 | ZNF804A, LOC101927196 | 0.4891 |
| rs1424536 | imm_2_185883474 | A | 0.4405 | 9.48E−03 | ZNF804A, LOC101927196 | 0.4886 |
| rs9808030 | imm_2_185883986 | G | 0.4405 | 9.48E−03 | ZNF804A, LOC101927196 | 0.4885 |
| rs10446439 | rs10446439 | A | 8.013 | 9.48E−03 | LINC01267, SLC6A6 | 0.05361 |
| rs2477858 | rs2477858 | G | 2.417 | 9.50E−03 | PCNXL2 | 0.436 |
| rs12695555 | rs12695555 | G | 2.142 | 9.50E−03 | NEK11 | 0.3807 |
| rs9813877 | rs9813877 | A | 2.142 | 9.50E−03 | NEK11 | 0.3797 |
| rs31607 | rs31607 | G | 0.2961 | 9.50E−03 | PJA2, MAN2A1 | 0.1236 |
| rs9262636 | rs9262636 | G | 2.89 | 9.53E−03 | HCG22 | 0.2305 |
| rs2394423 | rs2394423 | A | 2.89 | 9.53E−03 | HCG22, C6orf15 | 0.2306 |
| rs1484802 | 1kg_5_173326772 | C | 5.941 | 9.53E−03 | CPEB4, C5orf47 | 0.07051 |
| rs1388608 | imm_3_46093753 | A | 0.3537 | 9.56E−03 | XCR1, CCR1 | 0.2184 |
| rs1873616 | imm_3_46118606 | A | 0.3537 | 9.56E−03 | XCR1, CCR1 | 0.2183 |
| rs2373155 | imm_3_46147076 | A | 0.3537 | 9.56E−03 | XCR1, CCR1 | 0.2236 |
| rs4682811 | imm_3_46139799 | A | 0.3537 | 9.56E−03 | XCR1, CCR1 | 0.2186 |
| rs6808712 | imm_3_46106235 | G | 0.3537 | 9.56E−03 | XCR1, CCR1 | 0.2184 |
| rs6748538 | imm_2_102045141 | C | 2.855 | 9.60E−03 | IL1R2, ILIR1 | 0.1553 |
| rs2395165 | rs2395165 | G | 0.3037 | 9.61E−03 | BTNL2, HLA-DRA | 0.1864 |
| rs3135377 | rs3135377 | A | 0.3037 | 9.61E−03 | BTNL2, HLA-DRA | 0.1863 |
| rs4691153 | rs4691153 | G | 0.2344 | 9.64E−03 | TMEM192, KLHL2 | 0.1224 |
| rs2023623 | imm_1_170992698 | C | 0.4053 | 9.77E−03 | FASLG, TNFSF18 | 0.4547 |
| rs859630 | imm_1_170986074 | G | 0.4053 | 9.77E−03 | FASLG, TNFSF18 | 0.456 |
| rs12742784 | 1kg_1_22554953 | A | 2.918 | 9.79E−03 | MIR4418, ZBTB40 | 0.2074 |
| rs997351 | rs997351 | A | 2.93 | 9.80E−03 | PHOX2B, LINC00682 | 0.155 |
| rs11654788 | 1kg_17_29575627 | A | 3.386 | 9.81E−03 | LOC101927239, CCL2 | 0.1542 |
| rs62056376 | 1kg_17_29693134 | A | 0.2917 | 9.82E−03 | CCL8, CCL13 | 0.1475 |
| rs876493 | imm_17_35078071 | G | 0.4339 | 9.82E−03 | PNMT | 0.4154 |
| rs4751640 | rs4751640 | A | 2.234 | 9.85E−03 | EMX2, RAB11FIP2 | 0.3078 |
| rs4833833 | imm_4_123682070 | A | 2.544 | 9.91E−03 | IL2, IL21 | 0.2799 |
| rs7662182 | imm_4_123717881 | G | 2.544 | 9.91E−03 | IL2, IL21 | 0.2797 |
| rs6908100 | imm_6_127304631 | A | 2.22 | 9.95E−03 | MIR588, RSPO3 | 0.4576 |
| rs1047444 | imm_3_45935083 | C | 0.3678 | 9.96E−03 | FYCO1 | 0.2202 |
| rs1488374 | imm_3_45936846 | G | 0.3678 | 9.96E−03 | FYCO1 | 0.2204 |
| rs7130 | imm_3_45934519 | A | 0.3678 | 9.96E−03 | FYCO1 | 0.2203 |
| rs1488373 | imm_3_45932693 | G | 0.3678 | 9.96E−03 | LZTFL1 | 0.223 |
| rs9810934 | imm_3_45929356 | A | 0.3678 | 9.96E−03 | LZTFL1 | 0.22 |
| rs2034574 | rs2034574 | A | 0.4041 | 9.96E−03 | SIGLEC14, SPACA6P-AS | 0.2726 |
| rs3848726 | imm_20_44100002 | A | 2.681 | 9.98E−03 | SLC12A5 | 0.3716 |
| rs73209259 | imm_8_11365950 | G | 2.691 | 9.99E−03 | FAM167A, BLK | 0.2149 |
| rs8031294 | rs8031294 | A | 2.231 | 9.99E−03 | LINC00924, NR2F2-AS1 | 0.4459 |
| rs304723 | rs304723 | A | 2.569 | 1.00E−02 | ZNF576 | 0.306 |

TABLE 11
Polymorphisms associated with TL1A fold-change and Signal One Risk (linear model)

| | | Minor | | | | |
|---|---|---|---|---|---|---|
| | | Allele | | | | |
| Polymorphism | Illumina_id | (A1) | BETA | P | Gene | MAF |
| rs11600746 | imm_11_127851599 | G | 17.64 | 6.49E−06 | ETS1 | 0.1551 |
| rs11600915 | imm_11_127846698 | G | 17.64 | 6.49E−06 | ETS1 | 0.1542 |
| rs11606640 | imm_11_127840459 | A | 17.64 | 6.49E−06 | ETS1 | 0.1531 |
| rs12294634 | imm_11_127848372 | A | 17.64 | 6.49E−06 | ETS1 | 0.154 |
| rs61909068 | imm_11_127848167 | G | 17.64 | 6.49E−06 | ETS1 | 0.1544 |
| rs61909072 | imm_11_127855281 | A | 17.64 | 6.49E−06 | ETS1 | 0.1554 |
| rs73029052 | imm_11_127844385 | A | 17.64 | 6.49E−06 | ETS1 | 0.1539 |
| rs73029062 | imm_11_127849992 | G | 17.64 | 6.49E−06 | ETS1 | 0.1542 |
| rs116352370 | 1kg_2_241302416 | T | 40.99 | 6.82E−06 | KIF1A | 0.06014 |
| rs76887590 | imm_14_68364326 | A | 43.09 | 1.49E−05 | ZFP36L1, ACTN1 | 0.02328 |
| rs7713991 | rs7713991 | A | 15.8 | 1.66E−05 | LOC401177, CDH18 | 0.1978 |
| rs17031888 | imm_1_114163459 | G | 40.5 | 2.67E−05 | AP4B1-AS1 | 0.03863 |
| rs17031955 | imm_1_114212503 | A | 40.5 | 2.67E−05 | AP4B1-AS1 | 0.03644 |
| rs34209542 | imm_1_114174047 | G | 40.5 | 2.67E−05 | AP4B1-AS1 | 0.02318 |
| rs115870915 | imm_1_113935553 | A | 40.5 | 2.67E−05 | MAGI3 | 0.02177 |
| rs116347760 | imm_1_114002774 | | 40.5 | 2.67E−05 | MAGI3 | 0.01597 |
| rs33996649 | imm_1_114196212 | A | 40.5 | 2.67E−05 | PTPN22 | 0.02177 |
| rs2229136 | rs2229136 | G | 38.87 | 2.83E−05 | ALOX5 | 0.05768 |
| . | rs12013474 | A | 28.37 | 3.16E−05 | FMR1_FMRINB | 0.06752 |
| . | rs5904818 | A | 28.37 | 3.16E−05 | FMR1_FMRINB | 0.06703 |
| rs7812931 | rs7812931 | A | 24.86 | 3.96E−05 | ZHX2, DERL1 | 0.06666 |
| rs74675346 | imm_19_10343638 | A | 39.12 | 5.34E−05 | TYK2 | 0.02678 |
| rs191204 | imm_5_55463560 | A | 13.27 | 5.85E−05 | ANKRD55 | 0.4793 |
| rs6003160 | rs6003160 | G | 16.03 | 6.55E−05 | SCUBE1 | 0.295 |
| rs78103074 | imm_1_171126786 | A | 34.5 | 6.74E−05 | FASLG, TNFSF18 | 0.05048 |
| rs10461422 | imm_5_55468005 | C | 14.9 | 7.11E−05 | ANKRD55 | 0.2104 |
| rs17645980 | imm_5_55460497 | A | 14.9 | 7.11E−05 | ANKRD55 | 0.2127 |
| rs2940520 | rs2940520 | G | 30.43 | 7.12E−05 | UNC5A | 0.05085 |
| rs56086356 | imm_11_127881686 | C | 13.9 | 7.65E−05 | ETS1 | 0.1774 |
| rs10790957 | imm_11_127860440 | G | 14.61 | 8.04E−05 | ETS1 | 0.4149 |
| rs77984256 | imm_14_68259573 | A | 30.12 | 8.78E−05 | RAD51B, ZFP36L1 | 0.016615 |
| rs6056048 | rs6056048 | A | 12.87 | 9.11E−05 | PLCB1 | 0.3511 |
| rs747024 | rs747024 | A | 16.11 | 9.68E−05 | HERC4 | 0.1289 |
| rs4291387 | rs4291387 | A | 13.12 | 9.97E−05 | LOC158435 | 0.3516 |
| rs6928830 | rs6928830 | G | 13.44 | 1.10E−04 | ME1, PRSS35 | 0.1732 |
| rs5992462 | rs5992462 | G | 25.44 | 1.19E−04 | LINC00895, SEPT5 | 0.07632 |
| rs8137838 | rs8137838 | C | 25.44 | 1.19E−04 | LINC00895, SEPT5 | 0.07605 |
| rs17491714 | imm_12_56688746 | G | 33.42 | 1.25E−04 | XRCC6BP1, LOC10192765 3 | 0.0433 3 |
| rs4320976 | imm_11_75765492 | A | 15.4 | 1.33E−04 | PRKRIR | 0.1918 |
| rs7948288 | imm_11_75768491 | A | 15.4 | 1.33E−04 | PRKRIR | 0.1935 |
| rs4910068 | rs4910068 | G | 13.68 | 1.34E−04 | ST5 | 0.2834 |
| rs113514774 | ccc-12-56505633-G-A | A | 44.39 | 1.42E−04 | CTDSP2 | 0.02427 |
| rs115611397 | 1kg_8_79548346 | A | 44.39 | 1.42E−04 | LOC102724874, PKIA | 0.01008 |
| rs118001674 | imm_20_42669344 | A | 44.39 | 1.42E−04 | PKIG | 0.04463 |
| rs76540957 | imm_20_42679347 | G | 44.39 | 1.42E−04 | PKIG | 0.045116 |
| rs116432609 | imm_5_150340428 | G | 44.39 | 1.42E−04 | ZNF300P1, GPX3 | 0.02563 |
| rs878983 | rs878983 | A | 16.29 | 1.78E−04 | LAPTM4A, SDC1 | 0.2088 |
| rs76709465 | imm_5_132158742 | C | 26.77 | 2.03E−04 | SEPT8, SOWAHA | 0.02516 |
| rs11912198 | rs11912198 | A | 23.99 | 2.08E−04 | ZNRF3 | 0.01691 |
| rs16986990 | rs16986990 | G | 23.99 | 2.08E−04 | ZNRF3 | 0.018419 |
| rs4823000 | rs4823000 | G | 23.99 | 2.08E−04 | ZNRF3 | 0.0214 |
| rs8137391 | rs8137391 | A | 23.99 | 2.08E−04 | ZNRF3-AS1 | 0.01848 |
| rs10823062 | rs10823062 | A | 15.01 | 2.21E−04 | CTNNA3 | 0.2119 |
| rs2394411 | rs2394411 | A | 15.01 | 2.21E−04 | CTNNA3 | 0.2118 |
| rs117889858 | imm_16_67072555 | A | 34.84 | 2.23E−04 | SMPD3, ZFP90 | 0.0213 |
| rs117079792 | 1kg_8_79665101 | A | 35.03 | 2.29E−04 | PKIA | 0.04991 |
| rs74761562 | 1kg_8_79662196 | G | 35.03 | 2.29E−04 | PKIA | 0.03649 |
| rs75488794 | 1kg_8_79664297 | G | 35.03 | 2.29E−04 | PKIA | 0.05006 |
| rs75878904 | 1kg_8_79674210 | G | 35.03 | 2.29E−04 | PKIA | 0.050113 |
| rs117927932 | 1kg_8_79702265 | G | 35.03 | 2.29E−04 | PKIA, ZC2HCIA | 0.039015 |
| rs118136953 | 1kg_8_79710777 | A | 35.03 | 2.29E−04 | PKIA, ZC2HCIA | 0.0392 |
| rs118149281 | 1kg_8 79689940 | A | 35.03 | 2.29E−04 | PKIA, ZC2HCIA | 0.0389 |
| rs16905875 | 1kg_8 79714122 | A | 35.03 | 2.29E−04 | PKIA, ZC2HCIA | 0.04041 |
| rs74696769 | 1kg_8_79694906 | A | 35.03 | 2.29E−04 | PKIA, ZC2HCIA | 0.03881 |
| rs75901112 | 1kg_8_79696293 | A | 35.03 | 2.29E−04 | PKIA, ZC2HCIA | 0.039 |
| rs76195974 | 1kg_8_79708007 | A | 35.03 | 2.29E−04 | PKIA, ZC2HCIA | 0.03921 |
| rs76483342 | 1kg_8_79683854 | A | 35.03 | 2.29E−04 | PKIA, ZC2HCIA | 0.039 |
| rs77650073 | 1kg_8_79712780 | G | 35.03 | 2.29E−04 | PKIA, ZC2HCIA | 0.0392 |
| rs78100278 | 1kg_8_79681331 | G | 35.03 | 2.29E−04 | PKIA, ZC2HCIA | 0.0391 |
| rs78767737 | 1kg_8_79687223 | G | 35.03 | 2.29E−04 | PKIA, ZC2HCIA | 0.039 |
| rs79641310 | 1kg_8_79704013 | A | 35.03 | 2.29E−04 | PKIA, ZC2HCIA | 0.0392 |
| rs115984727 | imm_5_132143609 | A | 26.69 | 2.43E−04 | SEPT8, SOWAHA | 0.02534 |
| rs607660 | rs607660 | G | 10.92 | 2.55E−04 | CTAGE1, LOC101927571 | 0.4541 |
| rs1277016 | rs1277016 | G | 13.94 | 2.72E−04 | STXBP3 | 0.2562 |
| rs7895833 | rs7895833 | G | 13.9 | 2.75E−04 | DNAJC12, SIRT1 | 0.1969 |
| rs8036951 | rs8036951 | G | 15.78 | 2.83E−04 | FAM189A1 | 0.2253 |
| rs76321080 | imm_14_68299875 | A | 23.3 | 2.91E−04 | RAD51B, ZFP36L1 | 0.05847 |
| rs74792569 | imm_1_195719456 | A | 27.46 | 2.96E−04 | CRB1, DENND1B | 0.03247 |
| rs114979698 | imm_1_195931930 | G | 27.46 | 2.96E−04 | DENND1B | 0.0367 |
| rs75622950 | imm_1_196060621 | A | 27.46 | 2.96E−04 | DENND1B, C1orf53 | 0.03858 |
| rs1864577 | 1kg_8_79411977 | A | 25.35 | 2.97E−04 | LOC102724874, PKIA | 0.03639 |
| rs61394970 | 1kg_8_79494228 | G | 25.35 | 2.97E−04 | LOC102724874, PKIA | 0.03644 |
| rs73688944 | 1kg_8_79493024 | G | 25.35 | 2.97E−04 | LOC102724874, PKIA | 0.03649 |
| rs11779459 | rs11779459 | A | 12.32 | 2.98E−04 | ZHX2 | 0.3691 |
| rs6063456 | imm_20_48047586 | C | 12.75 | 3.03E−04 | SNAI1, TRERNA1 | 0.3958 |
| rs6125855 | imm_20_48057194 | A | 12.75 | 3.03E−04 | SNAI1, TRERNA1 | 0.3939 |
| rs6125864 | imm_20_48066512 | A | 12.75 | 3.03E−04 | SNAI1, TRERNA1 | 0.3962 |
| rs3920079 | rs3920079 | A | 13.66 | 3.08E−04 | CTNNA3 | 0.3193 |
| rs11769844 | rs11769844 | A | 13.98 | 3.09E−04 | STRA8 | 0.219 |
| rs17779592 | imm_17_23020853 | A | 34.32 | 3.25E−04 | LGALS9, NOS2 | 0.0374 |
| rs61732805 | imm_1_153675260 | A | 35.57 | 3.56E−04 | ASH1L | 0.013 |
| rs116547449 | imm_1_154148459 | A | 135.57 | 3.56E−04 | RIT1; KIAA0907 | 0.02444 |
| rs78029196 | imm_1_154247058 | C | 35.57 | 3.56E−04 | SSR2 | 0.01467 |
| rs114442346 | imm_1_154121831 | G | 35.57 | 3.56E−04 | SYT11 | 0.02433 |
| rs16924888 | rs16924888 | A | 14.93 | 3.89E−04 | DNAJC12 | 0.131 |
| rs17456400 | rs17456400 | C | 14.93 | 3.89E−04 | HERC4, MYPN | 0.1342 |
| rs2284665 | rs2284665 | A | 17.05 | 3.90E−04 | HTRA1 | 0.2158 |
| rs77498465 | imm_2_162718729 | A | 33.82 | 3.90E−04 | LOC101929532 | 0.046417 |
| rs34279840 | ccc-21-44452087-C-T | A | 30.31 | 3.92E−04 | C21orf33, ICOSLG | 0.02819 |
| rs74395031 | imm_1_113740022 | G | 30 | 3.94E−04 | MAGI3 | 0.01702 |
| rs1113283 | imm_17 23131525 | A | 14.41 | 4.05E−04 | NOS2 | 0.2454 |
| rs10760109 | imm_9_122437397 | A | 35.25 | 4.10E−04 | MEGF9 | 0.02328 |
| rs1886338 | imm_9_122451373 | G | 35.25 | 4.10E−04 | MEGF9 | 0.02325 |
| rs12428125 | rs12428125 | A | 46.06 | 4.16E−04 | BASP1P1, SGCG | 0.0427 |
| rs74334220 | imm_9_138275616 | G | 46.06 | 4.16E−04 | QSOX2 | 0.02261 |
| rs2287773 | rs2287773 | A | 46.06 | 4.16E−04 | SPINK5 | 0.02005 |
| rs2104517 | rs2104517 | A | 26.54 | 4.58E−04 | MIR548F5 | 0.05011 |
| rs6125877 | rs6125877 | C | 13.22 | 4.62E−04 | TRERNA1 | 0.4207 |
| rs9897780 | rs9897780 | A | 19.43 | 4.73E−04 | MYH10, CCDC42 | 0.1904 |
| rs4606022 | imm_8_11392342 | G | 12.04 | 4.84E−04 | BLK | 0.3851 |
| rs16949 | imm_17_23148826 | G | 14.17 | 4.93E−04 | NOS2 | 0.2491 |
| rs3794766 | imm_17_23146048 | A | 14.17 | 4.93E−04 | NOS2 | 0.2482 |
| rs4796080 | imm_17_23146864 | G | 14.17 | 4.93E−04 | NOS2 | 0.2482 |
| rs41278172 | imm_16_16162925 | A | 56.7 | 5.06E−04 | ABCC6 | 0.01874 |
| rs114797146 | imm_2_99955021 | A | 56.7 | 5.06E−04 | AFF3 | 0.01655 |
| rs76990532 | imm_2_99961776 | A | 56.7 | 5.06E−04 | AFF3 | 0.01498 |
| rs80055204 | imm_4_103235033 | G | 56.7 | 5.06E−04 | BANK1, SLC39A8 | 0.02109 |
| rs116767299 | imm_2_60785645 | A | 56.7 | 5.06E−04 | BCL11A, PAPOLG | 0.01545 |
| rs17011963 | rs17011963 | G | 56.7 | 5.06E−04 | BIRC6 | 0.059617 |
| rs79555446 | imm_6_34803126 | C | 56.7 | 5.06E−04 | C6orf106, SNRPC | 0.02008 |
| rs183396336 | imm_1_117099551 | A | 56.7 | 5.06E−04 | CD2 | 0.01034 |
| rs36027286 | imm_2_204351502 | G | 56.7 | 5.06E−04 | CD28, CTLA4 | 0.01958 |
| rs72832303 | ccc-6-20826759-A-G | G | 28.35 | 5.06E−04 | CDKAL1 | 0.02041 |
| rs12436392 | 1kg_14_80362854 | A | 56.7 | 5.06E−04 | CEP128 | 0.02542 |
| rs4496303 | imm_2_169021220 | A | 56.7 | 5.06E−04 | CERS6 | 0.012116 |
| rs76824122 | imm_2_204472857 | A | 56.7 | 5.06E−04 | CTLA4, ICOS | 0.01676 |
| rs73003218 | imm_11_118155373 | A | 28.35 | 5.06E−04 | DDX6 | 0.01665 |
| rs34764749 | imm_17_37228168 | G | 56.7 | 5.06E−04 | FKBP10 | 0.03383 |
| rs74567983 | imm_3_45944799 | A | 56.7 | 5.06E−04 | FYCO1 | 0.02396 |
| rs75326394 | imm_5_141398210 | A | 56.7 | 5.06E−04 | GNPDA1, NDFIP1 | 0.01566 |
| rs117073550 | 1kg_7_50636468 | A | 56.7 | 5.06E−04 | GRB10 | 0.01378 |
| rs117849753 | 1kg_7_50652297 | A | 56.7 | 5.06E−04 | GRB10 | 0.013719 |
| rs2190498 | 1kg_7_50657008 | G | 56.7 | 5.06E−04 | GRB10 | 0.01524 |
| rs74342530 | 1kg_7_50628359 | C | 56.7 | 5.06E−04 | GRB10 | 0.01383 |
| rs2158287 | 1kg_6_30314705 | C | 56.7 | 5.06E−04 | HCG17 | 0.03184 |
| rs2517808 | rs2517808 | A | 56.7 | 5.06E−04 | HCG9, ZNRD1-AS1 | 0.032 |
| rs113656426 | 1kg_5_173455093 | A | 56.7 | 5.06E−04 | HMP19 | 0.0105 |
| rs62048140 | chr16:22756562 | G | 56.7 | 5.06E−04 | HS3ST2 | 0.02778 |
| rs71459333 | imm_12_54711819 | A | 56.7 | 5.06E−04 | IKZF4 | 0.02756 |
| rs74357782 | imm_3_161133709 | A | 56.7 | 5.06E−04 | IL12A-AS1 | 0.01932 |
| rs76496898 | imm_3_161152680 | C | 56.7 | 5.06E−04 | IL12A-AS1 | 0.01926 |
| rs75280978 | imm_9_5192432 | G | 56.7 | 5.06E−04 | INSL6, INSL4 | 0.03059 |
| rs77576890 | imm_3_161093151 | G | 56.7 | 5.06E−04 | IQCJ-SCHIP1, SCHIP1 | 0.01968 |
| rs74605146 | imm_5_150214754 | G | 56.7 | 5.06E−04 | IRGM, ZNF300 | 0.02829 |
| rs35872871 | imm_7_107391402 | C | 56.7 | 5.06E−04 | LAMB1 | 0.03847 |
| rs35953236 | imm_7_107389083 | G | 56.7 | 5.06E−04 | LAMB1 | 0.03753 |
| rs114964491 | imm_1_150809019 | A | 56.7 | 5.06E−04 | LCE3E, LCE3D | 0.0177 |
| rs78498467 | imm_2_181832217 | C | 56.7 | 5.06E−04 | LOC101927156 | 0.01624 |
| rs75678669 | 1kg_14_87415296 | A | 56.7 | 5.06E−04 | LOC283585, GALC | 0.04108 |
| rs6886394 | rs6886394 | A | 56.7 | 5.06E−04 | LOC401177, CDH18 | 0.02501 |
| rs17465737 | imm_12_38906024 | G | 56.7 | 5.06E−04 | LRRK2 | 0.029013 |
| rs118136387 | 1kg_19_18269429 | A | 56.7 | 5.06E−04 | MIR3188, LSM4 | 0.0165 |
| rs148859834 | 1kg_19_18255426 | A | 56.7 | 5.06E−04 | MIR3188, LSM4 | 0.01791 |
| rs116918050 | imm_12_122176706 | G | 56.7 | 5.06E−04 | PITPNM2, MPHOSPH9 | 0.04469 |
| rs13157599 | rs13157599 | | 28.35 | 5.06E−04 | PRDM6, CEP120 | 0.039919 |
| rs2031723 | imm_10_6574701 | G | 56.7 | 5.06E−04 | PRKCQ | 0.04861 |
| rs73607015 | imm_10_6574092 | G | 56.7 | 5.06E−04 | PRKCQ | 0.048615 |
| rs77634074 | imm_10_6576542 | G | 56.7 | 5.06E−04 | PRKCQ | 0.04871 |
| rs117542910 | imm_17_35391423 | G | 56.7 | 5.06E−04 | PSMD3 | 0.0142 |
| rs12817671 | imm_12_54655491 | A | 56.7 | 5.06E−04 | RAB5B | 0.02146 |
| rs71427708 | imm_2_204201527 | C | 56.7 | 5.06E−04 | RAPH1, CD28 | 0.0201 |
| rs117270076 | imm_15_36751116 | G | 56.7 | 5.06E−04 | RASGRP1, C15orf53 | 0.01665 |
| rs77411382 | seq-VH-424 | A | 56.7 | 5.06E−04 | RGS21, RGS1 | 0.01697 |
| rs1150734 | 1kg_6_30153689 | G | 56.7 | 5.06E−04 | RNF39, TRIM31 | 0.03268 |
| rs71459335 | imm_12_54723184 | A | 56.7 | 5.06E−04 | RPS26 | 0.027416 |
| rs115942526 | 1kg_3_18606178 | G | 56.7 | 5.06E−04 | SATB1-AS1, KCNH8 | 0.0106 |
| rs113107898 | ccc-19-1134751-C-T | A | 56.7 | 5.06E−04 | SBNO2, STK11 | 0.01754 |
| rs79044169 | imm_17_37791339 | G | 56.7 | 5.06E−04 | STAT3 | 0.04307 |
| rs80084007 | imm_17_37709578 | C | 56.7 | 5.06E−04 | STAT5A | 0.01535 |
| rs76806513 | imm_17_37654238 | A | 56.7 | 5.06E−04 | STAT5B | 0.02448 |
| rs112243913 | imm_6_128090636 | A | 56.7 | 5.06E−04 | THEMIS | 0.02365 |
| rs9289130 | imm_3_120658887 | G | 56.7 | 5.06E−04 | TMEM39A | 0.01394 |
| rs2021730 | 1kg_6_30184404 | A | 56.7 | 5.06E−04 | TRIM31-AS1 | 0.033517 |
| rs2523991 | 1kg_6_30183625 | A | 56.7 | 5.06E−04 | TRIM31-AS1 | 0.03357 |
| rs2523993 | 1kg_6_30183046 | A | 56.7 | 5.06E−04 | TRIM31-AS1 | 0.03346 |
| rs2844794 | 1kg_6_30184707 | G | 56.7 | 5.06E−04 | TRIM31-AS1 | 0.03346 |
| rs2516687 | 1kg_6_30472566 | A | 56.7 | 5.06E−04 | TRIM39-RPP21, HLA-E | 0.03221 |
| rs2516687 | rs2516687 | A | 56.7 | 5.06E−04 | TRIM39-RPP21, HLA-E | 0.03221 |
| rs10146359 | rs10146359 | G | 56.7 | 5.06E−04 | TTC7B | 0.01362 |
| rs10150260 | rs10150260 | G | 56.7 | 5.06E−04 | TTC7B | 0.01597 |
| rs11847179 | rs11847179 | A | 56.7 | 5.06E−04 | TTC7B | 0.01352 |
| rs12591019 | rs12591019 | A | 56.7 | 5.06E−04 | TTC7B | 0.0142 |
| rs17094709 | rs17094709 | A | 56.7 | 5.06E−04 | TTC7B | 0.01362 |
| rs17126980 | rs17126980 | A | 56.7 | 5.06E−04 | TTC7B | 0.01378 |
| rs17126982 | rs17126982 | A | 56.7 | 5.06E−04 | TTC7B | 0.01289 |
| rs1998188 | rs1998188 | A | 56.7 | 5.06E−04 | TTC7B | 0.01354 |
| rs2401911 | rs2401911 | G | 56.7 | 5.06E−04 | TTC7B | 0.01425 |
| rs2896142 | rs2896142 | A | 56.7 | 5.06E−04 | TTC7B | 0.01362 |
| rs4900059 | rs4900059 | A | 56.7 | 5.06E−04 | TTC7B | 0.01446 |
| rs4904723 | rs4904723 | G | 56.7 | 5.06E−04 | TTC7B | 0.02013 |
| rs6575143 | rs6575143 | C | 56.7 | 5.06E−04 | TTC7B | 0.01357 |
| rs6575144 | rs6575144 | G | 56.7 | 5.06E−04 | TTC7B | 0.01357 |
| rs8004183 | rs8004183 | A | 56.7 | 5.06E−04 | TTC7B | 0.01357 |
| rs8019797 | rs8019797 | C | 56.7 | 5.06E−04 | TTC7B | 0.01357 |
| rs79165228 | imm_6_34904432 | G | 56.7 | 5.06E−04 | UHRF1BP1 | 0.0201 |
| rs115537678 | imm_5_150351522 | A | 56.7 | 5.06E−04 | ZNF300P1, GPX3 | 0.01122 |
| rs10497658 | imm_2_185354265 | C | 56.7 | 5.06E−04 | ZNF804A | 0.03659 |
| rs129905 19 | imm_2_185206214 | G | 56.7 | 5.06E−04 | ZNF804A | 0.034215 |
| rs13018902 | imm_2_185351125 | C | 56.7 | 5.06E−04 | ZNF804A | 0.03659 |
| rs72905734 | imm_2_185409951 | A | 56.7 | 5.06E−04 | ZNF804A | 0.01169 |
| rs259948 | 1kg_6_30129728 | A | 56.7 | 5.06E−04 | ZNRD1-AS1 | 0.03184 |
| rs28665311 | 9_133094869 | G | 40.95 | 5.26E−04 | NUP214 | 0.02683 |
| rs17712705 | rs17712705 | A | 13.12 | 5.26E−04 | DNAJC12, SIRT1 | 0.3229 |
| rs74431747 | imm_1_113814325 | G | 22.87 | 5.37E−04 | MAGI3 | 0.03085 |
| rs75948156 | imm_1_113767166 | A | 22.87 | 5.37E−04 | MAGI3 | 0.03054 |
| rs76975167 | imm_1_113754608 | A | 22.87 | 5.37E−04 | MAGI3 | 0.03059 |
| rs77128194 | imm_1_113741794 | A | 22.87 | 5.37E−04 | MAGI3 | 0.03049 |
| rs72904673 | 1kg_18_41090953 | C | 26.17 | 5.44E−04 | SLC14A2 | 0.05398 |
| rs6435959 | rs6435959 | A | -11.84 | 5.48E−04 | LOC101928327, | 0.3867 |
| rs72941667 | imm_6_106619387 | G | 20.68 | 5.81E−04 | PREP, PRDM1 | 0.1019 |
| rs72941674 | imm_6_106628903 | A | 20.68 | 5.81E−04 | PREP, PRDM1 | 0.1033 |
| rs72941675 | imm_6_106629629 | C | 20.68 | 5.81E−04 | PREP, PRDM1 | 0.1038 |
| rs1054839 | ccc-21-44454220-A-G | G | 26.29 | 5.83E−04 | C21orf33, ICOSLG | 0.05111 |
| rs28550609 | 1kg_8_79506636 | A | 23.03 | 5.84E−04 | LOC102724874, PKIA | 0.07173 |
| rs4145315 | 1kg_8_79562307 | A | 23.03 | 5.84E−04 | LOC102724874, PKIA | 0.07256 |
| rs10483739 | rs10483739 | A | 13.04 | 5.87E−04 | PRKCH | 0.2163 |
| rs1356122 | rs1356122 | G | 18.16 | 5.97E−04 | GPR149, MME | 0.1793 |
| rs4679735 | rs4679735 | G | 18.16 | 5.97E−04 | GPR149, MME | 0.1725 |
| rs17720798 | imm_6_127396930 | A | 26.05 | 6.00E−04 | MIR588, RSPO3 | 0.05967 |
| rs10750399 | imm_11_127807384 | A | 14.07 | 6.02E−04 | LOC101929497, ETS1 | 0.1627 |
| rs4285885 | imm_11_127824356 | A | 14.07 | 6.02E−04 | LOC101929497, ETS1 | 0.1603 |
| rs4936050 | imm_11_127826464 | A | 14.07 | 6.02E−04 | LOC101929497, ETS1 | 0.1485 |
| rs6590332 | imm_11_127827422 | A | 14.07 | 6.02E−04 | LOC101929497, ETS1 | 0.1492 |
| rs9665767 | imm_11_127819226 | G | 14.07 | 6.02E−04 | LOC101929497, ETS1 | 0.163 |
| rs12207890 | rs12207890 | G | 18.26 | 6.04E−04 | ELOVL4, TTK | 0.1881 |
| rs922483 | imm_8_11389321 | A | −12.77 | 6.53E−04 | BLK | 0.2842 |
| rs6067323 | rs6067323 | A | 12.49 | 6.58E−04 | SNAI1, TRERNA1 | 0.392 |
| rs116297428 | imm_2_162683961 | A | 32.65 | 6.87E−04 | LOC101929532 | 0.01535 |
| rs2163625 | rs2163625 | G | 11.17 | 6.92E−04 | TMEM9B | 0.4115 |
| rs10823120 | rs10823120 | A | 12.31 | 7.04E−04 | HERC4 | 0.3259 |
| rs11255111 | rs11255111 | A | 32.6 | 7.06E−04 | SFMBT2 | 0.03623 |
| rs2747181 | rs2747181 | A | 15.48 | 7.49E−04 | LINC01364, PKN2-AS1 | 0.1764 |
| rs1894216 | imm_11_127806969 | T | 13.75 | 7.55E−04 | LOC101929497, ETS1 | 0.1977 |
| rs4648892 | rs4648892 | G | 12.04 | 7.60E−04 | TCEA3 | 0.2663 |
| rs115566179 | imm_6_34895515 | A | 38.76 | 7.63E−04 | UHRF1BP1 | 0.02736 |
| rs2888456 | rs2888456 | G | −11.03 | 7.73E−04 | LOC101928327, DIRC3- | 0.4371 |
| rs3176595 | rs3176595 | A | 16.23 | 8.03E−04 | HUS1 | 0.104 |
| rs76923469 | imm_3_161148501 | A | 38.71 | 8.11E−04 | IL12A-AS1 | 0.02824 |
| rs77908676 | imm_3_161172814 | G | 38.71 | 8.11E−04 | IL12A-AS1 | 0.0285 |
| rs6122864 | imm_20_48078227 | A | 12.39 | 8.47E−04 | SNAI1, TRERNA1 | 0.3771 |
| rs1467483 | imm_20_48085587 | A | 12.39 | 8.47E−04 | SNAI1, TRERNA1 | 0.3896 |
| rs1973946 | imm_20_48082437 | G | 12.39 | 8.47E−04 | SNAI1, TRERNA1 | 0.3913 |
| rs8116609 | imm_20_48080480 | G | 12.39 | 8.47E−04 | SNAI1, TRERNA1 | 0.3901 |
| rs8119515 | imm_20_48078094 | T | 12.39 | 8.47E−04 | SNAI1, TRERNA1 | 0.391 |
| rs10088323 | imm_8_11338301 | G | −11.89 | 8.50E−04 | FAM167A | 0.4291 |
| rs6462484 | rs6462484 | A | −12.51 | 8.51E−04 | BBS9 | 0.3993 |
| rs12495880 | rs12495880 | G | 26.08 | 8.55E−04 | PRICKLE2, ADAMTS9 | 0.033918 |
| rs268875 | imm_2_65349391 | G | 19.91 | 8.66E−04 | ACTR2 | 0.1231 |
| rs6067309 | imm_20_48043700 | G | 11.48 | 9.11E−04 | SNAI1, TRERNA1 | 0.3965 |
| rs6990997 | rs6990997 | G | 19.76 | 9.27E−04 | ZFPM2-AS1 | 0.09115 |
| rs6067322 | imm_20_48073717 | G | 11.25 | 9.35E−04 | SNAI1, TRERNA1 | 0.3992 |
| rs12637133 | 1kg_3_18730712 | A | 38.23 | 9.50E−04 | SATB1-AS1, KCNH8 | 0.02897 |
| rs4468995 | 1kg_3_18778496 | A | 38.23 | 9.50E−04 | SATB1-AS1, KCNH8 | 0.030413 |
| rs114005859 | 1kg_3_28069430 | G | 32.84 | 9.77E−04 | LOC100996624, CMC1 | 0.01963 |
| rs1466085 | rs1466085 | A | 20.67 | 9.81E−04 | TFRC, LINC00885 | 0.07366 |

TABLE 12
Polymorphisms associated with high-low TL1A fold-change and Signal One Carrier (logistic model)

| | | Minor | | | | |
|---|---|---|---|---|---|---|
| | | Allele | | | | |
| Polymorphism | Illumina_id | (A1) | OR | P | Gene | MAF |
| . | rs2280964 | A | 4.42 | 3.48E−03 | CXCR3 | 0.2505 |
| rs196595 | rs196595 | G | 0.3641 | 5.79E−03 | EEPD1 | 0.3425 |
| rs7674333 | rs7674333 | A | 2.584 | 4.19E−03 | GABRB1 | 0.4324 |
| rs3763341 | rs3763341 | A | 0.2619 | 3.45E−03 | HLA-DOA, HLA-DPA1 | 0.1397 |
| rs2857201 | rs2857201 | C | 0.2295 | 1.81E−03 | HLA-DQB2, HLA-DOB | 0.2835 |
| rs140935661 | imm_5_40408209 | A | 5.104 | 5.68E−03 | LINC00603, PTGER4 | 0.1273 |
| rs6921610 | rs6921610 | G | 3.629 | 7.25E−04 | LY86, RREB1 | 0.4637 |
| rs2780786 | 1kg_1_241030758 | G | 12.733 | 6.34E−03 | PLD5, LINC01347 | 0.4012 |
| rs12114972 | rs12114972 | C | 0.3717 | 4.66E−03 | PSD3 | 0.427 |
| rs2548278 | rs2548278 | A | 2.906 | 5.65E−03 | ST8SIA4 | 0.3496 |
| rs12607033 | rs12607033 | C | 2.778 | 5.76E−03 | VAPA, LINC01254 | 0.3631 |
| rs59366011 | 1kg_2_206983501 | A | 0.3543 | 9.75E−03 | ZDBF2, ADAM23 | 0.2494 |
| 10061600650G0D | 10-6160065-G- | I | 7.849 | 9.43E−03 | IL2RA_RBM17 | 0.09007 |
| ELETION | DELETION | | | | | |
| 50404601310A0D5- | 40460131-A- | D | 5.104 | 5.68E−03 | LOC285634_LOC100127944 | 0.1298 |
| ELETION | DELETION | | | | | |
| rs10026884 | rs10026884 | G | 0.3729 | 5.36E−03 | GABRB1 | 0.3398 |
| rs1003533 | imm_5_131783550 | A | 0.2486 | 2.76E−03 | C5orf56 | 0.2059 |
| rs1005048 | imm_12_66786506 | A | 0.2716 | 9.67E−04 | IFNG-AS1, IFNG | 0.4023 |
| rs10055349 | imm_5_40477475 | A | 3.578 | 3.50E−03 | LINC00603, PTGER4 | 0.2207 |
| rs1005567 | rs1005567 | A | 0.3914 | 6.10E−03 | LMOD2, WASL | 0.4808 |
| rs10131232 | rs10131232 | A | 0.3379 | 9.30E−03 | GCH1 | 0.2987 |
| rs10169606 | rs10169606 | G | 2.713 | 2.28E−03 | ARHGAP15 | 0.3662 |
| rs1016988 | imm_5_131772473 | G | 0.3132 | 8.52E−03 | SLC22A5, C5orf56 | 0.2064 |
| rs10179483 | imm_2_204360509 | G | 2.598 | 8.69E−03 | CD28, CTLA4 | 0.2371 |
| rs10188460 | imm_2_61712172 | A | 3.526 | 9.97E−03 | XPO1, FAM161A | 0.1738 |
| rs10189240 | rs10189240 | G | 2.443 | 6.17E−03 | ARHGAP15 | 0.3637 |
| rs10190232 | imm_2_102041393 | G | 3.33 | 8.84E−03 | IL1R2, ILIR1 | 0.1539 |
| rs1025601 | rs1025601 | A | 0.3952 | 8.00E−03 | TSHZ1, SMIM21 | 0.3742 |
| rs10256927 | rs10256927 | A | 0.3209 | 5.58E−03 | LOC101928283, GRM8 | 0.2437 |
| rs10283808 | imm_9_34932073 | A | 3.857 | 4.74E−03 | FAM205C, PHF24 | 0.1917 |
| rs1044429 | rs1044429 | A | 0.3177 | 6.16E−03 | HLA-DOA | 0.1628 |
| rs1047444 | imm_3_45935083 | C | 0.316 | 5.72E−03 | FYCO1 | 0.2202 |
| rs10483658 | rs10483658 | A | 6.744 | 5.20E−03 | PELI2 | 0.1389 |
| rs10503636 | rs10503636 | G | 2.63 | 5.33E−03 | PSD3 | 0.4891 |
| rs10509690 | rs10509690 | A | 0.2868 | 8.68E−04 | SORBS1 | 0.2369 |
| rs10511456 | imm_9_4305442 | G | 2.875 | 8.07E−03 | GLIS3, SLC1A1 | 0.356 |
| rs10512737 | imm_5_40445800 | A | 5.104 | 5.68E−03 | LINC00603, PTGER4 | 0.1298 |
| rs10516615 | imm_4_123194057 | G | 5.277 | 5.29E−03 | TRPC3, KIAA1109 | 0.1309 |
| rs10733475 | imm_9_34859735 | A | 3.263 | 9.77E−03 | FAM205BP, FAM205C | 0.2256 |
| rs10736978 | rs10736978 | C | 3.152 | 5.08E−03 | LOC105376360 | 0.3041 |
| rs10751118 | seq-rs10751118 | C | 2.664 | 5.37E−03 | KRTAP5-11 | 0.3802 |
| rs10784670 | imm_12_66760362 | G | 2.526 | 9.54E−03 | IFNG-AS1, IFNG | 0.4187 |
| rs10814923 | imm_9_4305101 | A | 2.875 | 8.07E−03 | GLIS3, SLC1A1 | 0.3551 |
| rs10814929 | imm_9_4306859 | A | 2.875 | 8.07E−03 | GLIS3, SLC1A1 | 0.3558 |
| rs10814930 | imm_9_4307107 | A | 3.039 | 8.96E−03 | GLIS3, SLC1A1 | 0.3346 |
| rs10824740 | imm_10_80731730 | A | 0.3693 | 7.45E−03 | ZMIZ1 | 0.2883 |
| rs10826406 | rs10826406 | A | 0.1339 | 4.16E−03 | MPP7 | 0.09972 |
| rs10847699 | imm_12_127868986 | G | 3.31 | 6.29E−03 | SLC15A4 | 0.3009 |
| rs10878749 | imm_12_66793406 | T | 0.2767 | 1.28E−03 | IFNG-AS1, IFNG | 0.401 |
| rs10881582 | rs10881582 | A | 0.2841 | 4.04E−03 | RXRA | 0.2444 |
| rs10889401 | rs10889401 | C | 2.57 | 8.64E−03 | ATG4C, LINC00466 | 0.3394 |
| rs10892901 | rs10892901 | A | 0.3891 | 9.50E−03 | CNTN5 | 0.404 |
| rs10900807 | imm_5_131785379 | C | 0.2486 | 2.76E−03 | C5orf56 | 0.2041 |
| rs10918931 | rs10918931 | A | 2.591 | 6.06E−03 | XCL1, DPT | 0.3795 |
| rs10924249 | rs10924249 | A | 3.665 | 8.11E−03 | KIF26B | 0.1798 |
| rs10928195 | rs10928195 | C | 4.626 | 6.24E−03 | ARHGAP15 | 0.1343 |
| rs10946197 | imm_6_167268406 | A | 0.3552 | 8.20E−03 | RNASET2 | 0.2716 |
| rs10972251 | imm_9_34865445 | A | 3.263 | 9.77E−03 | FAM205BP, FAM205C | 0.2219 |
| rs10986432 | rs10986432 | G | 0.2996 | 6.54E−03 | OLFML2A | 0.1875 |
| rs11004384 | rs11004384 | C | 4.025 | 1.57E−03 | PCDH15 | 0.2936 |
| rs11059915 | imm_12_127850960 | A | 3.055 | 9.89E−03 | SLC15A4 | 0.2687 |
| rs11059934 | imm_12_127870813 | G | 3.31 | 6.29E−03 | SLC15A4 | 0.3002 |
| rs11059985 | rs11059985 | A | 3.162 | 9.05E−03 | GLT1D1 | 0.2692 |
| rs11073304 | rs11073304 | A | 4.909 | 5.43E−03 | SPRED1 | 0.1406 |
| rs11082436 | 1kg_18_41083040 | G | 3.799 | 3.40E−03 | SLC14A2 | 0.1949 |
| rs11098092 | rs11098092 | G | 2.581 | 7.81E−03 | PITX2, C4orf32 | 0.4373 |
| rs1 1140519 | rs11140519 | A | 5.086 | 7.34E−03 | SLC28A3 | 0.1152 |
| rs11156878 | 1kg_14_34805718 | G | 3.959 | 5.41E−03 | KIAA0391 | 0.1837 |
| rs1 1177049 | imm_12_66784143 | G | 0.2674 | 7.46E−04 | IFNG-AS1, IFNG | 0.3964 |
| rs11177050 | imm_12_66784252 | G | 0.2674 | 7.46E−04 | IFNG-AS1, IFNG | 0.3963 |
| rs11177053 | imm_12_66785504 | G | 0.2716 | 9.67E−04 | IFNG-AS1, IFNG | 0.4024 |
| rs11177059 | imm_12_66793735 | A | 0.2767 | 1.28E−03 | IFNG-AS1, IFNG | 0.4003 |
| rs11177060 | imm_12_66794543 | A | 0.3881 | 7.24E−03 | IFNG-AS1, IFNG | 0.3857 |
| rs11224827 | rs11224827 | A | 4.207 | 6.50E−03 | TRPC6 | 0.1086 |
| rs1124233 | imm_5_40425044 | A | 5.104 | 5.68E−03 | LINC00603, PTGER4 | 0.1272 |
| rs11544238 | imm_12_56156422 | A | 2.797 | 6.54E−03 | ARHGAP9 | 0.3652 |
| rs11610401 | imm_12_66773584 | T | 0.2571 | 5.94E−04 | IFNG-AS1, IFNG | 0.3964 |
| rs11610754 | imm_12_66772854 | C | 0.3936 | 7.12E−03 | IFNG-AS1, IFNG | 0.3721 |
| rs11614309 | imm_12_66789272 | LA | 0.4066 | 8.72E−03 | IFNG-AS1, IFNG | 0.3727 |
| rs11624462 | 1kg_14_34741437 | C | 4.265 | 2.34E−03 | KIAA0391 | 0.2311 |
| rs116258627 | imm_5_96222028 | G | 0.1839 | 6.33E−03 | ERAP1, ERAP2 | 0.06959 |
| rs11627958 | 1kg_14_34778760 | A | 4.325 | 2.33E−03 | KIAA0391 | 0.2358 |
| rs116583745 | imm_5_96208838 | A | 0.1839 | 6.33E−03 | ERAP1, ERAP2 | 0.06964 |
| rs11664603 | 1kg_18_41082724 | G | 3.144 | 7.04E−03 | SLC14A2 | 0.2203 |
| rs11679301 | imm_2_185855392 | G | 3.092 | 5.99E−03 | ZNF804A, LOC101927196 | 0.2495 |
| rs11690566 | rs11690566 | A | 0.3045 | 4.29E−03 | FAM136A, TGFA | 0.2698 |
| rs11711554 | rs11711554 | A | 2.367 | 6.99E−03 | ITPR1 | 0.4885 |
| rs117324436 | imm_9_4995771 | G | 19.211 | 5.21E−03 | JAK2 | 0.08959 |
| rs11739261 | imm_5_40446496 | A | 5.104 | 5.68E−03 | LINC00603, PTGER4 | 0.1298 |
| rs11739622 | imm_5_131897867 | A | 3.432 | 4.93E−03 | IRF1, IL5 | 0.1899 |
| rs11739725 | imm_5_40459216 | G | 5.104 | 5.68E−03 | LINC00603, PTGER4 | 0.1299 |
| rs11749040 | imm_5_40432182 | A | 5.104 | 5.68E−03 | LINC00603, PTGER4 | 0.1271 |
| rs117519281 | imm_16_11327090 | A | 0.1541 | 6.30E−03 | PRM1, RMI2 | 0.08942 |
| rs11761905 | rs11761905 | A | 13.077 | 8.66E−03 | JAZF1 | 0.2309 |
| rs11764513 | imm_7_27110917 | A | 0.1121 | 8.84E−03 | HOXA2, HOXA3 | 0.0925 |
| rs11793394 | imm_9_116611852 | G | 0.3245 | 8.08E−03 | TNFSF15, TNFSF8 | 0.4756 |
| rs1182218 | seq-rs1182218 | G | 0.3475 | 7.55E−03 | CD2, PTGFRN | 0.1678 |
| rs1182219 | seq-t1d-1-117162224-T-A | A | 0.3475 | 7.55E−03 | CD2, PTGFRN | 0.1679 |
| rs11835920 | imm_12_66794620 | A | 0.3051 | 1.95E−03 | IFNG-AS1, IFNG | 0.2876 |
| rs1 1922919 | rs11922919 | A | 0.2921 | 6.09E−03 | WNT7A | 0.1943 |
| rs12001305 | imm_9_34893675 | G | 3.263 | 9.77E−03 | FAM205C, PHF24 | 0.2267 |
| rs12002089 | imm_9_34917690 | A | 3.857 | 4.74E−03 | FAM205C, PHF24 | 0.1964 |
| rs12005235 | imm_9_34893527 | A | 3.263 | 9.77E−03 | FAM205C, PHF24 | 0.2218 |
| rs12187530 | imm_5_40425609 | A | 5.104 | 5.68E−03 | LINC00603, PTGER4 | 0.1271 |
| rs12191230 | rs12191230 | A | 0.3545 | 5.93E−03 | BRD2, HLA-DOA | 0.2748 |
| rs12203875 | rs12203875 | G | 2.354 | 9.27E−03 | LINC00271 | 0.4477 |
| rs12232990 | imm_2_102036465 | A | 3.33 | 8.84E−03 | IL1R2, ILIR1 | 0.1523 |
| rs12318183 | imm_12_66790103 | A | 0.3592 | 4.62E−03 | IFNG-AS1, IFNG | 0.3811 |
| rs1233651 | 1kg_17_29663474 | G | 0.2217 | 1.87E−03 | CCL11, CCL8 | 0.1864 |
| rs1233651 | rs1233651 | G | 0.2217 | 1.87E−03 | CCL11, CCL8 | 0.1864 |
| rs12339512 | imm_9_4306020 | A | 2.875 | 8.07E−03 | GLIS3, SLC1A1 | 0.3606 |
| rs12443188 | imm_15_65220995 | T | 2.89 | 7.32E−03 | SMAD3 | 0.2392 |
| rs12468414 | rs12468414 | G | 0.2635 | 4.98E−03 | XPO1, FAM161A | 0.1925 |
| rs12471529 | imm_2_101904784 | G | 3.153 | 3.94E−03 | MAP4K4, LINC01127 | 0.4223 |
| rs12535739 | 1kg_7_37384447 | A | 0.1875 | 8.80E−03 | ELMO1 | 0.07079 |
| rs12541603 | rs12541603 | G | 2.815 | 6.21E−03 | LINC00824 | 0.4047 |
| rs12590856 | rs12590856 | A | 6.744 | 5.20E−03 | PELI2 | 0.1336 |
| rs1265566 | imm_12_110200759 | G | 0.3286 | 7.74E−03 | CUX2 | 0.2867 |
| rs12695555 | rs12695555 | G | 2.289 | 9.95E−03 | NEK11 | 0.3807 |
| rs12727925 | rs12727925 | A | 0.1014 | 6.82E−03 | RNF186 | 0.08535 |
| rs12811446 | imm_12_66777049 | A | 0.3936 | 7.12E−03 | IFNG-AS1, IFNG | 0.3719 |
| rs12815372 | imm_12_66765480 | A | 0.3936 | 7.12E−03 | IFNG-AS1, IFNG | 0.3723 |
| rs12822844 | imm_12_66791307 | G | 0.4066 | 8.72E−03 | IFNG-AS1, IFNG | 0.3727 |
| rs12825700 | imm_12_66779247 | A | 0.3936 | 7.12E−03 | IFNG-AS1, IFNG | 0.372 |
| rs12831020 | imm_12_66785758 | G | 0.4066 | 8.72E−03 | IFNG-AS1, IFNG | 0.3738 |
| rs12897219 | rs12897219 | G | 2.999 | 3.76E−03 | PRKD1, G2E3 | 0.3429 |
| rs12908584 | rs12908584 | C | 2.621 | 3.39E−03 | LINC01584 | 0.3552 |
| rs12913742 | rs12913742 | G | 3.405 | 4.83E−04 | RGMA, LOC101927153 | 0.4576 |
| rs12915039 | imm_15_65221402 | C | 2.929 | 7.28E−03 | SMAD3 | 0.241 |
| rs13006027 | imm_2_101934959 | A | 3.232 | 4.30E−03 | MAP4K4, LINC01127 | 0.3454 |
| rs13147245 | imm_4_123742806 | A | 2.594 | 9.69E−03 | IL2, IL21 | 0.4048 |
| rs132001 | rs132001 | A | 4.015 | 3.09E−03 | PHF21B, NUP50-AS1 | 0.1615 |
| rs13290746 | imm_9_34858377 | G | 3.263 | 9.77E−03 | FAM205BP, FAM205C | 0.2259 |
| rs13420455 | rs13420455 | A | 0.3281 | 6.98E−03 | FAM136A, TGFA | 0.267 |
| rs1353280 | rs1353280 | G | 0.318 | 4.04E−03 | UGT2B28, UGT2B4 | 0.3291 |
| rs1373693 | imm_5_40466932 | G | 5.104 | 5.68E−03 | LINC00603, PTGER4 | 0.1299 |
| rs1373694 | imm_5_40438950 | A | 5.104 | 5.68E−03 | LINC00603, PTGER4 | 0.1271 |
| rs1376480 | rs1376480 | C | 2.443 | 7.10E−03 | SYNPR | 0.4888 |
| rs1388608 | imm_3_46093753 | A | 0.3064 | 6.17E−03 | XCR1, CCR1 | 0.2184 |
| rs1407308 | imm_9_116610044 | A | 0.3123 | 5.96E−03 | TNFSF15, TNFSF8 | 0.4745 |
| rs1425806 | 1kg_11_34992974 | G | 0.3392 | 3.43E−03 | PDHX, LOC100507144 | 0.3017 |
| rs1434254 | rs1434254 | G | 0.3603 | 7.19E−03 | PTPRD | 0.4741 |
| rs1437747 | rs1437747 | G | 0.3658 | 7.37E−03 | CAMKMT | 0.4635 |
| rs1444291 | rs1444291 | G | 3.757 | 2.29E−03 | LINC01584 | 0.2576 |
| rs1444300 | rs1444300 | A | 3.417 | 2.45E−03 | LINC01584 | 0.2677 |
| rs1445002 | imm_5_40355634 | A | 4.635 | 9.19E−03 | LINC00603, PTGER4 | 0.1243 |
| rs1455181 | rs1455181 | A | 0.3696 | 8.79E−03 | RFX3-AS1, GLIS3 | 0.3804 |
| rs1475041 | 1kg_14_34863301 | G | 3.959 | 5.41E−03 | PSMA6, NFKBIA | 0.1862 |
| rs1488373 | imm_3_45932693 | G | 0.316 | 5.72E−03 | LZTFL1 | 0.223 |
| rs1488374 | imm_3_45936846 | G | 0.316 | 5.72E−03 | FYCO1 | 0.2204 |
| rs149598 | imm_5_96195447 | A | 0.1839 | 6.33E−03 | ERAP1, ERAP2 | 0.07408 |
| rs1512973 | imm_4_123725506 | A | 2.731 | 8.08E−03 | IL2, IL21 | 0.3311 |
| rs1522764 | rs1522764 | C | 4.909 | 5.43E−03 | SPRED1 | 0.1397 |
| rs1529028 | rs1529028 | A | 0.1669 | 6.43E−03 | GBE1, NONE | 0.1035 |
| rs1558743 | imm_12_66790769 | C | 0.3592 | 4.62E−03 | IFNG-AS1, IFNG | 0.3814 |
| rs1558744 | imm_12_66790859 | A | 0.2716 | 9.67E−04 | IFNG-AS1, IFNG | 0.4023 |
| rs1570452 | 1kg_13_98867496 | G | 0.3555 | 2.68E−03 | MIR548AN, LINC01232 | 0.3054 |
| rs1607785 | rs1607785 | G | 0.3975 | 8.23E−03 | E2F7, NAV3 | 0.3327 |
| rs16863769 | rs16863769 | G | 0.3061 | 3.34E−03 | MTX2, MIR 1246 | 0.244 |
| rs16899792 | imm_6_167353485 | G | 6.102 | 8.65E−03 | FGFR1OP | 0.06949 |
| rs16927618 | rs16927618 | G | 0.2634 | 5.48E−03 | PAMR1 | 0.2355 |
| rs16927625 | rs16927625 | G | 0.2634 | 5.48E−03 | PAMR1 | 0.2371 |
| rs17006233 | rs17006233 | C | 0.174 | 7.35E−03 | ADD2 | 0.08791 |
| rs17006627 | imm_2_61243113 | G | 4.538 | 3.68E−03 | C2orf74 | 0.1807 |
| rs17026308 | imm_2_101932459 | A | 3.232 | 4.30E−03 | MAP4K4, LINC01127 | 0.3463 |
| rs17035663 | rs17035663 | A | 2.795 | 6.01E−03 | CHST11 | 0.3367 |
| rs17103104 | 1kg_14_34760729 | G | 0.3232 | 6.12E−03 | KIAA0391 | 0.1697 |
| rs17227583 | imm_5_40413623 | G | 5.104 | 5.68E−03 | LINC00603, PTGER4 | 0.1273 |
| rs17234657 | imm_5_40437266 | C | 5.104 | 5.68E−03 | LINC00603, PTGER4 | 0.1271 |
| rs17235132 | imm_5_40448114 | G | 5.104 | 5.68E−03 | LINC00603, PTGER4 | 0.1299 |
| rs172811 | imm_1_7962536 | A | 2.419 | 6.33E−03 | PARK7 | 0.4275 |
| rs17390873 | rs17390873 | A | 5.907 | 1.80E−03 | ATG4C, LINC00466 | 0.1236 |
| rs17458312 | 1kg_14_34829805 | A | 3.959 | 5.41E−03 | PSMA6 | 0.1837 |
| rs17461863 | rs17461863 | A | 0.2433 | 1.61E−04 | GABRB1 | 0.4427 |
| rs1761455 | seq-rs1761455 | G | 4.527 | 2.17E−03 | LILRA3, LILRA5 | 0.2835 |
| rs1761456 | seq-rs1761456 | A | 4.62 | 2.14E−03 | LILRA3, LILRA5 | 0.2703 |
| rs17623914 | seq-rs17623914 | G | 0.3144 | 8.46E−03 | PTPRC | 0.1239 |
| rs17624462 | rs17624462 | G | 6.076 | 9.40E−03 | ITGBL1 | 0.1023 |
| rs17650496 | imm_6_127312457 | G | 0.09775 | 5.15E−03 | MIR588, RSPO3 | 0.07131 |
| rs17673852 | rs17673852 | G | 6.168 | 8.16E−03 | BMP6 | 0.08145 |
| rs17712328 | imm_2_185817565 | A | 3.092 | 5.99E−03 | ZNF804A, LOC101927196 | 0.2488 |
| rs17730380 | rs1773 0380 | A | 0.3759 | 8.06E−03 | PTPN14 | 0.2934 |
| rs17762453 | imm_2_185776058 | G | 2.862 | 9.46E−03 | ZNF804A, LOC101927196 | 0.2476 |
| rs17771891 | imm_5_131772101 | A | 0.2807 | 4.93E−03 | SLC22A5, C5orf56 | 0.2052 |
| rs17806015 | imm_12_9796538 | G | 4.32 | 9.24E−03 | CD69 | 0.1699 |
| rs17826145 | imm_5_40433947 | A | 5.104 | 5.68E−03 | LINC00603, PTGER4 | 0.127 |
| rs1837 | imm_9_122658050 | A | 2.973 | 6.53E−03 | PHF19 | 0.2603 |
| rs1842399 | rs1842399 | C | 0.2295 | 1.81E−03 | HLA-DQB2, HLA-DOB | 0.2834 |
| rs1845931 | rs1845931 | A | 3.417 | 2.45E−03 | LINC01584 | 0.2688 |
| rs1860598 | rs1860598 | G | 2.995 | 2.61E−03 | FAM184B | 0.4222 |
| rs1872758 | rs1872758 | G | 2.237 | 8.65E−03 | LOC105376360 | 0.4606 |
| rs1873616 | imm_3_46118606 | A | 0.3064 | 6.17E−03 | XCR1, CCR1 | 0.2183 |
| rs1873617 | imm_3_46150984 | A | 0.3161 | 9.22E−03 | XCR1, CCR1 | 0.2099 |
| rs1873618 | imm_3_46150980 | G | 0.3161 | 9.22E−03 | XCR1, CCR1 | 0.21 |
| rs1900493 | rs1900493 | A | 3.226 | 1.47E−03 | PCDH15, MTRNR2L5 | 0.4954 |
| rs1915628 | rs1915628 | A | 2.465 | 8.39E−03 | REEP3, ANXA2P3 | 0.4411 |
| rs1922240 | rs1922240 | G | 2.649 | 7.20E−03 | ABCB1 | 0.3309 |
| rs1927907 | rs1927907 | A | 0.2309 | 9.86E−03 | TLR4 | 0.1424 |
| rs1930952 | imm_6_127275973 | A | 2.516 | 6.74E−03 | MIR588, RSPO3 | 0.4573 |
| rs1936811 | imm_6_127425553 | T | 3.431 | 8.98E−04 | MIR588, RSPO3 | 0.4041 |
| rs1936812 | imm_6_127432378 | G | 3.431 | 8.98E−04 | MIR588, RSPO3 | 0.4025 |
| rs1936814 | imm_6_127434157 | A | 3.431 | 8.98E−04 | \|MIR588, RSPO3 | 0.4028 |
| rs1938341 | rs1938341 | A | 0.4207 | 9.81E−03 | PLD5, LINC01347 | 0.46 |
| rs1948745 | imm_9_34857913 | A | 3.263 | 9.77E−03 | FAM205BP, FAM205C | 0.2258 |
| rs1965079 | rs1965079 | G | 2.966 | 9.12E−03 | CACNG3, RBBP6 | 0.3025 |
| rs196600 | rs196600 | G | 0.3641 | 5.79E−03 | EEPD1 | 0.3419 |
| rs1981524 | imm_5_131784405 | A | 0.2486 | 2.76E−03 | C5orf56 | 0.2057 |
| rs1983608 | rs1983 608 | G | 0.2965 | 5.94E−03 | PRDM2, KAZN | 0.3428 |
| rs1992820 | rs1992820 | C | 2.443 | 7.22E−03 | PCDH15, MTRNR2L5 | 0.4819 |
| rs1992821 | rs1992821 | C | 2.369 | 1.00E−02 | PCDH15, MTRNR2L5 | 0.4833 |
| rs1999805 | rs1999805 | G | 2.607 | 6.08E−03 | ESR1 | 0.4465 |
| rs201292440 | 9-116611115-GAA- | D | 0.305 | 7.83E−03 | TNFSF15_TNFSF8 | 0.2695 |
| | INSERTION | | | | | |
| rs2027033 | imm_6_127262945 | G | 2.516 | 6.74E−03 | MIR588, RSPO3 | 0.4579 |
| rs2048957 | rs2048957 | A | 2.381 | 8.52E−03 | ARHGAP15 | 0.3606 |
| rs2067577 | rs2067577 | C | 0.3151 | 4.25E−03 | HLA-DQB2, HLA-DOB | 0.3311 |
| rs2067644 | rs2067644 | C | 2.991 | 9.71E−03 | DHRS2, DHRS4-AS1 | 0.2345 |
| rs2077845 | rs2077845 | G | 2.949 | 3.84E−03 | GBP4, GBP5 | 0.4072 |
| rs2078610 | rs2078610 | C | 2.668 | 4.18E−03 | GABRB1 | 0.4154 |
| rs2090849 | rs2090849 | A | 2.465 | 8.39E−03 | REEP3, ANXA2P3 | 0.4409 |
| rs2108225 | rs2108225 | A | 0.3984 | 7.61E−03 | SLC26A3, DLD | 0.4214 |
| rs2111057 | imm_12_66787546 | C | 0.2716 | 9.67E−04 | IFNG-AS1, IFNG | 0.4024 |
| rs2113378 | 1kg_2_207039068 | G | 0.2697 | 7.15E−03 | ADAM23 | 0.1687 |
| rs2113496 | imm_2_185889220 | G | 2.518 | 8.44E−03 | ZNF804A, LOC101927196 | 0.3961 |
| rs2116585 | rs2116585 | A | 0.4017 | 5.07E−03 | TTC27 | 0.4424 |
| rs212664 | rs212664 | C | 3.172 | 5.19E−03 | HDAC9 | 0.2977 |
| rs213230 | rs213230 | G | 0.3834 | 8.22E−03 | ZKSCAN3 | 0.2742 |
| rs2157079 | rs2157079 | A | 0.3151 | 4.25E−03 | HLA-DQB2, HLA-DOB | 0.3308 |
| rs2162781 | rs2162781 | A | 2.966 | 9.12E−03 | CACNG3, RBBP6 | 0.3024 |
| rs2163625 | rs2163625 | G | 2.702 | 3.52E−03 | TMEM9B | 0.4115 |
| rs2175679 | imm_4_123743075 | A | 2.731 | 8.08E−03 | IL2, IL21 | 0.3311 |
| rs2193042 | imm_12_66794089 | C | 3.251 | 6.03E−03 | IFNG-AS1, IFNG | 0.2869 |
| rs2194476 | imm_2_185811060 | A | 3.092 | 5.99E−03 | ZNF804A, LOC101927196 | 0.2508 |
| rs2199870 | rs2199870 | G | 0.3377 | 6.40E−03 | HLA-DQB2, HLA-DOB | 0.3312 |
| rs2215185 | 1kg_17_29658015 | G | 0.2217 | 1.87E−03 | CCL11, CCL8 | 0.1868 |
| rs2227203 | imm_1_171145646 | A | 3.026 | 6.70E−03 | FASLG, TNFSF18 | 0.413 |
| rs2228224 | imm_12_56151588 | G | 2.674 | 7.64E−03 | GLI1 | 0.3718 |
| rs2235686 | rs2235686 | A | 0.2228 | 5.45E−03 | CBX7 | 0.1383 |
| rs2239186 | rs2239186 | G | 0.264 | 5.47E−03 | IVDR | 0.1897 |
| rs2241392 | rs2241392 | G | 0.3187 | 3.12E−03 | C3 | 0.3681 |
| rs2242046 | rs2242046 | G | 0.3761 | 5.38E−03 | SLC28A1 | 0.4892 |
| rs2243504 | rs2243504 | C | 2.622 | 9.09E−03 | LINC00926 | 0.4815 |
| rs2246638 | rs2246638 | A | 0.2512 | 3.69E−03 | HCG9, ZNRD1-AS1 | 0.2072 |
| rs225092 | imm_1_7958662 | G | 2.419 | 6.33E−03 | PARK7 | 0.4282 |
| rs225100 | imm_1_7989501 | A | 2.548 | 5.16E−03 | PARK7, ERRFI1 | 0.432 |
| rs225119 | imm_1_7966948 | A | 2.559 | 5.04E−03 | PARK7 | 0.4294 |
| rs226242 | imm_1_7956055 | G | 2.419 | 6.33E−03 | PARK7 | 0.4283 |
| rs226249 | imm_1_7944365 | A | 2.517 | 5.60E−03 | PARK7 | 0.4334 |
| rs226251 | imm_1_7947277 | A | 2.517 | 5.60E−03 | PARK7 | 0.4337 |
| rs226253 | imm_1_7950293 | A | 2.517 | 5.60E−03 | PARK7 | 0.4339 |
| rs2280728 | rs2280728 | C | 2.668 | 2.57E−03 | CASC23 | 0.4916 |
| rs228651 | imm_1_7833686 | A | 2.589 | 6.39E−03 | UTS2 | 0.3905 |
| rs229271 | rs229271 | A | 2.669 | 6.63E−03 | PRKD1, G2E3 | 0.3442 |
| rs2306390 | imm_12_56288866 | A | 2.693 | 8.00E−03 | DTX3 | 0.2555 |
| rs2316184 | rs2316184 | G | 0.2466 | 3.30E−03 | CDYL2 | 0.2381 |
| rs2371685 | imm_5_40427983 | T | 5.104 | 5.68E−03 | LINC00603, PTGER4 | 0.1271 |
| rs2373155 | imm_3_46147076 | A | 0.3064 | 6.17E−03 | XCR1, CCRI | 0.2236 |
| rs2383135 | rs2383135 | C | 0.2879 | 5.95E−03 | SLC24A2, MLLT3 | 0.2111 |
| rs2417306 | rs2417306 | C | 2.792 | 7.07E−03 | GRIN2B | 0.3513 |
| rs2477858 | rs2477858 | G | 3.081 | 3.75E−03 | PCNXL2 | 0.436 |
| rs249959 | imm_5_96190602 | A | 0.1839 | 6.33E−03 | ERAP1, ERAP2 | 0.07402 |
| rs2516470 | rs2516470 | C | 0.3008 | 2.53E−03 | MICA, HCP5 | 0.3161 |
| rs2528691 | rs2528691 | G | 2.833 | 5.72E−03 | IMMP2L, DOCK4 | 0.4921 |
| rs2544913 | rs2544913 | A | 2.747 | 8.38E−03 | ST8SIA4 | 0.3524 |
| rs2548276 | rs2548276 | A | 2.906 | 5.65E−03 | ST8SIA4 | 0.3498 |
| rs2548680 | rs2548680 | A | 2.966 | 9.12E−03 | CACNG3, RBBP6 | 0.3025 |
| rs259942 | 1kg_6_30123146 | A | 0.2137 | 1.83E−03 | ZNRD1-AS1 | 0.1749 |
| rs259942 | rs259942 | A | 0.2137 | 1.83E−03 | ZNRD1-AS1 | 0.1749 |
| rs2621332 | rs2621332 | G | 0.3377 | 6.40E−03 | HLA-DOB | 0.3311 |
| rs2621336 | rs2621336 | G | 0.3377 | 6.40E−03 | HLA-DQB2, HLA-DOB | 0.3311 |
| rs2621390 | rs2621390 | G | 0.2295 | 1.81E−03 | HLA-DQB2, HLA-DOB | 0.2839 |
| rs2621391 | rs2621391 | G | 0.2295 | 1.81E−03 | HLA-DQB2, HLA-DOB | 0.2839 |
| rs2621393 | rs2621393 | G | 0.2295 | 1.81E−03 | HLA-DQB2, HLA-DOB | 0.2834 |
| rs2621421 | rs2621421 | C | 0.2745 | 2.49E−03 | HLA-DQB2, HLA-DOB | 0.3388 |
| rs26519 | imm_5_96175859 | A | 0.148 | 2.29E−03 | ERAP1 | 0.08176 |
| rs2680344 | rs2680344 | G | 0.1663 | 3.70E−04 | HCN4 | 0.2237 |
| rs2700982 | 1kg_7_37361345 | G | 2.5 | 8.15E−03 | ELMO1 | 0.4571 |
| rs2700983 | 1kg_7_37360904 | C | 2.5 | 8.15E−03 | ELMO1 | 0.4571 |
| rs2700986 | 1kg_7_37356329 | A | 3.885 | 1.90E−03 | ELMO1 | 0.2047 |
| rs2700990 | 1kg_7_37349302 | A | 3.861 | 8.13E−04 | ELMO1 | 0.2521 |
| rs2717954 | 1kg_7_37361898 | G | 2.881 | 2.36E−03 | ELMO1 | 0.2877 |
| rs2723980 | 1kg_7_3733 1947 | A | 4.071 | 1.84E−03 | ELMO1 | 0.1898 |
| rs2724011 | 1kg_7_37365041 | A | 4.011 | 7.70E−04 | ELMO1 | 0.2354 |
| rs2724012 | 1kg_7_37355159 | A | 2.732 | 3.93E−03 | ELMO1 | 0.333 |
| rs2724018 | 1kg_7_37358537 | A | 3.885 | 1.90E−03 | ELMO1 | 0.2044 |
| rs2745358 | imm_6_127433163 | G | 3.308 | 7.59E−04 | \|MIR588, RSPO3 | 0.4553 |
| rs276677 | imm_9_34865554 | G | 3.263 | 9.77E−03 | FAM205BP, FAM205C | 0.2258 |
| rs276678 | imm_9_34865731 | G | 3.263 | 9.77E−03 | FAM205BP, FAM205C | 0.2259 |
| rs2767329 | seq-rs2767329 | A | 0.2874 | 2.97E−03 | CD2, PTGFRN | 0.167 |
| rs2777965 | rs2777965 | C | 0.3937 | 9.83E−03 | FCRL4 | 0.3574 |
| rs2780781 | 1kg_1_241020537 | A | 2.626 | 9.16E−03 | PLD5, LINC01347 | 0.4021 |
| rs2780784 | 1kg_1_241026399 | G | 2.733 | 6.34E−03 | PLD5, LINC01347 | 0.4019 |
| rs28567966 | vh_15_98510368 | G | 0.3014 | 9.84E−03 | ADAMTS17 | 0.1664 |
| rs2857114 | rs2857114 | G | 0.3377 | 6.40E−03 | HLA-DOB | 0.3415 |
| rs2857130 | rs2857130 | A | 0.3377 | 6.40E−03 | HLA-DQB2, HLA-DOB | 0.3311 |
| rs2857205 | rs2857205 | A | 0.2295 | 1.81E−03 | HLA-DQB2, HLA-DOB | 0.2836 |
| rs2870955 | imm_12_66788592 | A | 0.2716 | 9.67E−04 | IFNG-AS1, IFNG | 0.4023 |
| rs2913784 | rs2913784 | A | 3.741 | 1.23E−03 | COL23A1 | 0.3284 |
| rs304723 | rs304723 | A | 2.687 | 9.74E−03 | ZNF576 | 0.306 |
| rs3094228 | rs3094228 | G | 4.776 | 1.43E−03 | MICA, HCP5 | 0.2056 |
| rs3099840 | rs3099840 | G | 4.776 | 1.43E−03 | HCP5 | 0.2055 |
| rs3125037 | rs3125037 | G | 0.2192 | 3.88E−04 | ZMYND11 | 0.2784 |
| rs3128941 | rs3128941 | G | 3.782 | 8.45E−04 | HLA-DOA, HLA-DPA1 | 0.4577 |
| rs3129887 | rs3129887 | A | 4.53 | 9.34E−03 | HLA-DRA | 0.1628 |
| rs3130573 | rs3130573 | G | 4.044 | 1.17E−03 | PSORS1C1, PSORS1C2 | 0.3434 |
| rs3131296 | rs3131296 | A | 6.337 | 6.21E−03 | NOTCH4 | 0.121 |
| rs3132956 | rs3132956 | A | 6.337 | 6.21E−03 | NOTCH4 | 0.1212 |
| rs3134796 | rs3134796 | G | 6.337 | 6.21E−03 | NOTCH4 | 0.1218 |
| rs3134942 | rs3134942 | A | 6.337 | 6.21E−03 | NOTCH4 | 0.121 |
| rs3176793 | imm_12_9801987 | A | 4.32 | 9.24E−03 | CD69 | 0.1695 |
| rs31888 | rs31888 | A | 3.35 | 8.38E−03 | CTNND2 | 0.1962 |
| rs336451 | rs336451 | C | 2.632 | 4.36E−03 | TDRP, ERICH1 | 0.4396 |
| rs34733 | imm_5_96187950 | A | 0.1839 | 6.33E−03 | ERAP1, ERAP2 | 0.07413 |
| rs34734 | imm_5_96191025 | A | 0.1839 | 6.33E−03 | ERAP1, ERAP2 | 0.07414 |
| rs34736 | imm_5_96193646 | A | 0.1839 | 6.33E−03 | ERAP1, ERAP2 | 0.07408 |
| rs34902013 | imm_12_66785221 | G | 0.3592 | 4.62E−03 | IFNG-AS1, IFNG | 0.3814 |
| rs35246047 | imm_12_66787520 | A | 0.4066 | 8.72E−03 | IFNG-AS1, IFNG | 0.3729 |
| rs370812 | imm_1_7998481 | G | 2.36 | 8.36E−03 | ERRFI1 | 0.4349 |
| rs371452 | imm_1_8006638 | G | 2.36 | 8.36E−03 | ERRFI1 | 0.4343 |
| rs3732341 | 1kg_2_241304217 | G | 0.3671 | 7.31E−03 | KIF1A | 0.4995 |
| rs3851519 | rs3851519 | A | 3.657 | 6.88E−04 | LY86, RREBI | 0.3995 |
| rs400736 | imm_1_8000896 | A | 2.36 | 8.36E−03 | ERRFI1 | 0.445 |
| rs404032 | seq-rs404032 | C | 14.527 | 2.17E−03 | LILRA3, LILRA5 | 0.2834 |
| rs408320 | imm_1_8007915 | A | 2.36 | 8.36E−03 | ERRFI1 | 0.4336 |
| rs414135 | seq-rs414135 | A | 4.527 | 2.17E−03 | LILRA3, LILRA5 | 0.2833 |
| rs415595 | imm_16_11271193 | G | 0.4405 | 9.32E−03 | TNP2 | 0.474 |
| rs415595 | rs415595 | G | 0.4405 | 9.32E−03 | TNP2 | 0.474 |
| rs416603 | imm_16_11271580 | T | 0.4405 | 9.32E−03 | TNP2 | 0.4737 |
| rs4240842 | imm_1_204921129 | T | 0.3295 | 9.33E−03 | DYRK3, MAPKAPK2 | 0.2247 |
| rs4240845 | imm_1_204942320 | A | 0.3001 | 6.21E−03 | MAPKAPK2 | 0.2236 |
| rs4240847 | imm_1_204963245 | C | 0.2987 | 7.16E−03 | MAPKAPK2 | 0.2192 |
| rs4240848 | imm_1_204963373 | A | 0.2987 | 7.16E−03 | MAPKAPK2 | 0.2192 |
| rs4255613 | imm_12_66784937 | C | 0.2612 | 7.71E−04 | \|IFNG-AS1, IFNG | 0.4026 |
| rs4263302 | rs4263302 | G | 0.2096 | 7.07E−03 | GBE1, LINC00971 | 0.1378 |
| rs4303275 | rs4303275 | A | 3.25 | 2.66E−03 | TRHDE | 0.2789 |
| rs431159 | imm_6_167329832 | A | 6.102 | 8.65E−03 | RNASET2, MIR3939 | 0.07032 |
| rs434202 | rs434202 | G | 0.3196 | 6.03E−03 | GSG1L | 0.2361 |
| rs4381620 | imm_16_11385258 | A | 0.2696 | 8.22E−03 | RMI2, LOC101927131 | 0.1454 |
| rs4393358 | imm_11_118074307 | A | 0.3891 | 9.65E−03 | TREH, DDX6 | 0.3115 |
| rs4407639 | rs4407639 | A | 3.129 | 2.96E−03 | LOC340113, TARS | 0.4526 |
| rs442862 | imm_1_8002081 | A | 2.36 | 8.36E−03 | ERRFI1 | 0.4337 |
| rs4573488 | 1kg_1_22610470 | A | 0.1662 | 9.92E−03 | MIR4418, ZBTB40 | 0.1101 |
| rs4607880 | imm_1_204964104 | A | 0.2987 | 7.16E−03 | MAPKAPK2 | 0.2192 |
| rs4613763 | imm_5_40428485 | G | 5.104 | 5.68E−03 | LINC00603, PTGER4 | 0.1271 |
| rs4632362 | imm_2_102037034 | A | 3.33 | 8.84E−03 | IL1R2, IL1R1 | 0.1524 |
| rs4642322 | rs4642322 | G | 0.3822 | 9.59E−03 | LOC101928858, | 0.4239 |
| | | | | | LOC102467655 | |
| rs4682811 | imm_3_46139799 | A | 0.3064 | 6.17E−03 | XCR1, CCR1 | 0.2186 |
| rs4683158 | imm_3_45985081 | G | 0.334 | 7.94E−03 | FYCO1 | 0.2139 |
| rs4683182 | imm_3_46148940 | A | 0.3164 | 9.74E−03 | XCR1, CCR1 | 0.2117 |
| rs4684448 | rs4684448 | G | 0.3054 | 2.35E−03 | ITPR1, BHLHE40-AS1 | 0.4267 |
| rs4694846 | rs4694846 | G | 3.476 | 8.38E−04 | GABRB1 | 0.4309 |
| rs4734880 | rs4734880 | C | 3.118 | 3.16E−03 | ZFPM2 | 0.44 |
| rs4763299 | imm_12_9795716 | A | 4.32 | 9.24E−03 | CD69 | 0.1698 |
| rs4771332 | 1kg_13_98868458 | A | 0.3246 | 1.83E−03 | MIR548AN, LINC01232 | 0.2977 |
| rs4798791 | rs4798791 | A | 3.037 | 2.30E−03 | ANKRD12 | 0.3775 |
| rs4806768 | seq-rs4806768 | A | 2.977 | 2.07E−03 | LAIR2 | 0.4648 |
| rs4837462 | rs4837462 | C | 2.584 | 9.03E−03 | LOC101928797 | 0.4944 |
| rs4845130 | imm_1_204939110 | G | 0.3295 | 9.33E−03 | MAPKAPK2 | 0.2266 |
| rs4851535 | imm_2_102032725 | G | 3.33 | 8.84E−03 | IL1R2, IL1R1 | 0.152 |
| rs4851537 | imm_2_102039121 | A | 3.33 | 8.84E−03 | IL1R2, IL1R1 | 0.152 |
| rs488141 | imm_11_118076378 | G | 0.3891 | 9.65E−03 | TREH, DDX6 | 0.3112 |
| rs489126 | imm_11_118077957 | A | 0.3891 | 9.65E−03 | TREH, DDX6 | 0.3121 |
| rs4894717 | rs4894717 | G | 4.164 | 7.92E−03 | NAALADL2 | 0.1955 |
| rs4895819 | imm_6_127266989 | A | 2.516 | 6.74E−03 | MIR588, RSPO3 | 0.4575 |
| rs4945744 | imm_6_106720616 | A | 0.3222 | 7.45E−03 | PRDM1, ATG5 | 0.2513 |
| rs4946730 | imm_6_106719784 | A | 0.3222 | 7.45E−03 | PRDM1, ATG5 | 0.2535 |
| rs4946731 | imm_6_106720617 | C | 0.3222 | 7.45E−03 | PRDM1, ATG5 | 0.2513 |
| rs4948003 | rs4948003 | A | 2.927 | 3.86E−03 | ELDR, LANCL2 | 0.2852 |
| rs504215 | imm_19_53964296 | A | 2.957 | 7.09E−03 | FGF21, BCAT2 | 0.3304 |
| rs523715 | imm_11_118079398 | A | 0.3891 | 9.65E−03 | TREH, DDX6 | 0.312 |
| rs523793 | imm_11_118075907 | A | 0.3891 | 9.65E−03 | TREH, DDX6 | 0.3112 |
| rs544452 | imm_11_118076567 | A | 0.3891 | 9.65E−03 | TREH, DDX6 | 0.3112 |
| rs552079 | imm_11_118078232 | G | 0.3891 | 9.65E−03 | TREH, DDX6 | 0.3116 |
| rs55693281 | 1kg_14_34833986 | T | 3.959 | 5.41E−03 | PSMA6 | 0.1847 |
| rs55735886 | imm_2_102049163 | G | 3.33 | 8.84E−03 | IL1R2, IL1R1 | 0.152 |
| rs55782190 | imm_5_40449187 | G | 5.104 | 5.68E−03 | LINC00603, PTGER4 | 0.1299 |
| rs55955629 | imm_2_185878330 | C | 2.915 | 8.95E−03 | ZNF804A, LOC101927196 | 0.2493 |
| rs56244034 | imm_5_40411916 | A | 5.104 | 5.68E−03 | LINC00603, PTGER4 | 0.1272 |
| rs56277923 | imm_5_40719882 | A | 0.3065 | 7.55E−03 | PTGER4 | 0.2055 |
| rs56309786 | imm_5_40468984 | A | 5.104 | 5.68E−03 | LINC00603, PTGER4 | 0.1298 |
| rs56411893 | imm_3_48744859 | G | 3.347 | 8.39E−03 | IP6K2, PRKAR2A | 0.157 |
| rs570949 | imm_11_118077121 | A | 0.3891 | 9.65E−03 | TREH, DDX6 | 0.3112 |
| rs57275892 | 1kg_14_34817022 | A | 0.3232 | 6.12E−03 | PSMA6 | 0.1779 |
| rs57298362 | imm_5_96218620 | A | 0.1839 | 6.33E−03 | ERAP1, ERAP2 | 0.0698 |
| rs5766248 | rs5766248 | A | 3.349 | 4.96E−03 | PHF21B | 0.1716 |
| rs57663955 | imm_5_96220798 | G | 0.1839 | 6.33E−03 | ERAP1, ERAP2 | 0.06959 |
| rs58587603 | imm_5_96218383 | G | 0.1839 | 6.33E−03 | ERAP1, ERAP2 | 0.06969 |
| rs58626985 | imm_2_101932004 | A | 3.232 | 4.30E−03 | MAP4K4, LINC01127 | 0.3454 |
| rs59179941 | seq-rs59179941 | A | 0.2216 | 1.60E−03 | LAIR2, KIR3DX1 | 0.2432 |
| rs59197404 | imm_2_61707640 | G | 3.778 | 6.57E−03 | XPO1, FAM161A | 0.1898 |
| rs592625 | rs592625 | G | 0.3177 | 6.16E−03 | HLA-DOA | 0.1785 |
| rs59315630 | imm_2_102038604 | A | 3.33 | 8.84E−03 | IL1R2, IL1R1 | 0.1519 |
| rs595158 | rs595158 | A | 3.589 | 6.08E−04 | VPS37C | 0.4987 |
| rs6021233 | rs6021233 | A | 2.839 | 7.43E−03 | NFATC2 | 0.4285 |
| rs60376893 | imm_5_96218190 | G | 0.1839 | 6.33E−03 | ERAP1, ERAP2 | 0.06969 |
| rs6061720 | rs6061720 | A | 2.56 | 9.86E−03 | CDH4 | 0.4087 |
| rs61227121 | imm_5_96220610 | G | 0.1839 | 6.33E−03 | ERAP1, ERAP2 | 0.06959 |
| rs616340 | rs616340 | A | 3.367 | 1.37E−03 | CD5 | 0.3743 |
| rs617384 | imm_5_141397471 | C | 0.3802 | 7.91E−03 | GNPDA1, NDFIP1 | 0.4691 |
| rs61818748 | vh_1_156635016 | A | 0.1154 | 7.26E−03 | OR10T2 | 0.08112 |
| rs61988266 | 1kg_14 34781549 | C | 4.325 | 2.33E−03 | KIAA0391 | 0.237 |
| rs61988271 | 1kg_14_34816501 | G | 3.959 | 5.41E−03 | KIAA0391, PSMA6 | 0.1849 |
| rs61989546 | 1kg_14_34754403 | G | 4.265 | 2.34E−03 | KIAA0391 | 0.2358 |
| rs61989547 | 1kg_14_34761831 | G | 4.265 | 2.34E−03 | KIAA0391 | 0.2344 |
| rs62006055 | imm_15_65226683 | G | 2.924 | 8.12E−03 | SMAD3 | 0.2628 |
| rs62011167 | imm_15_77049780 | G | 0.2357 | 3.40E−03 | RASGRF1 | 0.1746 |
| rs62056381 | 1kg_17_29699681 | A | 0.2413 | 3.24E−03 | CCL8, CCL13 | 0.1897 |
| rs62198770 | imm_2_185888902 | G | 2.518 | 8.44E−03 | ZNF804A, LOC101927196 | 0.3961 |
| rs62200005 | imm_2_185836565 | G | 3.092 | 5.99E−03 | ZNF804A, LOC101927196 | 0.2492 |
| rs62200032 | imm_2_185875583 | A | 2.915 | 8.95E−03 | ZNF804A, LOC101927196 | 0.2493 |
| rs62200034 | imm_2_185887586 | T | 2.518 | 8.44E−03 | ZNF804A, LOC101927196 | 0.3961 |
| rs62385693 | imm_5_131801573 | G | 0.306 | 8.73E−03 | C5orf56 | 0.2068 |
| rs637174 | imm_19_53958748 | A | 2.989 | 6.70E−03 | FGF21, BCAT2 | 0.3205 |
| rs6428670 | seq-t1d-1-117168713-T-C | A | 0.3515 | 9.57E−03 | CD2, PTGFRN | 0.1663 |
| rs6428671 | seq-rs6428671 | A | 0.3515 | 9.57E−03 | CD2, PTGFRN | 0.1666 |
| rs6441996 | imm_3_46480270 | G | 0.3335 | 5.60E−03 | ILTF | 0.2595 |
| rs6447550 | rs6447550 | A | 0.2856 | 5.27E−04 | GABRB1 | 0.4839 |
| rs6462484 | rs6462484 | A | 0.3305 | 3.90E−03 | BBS9 | 0.3993 |
| rs647031 | imm_5_96184512 | A | 0.1839 | 6.33E−03 | ERAP1, ERAP2 | 0.07418 |
| rs6476470 | imm_9_34919071 | G | 3.857 | 4.74E−03 | FAM205C, PHF24 | 0.1964 |
| rs6478109 | imm_9_116608587 | A | 0.2537 | 3.04E−03 | TNFSF15 | 0.2995 |
| rs6481157 | rs6481157 | A | 2.766 | 2.98E−03 | PCDH15, MTRNR2L5 | 0.4866 |
| rs651279 | seq-rs651279 | G | 4.527 | 2.17E−03 | LILRA3, LILRA5 | 0.2841 |
| rs6562463 | rs6562463 | T | 0.363 | 5.23E−03 | PCDH9 | 0.4401 |
| rs657769 | imm_11_118076526 | A | 0.3891 | 9.65E−03 | TREH, DDX6 | 0.3112 |
| rs658676 | imm_11_118076333 | A | 0.3891 | 9.65E−03 | TREH, DDX6 | 0.3115 |
| rs6684369 | rs6684369 | G | 5.31 | 7.08E−03 | PLXNA2, MIR205HG | 0.1467 |
| rs6691768 | rs6691768 | G | 2.453 | 4.71E−03 | NFIA | 0.4094 |
| rs6708276 | rs6708276 | G | 2.668 | 4.57E−03 | ARHGAP15 | 0.3447 |
| rs6723737 | rs6723737 | A | 2.644 | 8.38E−03 | LINC00486 | 0.3937 |
| rs673547 | imm_11_118078549 | A | 0.3891 | 9.65E−03 | TREH, DDX6 | 0.3115 |
| rs6737109 | rs6737109 | G | 0.2239 | 1.65E−04 | LOC102723362, KLHL29 | 0.406 |
| rs6740218 | imm_2_61712593 | A | 3.778 | 6.57E−03 | XPO1, FAM161A | 0.1885 |
| rs6748538 | imm_2_102045141 | C | 3.685 | 4.90E−03 | IL1R2, IL1R1 | 0.1553 |
| rs6757588 | rs6757588 | G | 2.709 | 4.77E−03 | ARHGAP15 | 0.3473 |
| rs6768569 | rs6768569 | A | 2.64 | 7.75E−03 | ITPR1 | 0.3818 |
| rs6802312 | imm_3_46000945 | A | 0.334 | 7.94E−03 | FYCO1 | 0.2156 |
| rs6808712 | imm_3_46106235 | G | 0.3064 | 6.17E−03 | XCR1, CCR1 | 0.2184 |
| rs6819371 | imm_4_123770482 | A | 2.731 | 8.08E−03 | IL21-AS1 | 0.3346 |
| rs6820791 | imm_4_123741233 | A | 2.594 | 9.69E−03 | IL2, IL21 | 0.4047 |
| rs6820964 | imm_4_123741173 | A | 2.594 | 9.69E−03 | IL2, IL21 | 0.4048 |
| rs6826110 | imm_4_123741689 | G | 2.594 | 9.69E−03 | IL2, IL21 | 0.4048 |
| rs6828555 | rs6828555 | G | 2.34 | 9.32E−03 | HOPX.SPINK2 | 0.4272 |
| rs683028 | rs683028 | G | 3.375 | 2.85E−03 | DKFZp686K1684, | 0.4055 |
| | | | | | LOC100506675 | |
| rs6835457 | imm_4_123730576 | G | 2.731 | 8.08E−03 | IL2, IL21 | 0.3309 |
| rs6845976 | rs6845976 | G | 0.3741 | 6.62E−03 | TENM3 | 0.3747 |
| rs6862868 | rs6862868 | A | 0.3322 | 7.46E−03 | WWC1 | 0.4007 |
| rs687664 | imm_11_118079395 | A | 0.3891 | 9.65E−03 | TREH, DDX6 | 0.3127 |
| rs6879283 | imm_5_40437990 | G | 5.104 | 5.68E−03 | LINC00603, PTGER4 | 0.1271 |
| rs6883975 | imm_5_40438434 | A | 5.104 | 5.68E−03 | LINC00603, PTGER4 | 0.1271 |
| rs6889364 | imm_5_40383226 | A | 5.104 | 5.68E−03 | LINC00603, PTGER4 | 0.1274 |
| rs6902885 | imm_6_127422175 | A | 3.246 | 1.33E−03 | MIR588, RSPO3 | 0.4009 |
| rs6920606 | rs6920606 | A | 3.153 | 2.69E−03 | HLA-DOA, HLA-DPA1 | 0.4959 |
| rs6920701 | rs6920701 | G | 0.2663 | 4.82E−03 | MAS1, IGF2R | 0.2233 |
| rs7022658 | imm_9_4304965 | A | 2.875 | 8.07E−03 | GLIS3, SLC1A1 | 0.3554 |
| rs7030473 | rs7030473 | A | 2.705 | 5.93E−03 | RGS3, ZNF618 | 0.3209 |
| rs7033016 | imm_9_34901879 | G | 3.857 | 4.74E−03 | AM205C, PHF24 | 0.1978 |
| rs7034974 | imm_9_4307266 | C | 2.875 | 8.07E−03 | GLIS3, SLC1A1 | 0.3546 |
| rs7037909 | imm_9_4304427 | G | 3.108 | 5.02E−03 | GLIS3, SLC1A1 | 0.359 |
| rs7038304 | imm_9_4304752 | G | 2.875 | 8.07E−03 | GLIS3, SLC1A1 | 0.3563 |
| rs7040756 | imm_9_34919667 | T | 3.857 | 4.74E−03 | FAM205C, PHF24 | 0.1964 |
| rs7041922 | imm_9_34928198 | G | 3.857 | 4.74E−03 | FAM205C, PHF24 | 0.1958 |
| rs704847 | imm _1_170995554 | C | 2.382 | 9.43E−03 | FASLG, TNFSF18 | 0.3957 |
| rs7073883 | rs7073 883 | G | 3.798 | 3.10E−03 | PCDH15 | 0.2782 |
| rs7130 | imm_3_45934519 | A | 0.316 | 5.72E−03 | FYCO1 | 0.2203 |
| rs7133171 | imm_12_66789421 | G | 0.2716 | 9.67E−04 | IFNG-AS1, IFNG | 0.4024 |
| rs7134472 | imm_12_66786253 | A | 0.3592 | 4.62E−03 | IFNG-AS1, IFNG | 0.3812 |
| rs7134599 | imm_12_66786342 | A | 0.3592 | 4.62E−03 | IFNG-AS1, IFNG | 0.3812 |
| rs7137158 | imm_12_66790187 | G | 0.2716 | 9.67E−04 | IFNG-AS1, IFNG | 0.4023 |
| rs7138407 | imm_12_66787129 | A | 0.3592 | 4.62E−03 | IFNG-AS1, IFNG | 0.381 |
| rs714903 | rs714903 | A | 3.116 | 9.89E−03 | ESRRB, VASH1 | 0.2871 |
| rs714904 | rs714904 | G | 3.116 | 9.89E−03 | ESRRB, VASH1 | 0.2872 |
| rs7158151 | 1kg_14_34814773 | A | 3.959 | 5.41E−03 | KIAA0391, PSMA6 | 0.1843 |
| rs7158706 | 1kg_14_34806443 | A | 3.959 | 5.41E−03 | KIAA0391 | 0.1831 |
| rs7164805 | rs7164805 | A | 0.3072 | 1.44E−03 | BCL2A1, ZFAND6 | 0.4474 |
| rs7179025 | rs7179025 | G | 0.2697 | 5.67E−03 | SLC27A2 | 0.1883 |
| rs7180547 | rs7180547 | G | 2.463 | 8.95E−03 | RORA | 0.3919 |
| rs7180888 | 1595102199 | A | 0.3955 | 4.73E−03 | NR2F2, SPATA8-AS1 | 0.4605 |
| rs7183113 | rs7183113 | C | 4.909 | 5.43E−03 | SPRED1 | 0.1406 |
| rs7194404 | rs7194404 | A | 2.581 | 5.50E−03 | FENDRR | 0.4003 |
| rs722748 | imm_12_66786791 | LA | 0.2716 | 9.67E−04 | IFNG-AS1, IFNG | 0.4024 |
| rs722749 | imm_12_66786905 | G | 0.2716 | 9.67E−04 | IFNG-AS1, IFNG | 0.4024 |
| rs723403 | imm_12_66787721 | G | 0.3592 | 4.62E−03 | IFNG-AS1, IFNG | 0.3812 |
| rs723788 | seq-rs723788 | A | 0.3475 | 7.55E−03 | CD2, PTGFRN | 0.1677 |
| rs728294 | rs728294 | A | 3.344 | 6.82E−04 | GABRB1 | 0.4624 |
| rs7301797 | imm_12_66789157 | G | 0.2716 | 9.67E−04 | IFNG-AS1, IFNG | 0.4023 |
| rs7304878 | imm_12_66772251 | G | 0.2571 | 5.94E−04 | IFNG-AS1, IFNG | 0.3953 |
| rs7305123 | rs7305123 | G | 0.317 | 9.49E−03 | LOC100507195, RAP1B | 0.1948 |
| rs7306440 | imm_12_66790296 | G | 0.2716 | 9.67E−04 | IFNG-AS1, IFNG | 0.4023 |
| rs73090828 | imm_5_40473854 | A | 5.104 | 5.68E−03 | LINC00603, PTGER4 | 0.1299 |
| rs73099728 | imm_5_40368755 | G | 5.104 | 5.68E−03 | LINC00603, PTGER4 | 0.1275 |
| rs73099741 | imm_5_40382448 | A | 5.104 | 5.68E−03 | LINC00603, PTGER4 | 0.1274 |
| rs7311875 | imm_12_127859220 | G | 3.31 | 6.29E−03 | SLC15A4 | 0.3012 |
| rs73495567 | imm_9_34920450 | G | 3.857 | 4.74E−03 | FAM205C, PHF24 | 0.1963 |
| rs7370700 | imm_2_185898466 | A | 2.847 | 7.83E−03 | ZNF804A, LOC101927196 | 0.2517 |
| rs7404848 | rs7404848 | A | 0.1723 | 7.04E−04 | CDYL2 | 0.2421 |
| rs74343853 | imm_5_96211894 | A | 0.1839 | 6.33E−03 | ERAP1, ERAP2 | 0.06964 |
| rs74539718 | imm_5_96214560 | A | 0.1839 | 6.33E−03 | ERAP1, ERAP2 | 0.06964 |
| rs74554728 | imm_5_96217132 | A | 0.1839 | 6.33E−03 | ERAP1, ERAP2 | 0.06986 |
| rs74836438 | imm_5_96213604 | G | 0.1839 | 6.33E−03 | ERAP1, ERAP2 | 0.06964 |
| rs748569 | imm_2_61710681 | C | 4.184 | 3.85E−03 | XPO1, FAM161A | 0.1911 |
| rs748570 | imm_2_61711025 | G | 3.778 | 6.57E−03 | XPO1, FAM161A | 0.1893 |
| rs748571 | imm_2_61711589 | G | 3.778 | 6.57E−03 | XPO1, FAM161A | 0.1893 |
| rs74975998 | imm_5_96221291 | C | 0.1839 | 6.33E−03 | ERAP1, ERAP2 | 0.06955 |
| rs74999885 | imm_5_96219667 | A | 0.1839 | 6.33E−03 | ERAP1, ERAP2 | 0.06959 |
| rs75006507 | imm_5_96219612 | A | 0.1839 | 6.33E−03 | ERAP1, ERAP2 | 0.06965 |
| rs75245350 | imm_5_96221441 | A | 0.1839 | 6.33E−03 | ERAP1, ERAP2 | 0.06964 |
| rs75424572 | imm_6_127405932 | C | 0.1243 | 3.47E−03 | MIR588, RSPO3 | 0.06444 |
| rs7570465 | imm_2_102048456 | A | 3.33 | 8.84E−03 | IL1R2, IL1R1 | 0.1521 |
| rs7576335 | imm_2_185894196 | A | 2.518 | 8.44E−03 | ZNF804A, LOC101927196 | 0.396 |
| rs75784526 | imm_5_96207900 | G | 0.1839 | 6.33E−03 | ERAP1, ERAP2 | 0.06965 |
| rs75854356 | imm_5_96219380 | A | 0.1839 | 6.33E−03 | ERAP1, ERAP2 | 0.06964 |
| rs7590132 | imm_2_61713189 | A | 3.778 | 6.57E−03 | XPO1, FAM161A | 0.1886 |
| rs75945206 | imm_5_96212554 | C | 0.1839 | 6.33E−03 | ERAP1, ERAP2 | 0.06964 |
| rs759819 | seq-rs759819 | G | 4.527 | 2.17E−03 | LILRA3, LILRA5 | 0.2835 |
| rs7607342 | rs7607342 | A | 3.13 | 2.33E−03 | MIR4431, ASB3 | 0.4733 |
| rs7618618 | imm_3_45938501 | C | 0.3251 | 8.04E−03 | FYCO1 | 0.2331 |
| rs7634822 | imm_3_46149940 | C | 0.2932 | 5.97E−03 | XCR1, CCR1 | 0.21 |
| rs76530425 | imm_5_96222950 | G | 0.1839 | 6.33E−03 | ERAP1, ERAP2 | 0.06954 |
| rs7653682 | imm_3_46004275 | A | 0.334 | 7.94E−03 | FYCO1 | 0.2157 |
| rs76668981 | imm_5_96222630 | A | 0.1839 | 6.33E−03 | ERAP1, ERAP2 | 0.06953 |
| rs7669697 | imm_4_123741889 | T | 2.594 | 9.69E−03 | IL2, IL21 | 0.4045 |
| rs7669958 | rs7669958 | A | 3.655 | 6.65E−04 | GABRB1 | 0.3795 |
| rs7670387 | seq-rs7670387 | C | 2.594 | 9.69E−03 | IL2, IL21 | 0.4046 |
| rs7677890 | rs7677890 | A | 0.2433 | 1.61E−04 | \|GABRB1 | 0.4432 |
| rs7702091 | rs7702091 | A | 3.129 | 2.96E−03 | LOC340113, TARS | 0.4535 |
| rs7704457 | imm_5_131772689 | G | 0.3132 | 8.52E−03 | SLC22A5, C5orf56 | 0.2067 |
| rs7708451 | ccc-5-96206669-C-T | A | 0.1839 | 6.33E−03 | ERAP1, ERAP2 | 0.0702 |
| rs77130822 | imm_4_123232824 | G | 0.2649 | 4.45E−03 | TRPC3, KIAA1109 | 0.2005 |
| rs77166924 | imm_5_96205917 | A | 0.1839 | 6.33E−03 | ERAP1, ERAP2 | 0.07012 |
| rs77202274 | imm_5_96213649 | T | 0.1839 | 6.33E−03 | ERAP1, ERAP2 | 0.06964 |
| rs7726182 | imm 5 35850767 | C | 0.1289 | 9.20E−03 | SPEF2 | 0.09748 |
| rs77303760 | imm_5_96221226 | T | 0.1839 | 6.33E−03 | ERAP1, ERAP2 | 0.06959 |
| rs77307641 | imm_5_96216585 | A | 0.1839 | 6.33E−03 | ERAP1, ERAP2 | 0.06959 |
| rs7734434 | imm_5_40472455 | A | 5.104 | 5.68E−03 | LINC00603, PTGER4 | 0.1297 |
| rs77350916 | imm_5_96208901 | A | 0.1839 | 6.33E−03 | ERAP1, ERAP2 | 0.06965 |
| rs77387196 | imm_5_96208000 | G | 0.1839 | 6.33E−03 | ERAP1, ERAP2 | 0.06944 |
| rs77402415 | imm_5_96212846 | G | 0.1839 | 6.33E−03 | ERAP1, ERAP2 | 0.06995 |
| rs7743393 | imm_6_127437908 | A | 3.246 | 1.33E−03 | MIR588, RSPO3 | 0.3998 |
| rs77458442 | imm_5_96215811 | A | 0.1839 | 6.33E−03 | ERAP1, ERAP2 | 0.06965 |
| rs7748394 | imm_6_106732576 | G | 0.3222 | 7.45E−03 | PRDM1, ATG5 | 0.2538 |
| rs77570530 | imm_5_96216401 | G | 0.1839 | 6.33E−03 | ERAP1, ERAP2 | 0.06964 |
| rs7774158 | rs7774158 | A | 0.3555 | 3.57E−03 | HLA-DOA, HLA-DPA1 | 0.4 |
| rs77782465 | seq-NOVEL-1738 | G | 0.3075 | 8.13E−03 | PFKFB3 | 0.1709 |
| rs77829813 | imm_5_96221403 | C | 0.1839 | 6.33E−03 | ERAP1, ERAP2 | 0.06959 |
| rs77955889 | imm_5_96215472 | A | 0.1839 | 6.33E−03 | ERAP1, ERAP2 | 0.06965 |
| rs78378074 | imm_5_96219298 | G | 0.1839 | 6.33E−03 | ERAP1, ERAP2 | 0.06964 |
| rs7839434 | imm_8_ 11363051 | G | 3.088 | 7.90E−03 | FAM167A, BLK | 0.2173 |
| rs78426265 | 1kg_14_34832742 | A | 0.3363 | 6.97E−03 | PSMA6 | 0.1827 |
| rs7848647 | imm_9_116608867 | A | 0.2537 | 3.04E−03 | TNFSF15 | 0.2978 |
| rs78648967 | imm_20 44210993 | A | 0.1035 | 8.20E−03 | CD40, CDH22 | 0.04432 |
| rs78664442 | imm_3_161187500 | A | 0.1209 | 8.43E−03 | IL12A-AS1 | 0.0612 |
| rs78698613 | imm_6_127382349 | A | 0.1303 | 3.86E−03 | MIR588, R.SPO3 | 0.07251 |
| rs78721094 | imm_5_96217919 | G | 0.1839 | 6.33E−03 | ERAP1, ERAP2 | 0.06961 |
| rs79004828 | imm_5_96215381 | A | 0.1839 | 6.33E−03 | ERAP1, ERAP2 | 0.06964 |
| rs7908011 | imm_10_ 61673395 | G | 0.3248 | 9.81E−03 | ANK3 | 0.2395 |
| rs79281461 | imm_5_96221281 | A | 0.1839 | 6.33E−03 | ERAP1, ERAP2 | 0.06959 |
| rs79517864 | imm_6_127433740 | G | 0.09908 | 2.17E−03 | MIR588, RSPO3 | 0.06479 |
| rs79622368 | imm__5_96212384 | A | 0.1839 | 6.33E−03 | ERAP1, ERAP2 | 0.06964 |
| rs798009 | seq-rs798009 | A | 0.3475 | 7.55E−03 | CD2, PTGFRN | 0.1681 |
| rs798011 | seq-rs798011 | G | 0.3475 | 7.55E−03 | CD2, PTGFRN | 0.1682 |
| rs80099993 | seq-t1d-1-196889070-G-A | A | 0.2603 | 5.19E−03 | PTPRC | 0.1234 |
| rs80183034 | imm_5_96220474 | T | 0.1839 | 6.33E−03 | ERAP1, ERAP2 | 0.06959 |
| rs8018597 | 1kg_14_34841700 | A | 3.959 | 5.41E−03 | PSMA6 | 0.1854 |
| rs8026118 | rs8026118 | G | 4.909 | 5.43E−03 | SPRED1 | 0.1407 |
| rs8029728 | rs8029728 | G | 4.909 | 5.43E−03 | SPRED1 | 0.1406 |
| rs8081687 | rs8081687 | A | 0.2258 | 5.51E−04 | ABR, BHLHA9 | 0.3198 |
| rs859641 | imm_1_170973027 | A | 2.454 | 9.01E−03 | FASLG, TNFSF18 | 0.437 |
| rs86567 | rs86567 | C | 3.152 | 3.69E−03 | HLA-DOA | 0.3778 |
| rs885691 | 1kg_17_29665338 | A | 0.2217 | 1.87E−03 | CCL11, CCL8 | 0.1863 |
| rs888001 | imm_2_102042026 | A | 3.33 | 8.84E−03 | IL1R2, IL1R1 | 0.152 |
| rs888002 | imm_2_102041857 | A | 3.33 | 8.84E−03 | IL1R2, IL1R1 | 0.1521 |
| rs888003 | imm_2_102041430 | A | 3.33 | 8.84E−03 | IL1R2, IL1R1 | 0.1519 |
| rs895123 | imm_5_40419818 | G | 5.104 | 5.68E−03 | LINC00603, PTGER4 | 0.1272 |
| rs904634 | imm_3_45992308 | A | 0.334 | 7.94E−03 | FYCO1 | 0.2141 |
| rs911887 | rs911887 | G | 0.3828 | 7.06E−03 | SFTPD | 0.3975 |
| rs914842 | imm_9_122658792 | A | 3.225 | 6.05E−03 | PHF19 | 0.226 |
| rs914951 | 1kg_1_241018701 | A | 2.733 | 6.34E−03 | PLD5, LINC01347 | 0.4025 |
| rs9291908 | rs9291908 | G | 0.3822 | 9.59E−03 | LOC101928858, | 0.4245 |
| | | | | | LOC102467655 | |
| rs9321069 | imm_6_127434670 | A | 3.246 | 1.33E−03 | MIR588, RSPO3 | 0.3996 |
| rs9372856 | imm_6_127430145 | C | 3.431 | 8.98E−04 | MIR588, RSPO3 | 0.4038 |
| rs9375478 | imm_6_127274638 | G | 2.516 | 6.74E−03 | MIR588, RSPO3 | 0.4577 |
| rs9375487 | imm_6_127438933 | G | 3.521 | 6.58E−04 | MIR588, RSPO3 | 0.4033 |
| rs9388538 | imm_6_127271081 | G | 2.516 | 6.74E−03 | MIR588, RSPO3 | 0.4578 |
| rs9388546 | imm_6_127432542 | C | 3.246 | 1.33E−03 | MIR588, RSPO3 | 10.4 |
| rs9401938 | imm_6_127432412 | A | 3.431 | 8.98E−04 | MIR588, RSPO3 | 0.4024 |
| rs9402715 | rs9402715 | G | 2.354 | 9.27E−03 | LINC00271 | 0.4475 |
| rs9444259 | rs9444259 | G | 3.424 | 9.44E−04 | TBX18, NT5E | 0.3339 |
| rs9456815 | rs9456815 | A | 3.725 | 6.85E−03 | PACRG | 0.1667 |
| rs972275 | imm_6_127433537 | G | 3.431 | 8.98E−04 | MIR588, RSPO3 | 0.4024 |
| rs975403 | imm_4_123741090 | A | 2.594 | 9.69E−03 | IL2, IL21 | 0.4048 |
| rs975405 | imm_4_123740630 | G | 2.594 | 9.69E−03 | IL2, IL21 | 0.4049 |
| rs976183 | imm_4_123742180 | G | 2.594 | 9.69E−03 | IL2, IL21 | 0.4049 |
| rs976184 | imm_4_123742121 | G | 2.594 | 9.69E−03 | IL2, IL21 | 0.4048 |
| rs9807677 | 1kg_18_41082119 | A | 3.406 | 7.01E−03 | SLC14A2 | 0.1927 |
| rs9810934 | imm_3_45929356 | A | 0.316 | 5.72E−03 | LZTFL1 | 0.22 |
| rs9813877 | rs9813877 | A | 2.289 | 9.95E−03 | NEK11 | 0.3797 |
| rs9815671 | rs9815671 | A | 2.677 | 7.33E−03 | MIR548AY | 0.3802 |
| rs9973057 | 1kg_18_41078925 | G | 3.979 | 3.03E−03 | SLC14A2 | 0.2024 |
| rs9976328 | rs9976328 | G | 0.2846 | 6.47E−03 | DYRKIA | 0.1692 |

TABLE 13
Polymorphisms associated with high-low TL1A fold-change and Signal One Risk
(logistic model)

| | | Minor | | | | |
|---|---|---|---|---|---|---|
| Poly- | | Allele | | | | |
| morphism | Illumina_id | (A1) | OR | P | Gene | MAF |
| rs2129446 | rs2129446 | A | 0.1861 | 1.22E−03 | LOC105376360 | 0.3539 |
| rs10502034 | rs10502034 | A | 10.16 | 1.59E−03 | MIR7641-1, LOC102723895 | 0.4538 |
| rs8112975 | 1kg_19_18201910 | G | 10.87 | 1.67E−03 | PDE4C | 0.293 |
| rs4275832 | rs4275832 | G | 10.92 | 1.78E−03 | LOC101927286 | 0.3151 |
| rs62120394 | 1kg_19_18199709 | A | 10.05 | 1.92E−03 | PDE4C | 0.3046 |
| rs3130573 | rs3130573 | G | 11.54 | 1.94E−03 | PSORS1C1, PSORS1C2 | 0.3434 |
| rs2680344 | rs2680344 | G | 0.09747 | 2.30E−03 | HCN4 | 0.2237 |
| rs1872758 | rs1872758 | G | 6.145 | 2.31E−03 | LOC105376360 | 0.4606 |
| rs4350242 | rs4350242 | G | 8.525 | 2.48E−03 | LOC101927412, LOC101927434 | 0.4172 |
| rs56331483 | imm_15_36691116 | G | 0.1042 | 2.78E−03 | RASGRP1, C15orf53 | 0.09214 |
| rs259942 | 1kg_6_30123146 | A | 0.07234 | 2.95E−03 | ZNRD1-AS1 | 0.1749 |
| rs259942 | rs259942 | A | 0.07234 | 2.95E−03 | ZNRD1-AS1 | 0.1749 |
| rs1457020 | rs1457020 | A | 0.09518 | 2.99E−03 | LINC01467, NONE | 0.2842 |
| rs3850641 | imm_1_171442455 | G | 0.1185 | 3.09E−03 | TNFSF4 | 0.1741 |
| rs7404848 | rs7404848 | A | 0.04301 | 3.16E−03 | CDYL2 | 0.2421 |
| rs4729450 | rs4729450 | A | 0.1235 | 3.19E−03 | LOC101927243, PTPN12 | 0.4871 |
| rs1922240 | rs1922240 | G | 11.84 | 3.22E−03 | ABCB1 | 0.3309 |
| rs16967858 | rs16967858 | A | 0.09026 | 3.23E−03 | TMEM266 | 0.171 |
| rs2070851 | rs2070851 | G | 0.1089 | 3.26E−03 | F2 | 0.2137 |
| rs2256919 | 1kg_6_30048729 | C | 0.1719 | 3.35E−03 | HLA-A, HCG9 | 0.435 |
| rs2735079 | rs2735079 | A | 0.1703 | 3.39E−03 | HLA-A, HCG9 | 0.4354 |
| rs1910553 | rs1910553 | A | 0.1273 | 3.40E−03 | CREB5 | 0.3479 |
| rs11666033 | 1kg_19_18195805 | A | 8.384 | 3.59E−03 | PDE4C | 0.3006 |
| rs28666607 | 1kg_19_18194805 | A | 8.384 | 3.59E−03 | PDE4C | 0.3031 |
| rs4808765 | 1kg_19_18195443 | A | 8.384 | 3.59E−03 | PDE4C | 0.303 |
| rs55973594 | 1kg_19_18191668 | G | 8.384 | 3.59E−03 | PDE4C | 0.3034 |
| rs75576204 | imm_5_141405952 | A | 0.02289 | 3.68E−03 | GNPDA1, NDFIP1 | 0.112 |
| rs16940174 | rs16940174 | A | 0.1386 | 3.74E−03 | AQP9, LIPC | 0.1296 |
| rs6601556 | 1kg_8_10959792 | G | 7.412 | 3.78E−03 | XKR6 | 0.3418 |
| rs62011167 | imm_15_77049780 | G | 0.1115 | 3.78E−03 | RASGRF1 | 0.1746 |
| rs2163625 | rs2163625 | G | 8.117 | 3.86E−03 | TMEM9B | 0.4115 |
| rs26519 | imm_5_96175859 | A | 0.06137 | 3.89E−03 | ERAP1 | 0.08176 |
| rs7175099 | rs7175099 | A | 8.119 | 4.02E−03 | LOC101927286 | 0.3224 |
| rs77782465 | seq-NOVEL-1738 | G | 0.1364 | 4.03E−03 | PFKFB3 | 0.1709 |
| rs1420415 | rs1420415 | C | 5.87 | 4.08E−03 | SMIM23, FBXW11 | 0.4107 |
| rs223498 | rs223498 | C | 0.1443 | 4.10E−03 | MANBA | 0.4948 |
| rs7770557 | rs7770557 | G | 0.06802 | 4.11E−03 | ZNRD1 | 0.1222 |
| rs9261274 | 1kg_6_30138768 | A | 0.06802 | 4.11E−03 | ZNRD1 | 0.1222 |
| rs9261275 | rs9261275 | A | 0.06802 | 4.11E−03 | ZNRD1 | 0.1222 |
| rs9261277 | 1kg_6_30139070 | G | 0.06802 | 4.11E−03 | ZNRD1 | 0.1221 |
| rs9261278 | 1kg_6_30139341 | A | 0.06802 | 4.11E−03 | ZNRD1 | 0.1222 |
| rs3757329 | rs3757329 | C | 0.06802 | 4.11E−03 | ZNRD1-AS1 | 0.1222 |
| rs6917477 | 1kg_6_30133963 | C | 0.06802 | 4.11E−03 | ZNRD1-AS1 | 0.1222 |
| rs7761314 | 1kg_6_30130132 | A | 0.06802 | 4.11E−03 | ZNRD1-AS1 | 0.1222 |
| rs7761314 | rs7761314 | A | 0.06802 | 4.11E−03 | ZNRD1-AS1 | 0.1222 |
| rs7769930 | rs7769930 | C | 0.06802 | 4.11E−03 | ZNRD1-AS1 | 0.1222 |
| rs9261224 | 1kg_6_30121866 | A | 0.06802 | 4.11E−03 | ZNRD1-AS1 | 0.1222 |
| rs9261243 | 1kg_6_30125738 | G | 0.06802 | 4.11E−03 | ZNRD1-AS1 | 0.1221 |
| rs9261251 | 1kg_6_30127748 | A | 0.06802 | 4.11E−03 | ZNRD1-AS1 | 0.1222 |
| rs9261256 | 1kg_6_30129920 | C | 0.06802 | 4.11E−03 | ZNRD1-AS1 | 0.1222 |
| rs9261258 | 1kg_6_30130787 | A | 0.06802 | 4.11E−03 | ZNRD1-AS1 | 0.1222 |
| rs9261261 | 1kg_6_30132344 | G | 0.06802 | 4.11E−03 | ZNRD1-AS1 | 0.1218 |
| rs9261261 | rs9261261 | G | 0.06802 | 4.11E−03 | ZNRD1-AS1 | 0.1222 |
| rs9261262 | 1kg_6_30132373 | A | 0.06802 | 4.11E−03 | ZNRD1-AS1 | 0.1222 |
| rs75792394 | imm_2_62462582 | A | 0.07597 | 4.17E−03 | B3GNT2, TMEM17 | 0.1325 |
| rs1410088 | rs1410088 | G | 0.137 | 4.18E−03 | LOC101927412, LOC101927434 | 0.2478 |
| rs4808766 | 1kg_19_18196715 | C | 8.459 | 4.18E−03 | PDE4C | 0.2814 |
| | rs5925540 | A | 19.91 | 4.21E−03 | GPR50_LOC286456 | 0.4274 |
| rs1761455 | seq-rs1761455 | G | 76.12 | 4.24E−03 | LILRA3, LILRA5 | 0.2835 |
| rs404032 | seq-rs404032 | C | 76.12 | 4.24E−03 | LILRA3, LILRA5 | 0.2834 |
| rs414135 | seq-rs414135 | A | 76.12 | 4.24E−03 | LILRA3, LILRA5 | 0.2833 |
| rs651279 | seq-rs651279 | G | 76.12 | 4.24E−03 | LILRA3, LILRA5 | 0.2841 |
| rs759819 | seq-rs759819 | G | 76.12 | 4.24E−03 | LILRA3, LILRA5 | 0.2835 |
| rs6911737 | 1kg_6_30073480 | A | 0.09799 | 4.27E−03 | HCG9, ZNRD1-AS1 | 0.1288 |
| rs6911737 | rs6911737 | A | 0.09799 | 4.27E−03 | HCG9, ZNRD1-AS1 | 0.1288 |
| rs8082184 | rs8082184 | G | 0.2027 | 4.27E−03 | NXN | 0.3733 |
| rs11075293 | rs11075293 | A | 7.497 | 4.31E−03\| | ABCC1 | 0.45 |
| rs1010355 | seq-rs1010355 | G | 0.07455 | 4.44E−03 | LILRA2, LILRA1 | 0.0876 |
| rs2316184 | rs2316184 | G | 0.08596 | 4.51E−03 | CDYL2 | 0.2381 |
| rs1025601 | rs1025601 | A | 0.2511 | 4.55E−03 | TSHZ1, SMIM21 | 0.3742 |
| rs12186886 | rs12186886 | G | 6.357 | 4.61E−03 | LOC101929505 | 0.3806 |
| rs13183026 | rs13183026 | A | 6.357 | 4.61E−03 | LOC101929505 | 0.381 |
| rs11065564 | rs11065564 | A | 0.0668 | 4.64E−03 | RNF34 | 0.0947 |
| | rs180456 | G | 25.64 | 4.67E−03 | GPR50_LOC286456 | 0.4637 |
| rs13006027 | imm_2_10193495 | A | 7.081 | 4.77E−03 | MAP4K4, LINC01127 | 0.3454 |
| rs17026308 | imm_2_101932459 | A | 7.081 | 4.77E−03 | MAP4K4, LINC01127 | 0.3463 |
| rs58626985 | imm_2_1019320049 | A | 7.081 | 4.77E−03 | MAP4K4, LINC01127 | 0.3454 |
| rs10468612 | rs10468612 | A | 7.039 | 4.78E−03 | MRM1, LHX1 | 0.3348 |
| rs1860598 | rs1860598 | G | 6.022 | 5.01E−03 | FAM184B | 0.4222 |
| rs2186676 | imm_11_76016227 | G | 0.1187 | 5.04E−03 | EMSY, LRRC32 | 0.2692 |
| rs7264756 | imm_20_47937524 | G | 0.07732 | 5.06E−03 | SLC9A8 | 0.08118 |
| rs7270636 | imm_20_47936868 | G | 0.07732 | 5.06E−03 | SLC9A8 | 0.08561 |
| rs73125639 | imm_20_47893899 | A | 0.07732 | 5.06E−03 | SLC9A8 | 0.08102 |
| rs73125645 | imm_20_47899161 | A | 0.07732 | 5.06E−03 | SLC9A8 | 0.08093 |
| rs73125685 | imm_20_47935283 | C | 0.07732 | 5.06E−03 | SLC9A8 | 0.08212 |
| rs76161485 | imm_20_47936972 | A | 0.07732 | 5.06E−03 | SLC9A8 | 0.08071 |
| rs1030291 | imm_2_185852651 | C | 0.08784 | 5.10E−03 | ZNF804A, LOC101927196 | 0.1479 |
| rs10931168 | imm_2_185872668 | G | 0.08784 | 5.10E−03 | ZNF804A, LOC101927196 | 0.1497 |
| rs12466097 | imm_2_185893_161 | A | 0.08784 | 5.10E−03 | ZNF804A, LOC101927196 | 0.1488 |
| rs13408932 | imm_2_185843782 | A | 0.08784 | 5.10E−03 | ZNF804A, LOC101927196 | 0.1482 |
| rs13425009 | imm_2_185853227 | A | 0.08784 | 5.10E−03 | ZNF804A, LOC101927196 | 0.1478 |
| rs13429304 | imm_2_185845717 | T | 0.08784 | 5.10E−03 | ZNF804A, LOC101927196 | 0.1483 |
| rs13432852 | imm_2_185855786 | A | 0.08784 | 5.10E−03 | ZNF804A, LOC101927196 | 0.1479 |
| rs2059349 | imm_2_185873069 | G | 0.08784 | 5.10E−03 | ZNF804A, LOC101927196 | 0.1495 |
| rs3887388 | imm_2_185886932 | A | 0.08784 | 5.10E−03 | ZNF804A, LOC101927196 | 0.1486 |
| rs4667028 | imm_2_185874905 | A | 0.08784 | 5.10E−03 | ZNF804A, LOC101927196 | 0.1494 |
| rs55801101 | imm_2_185836939 | T | 0.08784 | 5.10E−03 | ZNF804A, LOC101927196 | 0.1483 |
| rs6730298 | imm_2_185856143 | G | 0.08784 | 5.10E−03 | ZNF804A, LOC101927196 | 0.149 |
| rs67548106 | imm_2_185840351 | A | 0.08784 | 5.10E−03 | ZNF804A, LOC101927196 | 0.1482 |
| rs164938 | rs164938 | A | 0.1859 | 5.12E−03 | TATDN2 | 0.4059 |
| rs2835709 | rs2835709 | G | 0.1507 | 5.14E−03 | DYRK1A | 0.1657 |
| rs9976328 | rs9976328 | G | 0.1507 | 5.14E−03 | DYRK1A | 0.1692 |
| rs12237465 | imm_9_116575086 | G | 0.1171 | 5.22E−03 | LOC100505478, TNFSF15 | 0.1809 |
| rs4479011 | rs4479011 | C | 0.07325 | 5.37E−03 | TMEM135, RAB38 | 0.06411 |
| rs4789949 | rs4789949 | A | 0.1135 | 5.39E−03 | RBFOX3 | 0.3231 |
| rs10847699 | imm_12_127868986 | G | 6.743 | 5.44E−03 | SLC15A4 | 0.3009 |
| rs11059934 | imm_12_127870813 | G | 6.743 | 5.44E−03 | SLC15A4 | 0.3002 |
| rs7311875 | imm_12_127859220 | G | 6.743 | 5.44E−03 | SLC15A4 | 0.3012 |
| rs7218139 | rs7218139 | G | 0.1422 | 5.51E−03 | FLJ45513, DLX4 | 0.1627 |
| rs6578008 | rs6578008 | T | 5.84 | 5.52E−03 | COL22A1, KCNK9 | 0.4191 |
| rs62120372 | 1kg_19_18166185 | A | 7.431 | 5.53E−03 | MPV17L2 | 0.2961 |
| rs16932710 | rs16932710 | G | 0.1473 | 5.60E−03 | LINC00967, RRS1-AS1 | 0.2109 |
| rs7774158 | rs7774158 | A | 0.1526 | 5.61E−03 | HLA-DOA, HLA-DPA1 | 0.4 |
| rs504215 | imm_19_53964296 | A | 8.034 | 5.71E−03 | FGF21, BCAT2 | 0.3304 |
| rs637174 | imm_19_53958748 | A | 8.034 | 5.71E−03 | FGF21, BCAT2 | 0.3205 |
| rs3747129 | 22_25192041 | A | 0.0919 | 5.72E−03 | HPS4 | 0.148 |
| rs3790093 | rs3790093 | A | 7.525 | 5.79E−03 | GNAO1 | 0.3215 |
| rs17623914 | seq-rs17623914 | G | 0.1282 | 5.93E−03 | PTPRC | 0.1239 |
| rs10736978 | rs10736978 | C | 20.59 | 5.96E−03 | LOC105376360 | 0.3041 |
| rs12532924 | rs12532924 | G | 5.021 | 5.99E−03 | DPP6 | 0.4567 |
| rs427366 | seq-rs427366 | A | 0.1968 | 6.03E−03 | MIR4752, LILRA3 | 0.3296 |
| rs9566964 | 1kg_13_41806350 | A | 6.373 | 6.06E−03 | AKAP11, TNFSF11 | 0.3012 |
| rs12471529 | imm_2_10190478 | G | 5.936 | 6.09E−03 | MAP4K4, LINC01127 | 0.4223 |
| rs2409767 | imm_8_11341398 | G | 0.1536 | 6.10E−03 | FAM167A | 0.4017 |
| rs2409770 | imm_8_11341526 | G | 0.1536 | 6.10E−03 | FAM167A | 0.4016 |
| rs2409771 | imm_8_11341553 | G | 0.1536 | 6.10E−03 | FAM167A | 0.4014 |
| rs17148752 | rs17148752 | G | 0.1116 | 6.13E−03 | HIP1 | 0.07585 |
| rs9427713 | imm_1_199307987 | A | 0.203 | 6.16E−03 | CACNA1S | 0.3541 |
| rs632798 | rs632798 | A | 0.1815 | 6.21E−03 | AJAP1, MIR4417 | 0.2497 |
| rs1122021 | rs1122021 | A | 0.1446 | 6.25E−03 | INSIG2, LOC101927709 | 0.1648 |
| rs1517531 | rs1517531 | A | 0.1446 | 6.25E−03 | INSIG2, LOC101927709 | 0.1651 |
| rs2624435 | rs2624435 | A | 0.1705 | 6.33E−03 | MYO10, LOC285696 | 0.2363 |
| rs7838605 | rs7838605 | A | 0.02648 | 6.37E−03 | C8orf34 | 0.08347 |
| rs2736320 | imm_8_11363935 | C | 8.896 | 6.39E−03 | FAM167A, BLK | 0.3916 |
| rs2777965 | rs2777965 | C | 0.1389 | 6.39E−03 | FCRL4 | 0.3574 |
| rs11637613 | rs11637613 | C | 0.1663 | 6.42E−03 | LOC440311, LINC01197 | 0.2816 |
| rs375912 | rs375912 | G | 0.2416 | 6.44E−03 | HLA-DOA, HLA-DPA1 | 0.3491 |
| rs11723291 | rs11723291 | A | 0.1947 | 6.50E−03 | PRSS48, FAM160A1 | 0.3304 |
| rs2306125 | imm_1_153291985 | G | 0.1358 | 6.52E−03 | LOC100505666 | 0.179 |
| rs4606022 | imm_8_11392342 | G | 5.439 | 6.53E−03 | BLK | 0.3851 |
| rs677618 | rs677618 | G | 0.2151 | 6.57E−03 | CACNA1E, ZNF648 | 0.3048 |
| rs1761456 | seq-rs1761456 | A | 34.3 | 6.60E−03 | LILRA3, LILRA5 | 0.2703 |
| rs7164805 | rs7164805 | A | 0.1631 | 6.66E−03 | BCL2A1, ZFAND6 | 0.4474 |
| rs2161396 | rs2161396 | C | 0.2678 | 6.66E−03 | CYFIP2, NIPAL4 | 0.3437 |
| rs914842 | imm_9_122658792 | A | 14.06 | 6.68E−03 | PHF19 | 0.226 |
| rs10784470 | imm_12_38949863 | A | 6.674 | 6.73E−03 | LRRK2 | 0.312 |
| rs191204 | imm_5_55463560 | A | 5.304 | 6.83E−03 | ANKRD55 | 0.4793 |
| rs11129795 | rs11129795 | A | 0.2134 | 6.85E−03\| | SCN5A | 0.2366 |
| rs12466923 | rs12466923 | C | 0.1875 | 6.91E−03 | KLF7 | 0.2097 |
| rs6500605 | rs6500605 | G | 5.232 | 6.92E−03 | DNAJA3 | 0.3286 |
| rs6500606 | rs6500606 | G | 5.232 | 6.92E−03 | DNAJA3 | 0.3273 |
| rs2270366 | rs2270366 | G | 5.232 | 6.92E−03 | HMOX2 | 0.3259 |
| | rs11095848 | G | 0.1038 | 6.94E−03 | CDR1_LOC100133171 | 0.1362 |
| rs13157314 | imm_5_40634460 | A | 0.1468 | 6.94E−03 | LINC00603, PTGER4 | 0.1472 |
| rs11800309 | 1_226458431 | A | 0.1863 | 7.02E−03 | C1orf145 | 0.2344 |
| rs4871600 | rs4871600 | G | 0.1494 | 7.06E−03 | MAL2 | 0.2309 |
| rs9277027 | rs9277027 | G | 0.2277 | 7.22E−03 | HLA-DOA, HLA-DPA1 | 0.304 |
| rs9277053 | rs9277053 | A | 0.2277 | 7.22E−03 | HLA-DOA, HLA-DPA1 | 0.3076 |
| rs13248300 | 1kg_8_10964085 | C | 6.216 | 7.22E−03 | XKR6 | 0.3318 |
| rs17779791 | 1kg_8_10977651 | G | 6.216 | 7.22E−03 | XKR6 | 0.3311 |
| rs1807510 | rs1807510 | C | 0.03237 | 7.22E−03 | MN1 | 0.09814 |
| rs990108 | imm_7_107264441 | G | 9.735 | 7.27E−03 | SLC26A3, DLD | 0.2044 |
| rs9855092 | rs9855092 | G | 0.2103 | 7.29E−03 | MFSD1, IQCJ | 0.2136 |
| rs10895692 | rs10895692 | G | 5.446 | 7.30E−03 | MIR7641-1, LOC102723895 | 0.3515 |
| rs4266238 | rs4266238 | A | 5.794 | 7.35E−03 | MIR378D1, JAKMIP1 | 0.4893 |
| | rs371298 | A | 0.2426 | 7.38E−03 | SLC25A5_CXorf56 | 0.3415 |
| | rs5985961 | A | 0.137 | 7.40E−03 | IL1RAPL1 | 0.1907 |
| rs6910898 | 1kg_6_30071158 | G | 0.1168 | 7.41E−03 | HCG9, ZNRD1-AS1 | 0.1437 |
| rs6911279 | 1kg_6_30073323 | G | 0.1168 | 7.41E−03 | HCG9, ZNRD1-AS1 | 0.1432 |
| rs6911279 | rs6911279 | G | 0.1168 | 7.41E−03 | HCG9, ZNRD1-AS1 | 0.1432 |
| rs6912080 | 1kg_6_30073542 | A | 0.1168 | 7.41E−03 | HCG9, ZNRD1-AS1 | 0.1432 |
| rs9260959 | 1kg_6_30068849 | A | 0.1168 | 7.41E−03 | HCG9, ZNRD1-AS1 | 0.143 |
| rs9260961 | 1kg_6_30068978 | A | 0.1168 | 7.41E−03 | HCG9, ZNRD1-AS1 | 0.143 |
| rs9260966 | 1kg_6_30069260 | A | 0.1168 | 7.41E−03 | HCG9, ZNRD1-AS1 | 0.143 |
| rs9260967 | 1kg_6_30069346 | A | 0.1168 | 7.41E−03 | HCG9, ZNRD1-AS1 | 0.143 |
| rs9260968 | 1kg_6_30069418 | A | 0.1168 | 7.41E−03 | HCG9, ZNRD1-AS1 | 0.143 |
| rs9260975 | 1kg_6_30069701 | A | 0.1168 | 7.41E−03 | HCG9, ZNRD1-AS1 | 0.143 |
| rs9260978 | 1kg_6_30069832 | A | 0.1168 | 7.41E−03 | HCG9, ZNRD1-AS1 | 0.143 |
| rs9260994 | 1kg_6_30070775 | C | 0.1168 | 7.41E−03 | HCG9, ZNRD1-AS1 | 0.1433 |
| rs9261016 | 1kg_6_30073285 | A | 0.1168 | 7.41E−03 | HCG9, ZNRD1-AS1 | 0.1432 |
| rs9261020 | 1kg_6_30073650 | A | 0.1168 | 7.41E−03 | HCG9, ZNRD1-AS1 | 0.1432 |
| rs9261026 | rs9261026 | A | 0.1168 | 7.41E−03 | HCG9, ZNRD1-AS1 | 0.1432 |
| rs3765604 | 1kg_6_30084003 | G | 0.1168 | 7.41E−03 | HLA-J | 0.1432 |
| rs9261105 | 1kg_6_30082479 | G | 0.1168 | 7.41E−03 | HLA-J, ZNRD1-AS1 | 0.1432 |
| rs2074482 | 1kg_6_30144450 | A | 0.1168 | 7.41E−03 | PPP1R11 | 0.1431 |
| rs2074482 | rs2074482 | A | 0.1168 | 7.41E−03 | PPP1R11 | 0.1431 |
| rs2074479 | 1kg_6_30148988 | G | 0.1168 | 7.41E−03 | RNF39 | 0.1431 |
| rs2074479 | rs2074479 | G | 0.1168 | 7.41E−03 | RNF39 | 0.1431 |
| rs2074480 | 1kg_6_30148789 | C | 0.1168 | 7.41E−03 | RNF39 | 0.1431 |
| rs2301753 | rs2301753 | A | 0.1168 | 7.41E−03 | RNF39 | 0.1431 |
| rs9261291 | rs9261291 | A | 0.1168 | 7.41E−03 | RNF39 | 0.1431 |
| rs9261294 | 1kg_6_30147620 | G | 0.1168 | 7.41E−03 | RNF39 | 0.1431 |
| rs9261297 | 1kg_6_30147824 | A | 0.1168 | 7.41E−03 | RNF39 | 0.1432 |
| rs9261298 | 1kg_6_30147880 | A | 0.1168 | 7.41E−03 | RNF39 | 0.1431 |
| rs9261299 | 1kg_6_30147987 | C | 0.1168 | 7.41E−03 | RNF39 | 0.1431 |
| rs9261300 | 1kg_6_30148164 | A | 0.1168 | 7.41E−03 | RNF39 | 0.1431 |
| rs9261302 | 1kg_6_30150328 | A | 0.1168 | 7.41E−03 | RNF39 | 0.143 |
| rs1048412 | 1kg_6_30140474 | G | 0.1168 | 7.41E−03 | ZNRD1 | 0.1431 |
| rs11965524 | rs11965524 | A | 0.1168 | 7.41E−03 | ZNRD1-AS1 | 0.1432 |
| rs2286405 | 1kg_6_30081371 | G | 0.1168 | 7.41E−03 | ZNRD1-AS1 | 0.143 |
| rs3734835 | 1kg_6_30087805 | G | 0.1168 | 7.41E−03 | ZNRD1-AS1 | 0.1432 |
| rs3869067 | 1kg_6_30111776 | G | 0.1168 | 7.41E−03 | ZNRD1-AS1 | 0.1431 |
| rs3869068 | 1kg_6_30112031 | A | 0.1168 | 7.41E−03 | ZNRD1-AS1 | 0.1432 |
| rs3869068 | rs3869068 | A | 0.1168 | 7.41E−03 | ZNRD1-AS1 | 0.1432 |
| rs6905157 | rs6905157 | G | 0.1168 | 7.41E−03 | ZNRD1-AS1 | 0.1431 |
| rs6919617 | rs6919617 | G | 0.1168 | 7.41E−03 | ZNRD1-AS1 | 0.1432 |
| rs6926792 | 1kg_6_30093828 | A | 0.1168 | 7.41E−03 | ZNRD1-AS1 | 0.1432 |
| rs6926792 | rs6926792 | A | 0.1168 | 7.41E−03 | ZNRD1-AS1 | 0.1432 |
| rs7746866 | 1kg_6_30106161 | G | 0.1168 | 7.41E−03 | ZNRD1-AS1 | 0.1432 |
| rs9261103 | 1kg_6_30081114 | G | 0.1168 | 7.41E−03 | ZNRD1-AS1 | 0.1432 |
| rs9261129 | 1kg_6_30087558 | G | 0.1168 | 7.41E−03 | ZNRD1-AS1 | 0.1432 |
| rs9261145 | 1kg_6_30092844 | A | 0.1168 | 7.41E−03 | ZNRD1-AS1 | 0.1431 |
| rs9261198 | 1kg_6_30110921 | A | 0.1168 | 7.41E−03 | ZNRD1-AS1 | 0.1431 |
| rs9261199 | 1kg_6_30111089 | G | 0.1168 | 7.41E−03 | ZNRD1-AS1 | 0.1431 |
| rs9261200 | rs9261200 | A | 0.1168 | 7.41E−03 | ZNRD1-AS1 | 0.1431 |
| rs9261201 | 1kg_6_30112238 | G | 0.1168 | 7.41E−03 | ZNRD1-AS1 | 0.1431 |
| rs9261205 | 1kg_6_30113290 | G | 0.1168 | 7.41E−03 | ZNRD1-AS1 | 0.1431 |
| rs9261216 | 1kg_6_30118118 | G | 0.1168 | 7.41E−03 | ZNRD1-AS1 | 0.1431 |
| rs72758134 | imm_9_122662067 | C | 0.1284 | 7.56E−03 | PHF19 | 0.154 |
| rs12379604 | imm_9_122656679 | C | 0.1284 | 7.56E−03 | PSMD5-AS1 | 0.1564 |
| rs10254800 | rs10254800 | G | 0.243 | 7.58E−03 | C7orf57 | 0.3757 |
| rs76643044 | imm_5_141409138 | A | 0.09632 | 7.60E−03 | GNPDA1, NDFIP1 | 0.1144 |
| rs10986432 | rs10986432 | G | 0.1702 | 7.62E−03 | OLFML2A | 0.1875 |
| rs7927515 | imm_11_75802978 | A | 5.544 | 7.62E−03 | LOC100506127, EMSY | 0.3767 |
| rs7653338 | rs7653338 | A | 0.1005 | 7.80E−03 | EPHA3, NONE | 0.07157 |
| rs2235383 | rs2235383 | G | 0.23 | 7.81E−03 | HLA-F | 0.1372 |
| rs2272874 | rs2272874 | G | 0.23 | 7.81E−03 | HLA-F-AS1 | 0.1372 |
| rs9258187 | rs9258187 | C | 0.23 | 7.81E−03 | HLA-F-AS1 | 0.1372 |
| rs3757324 | rs3757324 | A | 0.23 | 7.81E−03 | ZFP57, HLA-F | 0.1374 |
| rs9261132 | 1kg_6_30089042 | G | 0.102 | 7.83E−03 | HCG8 | 0.08812 |
| rs11670370 | 1kg_19_18202756 | A | 6.991 | 7.85E−03 | PDE4C | 0.3052 |
| rs712086 | rs712086 | G | 0.2505 | 7.87E−03 | WDR26, CNIH3 | 0.4655 |
| rs7001675 | imm_8_11334010 | G | 0.1612 | 7.87E−03 | FAM167A | 0.4346 |
| rs10903116 | imm_1_25155749 | G | 0.2162 | 7.91E−03 | RUNX3 | 0.3825 |
| rs10903117 | imm_1_25156179 | G | 0.2162 | 7.91E−03 | RUNX3 | 0.3825 |
| rs11249207 | imm_1_25155656 | G | 0.2162 | 7.91E−03 | RUNX3 | 0.3822 |
| rs11580845 | imm_1_25155943 | C | 0.2162 | 7.91E−03 | RUNX3 | 0.3823 |
| rs12031692 | imm_125155861 | A | 0.2162 | 7.91E−03 | RUNX3 | 0.382 |
| rs1848186 | imm_1_25155443 | C | 0.2162 | 7.91E−03 | RUNX3 | 0.3846 |
| rs4288539 | imm_1_25155580 | G | 0.2162 | 7.91E−03 | RUNX3 | 0.3825 |
| rs6600245 | imm_1_25157265 | A | 0.2162 | 7.91E−03 | RUNX3 | 0.3809 |
| rs838795 | rs838795 | A | 0.2686 | 7.96E−03 | SMIM23, FBXW11 | 0.432 |
| rs1941438 | rs1941438 | C | 6.136 | 7.96E−03 | FAT3 | 0.3183 |
| rs7583252 | rs7583252 | A | 0.1827 | 7.97E−03 | DAW1, SPHKAP | 0.4013 |
| rs2777491 | rs2777491 | C | 0.2472 | 7.97E−03 | RTF1 | 0.3242 |
| rs11233264 | rs11233264 | T | 0.1803 | 7.98E−03 | MIR4300HG, FAM181B | 0.2388 |
| rs4648888 | imm_1_25158738 | G | 0.2165 | 8.04E−03 | RUNX3 | 0.3864 |
| rs1452835 | rs1452835 | G | 0.1346 | 8.07E−03 | NONE, CTB-7E3.1 | 0.3127 |
| rs13057793 | rs13057793 | G | 0.2162 | 8.15E−03 | SMC1B | 0.4572 |
| rs9614457 | rs9614457 | G | 0.2162 | 8.15E−03 | SMC1B | 0.4572 |
| rs13438187 | imm_7_107263570 | C | 0.2446 | 8.30E−03 | SLC26A3, DLD | 0.3261 |
| rs57441319 | imm_11_76014480 | G | 0.1737 | 8.31E−03 | EMSY, LRRC32 | 0.2531 |
| rs1538957 | rs1538957 | A | 0.1573 | 8.31E−03 | KIF26B | 0.2656 |
| rs10088323 | imm_8_11338301 | G | 0.184 | 8.35E−03 | FAM167A | 0.4291 |
| rs7839434 | imm_8_11363051 | G | 9.23 | 8.40E−03 | FAM167A, BLK | 0.2173 |
| rs60813083 | imm_20_48012653 | C | 0.1264 | 8.43E−03 | RNF114, SNAI1 | 0.09053 |
| rs59922432 | imm_20_47963975 | G | 0.1264 | 8.43E−03 | SPATA2 | 0.09032 |
| rs73910338 | imm_20_47958624 | G | 0.1264 | 8.43E−03 | SPATA2 | 0.09036 |
| rs7751815 | rs7751815 | A | 0.2155 | 8.45E−03 | HLA-F-AS1 | 0.1292 |
| rs7755571 | rs7755571 | G | 0.2155 | 8.45E−03 | HLA-F-AS1 | 0.1292 |
| rs17659250 | rs17659250 | A | 0.192 | 8.48E−03 | ADAM19 | 0.2751 |
| rs10102823 | rs10102823 | A | 0.1858 | 8.50E−03 | C8orf34 | 0.1799 |
| rs74821015 | imm_20_44216530 | A | 0.06215 | 8.60E−03 | CD40, CDH22 | 0.05502 |
| rs2246638 | rs2246638 | A | 0.1286 | 8.67E−03 | HCG9, ZNRD1-AS1 | 0.2072 |
| rs10160382 | imm_11_75804862 | G | 5.578 | 8.72E−03 | LOC100506127, EMSY | 0.3764 |
| rs10483739 | rs10483739 | A | 9.111 | 8.74E−03 | PRKCH | 0.2163 |
| rs10411210 | rs10411210 | A | 0.1596 | 8.75E−03 | RHPN2 | 0.12 |
| rs11084329 | seq-rs11084329 | G | 11.75 | 8.76E−03 | LILRA5, LILRA4 | 0.2994 |
| rs1 6851319 | rs16851319 | G | 0.1907 | 8.82E−03 | BTG2, FMOD | 0.1815 |
| rs382571 | rs382571 | G | 0.141 | 8.83E−03 | VAT1 | 0.1785 |
| rs3128941 | rs3128941 | G | 9.027 | 8.84E−03 | HLA-DOA, HLA-DPA1 | 0.4577 |
| rs1232 | rs1232 | A | 8.485 | 8.85E−03 | GPR75, GPR75-ASB3 | 0.2658 |
| rs1990649 | rs1990649 | G | 0.1671 | 8.87E−03 | LYPD6 | 0.2065 |
| rs4891826 | rs4891826 | C | 5.307 | 8.95E−03 | RTTN, SOCS6 | 0.2791 |
| rs683028 | rs683028 | G | 4.741 | 8.99E−03 | DKFZp686K1684, LOC100506675 | 0.4055 |
| rs112711874 | imm_21_42735005 | G | 0.1363 | 9.01E−03 | UBASH3A | 0.09235 |
| rs56196737 | imm_21_42731675 | C | 0.1363 | 9.01E−03 | UBASH3A | 0.09209 |
| rs8127703 | imm_21_42730083 | A | 0.1363 | 9.01E−03 | UBASH3A | 0.09174 |
| rs17446667 | rs17446667 | A | 0.07422 | 9.02E−03 | KCNIP4 | 0.09417 |
| rs17786166 | imm_20_47909833 | G | 0.13 | 9.02E−03 | SLC9A8 | 0.1023 |
| rs59693166 | imm_20_47916454 | G | 0.13 | 9.02E−03 | SLC9A8 | 0.102 |
| rs73123871 | imm_20_47884626 | G | 0.13 | 9.02E−03 | SLC9A8 | 0.102 |
| rs73123872 | imm_20_47885261 | G | 0.13 | 9.02E−03 | SLC9A8 | 0.102 |
| rs73125682 | imm_20_47931385 | G | 0.13 | 9.02E−03 | SLC9A8 | 0.1021 |
| rs10168917 | rs10168917 | G | 0.1413 | 9.02E−03 | KCNS3, RDH14 | 0.2805 |
| rs17668708 | seq-rs17668708 | A | 0.1264 | 9.03E−03 | PTPRC | 0.1158 |
| rs17669032 | seq-rs17669032 | G | 0.1264 | 9.03E−03 | PTPRC | 0.115 |
| rs80099993 | seq-t1d-1- | A | 0.1264 | 9.03E−03 | PTPRC | 0.1234 |
| | 196889070-G-A | | | | | |
| rs8029903 | rs8029903 | A | 0.1707 | 9.04E−03 | LOC440311, LINC01197 | 0.2803 |
| rs8031623 | imm_15_36731344 | A | 0.1094 | 9.05E−03 | RASGRP1, C15orf53 | 0.1268 |
| rs5009448 | rs5009448 | A | 0.2222 | 9.12E−03 | HLA-A, HCG9 | 0.3093 |
| rs12608228 | rs12608228 | G | 0.1144 | 9.20E−03 | ZNF521, SS18 | 0.4746 |
| rs10509690 | rs10509690 | A | 0.2708 | 9.24E−03 | SORBS1 | 0.2369 |
| rs10824740 | imm_10_80731730 | A | 0.1711 | 9.30E−03 | ZMIZ1 | 0.2883 |
| rs4948003 | rs4948003 | A | 5.182 | 9.49E−03 | ELDR, LANCL2 | 0.2852 |
| rs12563828 | rs12563828 | A | 8.7 | 9.54E−03 | DPYD | 0.2828 |
| rs2346689 | rs2346689 | A | 32.9 | 9.56E−03 | ASIC2 | 0.2714 |
| rs67218200 | imm_2_185907859 | A | 0.1319 | 9.61E−03 | ZNF804A, LOC101927196 | 0.1523 |
| rs2239525 | rs2239525 | G | 0.1867 | 9.61E−03 | ATP6V1G2-DDX39B | 0.235 |
| rs2239526 | rs2239526 | G | 0.1867 | 9.61E−03 | ATP6V1G2-DDX39B | 0.2349 |
| rs2239528 | rs2239528 | A | 0.1867 | 9.61E−03 | DDX39B-AS1 | 0.2349 |
| rs2523504 | rs2523504 | A | 0.1867 | 9.61E−03 | DDX39B-AS1 | 0.235 |
| rs12579024 | rs12579024 | C | 0.1813 | 9.62E−03 | TBX3, MED13L | 0.1865 |
| rs292256 | rs292256 | A | 7.324 | 9.67E−03 | BACH2 | 0.344 |
| rs1033762 | rs1033762 | C | 0.1653 | 9.74E−03 | ATXN1, STMND1 | 0.2331 |
| rs6909872 | rs6909872 | A | 0.1653 | 9.74E−03 | ATXN1, STMND1 | 0.2327 |
| rs6925974 | rs6925974 | G | 0.1653 | 9.74E−03 | ATXN1, STMND1 | 0.2328 |
| rs2626528 | rs2626528 | A | 0.2347 | 9.75E−03 | PXMP4 | 0.4774 |
| rs10225158 | rs10225158 | G | 5.587 | 9.77E−03 | LOC101927243, PTPN12 | 0.3957 |
| rs6921610 | rs6921610 | G | 5.312 | 9.78E−03 | LY86, RREB1 | 0.4637 |
| rs2210611 | 1kg_1_241055802 | A | 0.1929 | 9.80E−03 | PLD5, LINC01347 | 0.2039 |
| rs2210612 | 1kg_1_241055780 | G | 0.1929 | 9.80E−03 | PLD5, LINC01347 | 0.2038 |
| rs6694819 | 1kg_1_241058609 | C | 0.1929 | 9.80E−03 | PLD5, LINC01347 | 0.2045 |
| rs12962096 | imm_18_12785875 | G | 0.1683 | 9.81E−03 | PTPN2 | 0.3278 |
| rs55948693 | imm_18_12785030 | G | 0.1683 | 9.81E−03 | PTPN2 | 0.3273 |
| rs68009022 | imm_18_12808588 | G | 0.1683 | 9.81E−03 | PTPN2 | 0.329 |
| rs2409772 | imm_8_11343926 | A | 0.1844 | 9.88E−03 | FAM167A | 0.4283 |
| rs2409774 | imm_8_11344174 | C | 0.1844 | 9.88E−03 | FAM167A | 0.4305 |
| rs4841534 | imm_8_11344092 | G | 0.1844 | 9.88E−03 | FAM167A | 0.4284 |
| rs4841536 | imm_8_11344864 | G | 0.1844 | 9.88E−03 | FAM167A | 0.4285 |
| rs4841537 | imm_8_11344982 | G | 0.1844 | 9.88E−03 | FAM167A | 0.4285 |
| rs4841538 | imm_8_11345092 | C | 0.1844 | 9.88E−03 | FAM167A | 0.429 |
| rs6983820 | imm_8_11343434 | A | 0.1844 | 9.88E−03 | FAM167A | 0.4262 |
| rs9792175 | imm_8_11344528 | A | 0.1844 | 9.88E−03 | FAM167A | 0.4218 |
| rs74875570 | imm_12_56166244 | A | 0.1138 | 9.91E−03 | ARHGAP9, MARS | 0.08118 |
| rs80161048 | imm_12_56248399 | G | 0.1138 | 9.91E−03 | KIF5A | 0.08547 |
| rs4672880 | seq-rs4672880 | G | 0.05056 | 9.93E−03 | CXCR1, ARPC2 | 0.08452 |
| rs78107966 | seq-t1d-2- | G | 0.05056 | 9.93E−03 | CXCR1, ARPC2 | 0.08149 |
| | 218770246-T-C | | | | | |
| rs12023499 | imm_1_153298000 | A | 0.1784 | 9.96E−03 | LOC100505666 | 0.1965 |
| rs13280447 | imm_8_11346189 | A | 4.606 | 9.98E−03 | FAM167A | 0.4578 |
| rs9277029 | rs9277029 | A | 0.1869 | 9.99E−03 | HLA-DOA, HLA-DPA1 | 0.296 |
| rs11704339 | rs11704339 | A | 5.389 | 1.00E−02 | SYN3, LARGE | 0.3446 |

Polymorphisms listed in SNP (rsID) column of above tables are associated with “FC” (fold change) of gene expression of genes listed in “Gene” column with a significance indicated by the P value (“P”). The positions of the polymorphisms are relative to human genome assembly GCh38; “CHR”=chromosome, “BP”=base pair. The “Illumina id” corresponds with the Infinium ImmunoAarray-24 v. 2 Bead-Chip. The presence of the minor allele (“A1”) is associated with a “risk” of the phenotype of interest (TL1A fold change, high-low fold change, Signal 1) in gene if the odds ratio (“OR”) or beta value (“BETA”) corresponding to the polymorphism is more than 1 (OR>1), whereas if the OR<1, A1 is associated with a reduced risk of the phenotype. The major allele (A2) for each polymorphism disclosed herein can be found in the dbSNP database curated by the National Center for Biotechnology Information (NCBI), which is hereby incorporated by reference in its entirety. The term “polymorphism” as used herein can refer to either the minor or the major allele at the polymorphism position indicated by the reference rsID or Illumina id for that polymorphism.

### Example 5

### Phase 1 Clinical Trial

A phase 1 clinical trial is performed to evaluate the safety, tolerability, pharmacokinetics and pharmacodynamics of an anti-TL1A antibody on subjects having an inflammatory disease or condition, or fibrostenotic or fibrotic disease.

Single ascending dose (SAD) arms: Subjects in each group (subjects are grouped based on the presence of two copies of a polymorphism at the TNFSF15 gene locus, and optionally, the presence of a polymorphism from the gene loci: ETS1, LY86, or SCUBE1, and subjects grouped based on the presence of one copy of a polymorphism at the TNFSF15 gene locus, and optionally, the presence of a polymorphism from the gene loci ARHGAP15) receive either a single dose of the antibody or a placebo. For example, doses are 1, 3, 10, 30, 100, 300, 600 and 800 mg of antibody. Safety monitoring and PK assessments are performed for a predetermined time. Based on evaluation of the PK data, and if the antibody is deemed to be well tolerated, dose escalation occurs, either within the same groups or a further group of healthy subjects. Dose escalation continues until the maximum dose has been attained unless predefined maximum exposure is reached or intolerable side effects become apparent.

Multiple ascending dose (MAD) arms: Subjects in each group (subjects are grouped based on the same criteria as above) receive multiple doses of the antibody or a placebo. The dose levels and dosing intervals are selected as those that are predicted to be safe from the SAD data. Dose levels and dosing frequency are chosen to achieve therapeutic drug levels within the systemic circulation that are maintained at steady state for several days to allow appropriate safety parameters to be monitored. Samples are collected and analyzed to determination PK profiles.

Inclusion Criteria: Healthy subjects of non-childbearing potential between the ages of 18 and 55 years. Healthy is defined as no clinically relevant abnormalities identified by a detailed medical history, full physical examination, including blood pressure and pulse rate measurement, 12 lead ECG and clinical laboratory tests. Female subjects of non-childbearing potential may meet at least one of the following criteria: (1) achieved postmenopausal status, defined as: cessation of regular menses for at least 12 consecutive months with no alternative pathological or physiological cause; and have a serum follicle stimulating hormone (FSH) level within the laboratory's reference range for postmenopausal females; (2) have undergone a documented hysterectomy or bilateral oophorectomy; (3) have medically confirmed ovarian failure. All other female subjects (including females with tubal ligations and females that do NOT have a documented hysterectomy, bilateral oophorectomy or ovarian failure) will be considered to be of childbearing potential. Body Mass Index (BMI) of 17.5 to 30.5 kg/m2; and a total body weight >50 kg (110 lbs). Evidence of a personally signed and dated informed consent document indicating that the subject (or a legal representative) has been informed of all pertinent aspects of the study.

Three groups of subjects are selected: subjects having two copies of the TNFSF15 polymorphism, and optionally, a polymorphism at the LY86, ETS1, or SCUBE1 gene loci, whose presence is associated with an increase in TL1A, subjects having one copy of the TNFSF15 polymorphism, and optionally, a polymorphism at the ARHGAP15 gene locus, whose presence is associated with an increase in TL1A, and subjects lacking the risk variant.

Exclusion Criteria: Evidence or history of clinically significant hematological, renal, endocrine, pulmonary, gastrointestinal, cardiovascular, hepatic, psychiatric, neurologic, or allergic disease (including drug allergies, but excluding untreated, asymptomatic, seasonal allergies at time of dosing). Subjects with a history of or current positive results for any of the following serological tests: Hepatitis B surface antigen (HBsAg), Hepatitis B core antibody (HBcAb), anti-Hepatitis C antibody (HCV Ab) or human immunodeficiency virus (HIV). Subjects with a history of allergic or anaphylactic reaction to a therapeutic drug. Treatment with an investigational drug within 30 days (or as determined by the local requirement, whichever is longer) or 5 half-lives or 180 days for biologics preceding the first dose of study medication. Pregnant females; breastfeeding females; and females of childbearing potential.

Primary Outcome Measures: Incidence of dose limiting or intolerability treatment related adverse events (AEs) [Time Frame: 12 weeks]. Incidence, severity and causal relationship of treatment emergent AEs (TEAEs) and withdrawals due to treatment emergent adverse events [Time Frame: 12 weeks]. Incidence and magnitude of abnormal laboratory findings [Time Frame: 12 weeks]. Abnormal and clinically relevant changes in vital signs, blood pressure (BP) and electrocardiogram (ECG) parameters [Time Frame: 12 weeks].

Secondary Outcome Measures: Single Ascending Dose: Maximum Observed Plasma Concentration (Cmax) [Time Frame: 12 weeks]. Single Ascending Dose: Time to Reach Maximum Observed Plasma Concentration (Tmax) [Time Frame: 12 weeks]. Single Ascending Dose: Area under the plasma concentration-time profile from time zero to 14 days (AUC14 days) [Time Frame: 12 weeks]. Single Ascending Dose: Area under the plasma concentration-time profile from time zero extrapolated to infinite time (AUCinf) [Time Frame: 12 weeks]. Single Ascending Dose: Area under the plasma concentration-time profile from time zero to the time of last quantifiable concentration (AUClast) [Time Frame: 12 weeks]. Single Ascending Dose: Dose normalized maximum plasma concentration (Cmax[dn]) [Time Frame: 12 weeks]. Single Ascending Dose: Dose normalized area under the plasma concentration-time profile from time zero extrapolated to infinite time (AUCinf[dn]) [Time Frame: 12 weeks]. Single Ascending Dose: Dose normalized area under the plasma concentration-time profile from time zero to the time of last quantifiable concentration (AUClast[dn]) [Time Frame: 12 weeks]. Single Ascending Dose: Plasma Decay Half-Life (t½) [Time Frame: 12 weeks]. Plasma decay half-life is the time measured for the plasma concentration to decrease by one half. Single Ascending Dose: Mean residence time (MRT) [Time Frame: 12 weeks]. Single Ascending Dose: Volume of Distribution at Steady State (Vss) [Time Frame: 6 weeks]. Volume of distribution is defined as the theoretical volume in which the total amount of drug may be uniformly distributed to produce the predetermined blood concentration of a drug. Steady state volume of distribution (Vss) is the apparent volume of distribution at steady-state. Single Ascending Dose: Systemic Clearance (CL) [Time Frame: 6]. CL is a quantitative measure of the rate at which a drug substance is removed from the body.

Multiple Ascending Dose First Dose: Maximum Observed Plasma Concentration (Cmax) [Time Frame: 12 weeks]. Multiple Ascending Dose First Dose: Time to Reach Maximum Observed Plasma Concentration (Tmax) [Time Frame: 12 weeks]. Multiple Ascending Dose First Dose: Area under the plasma concentration-time profile from time zero to time τ, the dosing interval where τ=2 weeks (AUCτ) [Time Frame: 12 weeks]. Multiple Ascending Dose First Dose: Dose normalized maximum plasma concentration (Cmax[dn]) [Time Frame: 12 weeks]. Multiple Ascending Dose First Dose: Dose normalized Area under the plasma concentration-time profile from time zero to time τ, the dosing interval where τ=2 weeks (AUCτ[dn]) [Time Frame: 12 weeks]. Plasma Decay Half-Life (t½) [Time Frame: 12 weeks]. Plasma decay half-life is the time measured for the plasma concentration to decrease by one half. Multiple Ascending Dose First Dose: Mean residence time (MRT) [Time Frame: 12 weeks]. Apparent Volume of Distribution (Vz/F) [Time Frame: 12 weeks]. Volume of distribution is defined as the theoretical volume in which the total amount of drug may be uniformly distributed to produce the predetermined plasma concentration of a drug Apparent volume of distribution after oral dose (Vz/F) is influenced by the fraction absorbed. Multiple Ascending Dose First Dose: Volume of Distribution at Steady State (Vss) [Time Frame: 12 weeks]. Volume of distribution is defined as the theoretical volume in which the total amount of drug may be uniformly distributed to produce the predetermined blood concentration of a drug. Steady state volume of distribution (Vss) is the apparent volume of distribution at steady-state. Multiple Ascending Dose First Dose: Apparent Oral Clearance (CL/F) [Time Frame: 12 weeks]. Clearance of a drug is a measure of the rate at which a drug is metabolized or eliminated by normal biological processes. Clearance obtained after oral dose (apparent oral clearance) is influenced by the fraction of the dose absorbed. Clearance is estimated from population pharmacokinetic (PK) modeling. Drug clearance is a quantitative measure of the rate at which a drug substance is removed from the blood. Multiple Ascending Dose First Dose: Systemic Clearance (CL) [Time Frame: 12 weeks]. CL is a quantitative measure of the rate at which a drug substance is removed from the body.

Multiple Ascending Dose Multiple Dose: Maximum Observed Plasma Concentration (Cmax) [Time Frame: 12 weeks]. Multiple Ascending Dose Multiple Dose: Time to Reach Maximum Observed Plasma Concentration (Tmax) [Time Frame: 12 weeks]. Multiple Ascending Dose Multiple Dose: Area under the plasma concentration-time profile from time zero to time τ, the dosing interval where τ=2 weeks (AUCτ) [Time Frame: 12 weeks]. Multiple Ascending Dose Multiple Dose: Dose normalized maximum plasma concentration (Cmax[dn]) [Time Frame: 12 weeks]. Multiple Ascending Dose Multiple Dose: Dose normalized Area under the plasma concentration-time profile from time zero to time τ, the dosing interval where τ=2 weeks (AUCτ [dn]) [Time Frame: 12 weeks]. Multiple Ascending Dose Multiple Dose: Plasma Decay Half-Life (t½) [Time Frame: 12 weeks]. Plasma decay half-life is the time measured for the plasma concentration to decrease by one half. Multiple Ascending Dose Multiple Dose: Apparent Volume of Distribution (Vz/F) [Time Frame: 12 weeks]. Volume of distribution is defined as the theoretical volume in which the total amount of drug may be uniformly distributed to produce the predetermined plasma concentration of a drug. Apparent volume of distribution after oral dose (Vz/F) is influenced by the fraction absorbed. Multiple Ascending Dose Multiple Dose: Volume of Distribution at Steady State (Vss) [Time Frame: 12 weeks]. Volume of distribution is defined as the theoretical volume in which the total amount of drug may be uniformly distributed to produce the predetermined blood concentration of a drug. Steady state volume of distribution (Vss) is the apparent volume of distribution at steady-state.

Multiple Ascending Dose Multiple Dose: Apparent Oral Clearance (CL/F) [Time Frame: 12 weeks]. Clearance of a drug is a measure of the rate at which a drug is metabolized or eliminated by normal biological processes. Clearance obtained after oral dose (apparent oral clearance) is influenced by the fraction of the dose absorbed. Clearance was estimated from population pharmacokinetic (PK) modeling. Drug clearance is a quantitative measure of the rate at which a drug substance is removed from the blood. Multiple Ascending Dose Multiple Dose: Systemic Clearance (CL) [Time Frame: 12 weeks]. CL is a quantitative measure of the rate at which a drug substance is removed from the body. Multiple Ascending Dose Multiple Dose: Minimum Observed Plasma Trough Concentration (Cmin) [Time Frame: 12 weeks]. Multiple Ascending Dose Multiple Dose: Average concentration at steady state (Cav) [Time Frame: 12 weeks]. Multiple Ascending Dose Multiple Dose: Observed accumulation ratio (Rac) [Time Frame: 12 weeks]. Multiple Ascending Dose Multiple Dose: Peak to trough fluctuation (PTF) [Time Frame: 12 weeks]. Multiple Ascending Dose Additional Parameter: estimate of bioavailability (F) for subcutaneous administration at the corresponding intravenous dose [Time Frame: 12 weeks]. Immunogenicity for both Single Ascending Dose and Multiple Ascending Dose: Development of anti-drug antibodies (ADA) [Time Frame: 12 weeks].

### Example 6

### Phase 1B Clinical Trial

A phase 1b open label clinical trial is performed to evaluate efficacy of an anti-TL1A antibody on subjects having an inflammatory disease or condition, or fibrostenotic or fibrotic disease. Arms: 5 patients positive for two copies of the TNFSF15 polymorphism, and optionally, a polymorphism at the LY86, ETS1, or SCUBE1 gene loci, whose presence is associated with an increase in TL1A are administered the antibody. 5 patients positive for one copy of the TNFSF15 polymorphism, and optionally, a polymorphism the ARHGAP15 gene locus, whose presence is associated with an increase in TL1A are administered the antibody. 5-10 patients negative for the polymorphism are administered the antibody. Patients are monitored in real-time. Central ready of endoscopy and biopsy is employed, with readers blinded to point of time of treatment and endpoints.

Inclusion Criteria: Three groups of subjects are selected: subjects having two copies of the TNFSF15 polymorphism, and optionally, a polymorphism at the LY86, ETS1, or SCUBE1 gene loci, whose presence is associated with an increase in TL1A, subjects having one copy of the TNFSF15 polymorphism, and optionally, a polymorphism at the ARHGAP15 gene locus, whose presence is associated with an increase in TL1A, and subjects lacking the risk variant.

Primary Outcome Measures: Simple Endoscopic Score for Crohn's Disease (SESCD), Crohn's Disease Activity Index (CDAI), and Patient Reported Outcome (PRO). If risk either positive group shows 50% reduction from baseline, a Phase 2a clinical trial is performed.

Inclusion Criteria: PRO entry criteria: Abdominal pain score of 2 or more or stool frequency score of 4 or more. Primary outcome can be pain core of 0 or 1 and stool frequency score of 3 or less with no worsening from baseline. Endoscopy entry criteria: SESCD ileum entry at score of 4 and 6 if colon is involved. Primary endoscopic outcome is 40-50% delta of mean SESCD.

### Example 7

### Phase 2A Clinical Trial

A phase 2a clinical trial is performed to evaluate the efficacy of an anti-TL1A antibody in subjects having an inflammatory disease or condition, or fibrostenotic or fibrotic disease.

Arms: 40 patients per arm (antibody and placebo arms) are treated with antibody or placebo for 12 weeks. An interim analysis is performed after 20 patients from each group are treated at the highest dose to look for a 40-50% delta between placebo and treated group in primary outcome (50% reduction from baseline in SESCD, CDAI, and PRO).

Primary Outcome Measures: Simple Endoscopic Score for Crohn's Disease (SESCD), Crohn's Disease Activity Index (CDAI), and Patient Reported Outcome (PRO).

Inclusion Criteria: PRO entry criteria: Abdominal pain score of 2 or more or stool frequency score of 4 or more. Primary outcome can be pain core of 0 or 1 and stool frequency score of 3 or less with no worsening from baseline. Endoscopy entry criteria: SESCD ileum entry at score of 4 and 6 if colon is involved. Primary endoscopic outcome is 40-50% delta of mean SESCD.

### Example 8

### Treating an Inflammatory Disease or Condition or Fibrostenotic or Fibrotic Disease

An inflammatory disease or condition or fibrostenotic or fibrotic disease is treated in a subject, by first, determining the genotype of the subject. Optionally, the subject is, or is susceptible to, non-response to the induction of certain therapies such as anti-TNF, steroids, or immunomodulators, or loses response to such therapies after a period of time. A sample of whole blood is obtained from the subject. An assay is performed on the sample obtained from the subject to detect a presence of a monoallelic or a biallelic presence of a TNFSF15 risk genotype comprising a “G” at rs6478109, or a polymorphism in linkage disequilibrium therewith, and at least a monoallelic presence of one or more polymorphisms comprising: a “G” at rs6921610 (SEQ ID NO: 33), a “G” allele at rs10790957 (SEQ ID NO: 34), a “G” allele at rs6757588 (SEQ ID NO: 35), and a “G” allele at rs6003160 (SEQ ID NO: 36), by Illumina ImmunoArray or polymerase chain reaction (PCR) under standard hybridization conditions. Linkage disequilibrium may be determined using a D′l value of at least 0.8, or a D′l value of 0 and an r² value of at least 0.90. Nucleic acid probes suitable for the detection of the above polymorphisms comprise SEQ ID NOS: 37-72.

The subject is determined to have increased TL1A fold-change if (i) a monoallelic (heterozygous) TNFSF15 genotype is detected, and a “G” at rs6757588 (SEQ ID NO: 35) is detected; or (ii) a biallelic (homozygous) TNFSF15 genotype is detected, and at least one polymorphism from the “G” at rs6921610 (SEQ ID NO: 33), the “G” at rs10790957 (SEQ ID NO: 34), and the “G” at rs6003160 (SEQ ID NO: 36), is detected. A therapeutically effective amount of an inhibitor of TL1A activity or expression is administered to the subject, provided the subject is determined to have increased TL1A fold change. The inhibitor of TL1A activity or expression may comprise an anti-TL1A antibody.

### Example 9

An analysis was performed using “LAMPLINK” tool to determine if statistically significant SNP combinations exist between any of the four SNPs (rs6757588 in ARHGAP15 locus, rs6003160 in SCUBE1 locus, rs10790957 in ETS1 locus and rs6921610 in LY86 locus) that comprise the patient selection criteria based on TL1A fold change levels and the lead TNFSF15 SNP, rs6478109.

The aim was to determine if there are high-order, non-linear interactions between any of the four SNPs (identified via single-SNP associations) and the TNFSF15 lead SNP. We used the case-control phenotype for Crohn's disease versus non-IBD population (n_CD=2924, n_nonIBD=7272) for the associations. The associations were performed using a negative control SNP, rs10186474 (reading/writing SNP for immunochip) which is not associated with IBD in single-SNP associations and hence not expected to be part of top significant combinations with rs6478109.

Using dominant model, all of the four SNPs mentioned above were found to exist in significant combinations with rs6478109 (adjusted pvalue of combination <0.05). We found two combinations (COMB1 and COMB2, see Table 1) with significance (adjusted pvalue of combination) better than rs6478109 SNP alone. COMB1 consisted of ARHGAP15, LY86 and TNFSF15 SNP and COMB2 consisted of ARHGAP15 and TNFSF15 SNP. Although significant, the combinations with SCUBE1 or ETS1 SNPs with rs6478109 did not exceed that of rs6478109 alone. In conclusion using LAMPLINK tool, this examples shows that there exist non-linear, high-order interactions between the four SNPs identified by enrichment analysis and rs6478109 SNP.

TABLE 14
Significant combinations of ARHGAP15 and LY86 SNPs with rs6478109 that
reached significance better than that of rs6478109 alone.

| COMBID | Raw_P | Adjusted_P | OR | L95 | U95 | COMB |
|---|---|---|---|---|---|---|
| COMB1 | 7.99E−08 | 5.04E−06 | 0.738884 | 0.661407 | 0.825436 | rs6757588, rs6921610, imm_9_116608587 |
| COMB2 | 1.44E−07 | 9.05E−06 | 0.771321 | 0.7001 | 0.849787 | rs6757588, imm_9_116608587 |
| COMB3 | 6.16E−07 | 3.88E−05 | 0.803691 | 0.737483 | 0.875843 | imm_9_116608587 |

*Imm_9_116608587 = rs6478109.

While embodiments of the present methods have been shown and described herein, it will be obvious to those skilled in the art that such embodiments are provided by way of example. Numerous variations, changes, and substitutions will now occur to those skilled in the art without departing from the methods. It can be understood that various alternatives to the embodiments of the methods described herein may be employed in practicing the methods.

What is claimed:

1. A method of treating a subject with an inflammatory disease or condition, the method comprising: administering a therapeutically effective amount of an inhibitor of TL1A expression or activity to the subject that has been determined to have an increased fold-change in TL1A expression based on detecting, in a sample obtained from the subject, a combination of genotypes that is associated with the increased fold-change in TL1A expression with a P value of at most about 10⁻³, wherein the increased fold-change in TL1A expression is relative to a baseline expression of TL1A in a reference subject.

2. The method of claim 1, wherein the reference subject is a subject that (i) does not have the inflammatory disease or condition, or (ii) has the inflammatory disease or condition, but does not have the combination of genotypes.

3. The method of claim 1, wherein the increased fold-change in TL1A expression comprises an increase of greater than or equal to about 20 fold-change in TL1A expression relative to the baseline expression of TL1A in the reference subject.

4. The method of claim 1, wherein the increased fold-change in TL1A expression comprises an increase of greater than or equal to about 40 fold-change in TL1A expression relative to the baseline expression of TL1A in the reference subject.

5. The method of claim 1, wherein the increased fold-change in TL1A expression comprises an increase of greater than or equal to about 90 fold-change in TL1A expression relative to the baseline expression of TL1A in the reference subject.

6. The method of claim 1, wherein the combination of genotypes comprises homozygous “G” at rs6478109, or a polymorphism in LD therewith as determined by an r² of at least 0.80.

7. The method of claim 1, wherein the combination of genotypes comprises: (i) a homozygous genotype at a TNFSF15 gene locus; and (ii) a heterozygous or homozygous genotype at an ETS1 gene locus, a LY86 gene locus, or a SCUBE1 gene locus.

8. The method of claim 7, wherein the homozygous genotype at the TNFSF15 gene locus is at a polymorphism comprising rs6478109, or a polymorphism in LD therewith as determined by an r² of at least 0.80.

9. The method of claim 8, wherein the homozygous genotype at the TNFSF15 gene locus comprises a “G” at rs6478109, or the polymorphism in LD therewith as determined by an r² of at least 0.80.

10. The method of claim 7, wherein the heterozygous or homozygous genotype at the ETS1 gene locus is at a polymorphism comprising rs10790957, or a polymorphism in LD therewith as determined by an r² of at least 0.80.

11. The method of claim 10, wherein the genotype at the ETS1 gene locus comprises a “G” at rs10790957, or the polymorphism in LD therewith as determined by an r² of at least 0.80.

12. The method of claim 7, wherein the heterozygous or homozygous genotype at the LY86 gene locus is at a polymorphism comprising rs6921610, or a polymorphism in LD therewith as determined by an r² of at least 0.80.

13. The method of claim 12, wherein the genotype at the LY86 gene locus comprises a “G” at rs6921610, or the polymorphism in LD therewith as determined by an r² of at least 0.80.

14. The method of claim 7, wherein the heterozygous or homozygous genotype at the SCUBE1 gene locus is at a polymorphism comprising rs6003160, or a polymorphism in LD therewith as determined by an r² of at least 0.80.

15. The method of claim 14, wherein the genotype at the SCUBE1 gene locus comprises a “G” at rs6003160, or the polymorphism in LD therewith as determined by an r² of at least 0.80.

16. The method of claim 7, wherein (i) the heterozygous or homozygous genotype at the ETS1 gene locus is at a polymorphism comprising r510790957, or a polymorphism in LD therewith; (ii) the heterozygous or homozygous genotype at the LY86 gene locus is at a polymorphism comprising rs6921610, or a polymorphism in LD therewith; and (iii) the heterozygous or homozygous genotype at the SCUBE1 gene locus is at a polymorphism comprising rs6003160, or a polymorphism in LD therewith, wherein the LD is determined by an r² of at least 0.80.

17. The method of claim 16, wherein:
(a) the genotype at the ETS1 gene locus comprises a “G” at rs10790957 or the polymorphism in LD therewith as determined by an r² of at least 0.80;
(b) the genotype at the LY86 gene locus comprises a “G” at rs6921610 or the polymorphism in LD therewith as determined by an r² of at least 0.80; and
(c) the genotype at the SCUBE1 gene locus comprises a “G” at rs6003160 or the polymorphism in LD therewith as determined by an r² of at least 0.80.

18. The method of claim 1, wherein the combination of genotypes comprises: (i) a heterozygous genotype at a TNFSF15 gene locus; and (ii) a heterozygous or homozygous genotype at an ARHGAP15 gene locus.

19. The method of claim 18, wherein the heterozygous genotype at the TNFSF15 gene locus is at a polymorphism comprising rs6478109, or a polymorphism in LD therewith as determined by an r² of at least 0.80.

20. The method of claim 19, wherein the heterozygous genotype at the TNFSF15 gene locus comprises a “G” at rs6478109, or the polymorphism in LD therewith as determined by an r² of at least 0.80.

21. The method of claim 18, wherein the heterozygous or homozygous genotype at the ARHGAP15 gene locus is at a polymorphism comprising rs6757588, or a polymorphism in LD therewith as determined by an r² of at least 0.80.

22. The method of claim 21, wherein the heterozygous or homozygous genotype at the ARHGAP15 gene locus comprises a “G” at rs6757588, or the polymorphism in LD therewith as determined by an r² of at least 0.80.

23. The method of claim 18, wherein: (i) the heterozygous genotype at the TNFSF15 gene locus is at a polymorphism comprising rs6478109, or a polymorphism in LD therewith as determined by an r² of at least 0.80; and (ii) the heterozygous or homozygous genotype at the ARHGAP15 gene locus is at a polymorphism comprising rs6757588, or a polymorphism in LD therewith as determined by an r² of at least 0.80.

24. The method of claim 23, wherein:
(a) the heterozygous genotype at the TNFSF15 gene locus comprises a “G” at rs6478109, or the polymorphism in LD therewith as determined by an r² of at least 0.80; and
(b) the heterozygous or homozygous genotype at the ARHGAP15 gene locus comprises a “G” at rs6757588, or the polymorphism in LD therewith as determined by an r² of at least 0.80.

25. The method of claim 1, further comprising characterizing the inflammatory disease or condition as an inflammatory bowel disease.

26. The method of claim 25, wherein the inflammatory bowel disease comprises Crohn's disease.

27. The method of claim 25, wherein the inflammatory bowel disease comprises ulcerative colitis.

28. The method of claim 26, wherein the TL1A expression comprises TL1A protein expression.

29. The method of claim 1, wherein the increased fold-change in TL1A expression is determined by:
(a) introducing immune complex to peripheral blood mononuclear cells (PBMCs) in vitro under conditions suitable to stimulate the PBMCs, wherein the PBMCs were obtained from subjects with the inflammatory disease or condition;
(b) measuring by ELISA, the TL1A expression at a plurality of sequential time points comprising a first time point, a second time point and a third time point; and
(c) calculating the increased fold-change in TL1A expression by dividing the TL1A expression at the second time point and the TL1A expression at the third time point by the TL1A expression at the first time point.

30. The method of claim 29, wherein the first time point is 6 hours following the introducing in (a), the second time point is 24 hours following the introducing in (a), and the third time point is 72 hours following the introducing in (a).
